(19) United States
(12) Patent Application Publication
(10) Pub. No.: US 2022/0025111 A1
(43) Pub. Date: Jan. 27, 2022

(54) NARROW ABSORPTION POLYMER NANOPARTICLES AND RELATED METHODS

(71) Applicant: University of Washington, Seattle, WA (US)

(72) Inventors: Daniel T. Chiu, Seattle, WA (US); Lei Chen, Seattle, WA (US); Jiangbo Yu, Seattle, WA (US)

(73) Assignee: University of Washington, Seattle, WA (US)

(21) Appl. No.: 17/276,690

(22) PCT Filed: Sep. 16, 2019
(86) PCT No.: PCT/US19/51335

§ 371 Date: Mar. 16, 2021

Related U.S. Application Data
(60) Provisional application No. 62/733,009, filed on Sep. 18, 2018.

(51) Int. Cl. C08G 61/12; G01N 33/543; C08G 79/08; C08G 61/02; G01N 21/64

(52) U.S. Cl. CPC C08G 61/122; G01N 33/54346; C08G 79/08; B82Y 20/00; G01N 21/6486; C08G 2261/1434; C08G 2261/3244; C08G 61/02

(57) ABSTRACT

Polymers, monomers, narrow-band absorbing polymers, narrow-band absorbing monomers, absorbing units, polymer dots, and related methods are provided. Bright, luminescent polymer nanoparticles with narrow-band absorptions are provided. Methods for synthesizing absorbing monomers, methods for synthesizing the polymers, preparation methods for forming the polymer nanoparticles, and applications for using the polymer nanoparticles are also provided.

### CROSS-REFERENCE(S) TO RELATED APPLICATION(S)

This application claims the benefit of U.S. Patent Application No. 62/733,009, filed Sep. 18, 2018, the disclosure of which is hereby incorporated by reference in its entirety.

### STATEMENT OF GOVERNMENT LICENSE RIGHTS

This invention was made with US Government support under grant number RO1MH115767, awarded by the NIH. The US Government has certain rights in this invention.

### BACKGROUND

Fluorescence imaging is a non-invasive, real-time, high-resolution, and radioactive-free modality for visualizing systems for basic research and clinical applications. Polymer nanoparticles are a class of photon-emitting probes of interest. However, most polymer nanoparticles have broad absorption bands. Additionally, most polymer nanoparticles require a trade-off between quantum yield and absorption cross-section, which may reduce overall brightness. Polymer dots may have fluorescence self-quenching in its condensed state, and low absorption cross-section limits improvements in brightness.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure provides polymer nanoparticles having narrow-band absorption, methods of making polymer nanoparticles having narrow-band absorption, and methods of using polymer nanoparticles having narrow-band absorption.

In one aspect, the present disclosure features a nanoparticle including a polymer, the polymer including an absorbing monomeric unit and an emitting monomeric unit; wherein the nanoparticle has an absorbance width of less than 150 nm at 10% (or in some embodiments, at 15%) of the absorbance maximum. The nanoparticle can further include one or more monomeric units different from (a third or additional monomeric unit that is not identical to) the absorbing monomeric unit and the emitting monomeric unit. In some aspects, the absorbing monomeric unit includes BODIPY, a BODIPY derivative, or any combination thereof. In some embodiments, the absorbing monomeric unit includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof.

In another aspect, the present disclosure provides a nanoparticle including a polymer, the polymer including an absorbing monomeric unit, the absorbing monomeric unit can includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof, and an emitting monomeric unit. In some aspects, the nanoparticle has an absorbance width of less than 150 nm at 10% (or in some embodiments, at 15%) of the absorbance maximum.

In yet another aspect, the present disclosure features a nanoparticle including a polymer, the polymer including a first absorbing monomeric unit; an emitting monomeric unit; and one or more monomeric units different from the absorbing monomeric unit and the emitting monomeric unit. The nanoparticle can have an absorbance width of less than 150 nm at 10% (or in some embodiments, at 15%) of the absorbance maximum.

In some embodiments, the polymer has a backbone including the absorbing monomeric unit, has a side chain including the absorbing monomeric unit, has a terminus including the absorbing monomeric unit, or any combination thereof. The absorbing monomeric unit is covalently bound to the polymer.

In various embodiments, the present disclosure provides a nanoparticle including a first polymer including an absorbing monomeric unit, and a second polymer including an emitting monomeric unit, wherein the nanoparticle has an absorbance width of less than 150 nm at 15% of the absorbance maximum. In some embodiments, the absorbing monomeric unit includes BODIPY, a BODIPY derivative, or any combination thereof. In some embodiments, the absorbing monomeric unit includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof.

In various embodiments, the present disclosure provides a nanoparticle including a first polymer including an absorbing monomeric unit, the absorbing monomeric unit includes BODIPY, a BODIPY derivative, or any combination thereof. In some embodiments, the absorbing monomeric unit includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof, and a second polymer including an emitting monomeric unit. In some embodiments, the nanoparticle has an absorbance width of less than 150 nm at 10% (or in some embodiments, at 15%) of the absorbance maximum.

In some embodiments, the first polymer and the second polymer are the same polymer. In certain embodiments, the first polymer has a backbone including the absorbing monomeric unit, has a side chain including the absorbing monomeric unit, has a terminus including the absorbing monomeric unit, or any combination thereof. In some embodiments, the first polymer is a semiconducting polymer, the second polymer is a semiconducting polymer, or both the first and the second polymers are semiconducting polymers. In certain embodiments, the mass ratio of the first polymer to the second polymer is greater than 1:1, greater than 2:1, greater than 3:1, greater than 4:1, greater than 5:1, greater than 6:1, greater than 7:1, greater than 8:1, greater than 9:1, greater than 10:1, greater than 20:1, greater than 30:1, greater than 40:1, greater than 50:1, or greater than 100:1.

In certain embodiments, the nanoparticle further includes a matrix, which can include a matrix polymer. In some embodiments, the matrix polymer is a non-semiconducting polymer. In certain embodiments, the matrix polymer is a semiconducting polymer.

In some embodiments, the nanoparticle has a diameter, as measured by dynamic light scattering, of less than 1000 nm, less than 900 nm, less than 800 nm, less than 700 nm, less than 600 nm, less than 500 nm, less than 400 nm, less than 300 nm, less than 200 nm, less than 150 nm, less than 100 nm, less than 90 nm, less than 80 nm, less than 70 nm, less than 60 nm, less than 50 nm, less than 40 nm, less than 30 nm, less than 20 nm, or less than 10 nm as measured by dynamic light scattering. In certain embodiments, the nanoparticle has a quantum yield of greater than 5%, greater than 10%, greater than 15%, greater than 20%, greater than 25%, greater than 30%, greater than 35%, greater than 40%, greater than 45%, or greater than 50%.

In some embodiments, the absorbing monomeric unit is 30% or less, 25% or less, 20% or less, 15% or less, 14% or less, 13% or less, 12% or less, 11% or less, 10% or less, 9% or less, 8%, 7% or less, 6% or less, or 5% or less of the total mass of the nanoparticle. In certain embodiments, the absorbing monomeric unit is 30% or more, 25% or more, 20% or more, 15% or more, 14% or more, 13% or more, 12% or more, 11% or more, 10% or more, 9% or more, 8% or more, 7% or more, 6% or more, or 5% or more of the total mass of the nanoparticle.

In certain embodiments, nanoparticle includes a blend of polymers. In some embodiments, the ratio of the emitting monomeric unit to the absorbing monomeric unit is less than 1:2, less than 1:3, less than 1:4, less than 1:5, less than 1:6, less than 1:7, less than 1:8, less than 1:9, less than 1:10, less than 1:11, less than 1:12, less than 1:13, less than 1:14, less than 1:15, less than 1:16, less than 1:17, less than 1:18, less than 1:19, less than 1:20, less than 1:25, less than 1:30, less than 1:35, less than 1:40, less than 1:50, less than 1:60, less than 1:70, less than 1:80, less than 1:90, or less than 1:100.

In some embodiments, the nanoparticle has an absorbance width of less than 150 nm at 15% of the absorbance maximum, at 14% of the absorbance maximum, at 13% of the absorbance maximum, at 12% of the absorbance maximum, at 11% of the absorbance maximum, at 10% of the absorbance maximum, at 9% of the absorbance maximum, at 8% of the absorbance maximum, at 7% of the absorbance maximum, at 6% of the absorbance maximum, at 5% of the absorbance maximum, at 4% of the absorbance maximum, at 3% of the absorbance maximum, at 2% of the absorbance maximum, or at 1% of the absorbance maximum. In certain embodiments, the nanoparticle has an absorbance width of less than 150 nm, less than 140 nm, less than 130 nm, less than 120 nm, less than 110 nm, less than 100 nm, less than 90 nm, less than 80 nm, or less than 70 nm at 10% of the absorbance maximum. In some embodiments, the nanoparticle has an absorbance width from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 40 nm to 80 nm, from 30 nm to 70 nm, from 30 nm to 60 nm, or from 10 nm to 50 nm at 10% of the absorbance maximum.

In certain embodiments, the nanoparticle is bioconjugated to a biomolecule. In some embodiments, the biomolecule includes a protein, a nucleic acid molecule, a lipid, a peptide, a carbohydrate, or any combination thereof. In some embodiments, the biomolecule includes an aptamer, a drug, an antibody, an enzyme, a nucleic acid, or any combination thereof. In certain embodiments, the biomolecule includes streptavidin.

In some embodiments, the nanoparticle has a brightness of greater than 1.0×10⁻¹³ cm², the brightness calculated as the product of quantum yield and absorption cross-section.

In some embodiments, the nanoparticle does not include a β-phase structure. In certain embodiments, the nanoparticle does not include a fluorene monomeric unit.

In various embodiments, the present disclosure provides a method of making the nanoparticles of the present disclosure, including providing a solution including a polymer, the polymer including an absorbing monomeric unit, the absorbing monomeric unit includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof, and an emitting monomeric unit; and collapsing the polymer to form the nanoparticles. In some embodiments, the absorbing monomeric unit can include, for example, a BODIPY, a BODIPY derivative, or any combination thereof. In certain embodiments, the nanoparticles have an absorbance width of less than 150 nm at 10% (or in some embodiments, at 15%) of the absorbance maximum. In some embodiments, the polymer has a backbone including the absorbing monomeric unit, has a side chain including the absorbing monomeric unit, has a terminus including the absorbing monomeric unit, or any combination thereof.

In various embodiments, the present disclosure provides a method of making nanoparticles of the present disclosure, the method including: providing a solution including a first polymer, the first polymer including an absorbing monomeric unit, and a second polymer, the second polymer including an emitting monomeric unit; and collapsing the first polymer and the second polymer to form the nanoparticles. In some embodiments, the absorbing monomeric unit includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof. In some embodiments, the absorbing monomeric unit includes BODIPY, a BODIPY derivative, or any combination thereof. In certain embodiments, the first polymer has a backbone including the absorbing monomeric unit, has a side chain including the absorbing monomeric unit, has a terminus including the absorbing monomeric unit, or any combination thereof.

In certain embodiments, the collapsing step includes combining the solution and an aqueous liquid. In some embodiments, the nanoparticles are formed by nanoprecipitation.

In certain embodiments, the solution includes 15% or less, 14% or less, 13% or less, 12% or less, 11% or less, 10% or less, 9% or less, 8% or less, 7% or less, 6% or less, 5% or less, 4% or less, 3% or less, 2% or less, or 1% or less of the absorbing monomeric unit by weight. In some embodiments, the solution includes 15% or more, 14% or more, 13% or more, 12% or more, 11% or more, 10% or more, 9% or more, 8% or more, 7% or more, 6% or more, 5% or more, 4% or more, 3% or more, 2% or more, or 1% or more of the absorbing monomeric unit by weight.

In certain embodiments, the nanoparticles have a quantum yield of greater than 5%, greater than 10%, greater than 15%, greater than 20%, greater than 25%, greater than 30%, greater than 35%, greater than 40%, greater than 45%, or greater than 50%.

In various embodiments, the present disclosure provides a method of analyzing a biomolecule, the method includes optically detecting the presence or absence of the biomolecule, wherein the biomolecule is attached to the nanoparticle as described above, and wherein the detecting uses a detector.

In some embodiments, the method further includes imaging the biomolecule, wherein the detector includes an imaging device. In certain embodiments, the detector is selected from a camera, an electron multiplier, a charge-coupled device (CCD) image sensor, a photomultiplier tube (PMT), an avalanche photodiode (APD), a single-photon avalanche diode (SPAD), and a complementary metal oxide semiconductor (CMOS) image sensor. In certain embodiments, the detector includes a photo, electro, acoustical, or magnetic detector. In some embodiment, the detector incorporates fluorescence microscopy imaging.

In some embodiments, the method further includes performing an assay. In certain embodiments, the assay is a digital assay. In some embodiments, the assay includes fluorescence activated sorting. In certain embodiments, the assay includes flow cytometry. In some embodiments, the assay includes RNA extraction (with or without amplification), cDNA synthesis (reverse transcription), gene microarrays, DNA extraction, Polymerase Chain Reaction (PCR) (single, nested, quantitative real-time, or linker-adapter), isothermal nucleic acid amplification, DNA-methylation analysis, cell culturing, comparative genomic hybridization (CGH) studies, electrophoresis, Southern blot analysis, enzyme-linked immunosorbent assay (ELISA), digital nucleic acid assay, digital protein assay, assays to determine the microRNA and siRNA contents, assays to determine the DNA/RNA content, assays to determine lipid contents, assays to determine protein contents, assays to determine carbohydrate contents, functional cell assays, or any combination thereof.

In certain embodiments, the method further includes amplifying the biomolecule to produce an amplified product, the amplifying including performing polymerase chain reaction (PCR), isothermal nucleic acid amplification, rolling circle amplification (RCA), nucleic acid sequence based amplification (NASBA), loop-mediated amplification (LAMP), strand displacement amplification (SDA), or any combination thereof. In certain embodiments, a plurality of biomolecules is analyzed, and at least a portion of the plurality of biomolecules is attached to the nanoparticle as described above.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIGS. 1A-1L are non-limiting examples of schematic structures of narrow-band absorbing polymers.

FIG. 1A shows the structure of a homopolymer that includes only one narrow-band absorbing monomeric unit.

FIG. 1B shows the structure of a two-unit copolymer that includes one absorbing monomeric unit (e.g., a narrow-band absorbing monomeric unit) and one general monomeric unit.

FIG. 1C shows the structure of a three-unit copolymer that includes one absorbing monomeric unit and two general monomeric units such as general monomeric unit 1 (G1) and general monomeric unit 2 (G2).

FIG. 1D shows the structure of a two-unit copolymer that includes the absorbing unit cross-linked with the side-chains.

FIG. 1E shows the structure of a homopolymer that includes the absorbing unit cross-linked with the side-chains.

FIG. 1F shows a structure of a polymer that includes an absorbing unit attached to a terminus of the polymer.

FIG. 1G shows an example schematic structure of an absorbing polymer that include a general monomeric unit, an absorbing monomeric unit, and a functional monomeric unit (or a functional group).

FIG. 1H shows an example schematic structure of an absorbing polymer that include a general monomeric unit, an absorbing monomeric unit, and a functional monomeric unit (or a functional group).

FIG. 1I shows an example schematic structure of an absorbing polymer that include a general monomeric unit, an absorbing monomeric unit, and a functional monomeric unit (or a functional group).

FIG. 1J shows an example schematic structure of an absorbing polymer that includes a general monomeric unit, an absorbing monomeric unit, and a functional monomeric unit (or a functional group).

FIG. 1K shows an example schematic structure of an absorbing polymer that includes a general monomeric unit, an absorbing monomeric unit, and a functional monomeric unit (or a functional group).

FIG. 1L shows an example schematic structure of an absorbing polymer that includes a general monomeric unit, an absorbing monomeric unit, and a functional monomeric unit (or a functional group).

FIGS. 2A-2L show non-limiting examples of schematic structures of luminescence emitting polymers.

FIG. 2A shows the structure of a homopolymer that includes only one narrow-band emitting monomeric unit.

FIG. 2B shows the structure of a two-unit copolymer that includes one emitting monomeric unit and one general monomeric unit.

FIG. 2C shows the structure of a three-unit copolymer that includes one emitting monomeric unit and two general monomeric units such as general monomeric unit 1 (G1) and general monomeric unit 2 (G2).

FIG. 2D shows the structure of a two-unit copolymer that includes the emitting unit cross-linked with the side-chains.

FIG. 2E shows the structure of a homopolymer that includes the emitting unit cross-linked with the side-chains.

FIG. 2F shows a structure of a polymer that includes an emitting unit attached to a terminus of the polymer.

FIG. 2G shows an example schematic structure of an emitting polymer that includes a general monomeric unit, an emitting monomeric unit, and a functional monomeric unit (or a functional group).

FIG. 2H shows an example schematic structure of an emitting polymer that includes a general monomeric unit, an emitting monomeric unit, and a functional monomeric unit (or a functional group).

FIG. 2I shows an example schematic structure of an emitting polymer that includes a general monomeric unit, an emitting monomeric unit, and a functional monomeric unit (or a functional group).

FIG. 2J shows an example schematic structure of an emitting polymer that includes a general monomeric unit, an emitting monomeric unit, and a functional monomeric unit (or a functional group).

FIG. 2K shows an example schematic structure of an emitting polymer that includes a general monomeric unit, an emitting monomeric unit, and a functional monomeric unit (or a functional group).

FIG. 2L shows an example schematic structure of an emitting polymer that includes a general monomeric unit, an emitting monomeric unit, and a functional monomeric unit (or a functional group).

FIGS. 3A-3K show non-limiting examples of schematic structures of absorbing and emitting polymers.

FIG. 3A shows the structure of a two-unit copolymer that includes one absorbing monomeric unit (e.g., a narrow-band absorbing monomeric unit) and one emitting monomeric unit.

FIG. 3B shows the structure of a two-unit alternating copolymer that includes one absorbing monomeric unit and one emitting monomeric unit.

FIG. 3C shows the structure of a three-unit alternating copolymer.

FIG. 3D shows the structure of a two-unit alternating copolymer with a terminal emitting monomeric unit.

FIG. 3E shows the structure of a two-unit alternating copolymer with a terminal absorbing monomeric unit.

FIG. 3F shows the structure of a general homopolymer with a terminal emitting monomeric unit and a terminal absorbing monomeric unit.

FIG. 3G shows the structure of a three-unit copolymer.

FIG. 3H shows the structure of a four-unit alternating copolymer that includes an absorbing monomeric unit, an emitting monomeric unit, and two general monomeric units such as general monomeric unit 1 (G1) and general monomeric unit 2 (G2).

FIG. 3I shows the structure of a four-unit alternating copolymer that includes an absorbing monomeric unit, an emitting monomeric unit, and two general monomeric units such as general monomeric unit 1 (G1) and general monomeric unit 2 (G2).

FIG. 3J shows the structure of a three-unit copolymer that includes an absorbing unit cross-linked with the side-chains.

FIG. 3K shows the structure of a four-unit copolymer that includes a functionalized general monomeric unit (e.g., wherein F is a functional group, a functional monomeric unit, or a functional unit).

FIG. 3L shows the structure of a four-unit copolymer that includes an absorbing monomeric unit (A1) present in the polymer backbone and an absorbing unit (A2) cross-linked to the polymer. The absorbing monomeric unit and absorbing unit can both be energy-donors, the general monomeric units can be both energy-donors and energy-acceptors, and the emitting monomeric unit can be an energy-acceptor.

FIG. 3M shows the structure of a four-unit copolymer that includes a functionalized general monomeric unit (G1), a second general monomeric unit (G2) cross-linked with an absorbing unit (A2), an absorbing monomeric unit (A1), and an emitting monomeric unit (E).

FIG. 3N shows the structure of a five-unit copolymer that includes an absorbing monomeric unit (A1), a functionalized first general monomeric unit (G1) (e.g., wherein F is a functional monomeric unit, a functional group, and/or a functional unit), a second general monomeric unit (G2) cross-linked with an absorbing unit (A2), a third general monomeric unit (G3), and an emitting monomeric unit (E).

FIG. 4 shows non-limiting examples of the general monomeric units.

FIGS. 5A-5E show non-limiting examples of the chemical structures of general G1 type monomeric units and G2 type monomeric units used for synthesizing polymers, e.g., as in FIGS. 1-3.

FIG. 5A shows example G1 monomeric units.

FIG. 5B shows example G2 monomeric units and example derivatives of G2 monomeric units. For FIGS. 5B to 5E, the derivatives of G2 monomeric units are marked as G2′ monomeric units in the figures. The general G1 type monomeric units can, e.g., be copolymerized with the G2 type (or G2′ type) and the monomeric units to obtain a luminescent polymer. Any, e.g., of the G1 type monomeric units, G2 type, or G2′ type monomeric units can also be separately used to copolymerize with one absorbing monomeric unit to obtain the polymers as in FIGS. 1-3. Rather than copolymerization, an absorbing unit and/or an emitting unit can, e.g., be attached to the side chains or termini of a polymer formed from any of the G1 type monomeric units, G2 type, or G2′ type monomeric units.

FIG. 5C shows example G2 monomeric units and example derivatives of G2 monomeric units.

FIG. 5D shows example G2 monomeric units and example derivatives of G2 monomeric units.

FIG. 5E shows example G2 monomeric units and example derivatives of G2 monomeric units. The derivatives of G2 monomeric units are marked as G2′ monomeric units in the figures. The general G1 type monomeric units can, e.g., be copolymerized with the G2 type (or G2′ type) and the monomeric units to obtain a luminescent polymer. Any, e.g., of the G1 type monomeric units, G2 type, or G2′ type monomeric units can also be separately used to copolymerize with one absorbing monomeric unit to obtain the polymers as in FIGS. 1-3. Rather than copolymerization, an absorbing unit and/or an emitting unit can, e.g., be attached to the side chains or termini of a polymer formed from any of the G1 type monomeric units, G2 type, or G2′ type monomeric units.

FIGS. 6A-6Z and 6AA-6GG show non-limiting examples of different BODIPY derivatives, dyes (e.g., Atto, Alexa, rhodamine, cyanine, coumarin type dyes), DIBODIPY, pyrene, squaraine, and derivatives thereof in absorbing monomeric units. Each of the derivatives can be used to synthesize an absorbing homopolymer. Each of the derivatives can also be copolymerized with any of the general monomers and/or polymers to synthesize an absorbing copolymer. Each of the derivatives can be used as an absorbing unit to cross-link with the side-chains of conventional semiconducting polymers to form absorbing polymers.

FIG. 6A shows non-limiting examples of different BODIPY derivatives as absorbing monomeric units.

FIG. 6B shows non-limiting examples of different BODIPY derivatives as absorbing monomeric units.

FIG. 6C shows non-limiting examples of different BODIPY derivatives as absorbing monomeric units.

FIG. 6D shows non-limiting examples of different BODIPY derivatives as absorbing monomeric units.

FIG. 6E shows non-limiting examples of different BODIPY derivatives as absorbing monomeric units.

FIG. 6F shows non-limiting examples of different BODIPY derivatives as absorbing monomeric units.

FIG. 6G shows non-limiting examples of different BODIPY derivatives as absorbing monomeric units.

FIG. 6H shows non-limiting examples of different BODIPY derivatives as absorbing monomeric units.

FIG. 6I shows non-limiting examples of different BODIPY derivatives as absorbing monomeric units.

FIG. 6J shows non-limiting examples of different BODIPY derivatives as absorbing monomeric units.

FIG. 6K shows non-limiting examples of different BODIPY derivatives as absorbing monomeric units.

FIG. 6L shows non-limiting examples of different BODIPY derivatives as absorbing monomeric units.

FIG. 6M shows non-limiting examples of dye-functionalized monomers that can be used as absorbing monomeric units, as well as an exemplary synthesis of an absorbing dye monomeric unit-containing polymer. The dyes can include, for example, Atto dye structures, Alexa dye structures, rhodamine dye structures, or coumarin dye structures.

FIG. 6N shows non-limiting examples of cyanine-functionalized monomers that can be used as absorbing monomeric units, as well as an exemplary synthesis of an absorbing cyanine monomeric unit-containing polymer.

FIG. 6O shows non-limiting examples of cyanine-functionalized monomers that can be used as absorbing monomeric units, as well as an exemplary synthesis of an absorbing cyanine monomeric unit-containing polymer.

FIG. 6P shows non-limiting examples of DIBODIPY containing monomers that can be used as absorbing monomeric units, as well as an exemplary synthesis of an absorbing DIBODIPY monomeric unit-containing polymer.

FIG. 6Q shows non-limiting examples of DIBODIPY containing absorbing monomeric units, as well as an exemplary synthesis of an absorbing DIBODIPY monomeric unit-containing polymer.

FIG. 6R shows non-limiting examples of polymers containing DIBODIPY containing monomers that can be used as absorbing monomeric units and general monomeric units, as well as an exemplary synthesis of an absorbing DIBODIPY monomeric unit-containing polymer.

FIG. 6S shows non-limiting examples of polymers containing BODIPY containing absorbing monomeric units and general monomeric units.

FIG. 6T shows non-limiting examples of polymers containing BODIPY containing absorbing monomeric units and general monomeric units.

FIG. 6U shows non-limiting examples of polymers containing BODIPY containing absorbing monomeric units and general monomeric units.

FIG. 6V shows non-limiting examples of polymers containing BODIPY containing absorbing monomeric units and general monomeric units.

FIG. 6W shows non-limiting examples of polymers containing BODIPY containing absorbing monomeric units and general monomeric units.

FIG. 6X shows non-limiting examples of polymers containing BODIPY containing absorbing monomeric units and general monomeric units.

FIG. 6Y shows non-limiting examples of polymers containing BODIPY containing absorbing monomeric units and general monomeric units.

FIG. 6Z shows non-limiting examples of polymers containing BODIPY containing absorbing monomeric units and general monomeric units.

FIG. 6AA shows non-limiting examples of pyrene containing monomers that can be used as absorbing monomeric units, as well as an exemplary synthesis of an absorbing pyrene monomeric unit-containing polymer.

FIG. 6BB shows non-limiting examples of pyrene containing monomers that can be used as absorbing monomeric units, as well as an exemplary synthesis of an absorbing pyrene monomeric unit-containing polymer.

FIG. 6CC shows non-limiting examples of pyrene containing monomers that can be used as absorbing monomeric units, as well as an exemplary synthesis of an absorbing pyrene monomeric unit-containing polymer.

FIG. 6DD shows non-limiting examples of pyrene containing monomers that can be used as absorbing monomeric units, as well as an exemplary synthesis of an absorbing pyrene monomeric unit-containing polymer.

FIG. 6EE shows non-limiting examples of pyrene containing monomers that can be used as absorbing monomeric units, as well as an exemplary synthesis of an absorbing pyrene monomeric unit-containing polymer.

FIG. 6FF shows non-limiting examples of squaraine-containing monomers that can be used as absorbing monomeric units, as well as an exemplary synthesis of an absorbing squaraine monomeric unit-containing polymer.

FIG. 6GG shows non-limiting examples of pyrene containing monomers that can be used as absorbing monomeric units, as well as an exemplary synthesis of an absorbing pyrene monomeric unit-containing polymer.

FIG. 7A shows a non-limiting list of polymers including metal complexes and their derivatives. For FIGS. 7A-7C, different Pt complexes were used in the listed polymers as absorbing and/or emitting monomeric units, and other metal complexes can also be used. Each of the metal complexes can be copolymerized with any of the general polymers to synthesize an absorbing and/or emitting copolymer. Each of the metal complexes can be used as an absorbing and/or emitting unit to cross-link with the side-chains of conventional semiconducting polymers to form polymers.

FIG. 7B shows a non-limiting list of polymers including metal complexes and their derivatives.

FIG. 7C shows a non-limiting list of polymers including metal complexes and their derivatives.

FIG. 8 shows a non-limiting list of polymers including porphyrin, metalloporphyrin and their derivatives as monomeric units, as well as an exemplary synthesis of a polymer containing a porphyrin repeating unit. Each of the porphyrin derivatives can be copolymerized with any of the general polymers to synthesize an absorbing and/or emitting copolymer. Each of the porphyrin derivatives can be used as an absorbing and/or emitting unit to cross-link with the side-chains of conventional semiconducting polymers.

FIGS. 9A-9D show examples of how the maximum absorbance of a polymer or nanoparticle can be determined.

FIG. 9A shows an absorbance peak having a perfect baseline.

FIG. 9B shows an absorbance peak wherein a corrected baseline is used to calculate the maximum absorbance.

FIG. 9C shows two absorbance peaks, wherein the maximum absorbance is calculated from the main absorbance peak, and the absorption peaks are distinct from one another.

FIG. 9D shows two absorbance peaks, wherein the maximum absorbance is calculated from the main absorbance peak, and the absorption peaks are distinct from one another, as shown using a corrected baseline.

FIGS. 10A-10C show a multi-step synthesis of a series of monomers and the synthesis of narrow-band absorbing polymer P2.

FIG. 10A shows the synthesis of benzoxazolyl-based Monomer 1.

FIG. 10B shows the synthesis of BODIPY-based Monomer 2.

FIG. 10C shows the polymerization reaction to form polymer P2.

FIGS. 11A-11C show a multi-step synthesis of a monomers and the narrow-band absorbing polymer P7.

FIG. 11A shows the synthesis of BODIPY-based Monomer 5.

FIG. 11B shows the synthesis of fluorene-based Monomer 6.

FIG. 11C shows the polymerization reaction to form polymer P7.

FIG. 12 shows a schematic illustration of BODIPY based narrow absorbing polymer dots and Pdot-bioconjugates for specific cellular targeting.

FIG. 13 shows schematic illustration of a non-limiting example for forming Pdots using a general absorbing polymer and Eu complexes.

FIGS. 14A-14D show the photophysical properties of a polymer (Polymer P1).

FIG. 14A shows the absorbance of the polymer dissolved in THF.

FIG. 14B shows the emission of the polymer in THF.

FIG. 14C shows the absorbance of the polymer in its Pdot state.

FIG. 14D shows the emission of the polymer in its Pdot state.

FIGS. 15A-15D show the photophysical properties of a polymer (Polymer P2).

FIG. 15A shows the absorbance of the polymer dissolved in THF.

FIG. 15B shows the emission of the polymer in THF.

FIG. 15C shows the absorbance of the polymer in its Pdot state.

FIG. 15D shows the emission of the polymer in its Pdot state.

FIGS. 16A-16D show the photophysical properties of a polymer (Polymer P3).

FIG. 16A shows the absorbance of the polymer dissolved in THF.

FIG. 16B shows the emission of the polymer in THF.

FIG. 16C shows the absorbance of the polymer in its Pdot state.

FIG. 16D shows the emission of the polymer in its Pdot state.

FIGS. 17A-17D show the photophysical properties of a polymer (Polymer P4).

FIG. 17A shows the absorbance of the polymer dissolved in THF.

FIG. 17B shows the emission of the polymer in THF.

FIG. 17C shows the absorbance of the polymer in its Pdot state.

FIG. 17D shows the emission of the polymer in its Pdot state.

FIGS. 18A-18D show the photophysical properties of a polymer (Polymer P5).

FIG. 18A shows the absorbance of the polymer dissolved in THF.

FIG. 18B shows the emission of the polymer in THF.

FIG. 18C shows the absorbance of the polymer in its Pdot state.

FIG. 18D shows the emission of the polymer in its Pdot state.

FIG. 19A-19B shows the photophysical properties of a polymer (Polymer P6).

FIG. 19A shows the absorbance of the polymer dissolved in THF.

FIG. 19B shows the emission of the polymer in THF.

FIG. 19C shows the absorbance of the polymer in its Pdot state.

FIG. 19D shows the emission of the polymer in its Pdot state.

FIGS. 20A-20D show the photophysical properties of a polymer (Polymer P7).

FIG. 20A shows the absorbance of the polymer dissolved in THF.

FIG. 20B shows the emission of the polymer in THF.

FIG. 20C shows the absorbance of the polymer in its Pdot state.

FIG. 20D shows the emission of the polymer in its Pdot state.

FIGS. 21A-21B show the photophysical properties of polymer dots including 80 wt % polymer P8 and 20 wt % polymer P9.

FIG. 21A shows the absorbance of the polymer in its Pdot state.

FIG. 21B shows the emission of the polymer in its Pdot state.

FIG. 22 shows a comparison of PFGBDP Pdots, PFDHTBT-BDP720 Pdots, and Pdots including a blend of both PFGBDP and PFDHTBT-BDP720.

FIGS. 23A-23C show spectral properties of nanoparticles including polymer P8, polymer P9, and blended polymers.

FIG. 23A shows the absolute absorption (Abs; solid lines) and fluorescence (FL; dashed lines) of 0.005 g I⁻¹ PFGBDP Pdots, PFDHTBT-BDP720 Pdots, and blended Pdots.

FIG. 23B shows normalized absorption and photoluminescence spectra of PFGBDP and PFDHTBT Pdots, and BDP720 dyes in nanoparticle state.

FIG. 23C shows energy levels of GBDP monomer, GBDP H-dimer, PFDHTBT, and BDP720 in Pdot state, as well as the cascade energy transfer between them.

### DETAILED DESCRIPTION

It is desirable to achieve polymer dots (Pdot) with narrow-band absorption, but this can be difficult to achieve. It is beneficial to have narrow-band absorbing nanoparticles with high quantum yield, but this can be difficult because of fluorescence self-quenching of monomeric units or emitting units in the condensed polymer state of the polymer nanoparticle. When enhanced quantum yield or narrow-band absorption from nanoparticles has been achieved, it can come at the cost of lower absorption cross-section or brightness. The present disclosure presents an enhanced network of absorbing monomeric units and/or absorbing units, along with emitting monomeric units and/or emitting units, and/or general monomeric units that can improve energy transfer can aid to simultaneously improve quantum yield and brightness while achieving narrow-band absorption. In some embodiments, the general monomeric unit provides other functions, such as providing hydrophilic or amphiphilic properties, or reactive functional groups. For example, the general monomeric unit can include an energy transfer monomeric unit and/or can include a functional monomeric unit.

The brightness or narrow-band absorption of polymer nanoparticles relies, in part, on the structural aspects within the polymer nanoparticle. For example, a polymer dissolved in organic solution can have a high quantum yield, but the same polymer can have significantly decreased quantum yield following collapse into a nanoparticle state. It is therefore beneficial to introduce additional polymers or monomeric units to provide structural and/or energy-transferring support in the polymer nanoparticles.

Embodiments of the present application relate to a novel class of luminescent nanoparticles, referred to as narrow-band absorption polymer dots, and their biomolecular conjugates for a variety of applications, including but not limited to flow cytometry, fluorescence activated sorting, immunofluorescence, immunohistochemistry, fluorescence multiplexing, single molecule imaging, single particle tracking, protein folding, protein rotational dynamics, DNA and gene analysis, protein analysis, metabolite analysis, lipid analysis, FRET based sensors, high throughput screening, cell detection, bacteria detection, virus detection, biomarker detection, cellular imaging, in vivo imaging, bioorthogonal labeling, click reactions, fluorescence-based biological assays such as immunoassays and enzyme-based assays, and a variety of fluorescence techniques in biological assays and measurements.

While not limited to any particular theory or concept, the present disclosure is based at least in part on the fact that luminescent Pdots based on semiconducting polymers typically possess broad absorption spectra with absorbance peak width of greater than 200 nm at 10% (or in some embodiments, at 15%) of the absorbance maximum. Such broad-band absorption can be a significant drawback for fluorescence techniques in biology and fluorescence multiplexing. To overcome this challenge with the current Pdots, the present disclosure provides compositions and methods to obtain next-generation Pdots with narrow-band absorptions. Furthermore, the present disclosure provides compositions and methods that allow bioconjugation to polymer dots while also maintaining their narrow-band absorptions.

In some aspects, the properties of the narrow-band absorption polymers and polymer dots can be dependent on the polymer structures. Therefore, the polymer backbone (main chain), side chains, terminal units, and substituted groups can be varied to obtain specific properties. In some embodiments, the optical properties of the narrow-band polymer and polymer dots can be tuned by varying the structures of the polymer backbone (main chain). For example, the absorption and fluorescence emission can be red-shifted by increasing the conjugation length of the polymer backbone, or the absorption and fluorescence emission can be blue-shifted by decreasing the conjugation length of the polymer backbone. For example, the inclusion of benzothiadiazole (BT) or BT derivative monomeric unit can increase the photostability of certain types of resulting polymer dot compared with polymers that do not have BT or BT derivative in their polymer backbone.

In some embodiments, the optical properties of the narrow-band absorption polymer and polymer dots can be modified by varying the side chains, terminal units, and substituent groups. For example, the absorption band or fluorescence emission wavelength can be tuned by attaching chromophoric units to the side-chains and/or termini. The absorption bandwidth, absorption peak, emission bandwidth, fluorescence quantum yield, fluorescence lifetime, photostability, and other properties can also be modified by varying the polymer side-chain and/or terminal units in addition to the polymer backbone. In another example, the attachment and presence of anti-fade agents, such as derivatives of butylated hydroxytoluene, trolox, carotenoids, ascorbate, reduced glutathione, propyl gallate, propionic acid stearyl ester, hydroxyquinone, p-phenylenediamine, triphenylamine, beta mercaptoethanol, trans-stilbene, imidazole, Mowiol, or combinations thereof, or any other combinations of anti-fade agents known in the art, to the polymer via side chains, terminal units, backbone, and/or substituent groups, can increase quantum yield, photostability, or both. These anti-fade agents generally act as anti-oxidants to reduce oxygen, and/or act as scavengers of reactive oxygen species, and/or act to suppress photogenerated hole polarons within the polymer dot. In a preferred embodiment, the anti-fade agent is hydrophobic in nature so as not to adversely affect the packing and/or colloidal stability of the polymer dot. In some embodiments, the absorption peak, absorption bandwidth, emission peak, emission bandwidth, fluorescence quantum yield, fluorescence lifetime, photostability, and other properties of the narrow-band absorption polymer and polymer dots can also be modified by substituent groups on the polymers. For example, the degree of electron-donating or electron-withdrawing capability of the substituent groups can be used to tune the optical properties. For example, the two-photon absorption cross sections can be increased by modular structures such as donor-pi-donor or donor-acceptor-donor units.

In some embodiments, the colloidal properties of the polymer dots can be improved by varying the polymer backbone (main chain), side chains, terminal units, and substituent groups. In some embodiments, the polymer dots can include hydrophobic functional groups in the side-chains, terminal units, and/or substituent groups. In other embodiments, the polymer dots can include hydrophilic functional groups in the side-chains, terminal units, and/or substituent groups. The length, size, and nature of the hydrophobic/hydrophilic side chains can modify the chain-chain interactions, and control the packing of the polymers, and affect the colloidal stability and size of the polymer dots. The length, size, and nature of the hydrophobic/hydrophilic side chains can also affect the absorption bandwidth, absorption peak, emission peak, emission bandwidth, fluorescence quantum yield, fluorescence lifetime, photostability, and other properties of the narrow-band absorption polymer and polymer dots. For example, a large number of very hydrophilic functional groups can reduce the brightness of the polymer dots, and/or broaden the emission spectrum, and/or adversely affect their colloidal stability and non-specific binding properties.

### Definitions

As used herein, a “monomeric unit” refers to a group of atoms, derived from a molecule of a given monomer, that includes a constitutional unit of a polymer or a macromolecule.

As used herein, a monomer refers to a molecule which can undergo polymerization thereby contributing constitutional units to the essential structure of a macromolecule. As used herein, when a monomer forms part of a polymer chain, it is understood that the monomer refers to a monomeric unit.

As used herein, the term “constitutional unit” of a polymer refers to an atom or group of atoms in a polymer, including a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

As used herein, the term “repeat unit” corresponds to the smallest constitutional unit, the repetition of which constitutes a regular macromolecule (or oligomer molecule or a block).

As used herein, the term “end group” refers to a constitutional unit with only one attachment to a polymer chain, located at the end of a polymer. For example, the end group can be derived from a monomeric unit at the end of the polymer, once the monomer has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “biodegradable” refers to a process that degrades a material via hydrolysis and/or a catalytic degradation process, such as enzyme-mediated hydrolysis and/or oxidation. For example, polymer side chains can be cleaved from the polymer backbone via either hydrolysis or a catalytic process (e.g., enzyme-mediated hydrolysis and/or oxidation).

As used herein, “biocompatible” refers to a property of a molecule characterized by it, or its in vivo degradation products, being not, or at least minimally and/or reparably, injurious to living tissue; and/or not, or at least minimally and controllably, causing an immunological reaction in living tissue. As used herein, “physiologically acceptable” is interchangeable with biocompatible.

As used herein, the term “hydrophobic” refers to a moiety that is not attracted to water with significant apolar surface area. This phase separation can be observed via a combination of dynamic light scattering and aqueous NMR measurements. Hydrophobic constitutional units tend to be non-polar in aqueous conditions. Examples of hydrophobic moieties include alkyl groups, aryl groups, etc.

As used herein, the term “hydrophilic” refers to a moiety that is attracted to and tends to be dissolved by water. The hydrophilic moiety is miscible with an aqueous phase. Hydrophilic constitutional units can be polar and/or ionizable in aqueous conditions. Hydrophilic constitutional units can be ionizable under aqueous conditions and/or contain polar groups such as amines, hydroxyl groups, or ethylene glycol residues. Examples of hydrophilic moieties include carboxylic acid groups, amino groups, hydroxyl groups, etc.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “chromophoric polymer nanoparticle” or “chromophoric polymer dot” refers to a structure including one or more polymers (e.g., chromophoric polymers, semiconducting polymers) that have been formed into a stable sub-micron sized particle. The chromophoric polymer nanoparticles or chromophoric polymer dots of the present disclosure can, e.g., include a single polymer or a plurality of polymers that can be, e.g., chemically crosslinked and/or physically blended. “Polymer dot” and “Pdot” can be used interchangeably to represent “nanoparticle” or “polymer dot”. In certain embodiments, the polymer nanoparticle includes one or more chromophoric polymers (e.g., semiconducting polymers), and can be referred to as chromophoric polymer dots, chromophoric polymer nanoparticles, or chromophoric nanoparticles. The polymer dots provided herein may be formed by any method known in the art, including without limitation, methods relying on precipitation, methods relying on the formation of emulsions (e.g. mini or micro emulsion), and methods relying on condensation. Pdots described herein are different and distinct from nanoparticles formed from an aggregate of polyelectrolytes. Unless specified otherwise, a “polymer dot”, “Pdot”, or “nanoparticle”, refers herein to a narrow-band absorption polymer dot.

As used herein, “polymer” is a molecule composed of at least 2 repeating structural units typically connected by covalent chemical bonds. The repeating structural unit may be one type of monomeric unit, and the resulting polymer is a homopolymer. In some embodiments, the polymers can include two different types of monomeric units, or three different types of monomeric units, or more types of monomeric units, to result in a heteropolymer. One of ordinary skill in the art will appreciate that the different types of monomeric units can be distributed along a polymer chain in a variety of ways. For example, three different types of monomeric units can be randomly distributed along the polymer. It will similarly be appreciated that the distribution of monomeric units along the polymer can be represented in different ways. The number of repeating structural units (e.g., monomeric units) along the length of a polymer can be represented by “n.” In some embodiments, n can range, e.g., from at least 2, from at least 100, from at least 500, from at least 1000, from at least 5000, or from at least 10,000, or from at least 100,000, or higher. In certain embodiments, n can range from 2 to 10000, from 20 to 10000, from 20 to 500, from 50 to 300, from 100 to 1000, or from 500 to 10,000.

Polymers generally have extended molecular structures including backbones that optionally contain pendant side groups. The polymers provided herein can include, but are not limited to, linear polymers and branched polymers such as star polymers, comb polymers, brush polymers, ladders, and dendrimers. As described further herein, the polymers can include semiconducting polymers generally well known in the art.

As used herein, the term “chromophoric polymer” is a polymer in which at least a portion of the polymer includes chromophoric units. The term “chromophore” is given its ordinary meaning in the art. A chromophore absorbs certain wavelength of light from UV to near infrared region, and may be or may not be emissive. The chromophoric polymer can, e.g. be a “conjugated polymer”. The term “conjugated polymer” is recognized in the art. Electrons, holes, or electronic energy, can be conducted along the conjugated structure. In some embodiments, a large portion of the polymer backbone can be conjugated. In some embodiments, the entire polymer backbone can be conjugated. In some embodiments, the polymer can include conjugated structures in their side chains or termini. In some embodiments, the conjugated polymer can have conducting properties, e.g. the polymer can conduct electricity. In some embodiments, the conjugated polymer can have semiconducting properties and is referred to as a “semiconducting polymer,” e.g., the polymers can exhibit a direct band gap, leading to an efficient absorption or emission at the band edge.

A “chromophoric unit” in this disclosure includes, but is not limited to, a unit of structures with delocalized pi-electrons, a unit of small organic dye molecules, and/or a unit of metal complexes. Examples of chromophoric polymers can include polymers including units of structures with delocalized pi-electrons such as semiconducting polymers, polymers including units of small organic dye molecules, polymers including units of metal complexes, and polymers including units of any combinations thereof. The chromophoric unit can be incorporated into the polymer backbone. The chromophoric unit can also be covalently attached to the side chain, or the terminal unit of the polymer.

An “emission spectrum” of a polymer dot is defined as the spectrum of wavelengths (or frequencies) of electromagnetic radiation emitted by the polymer dot when it is excited to a higher energy state and then returned to a lower energy state. The width of the emission spectrum can be characterized by its full width at half maximum (FWHM). The FWHM of an emission spectrum is defined as the distance between points on the emission curve at which the emission intensity reaches half its maximum value. The emission properties of a polymer dot can also be characterized by fluorescence quantum yield and fluorescence lifetime. The fluorescence quantum yield gives the efficiency of the fluorescence process. It is defined as the ratio of the number of photons emitted to the number of photons absorbed by the Pdots. The fluorescence lifetime is defined as the average time the polymer dot stays in its excited state before emitting a photon. All the above defined parameters, such as emission spectrum, FWHM, fluorescence quantum yield, and fluorescence lifetime can be experimentally measured. In this disclosure, these parameters can be specifically used to characterize the narrow-band emissive Pdots.

An “absorption spectrum” of a polymer dot is defined as the spectrum of wavelengths (or frequencies) of electromagnetic radiation absorbed by the polymer dot which excite it to a higher energy state before it is returned to a lower energy state. In certain embodiments, the energy state corresponding to the absorption spectrum is an electronic transition.

As used herein, the term “alkyl” refers to a straight or branched, saturated, aliphatic radical having the number of carbon atoms indicated. For example, C₁-C₆ alkyl includes, but is not limited to, methyl, ethyl, propyl, isopropyl, butyl, isobutyl, sec-butyl, tert-butyl, pentyl, isopentyl, hexyl, etc. Other alkyl groups include, but are not limited to heptyl, octyl, nonyl, decyl, etc. Alkyl can include any number of carbons, such as 1-2, 1-3, 1-4, 1-5, 1-6, 1-7, 1-8, 1-9, 1-10, 2-3, 2-4, 2-5, 2-6, 3-4, 3-5, 3-6, 4-5, 4-6 and 5-6. Alkyl can include, as a non-limiting example, 100-1, 50-40, 50-30, 50-20, 50-10, 50-1, 40-30, 40-20, 40-10, 40-1, 30-25, 30-20, 30-15, 30-10, 30-5, 30-1, 25-20, 25-15, 25-10, 25-5, 25-1, 20-15, 20-10, 20-5, 20-1, 15-10, 15-5, 15-1, 10-5, 1-2, 1-3, 1-4, 1-5, 1-6, 1-7, 1-8, 1-9, 1-10, 2-3, 2-4, 2-5, 2-6, 3-4, 3-5, 3-6, 4-5, 4-6, or 5-6 carbon atoms. The alkyl group is typically monovalent, but can be divalent, such as when the alkyl group links two moieties together. As used herein, the term “heteroalkyl” refers to a straight or branched, saturated, aliphatic radical of carbon atoms, where at least one of the carbon atoms is replaced with a heteroatom, such as N, O, or S. Additional heteroatoms can also be useful, including, but not limited to, B, Al, Si, and P. The alkyl group can be halogenated, wherein at least one of the carbon atoms is attached covalently to a halogen, such as F, Cl, Br, or I.

The term “lower” referred to above and hereinafter in connection with organic radicals or compounds respectively defines a compound or radical which can be branched or unbranched with up to and including 7, preferably up to and including 4 and (as unbranched) one or two carbon atoms.

As used herein, the term “alkylene” refers to an alkyl group, as defined above, linking at least two other groups, i.e., a divalent hydrocarbon radical. The two moieties linked to the alkylene can be linked to the same atom or different atoms of the alkylene. For instance, a straight chain alkylene can be the bivalent radical of —(CH₂), where n is 1, 2, 3, 4, 5 or 6. Alkylene groups include, but are not limited to, methylene, ethylene, propylene, isopropylene, butylene, isobutylene, sec-butylene, pentylene and hexylene.

The groups described herein can be substituted or unsubstituted. Substituents for the alkyl and heteroalkyl radicals (including those groups often referred to as alkylene, alkenyl, heteroalkylene, heteroalkenyl, alkynyl, cycloalkyl, heterocycloalkyl, cycloalkylene, heterocycloalkylene, cycloalkenyl, and heterocycloalkenyl) can be a variety of groups, such as alkyl, aryl, cyano (CN), amino, sulfide, aldehyde, ester, ether, acid, hydroxyl or halide. Substituents can be a reactive group, such as but not limited to fluoro, chloro, bromo, iodo, hydroxyl, or amino. Suitable substituents can be selected from, for example: —OR′, ═O, ═NR′, ═N—OR′, —NR′R″, —SR′, -halogen, —SiR′R″R′″, —OC(O)R′, —C(O)R′, —CO₂R′, —CONR′ R″, —OC(O)NR′R″, —NR″C(O)R′, —NR′—C(O)NR″R′″, —NR″C(O)₂R′, —NH—C(NH₂)═NH, —N R′C(NH₂)═NH, —NH—C(NH₂)═NR′, —S(O)R′, —S(O)₂R′, —S(O)₂NR′R″, —CN and —NO₂ in a number ranging from zero to (2m′+1), where m′ is the total number of carbon atoms in such radical. R′, R″ and R′″ each independently refer to hydrogen, unsubstituted (C₁-C₅) alkyl and heteroalkyl, unsubstituted aryl, alkoxy or thioalkoxy groups, or aryl-(C₁-C₄)alkyl groups. When R′ and R″ are attached to the same nitrogen atom, they can be combined with the nitrogen atom to form a 5-, 6-, or 7-membered ring. For example, —NR′R″ is meant to include 1-pyrrolidinyl and 4-morpholinyl. From the above discussion of substituents, one of skill in the art will understand that the term “alkyl” is meant to include groups such as haloalkyl (e.g., —CF₃ and —CH₂CF₃) and acyl (e.g., —C(O)CH₃, —C(O)CF₃, —C(O)CH₂OCH₃, and the like).

As used herein, the term “alkoxy” refers to an alkyl group having an oxygen atom that either connects the alkoxy group to the point of attachment or is linked to two carbons of the alkoxy group. Alkoxy groups include, for example, methoxy, ethoxy, propoxy, iso-propoxy, butoxy, 2-butoxy, iso-butoxy, sec-butoxy, tert-butoxy, pentoxy, hexoxy, ether, polyether (e.g., polyethylene glycol (PEG)), etc. The alkoxy groups can be further substituted with a variety of substituents described within. For example, the alkoxy groups can be substituted with halogens to form a “halo-alkoxy” group. Alkoxy can include, as a non-limiting example, 100-1, 50-40, 50-30, 50-20, 50-10, 50-1, 40-30, 40-20, 40-10, 40-1, 30-25, 30-20, 30-15, 30-10, 30-5, 30-1, 25-20, 25-15, 25-10, 25-5, 25-1, 20-15, 20-10, 20-5, 20-1, 15-10, 15-5, 15-1, 10-5, 1-2, 1-3, 1-4, 1-5, 1-6, 1-7, 1-8, 1-9, 1-10, 2-3, 2-4, 2-5, 2-6, 3-4, 3-5, 3-6, 4-5, 4-6, or 5-6 carbon atoms.

As used herein, the term “alkenyl” refers to either a straight chain or branched hydrocarbon of 2 to 6 carbon atoms, having at least one double bond. Examples of alkenyl groups include, but are not limited to, vinyl, propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

As used herein, the term “alkenylene” refers to an alkenyl group, as defined above, linking at least two other groups, i.e., a divalent hydrocarbon radical. The two moieties linked to the alkenylene can be linked to the same atom or different atoms of the alkenylene. Alkenylene groups include, but are not limited to, ethenylene, propenylene, isopropenylene, butenylene, isobutenylene, sec-butenylene, pentenylene and hexenylene.

As used herein, the term “alkynyl” refers to either a straight chain or branched hydrocarbon of 2 to 6 carbon atoms, having at least one triple bond. Examples of alkynyl groups include, but are not limited to, acetylenyl, propynyl, 1-butynyl, 2-butynyl, isobutynyl, sec-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl.

As used herein, the term “alkynylene” refers to an alkynyl group, as defined above, linking at least two other groups, i.e., a divalent hydrocarbon radical. The two moieties linked to the alkynylene can be linked to the same atom or different atoms of the alkynylene. Alkynylene groups include, but are not limited to, ethynylene, propynylene, isopropynylene, butynylene, sec-butynylene, pentynylene and hexynylene.

As used herein, the term “alkyl amine” refers to an alkyl groups as defined within, having one or more amino groups. The amino groups can be primary, secondary or tertiary. The alkyl amine can be further substituted with a hydroxy group. Alkyl amines can include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group.

As used herein, the term “halogen” or “halide” refers to fluorine, chlorine, bromine and iodine. As used herein, the term “haloalkyl” refers to alkyl as defined above where some or all of the hydrogen atoms are substituted with halogen atoms. Halogen (halo) preferably represents chloro or fluoro, but can also be bromo or iodo. As used herein, the term “halo-alkoxy” refers to an alkoxy group having at least one halogen. Halo-alkoxy is as defined for alkoxy where some or all of the hydrogen atoms are substituted with halogen atoms. The alkoxy groups can be substituted with 1, 2, 3, or more halogens. When all the hydrogens are replaced with a halogen, for example by fluorine, the compounds are per-substituted, for example, perfluorinated. Halo-alkoxy includes, but is not limited to, trifluoromethoxy, 2,2,2-trifluoroethoxy, perfluoroethoxy, etc.

As used herein, the term “cycloalkyl” refers to a saturated or partially unsaturated, monocyclic, fused bicyclic or bridged polycyclic ring assembly containing from 3 to 12 ring atoms, or the number of atoms indicated. Monocyclic rings include, for example, cyclopropyl, cyclobutyl, cyclopentyl, cyclohexyl, and cyclooctyl. Bicyclic and polycyclic rings include, for example, norbornane, decahydronaphthalene and adamantane. For example, C₃₋₈ cycloalkyl includes cyclopropyl, cyclobutyl, cyclopentyl, cyclohexyl, cyclooctyl, and norbornane.

As used herein, the term “cycloalkylene” refers to a cycloalkyl group, as defined above, linking at least two other groups, i.e., a divalent hydrocarbon radical. The two moieties linked to the cycloalkylene can be linked to the same atom or different atoms of the cycloalkylene. Cycloalkylene groups include, but are not limited to, cyclopropylene, cyclobutylene, cyclopentylene, cyclohexylene, and cyclooctylene.

As used herein, the term “heterocycloalkyl” refers to a ring system having from 3 ring members to about 20 ring members and from 1 to about 5 heteroatoms such as N, O and S. Additional heteroatoms can also be useful, including, but not limited to B, Al, Si and P. The heteroatoms can also be oxidized, such as, but not limited to, —S(O)— and —S(O)₂—.

As used herein, the term “heterocycloalkylene” refers to a heterocycloalkyl group, as defined above, linking at least two other groups. The two moieties linked to the heterocycloalkylene can be linked to the same atom or different atoms of the heterocycloalkylene.

As used herein, the term “aryl” refers to a monocyclic or fused bicyclic, tricyclic or greater, aromatic ring assembly containing 6 to 16 ring carbon atoms. For example, aryl can be phenyl, benzyl, azulenyl, or naphthyl. “Arylene” means a divalent radical derived from an aryl group. Aryl groups can be mono-, di- or tri-substituted by one, two, or three radicals selected from alkyl, alkoxy, aryl, hydroxy, halogen, cyano, amino, amino-alkyl, trifluoromethyl, alkylenedioxy, and oxy-C₂-C₃-alkylene; all of which are optionally further substituted, for instance as hereinbefore defined; or 1- or 2-naphthyl; or 1- or 2-phenanthrenyl. Alkylenedioxy is a divalent substitute attached to two adjacent carbon atoms of phenyl, e.g., methylenedioxy or ethylenedioxy. Oxy-C₂-C₃-alkylene is also a divalent substituent attached to two adjacent carbon atoms of phenyl, e.g., oxyethylene or oxypropylene. An example for oxy-C₂-C₃-alkylene-phenyl is 2,3-dihydrobenzofuran-5-yl.

Aryl groups can include, but are not limited to, naphthyl, phenyl or phenyl mono- or disubstituted by alkoxy, phenyl, halogen, alkyl or trifluoromethyl, phenyl or phenyl-mono- or disubstituted by alkoxy, halogen or trifluoromethyl, and in particular phenyl.

As used herein, the term “arylene” refers to an aryl group, as defined above, linking at least two other groups. The two moieties linked to the arylene are linked to different atoms of the arylene. Arylene groups include, but are not limited to, phenylene.

As used herein, the terms “alkoxy-aryl” or “aryloxy” refers to an aryl group, as defined above, where one of the moieties linked to the aryl is linked through an oxygen atom. Alkoxy-aryl groups include, but are not limited to, phenoxy (C₆HsO—). The present disclosure also includes alkoxy-heteroaryl or heteroaryloxy groups.

As used herein, the term “heteroaryl” refers to a monocyclic or fused bicyclic or tricyclic aromatic ring assembly containing 5 to 16 ring atoms, where from 1 to 4 of the ring atoms are a heteroatom each N, O or S. For example, heteroaryl includes pyridyl, indolyl, indazolyl, quinoxalinyl, quinolinyl, isoquinolinyl, benzothienyl, benzofuranyl, furanyl, pyrrolyl, thiazolyl, benzothiazolyl, oxazolyl, isoxazolyl, triazolyl, tetrazolyl, pyrazolyl, imidazolyl, thienyl, or any other radicals substituted, especially mono- or di-substituted, by e.g., alkyl, nitro or halogen. Suitable groups for the present disclosure can also include heteroarylene and heteroarylene-oxy groups similar to the description above for arylene and arylene-oxy groups.

Similarly, aryl and heteroaryl groups described herein can be substituted or unsubstituted. Substituents for the aryl and heteroaryl groups are varied, such as alkyl, aryl, CN, amino, sulfide, aldehyde, ester, ether, acid, hydroxyl or halide. Substituents can be a reactive group, such as but not limited to chloro, bromo, iodo, hydroxyl, or amino.

Substituents can be selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —N R′C(NH₂)═NH, —NH—C(NH₂)═NR′, —S(O)R′, —S(O)₂R′, —S(O)₂NR′R″, —N₃, —CH(Ph)₂, in a number ranging from zero to the total number of open valences on the aromatic ring system; and where R′, R″ and R′″ are independently selected from hydrogen, (C₁-C₅) alkyl and heteroalkyl, unsubstituted aryl and heteroaryl, (unsubstituted aryl)-(C₁-C₄)alkyl, and (unsubstituted aryl)oxy-(C₁-C₄)alkyl.

As used herein, the term “alkyl-aryl” refers to a radical having an alkyl component and an aryl component, where the alkyl component links the aryl component to the point of attachment. The alkyl component is as defined above, except that the alkyl component is at least divalent in order to link to the aryl component and to the point of attachment. In some instances, the alkyl component can be absent. The aryl component is as defined above. Examples of alkyl-aryl groups include, but are not limited to, benzyl. The present disclosure also includes alkyl-heteroaryl groups.

As used herein, the term “alkenyl-aryl” refers to a radical having both an alkenyl component and an aryl component, where the alkenyl component links the aryl component to the point of attachment. The alkenyl component is as defined above, except that the alkenyl component is at least divalent in order to link to the aryl component and to the point of attachment. The aryl component is as defined above. Examples of alkenyl-aryl include ethenyl-phenyl, among others. The present disclosure also includes alkenyl-heteroaryl groups.

As used herein, the term “alkynyl-aryl” refers to a radical having both an alkynyl component and an aryl component, where the alkynyl component links the aryl component to the point of attachment. The alkynyl component is as defined above, except that the alkynyl component is at least divalent in order to link to the aryl component and to the point of attachment. The aryl component is as defined above. Examples of alkynyl-aryl include ethynyl-phenyl, among others. The present disclosure also includes alkynyl-heteroaryl groups.

As will be appreciated by one of ordinary skill in the art, the various chemical terms defined herein can be used for describing chemical structures of the polymers and monomeric units of the present disclosure. For example, a variety of the monomeric unit derivatives (e.g., a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof) can include a variety of the chemical substituents and groups described herein. For example, in some embodiments, derivatives of the various monomeric units can be substituted with hydrogen, deuterium, alkyl, aralkyl, aryl, alkoxy-aryl, N-dialkyl-4-phenyl, N-diphenyl-4-phenyl, N-dialkoxyphenyl-4-phenyl, amino, sulfide, aldehyde, ester, ether, acid, and/or hydroxyl.

The compounds described herein can be asymmetric (e.g., having one or more stereocenters). All stereoisomers, such as enantiomers and diastereomers, are intended unless otherwise indicated.

Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and can be isolated as a mixture of isomers or as separated isomeric forms.

Compounds of the disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the disclosure can also include all isotopes of atoms occurring in the intermediates or final compounds. Isotopes include those atoms having the same atomic number but different mass numbers. For example, isotopes of hydrogen include tritium and deuterium.

In some embodiments, the compounds of the disclosure, and salts thereof, are substantially isolated. By “substantially isolated” is meant that the compound is at least partially or substantially separated from the environment in which it was formed or detected. Partial separation can include, for example, a composition enriched in the compound of the disclosure. Substantial separation can include compositions containing at least about 50%, at least about 60%, at least about 70%, at least about 80%, at least about 90%, at least about 95%, at least about 97%, or at least about 99% by weight of the compound of the disclosure, or salt thereof. Methods for isolating compounds and their salts are routine in the art.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

It will be readily understood that the aspects of the present disclosure, as generally described herein, and illustrated in the FIGURES, can be arranged, substituted, combined, separated, and designed in a wide variety of different configurations, all of which are explicitly contemplated herein.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES. As used herein, with respect to measurements, “about” means +/−5%. As used herein, a recited range includes the end points, such that from 0.5 mole percent to 99.5 mole percent includes both 0.5 mole percent and 99.5 mole percent.

### Absorption and Emission of Narrow-Band Absorption Nanoparticles

The present disclosure provides, in at least one embodiment, polymer dots with at least one narrow-band absorption (also referred to herein as “narrow absorption bandwidth” and “narrow-band absorbance”). A narrow-band absorption can have, for example, an absorbance width of less than 150 nm at 10% (or in some embodiments, at 15%) of the absorbance maximum.

The present disclosure provides, in some embodiments, polymer dots including a polymer, the polymer including an absorbing monomeric unit and an emitting monomeric unit. An “absorbing monomeric unit” is a unit that absorbs electromagnetic radiation, which can change the state of the monomeric unit, the polymer, and/or the polymer dots. In some embodiments, the absorbing monomeric unit, the polymer, and/or the polymer dots have an absorption band, which is the range of wavelengths, frequencies, or energies from the electromagnetic radiation spectrum that are absorbed (i.e., the “absorption spectrum”).

In some embodiments, energy absorbed by an absorbing monomeric unit is transferred to an emitting monomeric unit. The polymer can include an absorbing monomeric unit, an emitting monomeric unit, and an energy transfer monomeric unit. For example, the energy absorbed by an absorbing monomeric unit can be transferred to an energy transfer monomeric unit and then from the energy transfer monomeric unit to an emitting monomeric unit. The energy can be transferred from absorbing monomeric unit to emitting monomeric unit, or first to the energy transfer monomeric unit and then to an emitting monomeric unit, via intermolecular or intramolecular energy transfer. Non-limiting examples of intermolecular and intramolecular energy transfer include, e.g., through-chain energy transfer, through-bond energy transfer, Forster resonance energy transfer (FRET), Dexter energy transfer, cascade energy transfer, and fluorescence energy transfer. The transferred energy can excite an emitting monomeric unit from its ground (initial) state to an excited state. An “emitting monomeric unit” is a unit that emits electromagnetic radiation, the emission of which returns the monomeric unit from an excited state to a ground state. In some embodiments, the emitting monomeric unit, the polymer, and/or the polymer dots have an emission band, which is the range of wavelengths, frequencies, or energies from the electromagnetic radiation spectrum that are emitted (i.e., the “emission spectrum”). In some embodiments, the emission spectrum can vary from ultraviolet to the infrared region. As used here, an “energy transfer monomeric unit” is a monomeric unit that is different from (e.g., a third or additional monomeric unit in the polymer that transfers energy and that is not identical to) the absorbing monomeric unit and the emitting monomeric unit, that can transfer energy via intra-chain or inter-chain mechanisms to emitting monomeric units. For example, the energy transfer can occur via FRET (Forster resonance energy transfer), inter-chain energy transfer, through-bond energy transfer.

In some embodiments, an absorbing unit includes an absorbing monomeric unit. In certain embodiments, an absorbing unit includes a narrow-band absorbing monomeric unit. An absorbing unit including a narrow-band absorbing monomeric unit can be referred to as a narrow-band absorbing unit.

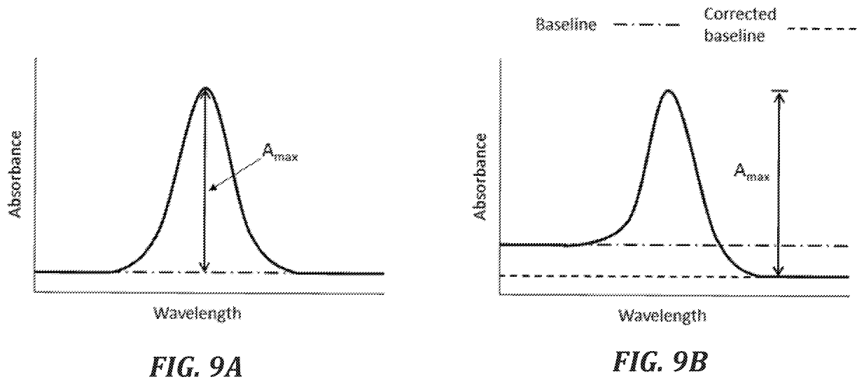

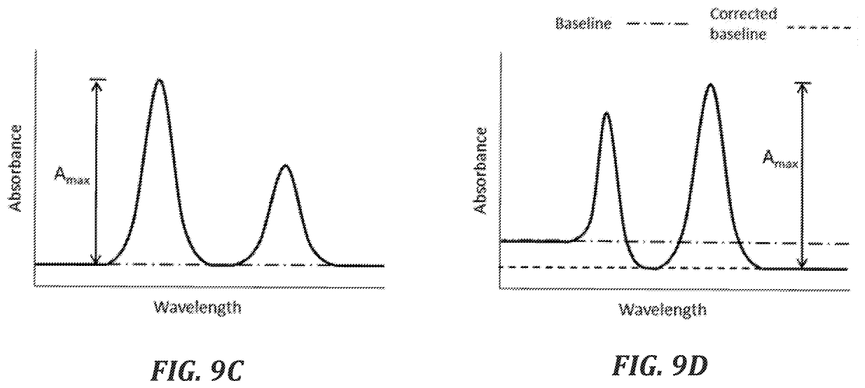

The polymers of the present disclosure have a narrow absorption spectrum. In some embodiments, the width of the absorption spectrum (also referred to herein as the “absorbance width”) can be characterized by its full width at a percentage of its maximum (e.g., full width at 15% of the absorbance maximum, or full width at 10% of the absorbance maximum). The absorbance maximum of an absorption spectrum is defined as the maximum height the absorbance intensity reaches over a baseline of the absorption peak. In certain embodiments, the true baseline is used, and the maximum absorbance is calculated as the difference in intensity from the main peak of the absorbance curve and the baseline (FIG. 9A). The maximum absorbance can be represented as Aₘₐₓ. In some embodiments, the absorbance curve is a perfect Gaussian curve. In other embodiments, the absorbance curve is not a perfect Gaussian curve, and can have a starting intensity value that is different from the ending intensity value (i.e., the intensity at the start of the absorbance curve may be higher than the intensity at the end of the absorbance curve) (FIG. 9B). In some embodiments a corrected baseline is used, and the maximum absorbance is calculated as the difference in intensity from the peak of the absorbance curve and the corrected baseline (FIG. 9B). The corrected baseline can be set as the lowest value of intensity of the absorbance curve, as shown in FIG. 9B. In specific embodiments, the corrected baseline value can be set as the lowest value of intensity of the absorbance curve within the region from 350 nm to 1000 nm. The maximum absorbance peak can be within the wavelength region from ultraviolet to infrared. In certain embodiments, the maximum absorbance peak is within the region from 380 nm to 1200 nm. In specific embodiments, the maximum absorbance peak is within the region from 380 nm to 1200 nm, from 400 nm to 1100 nm, from 500 nm to 1000 nm, from 600 nm to 900 nm, from 380 nm to 1100 nm, from 380 nm to 1000 nm, from 380 nm to 950 nm, from 380 nm to 900 nm, from 380 nm to 850 nm, from 380 nm to 800 nm, from 380 nm to 750 nm, from 380 nm to 700 nm, or from 400 to 700 nm.

As a non-limiting example, a sample having a perfect Gaussian curve may have a maximum absorbance of 1.00 AU, and a baseline value that is consistently 0 AU. The full width at 15% of the maximum absorbance would be the width of the curve at 0.15 AU (i.e., at 15% of the maximum value). Similarly, the full width at 10% of the maximum absorbance would be the width at 0.10 AU. The full width at 17% of the maximum absorbance would be the width at 0.17 AU. Accordingly, the full width at various percentages of the absorbance maximum may be calculated. All the above defined parameters, such as absorption spectrum and full width at a percentage of the maximum absorbance can be experimentally measured. In this disclosure, these parameters can be specifically used to characterize the narrow-band absorption Pdots.

In certain embodiments the absorption spectrum has a distinct absorbance maximum curve. The distinct absorbance maximum curve may not overlap with other absorbance curves, allowing for improved targeted excitation and multiplex applications. In some embodiments, the distinct absorbance curve can be characterized by not having significant spectral overlap with other absorbance curves (i.e., the absorption peak has less than 1% of an integrated area that overlaps with a neighboring absorption peak). In certain embodiments, the distinct absorbance curve can have minor spectral overlap. In some embodiments, the distinct absorbance maximum curve has an overlapped area that is less than 5% of the integrated area of any one of the neighboring peaks, less than 10% of the integrated area of any one of the neighboring peaks, less than 15% of the integrated area of any one of the neighboring peaks, less than 20% of the integrated area of any one of the neighboring peaks, less than 25% of the integrated area of any one of the neighboring peaks, less than 30% of the integrated area of any one of the neighboring peaks, less than 35% of the integrated area of any one of the neighboring peaks, or less than 40% of the integrated area of any one of the neighboring peaks. In some embodiments, the distinct absorbance curve can be baseline resolved. In specific embodiments, the distinct absorbance curve can be 100% baseline resolved, greater than 99% baseline resolved, greater than 98% baseline resolved, greater than 97% baseline resolved, greater than 96% baseline resolved, greater than 95% baseline resolved, greater than 90% baseline resolved, greater than 85% baseline resolved, greater than 80% baseline resolved, greater than 75% baseline resolved, greater than 70% baseline resolved, greater than 65% baseline resolved, or greater than 60% baseline resolved. In specific embodiments, the distinct absorbance curve is baseline separated (i.e., the spectrum returns to the baseline between peaks).

In some embodiments, the absorption spectrum includes a plurality of distinct curves. For example, the absorption spectrum can have 2 distinct curves, 3 distinct curves, or more than 3 distinct curves. In some embodiments, the maximum absorbance is calculated as the difference in intensity from the peak of the greatest absorbance curve and the baseline (FIG. 9C). The maximum absorbance curve and other distinct curves can be within the wavelength region from ultraviolet to infrared. In certain embodiments, the maximum absorbance curve and other distinct curves are within the region from 380 nm to 1200 nm. In specific embodiments, the maximum absorbance curve and other distinct curves are within the region from 380 nm to 1200 nm, from 400 nm to 1100 nm, from 500 nm to 1000 nm, from 600 nm to 900 nm, from 380 nm to 1100 nm, from 380 nm to 1000 nm, from 380 nm to 950 nm, from 380 nm to 900 nm, from 380 nm to 850 nm, from 380 nm to 800 nm, from 380 nm to 750 nm, from 380 nm to 700 nm, or from 400 to 700 nm. In some embodiments, the greatest absorbance curve can have a starting intensity value that is different from the ending intensity value (i.e., the intensity at the start of the absorbance curve may be higher than the intensity at the end of the absorbance curve) (FIG. 9D). In some embodiments a corrected baseline is used, and the maximum absorbance is calculated as the difference in intensity from the peak of the absorbance curve and the corrected baseline (FIG. 9D).

The corrected baseline can be set as the lowest value of intensity of the absorbance curve. In certain embodiments, the corrected baseline is set as the lowest value of intensity of the absorbance curve that is flat (i.e., has a slope of approximately 0). Generally, the lowest value of intensity of the absorbance curve is in the red wavelength section of the spectrum relative to the absorbance curve (i.e., to the right of the absorbance curve peak, having a higher wavelength value than the absorbance curve peak). In specific embodiments, the corrected baseline value can be set as the lowest value of intensity of the absorbance curve within the region from 350 nm to 1000 nm.

In certain embodiments, the absorbance peaks of multiple distinct absorbance curves on a spectrum are separated by a wavelength value. In some embodiments, the peaks of multiple distinct absorbance curves on a spectrum are separated by more than 20 nm, more than 30 nm, more than 40 nm, more than 50 nm, more than 60 nm, more than 70 nm, more than 80 nm, more than 90 nm, more than 100 nm, more than 110 nm, more than 120 nm, more than 130 nm, more than 140 nm, more than 150 nm, more than 200 nm, more than 250 nm, more than 300 nm, more than 350 nm, more than 400 nm, more than 450 nm, or more than 500 nm.

In some embodiments, the plurality of distinct curves can be characterized by not having significant spectral overlap with other distinct absorbance curves (i.e., each distinct absorption peak has less than 1% of an integrated area that overlaps with a neighboring absorption peak). In certain embodiments, each of the distinct absorbance curves can have minor spectral overlap. In some embodiments, each distinct absorbance maximum curve of the plurality of distinct curves has an overlapped area that is less than 5% of the integrated area of any one of the neighboring peaks, less than 10% of the integrated area of any one of the neighboring peaks, less than 15% of the integrated area of any one of the neighboring peaks, less than 20% of the integrated area of any one of the neighboring peaks, less than 25% of the integrated area of any one of the neighboring peaks, less than 30% of the integrated area of any one of the neighboring peaks, less than 35% of the integrated area of any one of the neighboring peaks, or less than 40% of the integrated area of any one of the neighboring peaks. In some embodiments, each of the distinct absorbance curves can be baseline resolved. In specific embodiments, each distinct absorbance curve can be 100% baseline resolved, greater than 99% baseline resolved, greater than 98% baseline resolved, greater than 97% baseline resolved, greater than 96% baseline resolved, greater than 95% baseline resolved, greater than 90% baseline resolved, greater than 85% baseline resolved, greater than 80% baseline resolved, greater than 75% baseline resolved, greater than 70% baseline resolved, greater than 65% baseline resolved, or greater than 60% baseline resolved. In specific embodiments, each distinct absorbance curve is baseline separated (i.e., the spectrum returns to the baseline between peaks).

The absorption wavelength of the polymer dots can vary from ultraviolet to the infrared region. In preferred embodiments, the polymer dots include an absorbing monomeric unit and an emitting monomeric unit. As provided herein, the chemical composition and structure of the polymer dots can be tuned to obtain small bandwidth of nanoparticle absorption. Other species such as narrow-band absorption units, narrow-band absorbing monomeric units, metal complexes, inorganic materials, or emissive units can be blended or chemically cross-linked within the polymer dots to obtain small bandwidths of the nanoparticle absorption.

### Narrow-Band Absorption Polymer Dots Including at Least One Polymer

The present disclosure provides, in certain embodiments, a nanoparticle including a polymer, wherein the polymer includes both an absorbing monomeric unit and an emitting monomeric unit, and the nanoparticle has an absorbance width of less than 150 nm at 10% (or in some embodiments, at 15%) of the absorbance maximum. In some embodiments, the absorbing monomeric unit includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof. In some embodiments, the absorbing monomeric unit includes BODIPY, a BODIPY derivative, or any combination thereof.

The present disclosure provides, in some embodiments, a nanoparticle including a polymer, wherein the polymer includes both an absorbing monomeric unit and an emitting monomeric unit, the absorbing monomeric unit includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof (e.g., the absorbing monomeric unit includes BODIPY, a BODIPY derivative, or any combination thereof). In some embodiments, the absorbing monomeric unit includes BODIPY, a BODIPY derivative, or any combination thereof. In specific embodiments, the nanoparticle has an absorbance width of less than 150 nm at 10% (or in some embodiments, at 15%) of the absorbance maximum.

In some embodiments, the nanoparticle further includes a polymer including one or more monomeric units different from the absorbing monomeric unit and the emitting monomeric unit. When the polymer further includes one or more monomeric units different from the absorbing monomeric unit and the emitting monomeric unit, the nanoparticle can have an absorbance width of less than 150 nm at 10% of the absorbance maximum. The one or more monomeric units different from the absorbing monomeric unit and the emitting monomeric can include a general monomeric unit; a functional monomeric unit; an energy transfer monomeric unit; an additional, second absorbing monomeric unit (different from the adsorbing monomeric unit above); or any combination thereof. The general monomeric unit can be, for example, a functional monomeric unit and/or an energy transfer monomeric unit. The functional monomeric unit provides a specific function, such as providing the monomeric unit with hydrophilic properties, hydrophobic properties, amphiphilic properties, fluorophilic properties, reactive functional groups, or any combinations thereof. For example, the functional monomeric unit can include a reactive functional group that can be used, for example, to conjugate a biomolecule. In some embodiments, the functional monomeric unit can provide hydrophilic properties to, hydrophobic properties to, and/or improve biocompatibility of the polymer. For example, the functional monomeric unit can be a hydrophilic monomeric unit. In some embodiments, the functional monomeric unit can be a hydrophilic monomeric unit that does not have a reactive functional group suitable for bioconjugation (e.g., conjugation under conditions that do not adversely affect the structure or function of the biomolecule).

In some embodiments, the narrow-band absorbing polymer includes a first absorbing monomeric unit, an emitting monomeric unit, and an energy transfer unit. In certain embodiments, the narrow-band absorbing polymer includes a first absorbing monomeric unit, an emitting monomeric unit, an energy transfer unit, and a functional monomeric unit. In some embodiments, the narrow-band absorbing polymer includes a first absorbing monomeric unit, an emitting monomeric unit, and a functional monomeric unit. In certain embodiments, the narrow-band absorbing polymer includes a first absorbing monomeric unit, a second absorbing monomeric unit, and an emitting monomeric unit. In some embodiments, the narrow-band absorbing polymer includes 2 monomeric units different from the absorbing monomeric unit and the emitting monomeric unit.

The polymer including the absorbing monomeric unit and the emitting monomeric unit can be referred to as an “absorbing and emitting polymer.”

In certain embodiments, the polymer has a backbone including the absorbing monomeric unit, has a side chain including the absorbing monomeric unit, has a terminus including the absorbing monomeric unit, or any combination thereof. In certain embodiments, the polymer has a backbone including the emitting monomeric unit, has a side chain including the emitting monomeric unit, has a terminus including the emitting monomeric unit, or any combination thereof. In certain embodiments, the polymer has a backbone including an absorbing unit, has a side chain including the absorbing unit, has a terminus including the absorbing unit, or any combination thereof. In certain embodiments, the polymer has a backbone including an emitting unit, has a side chain including the emitting unit, has a terminus including the emitting unit, or any combination thereof. In some embodiments, an absorbing unit can include one or more monomeric units that together function as an absorbing moiety. In some embodiments, an emitting unit can include one or more monomeric units that together function as an emitting moiety.

These polymers can be linear, branched, hyperbranched, dendritic, crosslinked, random, block, graft, or any structural type. In specific embodiments, the polymers are copolymers, and can be a block copolymer, a random copolymer, a periodic copolymer, a statistical copolymer, a gradient copolymer, an alternating copolymer, or any combination thereof.

In certain embodiments, the polymer is a semiconducting polymer. In specific embodiments, the polymer backbone is semiconducting.

In some embodiments, the narrow-band absorbing polymer does not include a β-phase structure. In certain embodiments, the narrow-band absorbing polymer does not include a fluorene or a fluorene-based monomeric unit. In some embodiments, the Pdot nanoparticle does not include any polymer having a β-phase structure. In certain embodiments, the Pdot nanoparticle does not include any polymer having a fluorene or a fluorene-based monomeric unit.

### Narrow-Band Absorption Polymer Dots Including at Least Two Polymers

The present disclosure provides, in certain embodiments, a nanoparticle including a first polymer including an absorbing monomeric unit and a second polymer including an emitting monomeric unit. The nanoparticle can have an absorbance width of less than 150 nm at 10% (or in some embodiments, at 15%) of the absorbance maximum. In some embodiments, the absorbing monomeric unit includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof. In some embodiments, the absorbing monomeric unit includes BODIPY, a BODIPY derivative, or any combination thereof. In some embodiments, the first polymer and the second polymer are the same polymer.

The present disclosure provides, in certain embodiments, a nanoparticle including a first polymer including an absorbing monomeric unit, the absorbing monomeric unit includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof, and a second polymer including an emitting monomeric unit. In some embodiments, the absorbing monomeric unit includes BODIPY, a BODIPY derivative, or any combination thereof. In certain embodiments, the nanoparticle has an absorbance width of less than 150 nm at 10% (or in some embodiments, at 15%) of the absorbance maximum. In some embodiments, the first polymer and the second polymer are the same polymer.

The polymer including the absorbing monomeric unit can be referred to as an “absorbing polymer” and the polymer including the emitting polymer can be referred to as an “emitting polymer.”

In some embodiments, the first polymer has a backbone including the absorbing monomeric unit, has a side chain including the absorbing monomeric unit, has a terminus (i.e., terminal end) including the absorbing monomeric unit, or any combination thereof. The absorbing monomeric unit can be cross-linked to the polymer backbone. An absorbing unit can include the absorbing monomeric unit, and can be cross-linked and/or covalently attached to the polymer backbone.

These polymers can be linear, branched, hyperbranched, dendritic, crosslinked, random, block, graft, or any structural type. In specific embodiments, the polymers are copolymers, and can be a block copolymer, a random copolymer, a periodic copolymer, a statistical copolymer, a gradient copolymer, an alternating copolymer, or any combination thereof.

In some embodiments, the first polymer is a semiconducting polymer. In certain embodiments, the second polymer is a semiconducting polymer. In some embodiments, the first polymer and the second polymer are each semiconducting polymers. In specific embodiments, the polymer backbones are semiconducting.

In some embodiments, the narrow-band absorbing nanoparticle has a mass ratio of the first polymer including the absorbing monomeric unit to the second polymer including the emitting monomeric unit. In certain embodiments, the mass ratio of the first polymer to the second polymer is greater than 1:1, greater than 2:1, greater than 3:1, greater than 4:1, greater than 5:1, greater than 6:1, greater than 7:1, greater than 8:1, greater than 9:1, greater than 10:1, greater than 20:1, greater than 30:1, greater than 40:1, greater than 50:1, or greater than 100:1. In certain embodiments, the mass ratio of the first polymer to the second polymer is 1:1 or more, 2:1 or more, 3:1 or more, 4:1 or more, 5:1 or more, 6:1 or more, 7:1 or more, 8:1 or more, 9:1 or more, 10:1 or more, 20:1 or more, 30:1 or more, 40:1 or more, 50:1 or more, or 100:1 or more. As a non-limiting example, a nanoparticle including 1 μg of the absorbing (first) polymer and 0.5 μg of the emitting (second) polymer would have a mass ratio of the first polymer to the second polymer of 2:1.

Compositions of Polymers

In certain embodiments, the nanoparticles include a first polymer and a second polymer, wherein the first polymer includes the absorbing monomeric unit and the second polymer includes the emitting monomeric unit. The first polymer can be referred to as an “absorbing polymer,” an “absorption polymer,” or an “absorptive polymer” and the second polymer can be referred to as an “emitting polymer,” an “emission polymer,” or an “emissive polymer.” In certain embodiments, the first polymer is a narrow-band absorbing polymer.

In some embodiments, the narrow-band absorbing polymer does not include a β-phase structure. In certain embodiments, the narrow-band absorbing polymer does not include a fluorene or a fluorene-based monomeric unit. In some embodiments, the Pdot nanoparticle does not include any polymer having a β-phase structure. In certain embodiments, the Pdot nanoparticle does not include any polymer having a fluorene or a fluorene-based monomeric unit.

In certain embodiments, the nanoparticle includes an absorbing polymer and an emitting polymer, wherein the polymers are physically blended and/or chemically crosslinked. In some embodiments, the nanoparticles have both intrachain and interchain energy transfer. In certain embodiments, the combination of intrachain and interchain energy transfer can increase the quantum yield of the polymer dots. In certain embodiments, the nanoparticles exhibit narrow-band absorption. In various embodiments, a polymer nanoparticle includes a blend of polymers provides structural and/or energy transfer support. For example, a Pdot including a semiconducting polymer, or a polymer including an emitting monomeric unit and an absorbing monomeric unit connected by a semiconducting backbone, can have enhanced energy transfer by, for example, fluorescence resonance energy transfer, through-bond energy transfer, and/or through-chain energy transfer.

Absorbing Polymers

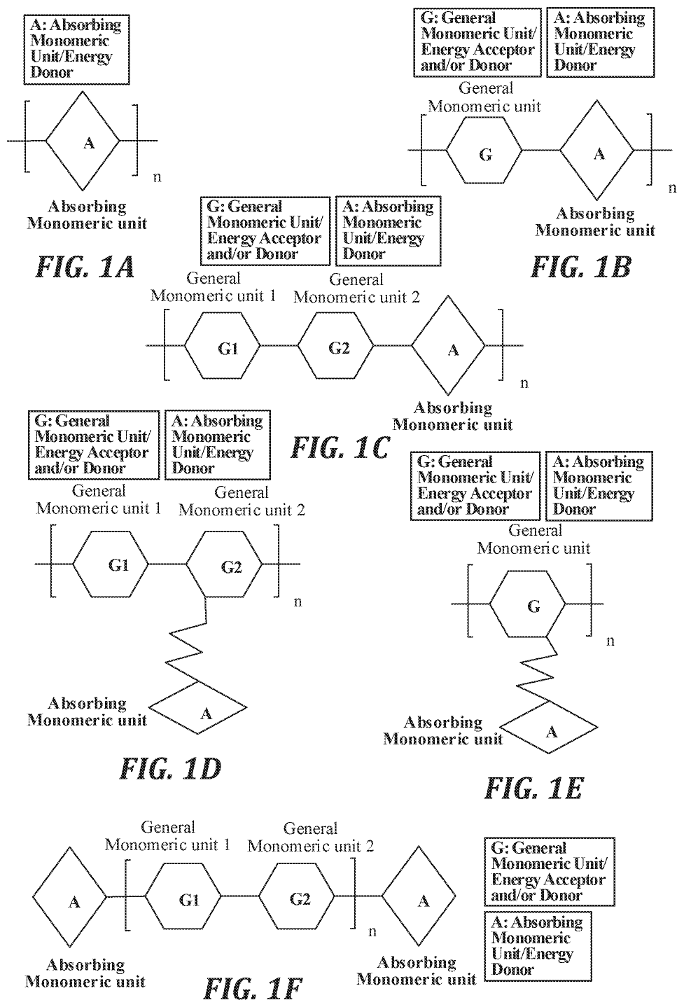

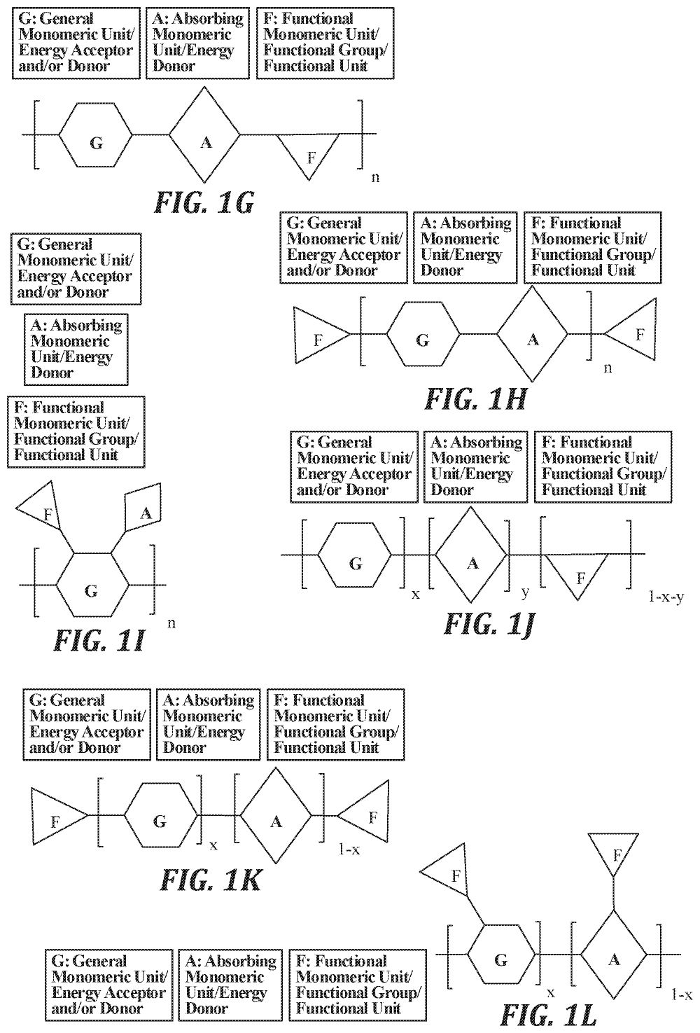

In some embodiments, the absorbing polymer is a homopolymer that includes only absorbing monomeric units (e.g., FIG. 1A). In some embodiments, the absorbing polymer is a two-unit copolymer that includes one absorbing monomeric unit and one general monomeric unit (e.g., G, G1, G2, and/or G2′) (FIG. 1B). The general monomeric unit can include a functional monomeric unit and/or an energy transfer monomeric unit. In some embodiments, the general monomeric units can be broad-band emissive (e.g., over a wavelength range of from about 400 nm to about 1000 nm). In some embodiments, the general monomeric units can be broad-band absorbing (e.g., over a wavelength range of from about 350 nm to about 800 nm). In some embodiments, the general monomeric units can be semiconductive. The general monomeric unit can be an energy acceptor and the absorbing monomeric unit can be an energy-donor. Energy-transfer inside Pdots can result in luminescent emissions. In some embodiments, energy-transfer inside Pdots can result in fluorescent emissions. In some embodiments, the absorbing polymer is a three-unit copolymer that includes one absorbing monomeric unit and two general monomeric units such as general monomeric unit 1 and general monomeric unit 2 (e.g., selected from G, G1, G2, and/or G2′) (FIG. 1C). The absorbing monomeric unit can be an energy-donor, general monomeric unit 1 can be an energy-acceptor and/or energy donor, general monomeric unit 2 can also be an acceptor from the absorbing monomeric unit and/or energy donor to an emitter. In some embodiments, general monomeric unit 2 can be an energy-donor to general monomeric unit 1 or an emitter and simultaneously an energy-acceptor from the absorbing monomeric unit. Both general monomeric unit 1 and general monomeric unit 2 can be semiconducting. Both general monomeric unit 1 and general monomeric unit 2 can be emissive. However, multi-step energy-transfer inside Pdots can result in emissions with high quantum yield. In certain embodiments, the absorbing polymer can be a heteropolymer, such as a multi-unit (≥3) copolymer, that includes at least one type of absorbing monomeric unit so that the final Pdots give narrow-band absorptions.

In some embodiments, the absorbing polymer is a copolymer that includes the absorbing unit cross-linked with the side-chains (FIG. 1D). The copolymer can include 2 types of general monomeric units, 3 types of general monomeric units, 4 types of general monomeric units, 5 types of general monomeric units, or more than 5 types of general monomeric units (e.g., selected from G, G1, G2, and/or G2′). However, the absorbing polymer can include at least one type of absorbing unit in the side-chains. The copolymer backbone can be an energy-acceptor, and the absorbing unit can be an energy-donor. Energy-transfer inside Pdots results in luminescent emissions. In some embodiments, the absorbing polymer is a homopolymer that includes the absorbing unit cross-linked with the side-chains (FIG. 1E). The homopolymer backbone can be an energy-acceptor, and the absorbing unit can be an energy-donor. Energy-transfer inside Pdots can result in luminescent emissions. In some embodiments, the luminescent emissions can have narrow-band emissions. In certain embodiments, the narrow-band absorbing nanoparticle includes a narrow-band emitting monomeric unit, a narrow-band emitting polymer, or any combination thereof. Examples of narrow-band emitting monomeric units, narrow-band emitting polymers, and general monomeric units are provided herein and can be found in international application PCT/US2012/071767, which is incorporated herein by reference.

In some embodiments, the absorbing polymer can be a polymer that includes an absorbing monomeric unit attached to at least one terminus, or both termini of the polymer (FIG. 1F) in the case of a linear polymer, or all termini in the case of a branched polymer. The polymer can, e.g., include one type of a general monomeric unit (e.g., any one of G, G1, G2, or G2′), or two types of general monomeric units (e.g., any one of G, G1, G2 or G2′), or three types of general monomeric units, or more than three types of general monomeric units. The polymer backbone can be an energy-acceptor, and the absorbing unit can be an energy-donor. Energy-transfer inside Pdots results in luminescent emissions. In some embodiments, the absorbing polymer can be a homopolymer or heteropolymer that includes the absorbing unit attached to the terminus of the polymer. The homopolymer or heteropolymer backbone can be an energy-acceptor, and the absorbing unit can be an energy-donor. Energy-transfer inside Pdots can result in luminescent emissions.

FIGS. 1G-1L show other examples of schematic structures for the absorbing polymers that can include, e.g., general monomeric units as acceptors and donors (G) and absorbing monomeric units as donors (A). In some aspects, the donors can absorb energy and transfer the energy, either directly or indirectly (e.g. by cascade energy transfer), to the emitting monomeric units or emitting polymer. Besides the general monomeric unit and absorbing monomeric unit, these polymers can also include functional monomeric units, functional groups, and/or functional units (F) that provide reactive functional groups for, e.g., chemical reactions and bioconjugation reactions, or provide other functions unrelated to chemical reactions, such as endowing some of the monomeric unit with hydrophilic properties or amphiphilic properties. The functional monomeric units, functional groups, and/or functional units can include, for example, haloformyl, hydroxyl, aldehyde, alkenyl, alkynyl, anhydride, carboxamide, amines, azo compound, carbonate, carboxylate, carboxyl, cyanates, ester, haloalkane, imine, isocyanates, nitrile, nitro, phosphino, phosphate, phosphate, pyridyl, sulfonyl, sulfonic acid, sulfoxide, thiol groups, or any combination thereof, and reactive groups that can react via click-chemistry, such as alkyne, strained alkyne, azide, diene, alkene, cyclooctyne, phosphine groups, or any combination thereof. The functional monomeric units can be copolymerized with the general monomeric units and absorbing monomeric units (e.g., FIG. 1G), or cross-linked with these two kinds of monomeric units. The functional monomeric units can be used as a terminus (or for both termini) of the polymers (e.g., FIG. 1H and FIG. 1K). Functional groups can be included either in the general monomeric unit or the absorbing monomeric units (e.g., FIG. 1I). In some embodiments, the absorbing monomeric units can also be copolymerized with any of the general polymers to synthesize an absorbing copolymer or heteropolymer that contains more than two types of monomeric units (e.g., FIG. 1J). The absorbing monomeric unit can be covalently attached to the side-chains of the polymer (e.g., FIG. 1L). In some embodiments, the absorbing units can be covalently attached to the terminus of the polymer. In some embodiments, the absorbing units can be physically mixed or blended with conventional semiconducting polymers to form narrow-band absorbing polymer dots. In one embodiment, the absorbing units can be covalently cross-linked with conventional semiconducting polymers to form narrow-band absorbing polymer dots. The conventional semiconducting polymers can absorb energy and transfer the energy, either directly or indirectly (e.g. by cascade energy transfer) to the emitting monomeric unit or emitting polymer.

All the absorbing polymers described above in FIGS. 1A-IL can, e.g., be physically blended or chemically cross-linked with one or more general broad-band absorbing and/or emitting polymers. In some aspects, the polymers can be energy donors and acceptors, and the absorbing polymers can be energy donors. Multi-step energy transfer can occur from the absorbing polymer to the emissive polymer so that the polymer dots give luminescent emissions. The chemical cross-linking between polymers can use the functional reactive groups such as haloformyl, hydroxyl, aldehyde, alkenyl, alkynyl, anhydride, carboxamide, amines, azo compound, carbonate, carboxylate, carboxyl, cyanates, ester, haloalkane, imine, isocyanates, nitrile, nitro, phosphino, phosphate, phosphate, pyridyl, sulfonyl, sulfonic acid, sulfoxide, thiol groups, or any combination thereof, and reactive groups that can react via click-chemistry, such as alkyne, strained alkyne, azide, diene, alkene, cyclooctyne, phosphine groups, or any combination thereof. These functional groups can be attached to the side chains and/or the terminus of each polymer chain.

Emitting Polymers

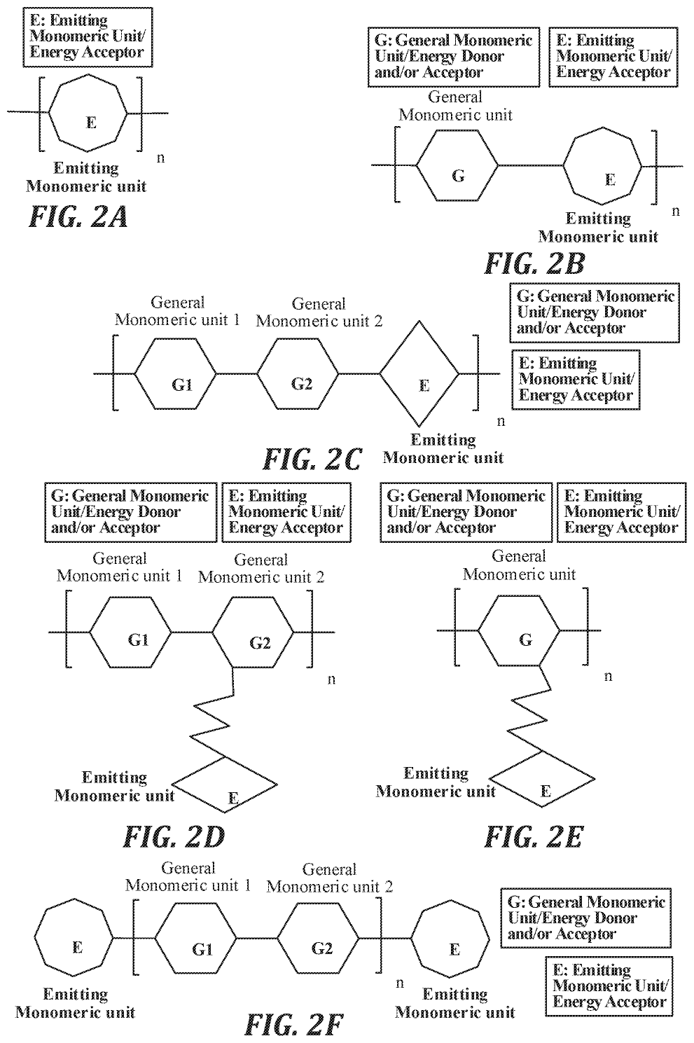

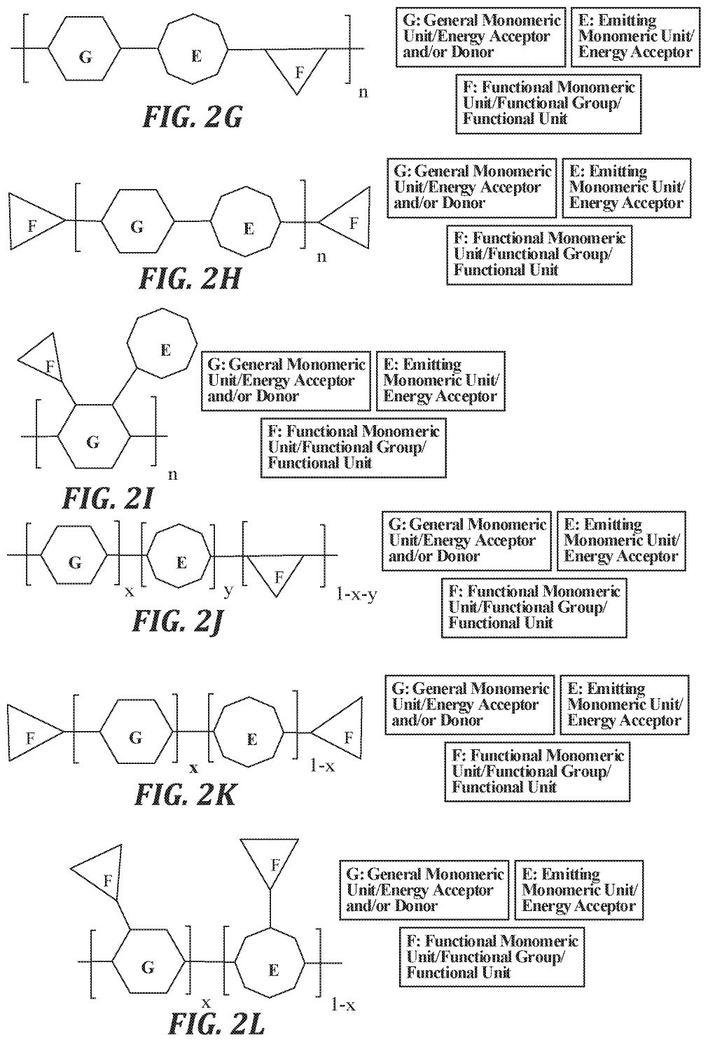

In some embodiments, the emitting polymer is a homopolymer that includes only emitting monomeric units (e.g., FIG. 2A). In some embodiments, the emitting polymer is a two-unit copolymer that includes one emitting monomeric unit and one general monomeric unit (e.g., G, G1, G2, and/or G2′) (FIG. 2B). The general monomeric unit can include a functional monomeric unit and/or an energy transfer monomeric unit. In some embodiments, the general monomeric units can be broad-band absorbing. In some embodiments, the general monomeric units can be broad-band emitting. In some embodiments, the general monomeric units can be semiconductive. The general monomeric unit can be an energy donor and the emitting monomeric unit can be an energy-acceptor. Energy-transfer inside Pdots can result in luminescent emissions. In some embodiments, energy-transfer inside Pdots can result in fluorescent emissions. In some embodiments, the emitting polymer is a three-unit copolymer that includes one emitting monomeric unit and two general monomeric units such as general monomeric unit 1 and general monomeric unit 2 (e.g., selected from G, G1, G2, and/or G2′) (FIG. 2C). The emitting monomeric unit can be an energy-acceptor, general monomeric unit 1 can be an energy-donor, and general monomeric unit 2 can also be a donor to the emitting monomeric unit. In some embodiments, general monomeric unit 2 can be an energy-acceptor from general monomeric unit 1 and simultaneously an energy-donor to the emitting monomeric unit. Both general monomeric unit 1 and general monomeric unit 2 can be semiconducting. Both general monomeric unit 1 and general monomeric unit 2 can be emissive. Multi-step energy-transfer inside Pdots can result in narrow-band emissions. In certain embodiments, the emitting polymer can be a heteropolymer, such as a multi-unit (≥3) copolymer, that includes at least one type of emitting monomeric unit so that the final Pdots give luminescent emissions.

In some embodiments, the emitting polymer is a copolymer that includes the emitting unit cross-linked with the side-chains (FIG. 2D). The copolymer can include 2 types of general monomeric units, 3 types of general monomeric units, 4 types of general monomeric units, 5 types of general monomeric units, or more than 5 types of general monomeric units (e.g., selected from G, G1, G2, and/or G2′). However, the emitting polymer can include at least one type of emitting unit in the side-chains. The copolymer backbone can be an energy-donor, and the emitting unit can be an energy-acceptor. Energy-transfer inside Pdots results in luminescent emissions. In some embodiments, the emitting polymer is a homopolymer that includes the emitting unit cross-linked with the side-chains (FIG. 2E). The homopolymer backbone can be an energy-donor, and the emitting unit can be an energy-acceptor. Energy-transfer inside Pdots can result in luminescent emissions. In some embodiments, the luminescent emissions are narrow-band emissions.

In some embodiments, the emitting polymer can be a polymer that includes an emitting monomeric unit attached to at least one terminus, or both termini of the polymer (FIG. 2F) in the case of a linear polymer, or all termini in the case of a branched polymer. The polymer can, e.g., include one type of a general monomeric unit (e.g., any one of G, G1, G2, or G2′), or two types of general monomeric units (e.g., any one of G, G1, G2 or G2′), or three types of general monomeric units, or more than three types of general monomeric units. The polymer backbone can be an energy-donor, and the emitting unit can be an energy-acceptor. Energy-transfer inside Pdots results in luminescent emissions. In some embodiments, the emitting polymer can be a homopolymer or heteropolymer that includes the emitting unit attached to the terminus of the polymer. The homopolymer or heteropolymer backbone can be an energy-donor, and the emitting unit can be an energy-acceptor. Energy-transfer inside Pdots can result in luminescent emissions.

FIGS. 2G-2L show other examples of schematic structures for the emitting polymers that can include, e.g., general monomeric units as acceptors and donors (G) and emitting monomeric units as acceptors (E). In some aspects, the general monomeric units can absorb energy and transfer the energy, either directly or indirectly (e.g. by cascade energy transfer), to the emitting monomeric units or emitting polymer. Besides the general monomeric unit and emitting monomeric unit, these polymers can also include functional monomeric units, functional groups, and/or functional units (F) that provide reactive functional groups for, e.g., chemical reactions and bioconjugation reactions. The functional monomeric units, functional groups, and/or functional units can include, for example, haloformyl, hydroxyl, aldehyde, alkenyl, alkynyl, anhydride, carboxamide, amines, azo compound, carbonate, carboxylate, carboxyl, cyanates, ester, haloalkane, imine, isocyanates, nitrile, nitro, phosphino, phosphate, phosphate, pyridyl, sulfonyl, sulfonic acid, sulfoxide, thiol groups, or any combination thereof, and reactive groups that can react via click-chemistry, such as alkyne, strained alkyne, azide, diene, alkene, cyclooctyne, phosphine groups, or any combination thereof. The functional monomeric units can be copolymerized with the general monomeric units and absorbing monomeric units (e.g., FIG. 2G), or cross-linked with these two kinds of monomeric units. The functional monomeric units can be used as a terminus (or for both termini) of the polymers (e.g., FIG. 2H and FIG. 2K). The functional monomeric unit can provide a specific function, such as providing the monomeric unit with hydrophilic properties, hydrophobic properties, amphiphilic properties, fluorophilic properties, reactive functional groups, or any combinations thereof. Functional groups can be included either in the general monomeric unit or the emitting monomeric units (e.g., FIG. 2I). In some embodiments, the emitting monomeric units can also be copolymerized with any of the general polymers to synthesize an emitting copolymer or heteropolymer that contains more than two types of monomeric units (e.g., FIG. 2J). The emitting monomeric unit can be covalently attached to the side-chains of the polymer (e.g., FIG. 2L). In some embodiments, the emitting units can be covalently attached to the terminus of the polymer. In some embodiments, the emitting units can be physically mixed or blended with conventional semiconducting polymers to form narrow-band absorbing polymer dots with luminescent emission. In one embodiment, the emitting units can be covalently cross-linked with conventional semiconducting polymers to form luminescent polymer dots. The conventional semiconducting polymers can absorb energy and transfer the energy, either directly or indirectly (e.g. by cascade energy transfer) to the emitting monomeric unit or emitting polymer.

All the emitting polymers described above in FIGS. 2A-2L can, e.g., be physically blended or chemically cross-linked with one or more general emitting and/or absorbing polymers. In some aspects, the polymers can be energy donors and acceptors, and the emitting polymers can be energy acceptors. Multi-step energy transfer can occur from the absorbing polymer to the emissive polymer so that the polymer dots give luminescent emissions. The chemical cross-linking between polymers can use the functional reactive groups such as haloformyl, hydroxyl, aldehyde, alkenyl, alkynyl, anhydride, carboxamide, amines, azo compound, carbonate, carboxylate, carboxyl, cyanates, ester, haloalkane, imine, isocyanates, nitrile, nitro, phosphino, phosphate, phosphate, pyridyl, sulfonyl, sulfonic acid, sulfoxide, thiol groups, or any combination thereof, and reactive groups that can react via click-chemistry, such as alkyne, strained alkyne, azide, diene, alkene, cyclooctyne, phosphine groups, or any combination thereof. These functional groups can be attached to the side chains and/or the terminus of each polymer chain.

Absorbing and Emitting Polymers

In certain embodiments, the nanoparticles include a polymer including both an absorbing monomeric unit and an emitting monomeric unit. In some embodiments, a polymer including both an absorbing monomeric unit and an emitting monomeric unit is referred to as an “absorbing and emitting polymer” or an “emitting and absorbing polymer.” In certain embodiments, the absorbing and emitting polymer is a narrow-band absorbing polymer.

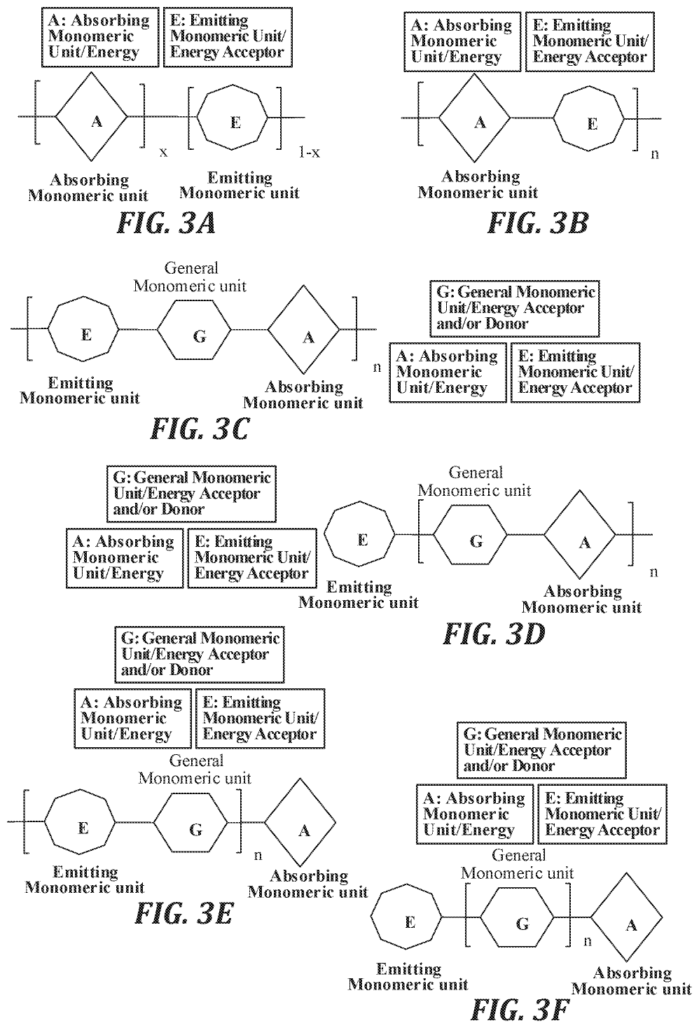

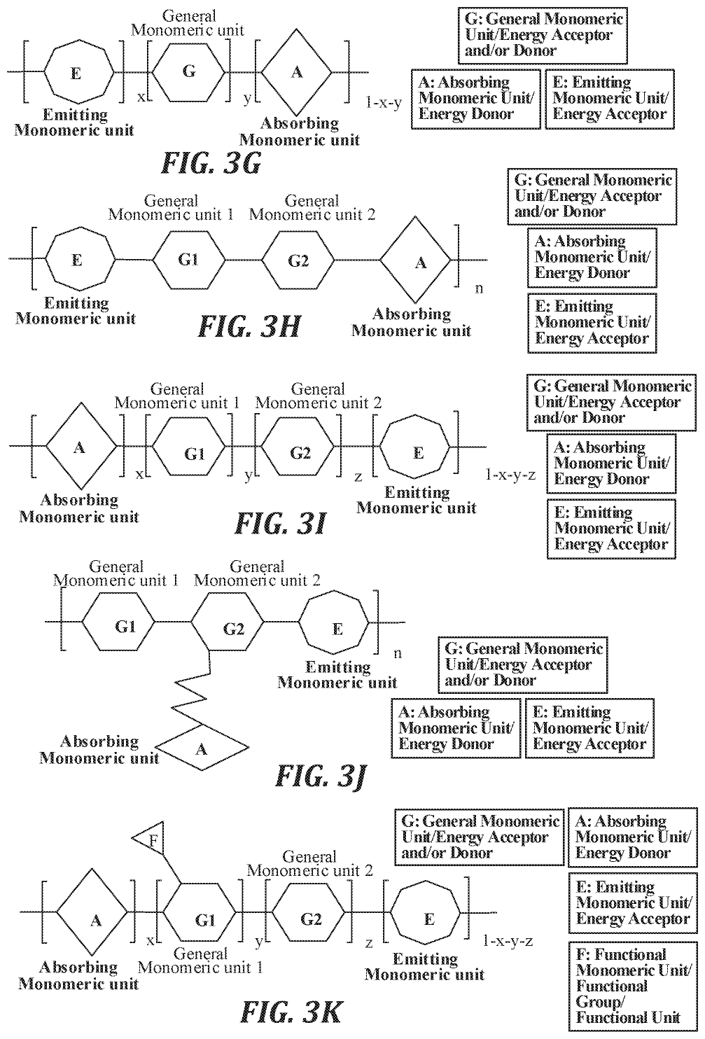

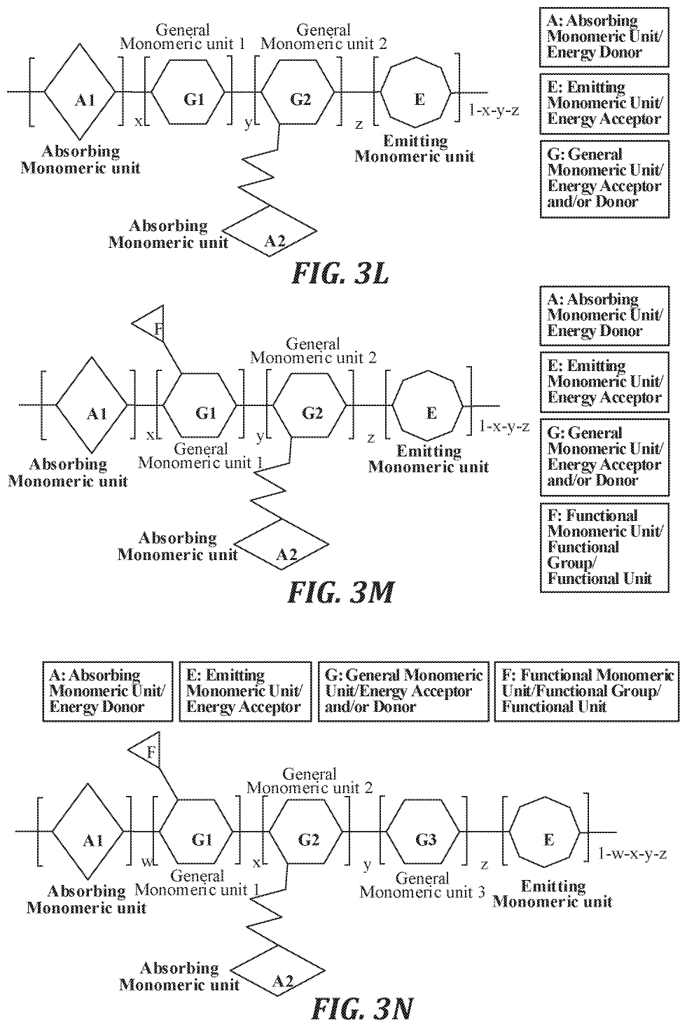

In some embodiments, the polymer is a two-unit random copolymer, and includes the absorbing monomeric unit and the emitting monomeric unit (FIG. 3A). In certain embodiments, the polymer is a two-unit alternating copolymer including the absorbing monomeric unit and the emitting monomeric unit (FIG. 3B). In some embodiments, the absorbing monomeric unit acts as an energy donor and the emitting monomeric unit acts as an energy acceptor. Energy can be transferred from the absorbing monomeric unit to the emitting monomeric unit, resulting in the emission of luminescence.

In some embodiments, the polymer includes the absorbing monomeric unit and the emitting monomeric unit, and further includes at least one general monomeric unit (e.g., G, G1, G2, and/or G2′) (FIGS. 3C-3N). The general monomeric unit can include a functional monomeric unit and/or an energy transfer monomeric unit. In certain embodiments, the polymer is a three-unit alternating copolymer including the emitting monomeric unit, the general monomeric unit, and the absorbing monomeric unit (FIG. 3C). In other embodiments, the polymer is a two-unit alternating copolymer including the absorbing monomeric unit and the general monomeric unit, and the emitting monomeric unit is located on a terminus (FIG. 3D). In some embodiments, the polymer is a two-unit alternating copolymer including the emitting monomeric unit and the general monomeric unit, and the absorbing monomeric unit is located on a terminus (FIG. 3E). In some embodiments, the polymer includes a repeating general monomeric unit with the emitting monomeric unit and absorbing monomeric unit each located on a terminus (FIG. 3F). In other embodiments, the polymer is a three-unit random copolymer, and includes the emitting monomeric unit, the general monomeric unit, and the absorbing monomeric unit (FIG. 3G). In some embodiments, the general monomeric units can be broad-band absorbing. In some embodiments, the general monomeric units can be broad-band emitting. In some embodiments, the general monomeric units can be semiconductive. The general monomeric unit can be an energy donor and an energy acceptor, the absorbing monomeric unit can be an energy donor, and the emitting monomeric unit can be an energy acceptor. Energy-transfer inside Pdots can result in luminescent emissions. In some embodiments, energy-transfer inside Pdots can result in fluorescent emissions. Multi-step energy-transfer inside Pdots can result in narrow-band emissions. As a non-limiting example, energy absorbed by the absorbing monomeric unit (acting as an energy donor) can be transferred to the general monomeric unit (acting as an energy acceptor), then further transferred from the general monomeric unit (acting as an energy donor) to the emitting monomeric unit (acting as an energy acceptor). In some embodiments, the general monomeric units can include a functional monomeric unit, to provide a specific function, such as providing the monomeric unit with hydrophilic properties, hydrophobic properties, amphiphilic properties, fluorophilic properties, reactive functional groups, or any combinations thereof. In certain embodiments, the emitting polymer can be a heteropolymer, such as a multi-unit (≥3) copolymer, that includes at least one type of emitting monomeric unit so that the final Pdots give luminescent emissions.

In certain embodiments, the polymer includes the absorbing monomeric unit, the emitting monomeric unit, and at least two general monomeric units (e.g., G, G1, G2, and/or G2′) (FIGS. 3H-3N). The general monomeric unit can include a functional monomeric unit and/or an energy transfer monomeric unit. In some embodiments, the polymer is a four-unit alternating copolymer including the emitting monomeric unit, a first general monomeric unit, a second general monomeric unit, and the absorbing monomeric unit (FIG. 3H). In certain embodiments, the general monomeric units act as energy donors and acceptors, and can transfer energy along the polymer backbone. In other embodiments, the polymer is a four-unit random copolymer including the emitting monomeric unit, a first general monomeric unit, a second general monomeric unit, and the absorbing monomeric unit (FIG. 3I). The absorbing monomeric unit can be an energy-donor, the emitting monomeric unit can be an energy-acceptor, general monomeric unit 1 can be an energy-donor and acceptor, and general monomeric unit 2 can also be an energy-donor and acceptor. In some embodiments, general monomeric unit 1 can be an energy-acceptor from the absorbing monomeric unit and simultaneously an energy-donor to general monomeric unit 2, while general monomeric unit 2 can be an energy-acceptor from general monomeric unit 1 and simultaneously an energy-donor to the emitting monomeric unit. Both general monomeric unit 1 and general monomeric unit 2 can be semiconducting. Both general monomeric unit 1 and general monomeric unit 2 can be emissive. Multi-step energy-transfer inside Pdots can result in narrow-band emissions. In certain embodiments, the absorbing and emitting polymer can be a heteropolymer, such as a multi-unit (≥3) copolymer, that includes at least one type of emitting monomeric unit so that the final Pdots give luminescent emissions.

In some embodiments, the absorbing polymer is a copolymer that includes an absorbing unit and/or an emitting unit cross-linked with the side-chains (FIG. 3J). The copolymer can include 2 types of general monomeric units, 3 types of general monomeric units, 4 types of general monomeric units, 5 types of general monomeric units, or more than 5 types of general monomeric units (e.g., selected from G, G1, G2, and/or G2′). The polymer can include at least one type of absorbing unit and/or emitting unit in the side-chains. The copolymer backbone can be an energy-acceptor, the absorbing unit can be an energy-donor and an energy-acceptor, and the emitting monomeric unit can be an energy-acceptor. Energy-transfer inside Pdots results in luminescent emissions. In some embodiments, the luminescent emissions are narrow-band emissions.

In some embodiments, the polymer is a copolymer that includes a functional monomeric unit, a functional group, and/or a functional unit. In specific embodiments, the functional monomeric unit, functional group, and/or functional unit is attached to a general monomeric unit (FIG. 3K). Absorbing and emitting polymers can include, e.g., general monomeric units as acceptors and donors (G), emitting monomeric units as acceptors (E), and absorbing monomeric units as donors (A). In some aspects, the general monomeric units can absorb energy and transfer the energy, either directly or indirectly (e.g. by cascade energy transfer), to the emitting monomeric unit. Besides the general monomeric unit, the absorbing monomeric unit, and the emitting monomeric unit, these polymers can also include functional monomeric units, functional groups, and/or functional units (F) that provide reactive functional groups for, e.g., chemical reactions and bioconjugation reactions, or provide other functions unrelated to chemical reactions, such as endowing some of the monomeric unit with hydrophilic properties or amphiphilic properties. Functional monomeric units, functional groups, and/or functional units are as described above for FIGS. 2A-2L. The functional monomeric units can be copolymerized with the general monomeric units and absorbing monomeric units, or cross-linked monomeric units (e.g., FIG. 3K). The functional monomeric units can be used as a terminus (or for both termini) of the polymers. Functional groups can be included either in the general monomeric unit or the emitting monomeric units.

In some embodiments, the functional monomeric unit is a monomeric unit having a specific function, such as providing hydrophilic properties to, hydrophobic properties to, amphiphilic properties to, and/or improve biocompatibility of, the polymer. For example, the functional monomeric unit can be functionalized with hydrophilic, hydrophobic, amphiphilic groups, which can be reactive (e.g., suitable for bioconjugation), or non-reactive (e.g., unsuitable for bioconjugation). The length, size, and nature of the hydrophilic, hydrophobic, and/or amphiphilic side chains can modify the chain-chain interactions, and control the packing of the polymers, and affect the colloidal stability and size of the polymer dots. The length, size, and nature of the hydrophilic, hydrophobic, and/or amphiphilic side chains can also affect the absorption, emission peak, emission bandwidth, fluorescence quantum yield, fluorescence lifetime, photostability, and other properties of the polymer and polymer dots. For example, many very hydrophilic functional groups can reduce the brightness of the polymer dots, and/or broaden the emission spectrum, and/or also adversely affect their colloidal stability and non-specific binding properties. In some embodiments, the functional monomeric unit includes hydrophilic groups such as oligo(ethylene glycol), poly(ethylene glycol), poly(propylene glycol) (which are less hydrophilic than poly(ethylene glycol), poly(ethers), hydroxyl, and/or sulphate. In some embodiments, the functional monomeric unit includes hydrophobic functional groups such as styrene, alkyl, and/or fatty acid chains.

In some embodiments, the emitting monomeric units can also be copolymerized with any of the general polymers to synthesize an emitting copolymer or heteropolymer that contains more than two types of monomeric units. The emitting monomeric unit can be covalently attached to the side-chains of the polymer. In some embodiments, the emitting units can be covalently attached to the terminus of the polymer. In some embodiments, the emitting units can be physically mixed or blended with conventional semiconducting polymers to form narrow-band absorbing polymer dots with luminescent emission. In one embodiment, the emitting units can be covalently cross-linked with conventional semiconducting polymers to form luminescent polymer dots. The conventional semiconducting polymers can absorb energy and transfer the energy, either directly or indirectly (e.g. by cascade energy transfer) to the emitting monomeric unit or emitting polymer.

In certain embodiments, the polymer is a copolymer that includes more than one absorbing unit. The copolymer can include an absorbing monomeric unit attached to the backbone of the polymer, as well as an absorbing unit attached to the polymer via cross-link (FIG. 3L). In some embodiments, the copolymer including both an absorbing monomeric unit and an absorbing unit can further include a functional monomeric unit, functional group, and/or functional unit attached to the polymer (FIG. 3M). The polymer can include, for example, an absorbing monomeric unit, a functionalized first general monomeric unit, a second general monomeric unit cross-linked with an absorbing and/or emitting unit, a third general monomeric unit, and an emitting monomeric unit (FIG. 3N). The copolymer can include 3 types of general monomeric units, 4 types of general monomeric units, 5 types of general monomeric units, 6 types of general monomeric units, or more than 6 types of general monomeric units (e.g., selected from G, G1, G2, G3, and/or G2′). The polymer can include at least one type of absorbing unit in the side-chains. The copolymer backbone can be an energy-acceptor, the absorbing unit can be an energy-donor and an energy-acceptor, and the emitting monomeric unit can be an energy-acceptor. Energy-transfer inside Pdots results in luminescent emissions. In some embodiments, the luminescent emissions are narrow-band emissions.

In certain embodiments, the polymer is a copolymer that includes more than one emitting unit. The copolymer can include an emitting monomeric unit attached to the backbone of the polymer, as well as an emitting unit attached to the polymer via cross-link. In certain embodiments, the copolymer can include more than one emitting unit attached to the polymer via cross-link. In some embodiments, the copolymer including both an emitting monomeric unit and an emitting unit can further include a functional monomeric unit, functional group, and/or functional unit attached to the polymer. The polymer can include, for example, an emitting monomeric unit, a functionalized first general monomeric unit, a second general monomeric unit cross-linked with an emitting and/or absorbing unit, a third general monomeric unit, and an absorbing monomeric unit. The copolymer can include 3 types of general monomeric units, 4 types of general monomeric units, 5 types of general monomeric units, 6 types of general monomeric units, or more than 6 types of general monomeric units (e.g., selected from G, G1, G2, G3, and/or G2′). The polymer can include at least one type of emitting unit in the side-chains. The copolymer backbone can be an energy-acceptor, the absorbing monomeric unit can be an energy-donor and an energy-acceptor, the emitting unit can be an energy-acceptor, and the emitting monomeric unit can be an energy-acceptor. Energy-transfer inside Pdots results in luminescent emissions. In some embodiments, the luminescent emissions are narrow-band emissions.

These polymers can also include functional monomeric units, functional units, and/or functional groups that provide reactive functional groups for, e.g., chemical reactions and bioconjugation reactions. The functional monomeric units can be copolymerized, or cross-linked with the polymers. All the emitting polymers described above and in FIGS. 3A-N can, e.g., be physically blended or chemically cross-linked with one or more emitting and/or absorbing polymers. In some aspects, the polymers can be energy donors and acceptors, and the emitting polymers can be energy acceptors. Multi-step energy transfer can occur from the absorbing polymer to the emissive polymer so that the polymer dots give luminescent emissions. The chemical cross-linking between polymers can use the functional reactive groups such as haloformyl, hydroxyl, aldehyde, alkenyl, alkynyl, anhydride, carboxamide, amines, azo compound, carbonate, carboxylate, carboxyl, cyanates, ester, haloalkane, imine, isocyanates, nitrile, nitro, phosphino, phosphate, phosphate, pyridyl, sulfonyl, sulfonic acid, sulfoxide, thiol groups, or any combination thereof. These functional groups can be attached to the side chains and/or the terminus of each polymer chain.

General Monomeric Units

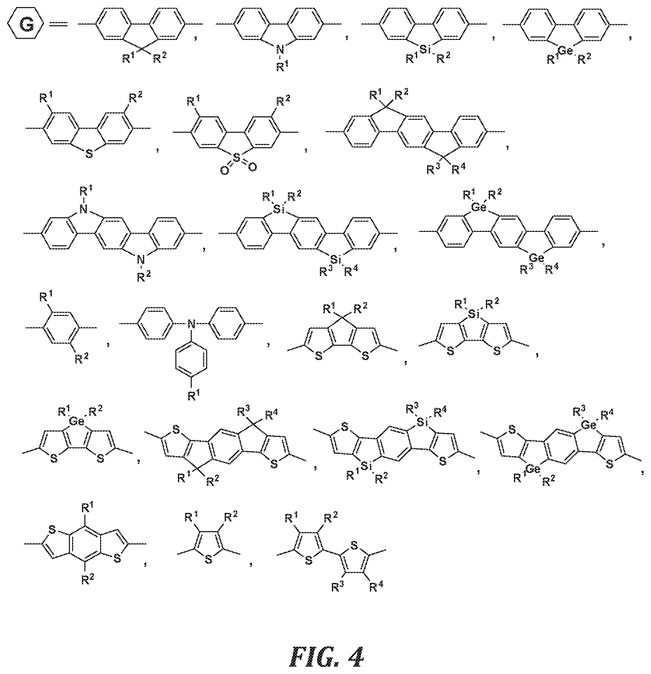

As described herein, the present disclosure can include general monomeric units that can be polymerized with the emitting monomeric units and/or absorbing monomeric units disclosed herein. FIG. 4 provides a non-limited list of example general monomeric units (G). In some embodiments, the general monomeric unit can act as an energy donor to an emitting monomeric unit. In some embodiments, the general monomeric unit can act as an energy acceptor for an absorbing monomeric unit. In some embodiments, the general monomeric unit can act as a functional monomeric unit. A variety of derivatized monomeric units can be used. For example, for the structures shown in FIG. 4, each of R¹, R², R³ and R⁴ can be independently selected from, but are not limited to, alkyl, phenyl, alkyl-substituted phenyl, alkyl-substituted fluorenyl and alkyl-substituted carbazolyl. Alkyl substituted phenyl can include 2-alkylphenyl, 3-alkylphenyl, 4-alkylphenyl, 2,4-dialkylphenyl, 3,5-dialkylphenyl, and 3,4-dialkylphenyl. Alkyl-substituted fluorenyl can include 9,9-dialkyl-substituted fluorenyl, 7-alkyl-9,9-dialkyl-substituted fluorenyl, 7-triphenylaminyl-9,9-dialkyl-substitute fluorenyl and 7-diphenylaminyl-9,9-dialkyl-substitute fluorenyl. The alkyl substituents can include CₙH₂ₙ₊₁, or CₙF₂ₙ₊₁ or —CH₂CH₂[OCH₂CH₂]ₙ—OCH₃ wherein n is 1 to 20. In some embodiments, n can be between 1 to 50 or higher. The general monomeric units can also be substituted with other substituents as defined herein.

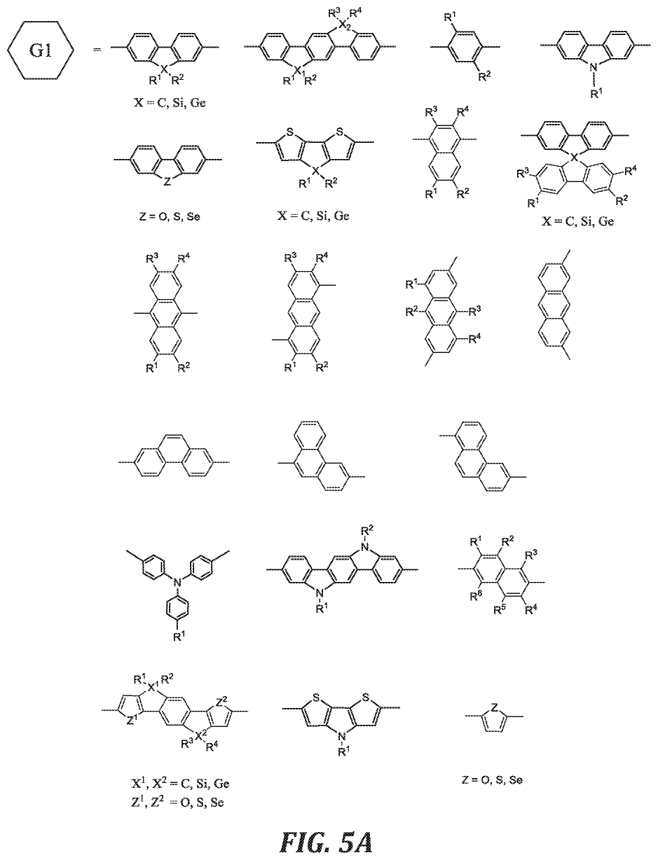

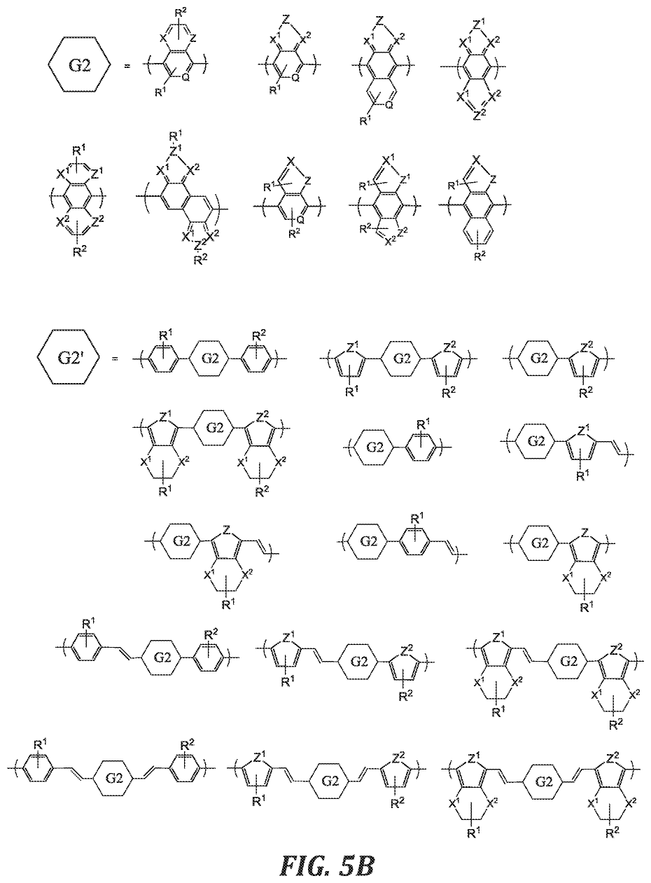

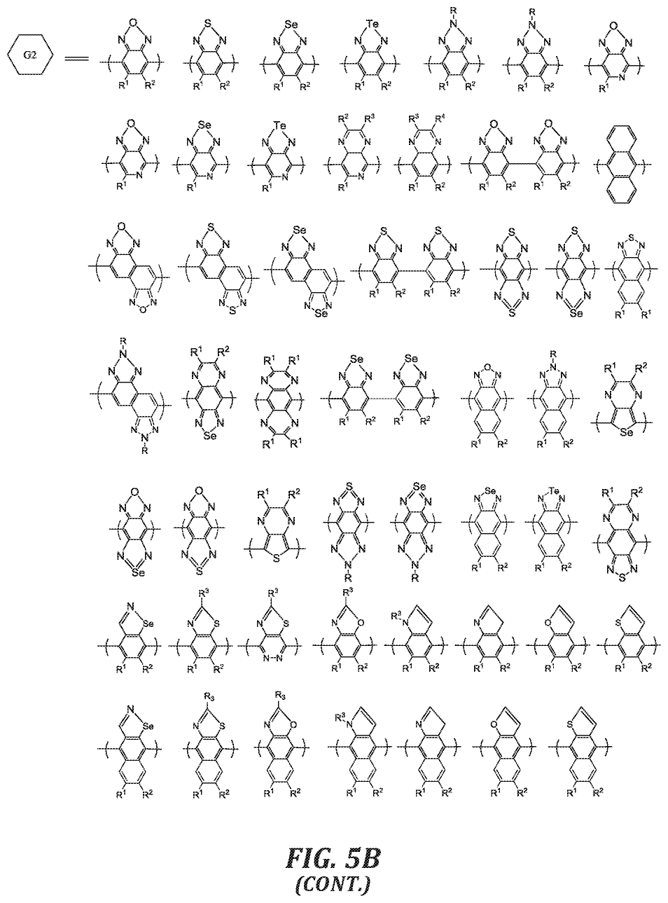

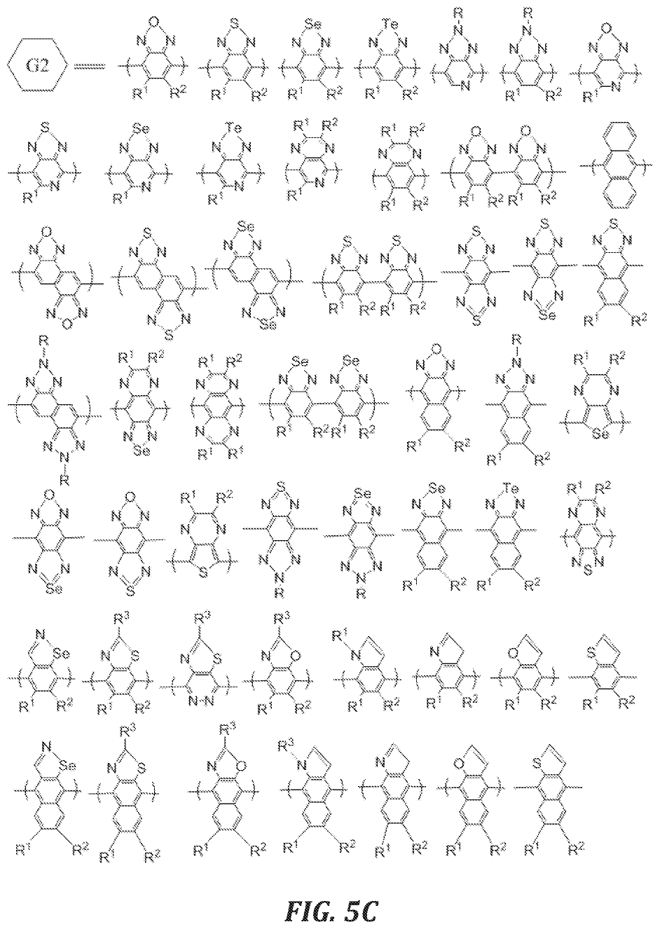

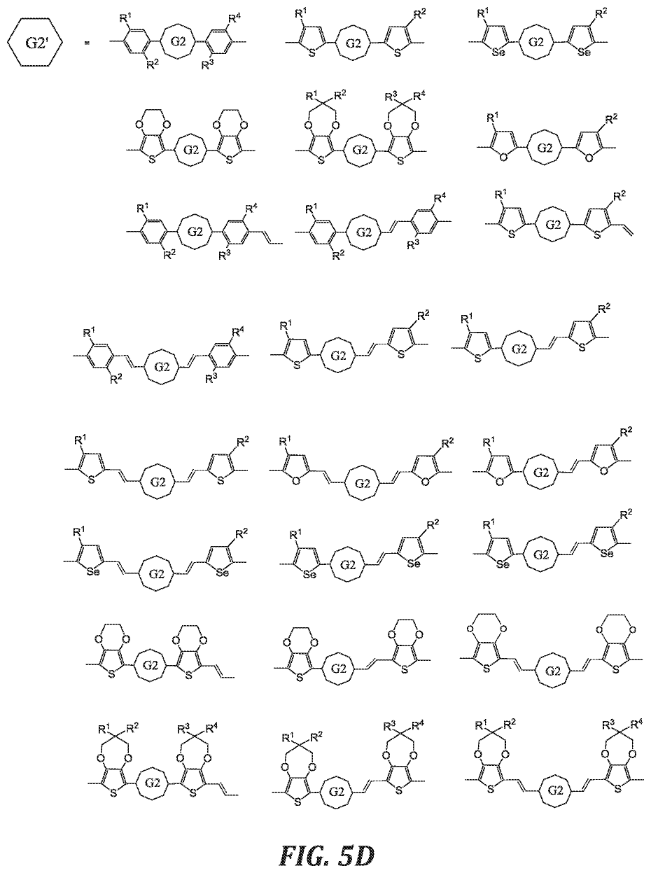

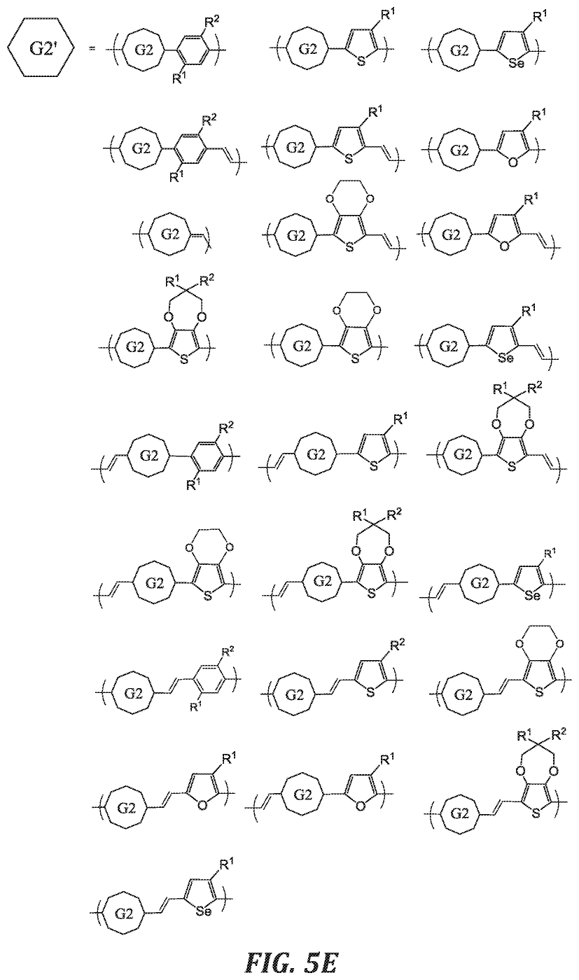

In certain embodiments, a polymer can include one or more types of general monomeric units. As shown in FIGS. 5A-E, three example types of general monomeric units are shown, G1, G2 and G2′. Each of the general G1 type monomeric units can be copolymerized with each of the G2 and G2′ type monomeric units and an emitting monomeric unit and/or absorbing monomeric unit to obtain an emitting polymer, an absorbing monomeric unit, and/or an emitting and absorbing polymer. Any of the G1 type monomeric units or G2 type monomeric units can also be separately used to copolymerize with an emitting monomeric unit and/or absorbing monomeric unit to obtain an emitting polymer, an absorbing monomeric unit, and/or an emitting and absorbing polymer. For the structures shown in FIG. 5A, a variety of substituents can be attached to the base structures. For example, each of R¹, R², R³, R³, R⁴, R⁵, and R⁶ can be independently selected from the group consisting of, but not limited to, hydrogen (H), deuterium (D), halogen, direct or branched alkyl, heteroalkyl, heterocycloalkyl, heterocycloalkylene, alkoxy, aryl, hydroxyl, cyano, nitro, ether and its derivatives, ester and its derivatives, alkyl ketone, alkyl esteralkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-) substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, alkyl-substituted carbazolyl, alkyl-substituted triphenylaminyl and alkyl-substituted thiophenyl. As exemplary embodiments, alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9,9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, 4-alkylthiophenyl, N-dialkyl-4-phenyl, N-diphenyl-4-phenyl, and N-dialkoxyphenyl-4-phenyl. The alkyl substituents can include CₙH₂ₙ₊₁, or CₙF₂ₙ₊₁ or —CH₂CH₂[OCH₂CH₂]ₙ—OCH₃ wherein n is 1 to 20. In some embodiments, n can be between 1 to 50 or higher. The general monomeric units can also be substituted with other substituents as defined herein. As shown in FIG. 5A, each of X, X¹, and X² can be independently selected from the group consisting of carbon (C), silicon (Si), and germanium (Ge). Z, Z¹, Z² can be selected from the group consisting of oxygen (O), sulfur (S), and selenium (Se).

FIG. 5B shows a non-limiting list of general donors in the absorbing polymers, emitting polymers, and/or absorbing and emitting polymers. As shown in the chemical structures of donors in FIG. 5B, each of X, X¹, X², X³, X⁴, Q, Z, Z¹, and Z² can be heteroatoms, and e.g., can be independently selected from the group consisting of O, S, Se, Te, N, and so on. Each of R¹ and R² is independently selected from non-limiting examples of hydrogen (H), deuterium (D), halogen, direct or branched alkyl, heteroalkyl, heterocycloalkyl, heterocycloalkylene, alkoxy, aryl, hydroxyl, cyano, nitro, ether and its derivatives, ester and its derivatives, alkyl ketone, alkyl esteralkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, alkyl-substituted carbazolyl, alkyl-substituted triphenylaminyl and alkyl-substituted thiophenyl. As exemplary embodiments, alkyl substituted phenyl can include 2-alkylphenyl, 3-alkylphenyl, 4-alkylphenyl, 2,4-dialkylphenyl, 3,5-dialkylphenyl, 3,4-dialkylphenyl, alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl.

In some embodiments, the general donors can be selected from (but are not limited to) the group shown in FIG. 5C, FIG. 5D, and FIG. 5E. As shown in the various G2 and G2′ structures in FIG. 5C, FIG. 5D, and FIG. 5E, each of R¹, R², R³, and R⁴ can be independently selected from non-limiting examples of hydrogen (H), deuterium (D), halogen, direct or branched alkyl, heteroalkyl, heterocycloalkyl, heterocycloalkylene, alkoxy, aryl, hydroxyl, cyano, nitro, ether and its derivatives, ester and its derivatives, alkyl ketone, alkyl esteralkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, alkyl-substituted carbazolyl, alkyl-substituted triphenylaminyl and alkyl-substituted thiophenyl. As exemplary embodiments, alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl, alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl.

### Properties of Narrow-Band Absorption Polymer Dots

In some embodiments, the chemical composition and structure of the chromophoric polymer in the polymer dots can affect the absorption spectrum of the narrow-band absorption Pdots. The absorption peak can shift from ultra-violet region to infrared region. In some embodiments, the absorption peak of the narrow-band absorption polymer dots can be tuned to a certain laser wavelength. In some embodiments, for example, the absorption peak can be tuned to 405 nm. In some embodiments, the absorption peak can be tuned to around 450 nm. In some embodiments, the absorption peak can be tuned to around 488 nm. In some embodiments, the absorption peak can be tuned to around 532 nm. In some embodiments, the absorption peak can be tuned to around 561 nm. In some embodiments, the absorption peak can be tuned to around 633 nm. In some embodiments, the absorption peak can be tuned to around 635 nm. In some embodiments, the absorption peak can be tuned to around 640 nm. In some embodiments, the absorption peak can be tuned to around 655 nm. In some embodiments, the absorption peak can be tuned to around 700 nm. In some embodiments, the absorption peak can be tuned to around 750 nm. In some embodiments, the absorption peak can be tuned to around 800 nm. In some embodiments, the absorption peak can be tuned to around 850 nm. In some embodiments, the absorption peak can be tuned to around 900 nm. In some embodiments, the absorption peak can be tuned to around 980 nm. In some embodiments, the absorption peak can be tuned to the near-infrared region of the wavelength spectrum (e.g., from 750 nm to 1200 nm). In some embodiments, the absorption peak can be tuned to around 1064 nm. In some embodiments, for example, the absorption peak can be tuned to between 380 and 420 nm. In some embodiments, the absorption peak can be tuned to between 440 and 460 nm. In some embodiments, the absorption peak can be tuned to between 478 and 498 nm. In some embodiments, the absorption peak can be tuned to between 522 and 542 nm. In some embodiments, the absorption peak can be tuned to between 550 and 570 nm. In some embodiments, the absorption peak can be tuned to between 625 and 645 nm. In some embodiments, the absorption peak can be tuned to between 645 and 665 nm. In some embodiments, the absorption peak can be tuned to between 690 and 710 nm. In some embodiments, the absorption peak can be tuned to between 740 and 760 nm. In some embodiments, the absorption peak can be tuned to between 790 and 810 nm. In some embodiments, the absorption peak can be tuned to between 890 and 910 nm. In some embodiments, the absorption peak can be tuned to between 970 and 990 nm. In some embodiments, the absorption peak can be tuned to between 1054 and 1074 nm.

In certain embodiments, the chemical composition and structure of the polymer in the polymer dots can affect the fluorescence quantum yield of the narrow-band absorption Pdots. The fluorescence quantum yield, for example, can vary from 100% to 0.1%. In some embodiments, the quantum yield is greater than 90%. In some embodiments, the quantum yield is greater than 80%. In some embodiments, the quantum yield is greater than 70%. In some embodiments, the quantum yield is greater than 60%. In some embodiments, the quantum yield is greater than 50%. In some embodiments, the quantum yield is greater than 45%. In some embodiments, the quantum yield is greater than 40%. In some embodiments, the quantum yield is greater than 35%. In some embodiments, the quantum yield is greater than 30%. In some embodiments, the quantum yield is greater than 25%. In some embodiments, the quantum yield is greater than 20%. In some embodiments, the quantum yield is greater than 15%. In some embodiments, the quantum yield is greater than 10%. In some embodiments, the quantum yield is greater than 5%. In some embodiments, the quantum yield is greater than 1%.

A narrow band absorption nanoparticle can have an absorbance width measured at a percent value of the absorbance maximum. For example, the nanoparticle can have an absorbance width of less than 150 nm at 10% (or in some embodiments, at 15%) of the absorbance maximum.

In certain embodiments the nanoparticle absorbance width is measured at from 20% to 16% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 20% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 19% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 18% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 17% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 16% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 15% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 14% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 13% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 12% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 11% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 10% of the absorbance maximum.

In certain embodiments the nanoparticle absorbance width is measured at from 15% to 11% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 15% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 14% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 13% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 12% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 11% of the absorbance maximum.

In certain embodiments the nanoparticle absorbance width is measured at from 10% to 6% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 10% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 9% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 8% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 7% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 6% of the absorbance maximum.

In certain embodiments the nanoparticle absorbance width is measured at from 5% to 1% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 5% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 4% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 3% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 2% of the absorbance maximum. In some embodiments the nanoparticle has an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 1% of the absorbance maximum.

In certain embodiments the nanoparticle absorbance width is measured at from 20% to 16% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 20% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 19% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 18% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 17% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 16% of the absorbance maximum.

In certain embodiments the nanoparticle absorbance width is measured at from 15% to 11% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 15% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 14% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 13% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 12% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 11% of the absorbance maximum.

In certain embodiments the nanoparticle absorbance width is measured at from 10% to 6% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 10% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 9% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 8% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 7% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 6% of the absorbance maximum.

In certain embodiments the nanoparticle absorbance width is measured at from 5% to 1% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 5% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 4% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 3% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 2% of the absorbance maximum. In some embodiments the nanoparticle has an absorbance width from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm at 1% of the absorbance maximum.

In some embodiments, the absorbance width at half absorbance maximum (full width half max, FWHM) of the nanoparticle is from 10 nm to 200 nm, from 50 nm to 200 nm, from 80 nm to 100 nm, from 100 nm to 200 nm, from 120 nm to 200 nm, from 150 nm to 200 nm, from 10 nm to 150 nm, from 50 nm to 150 nm, from 80 nm to 150 nm, from 90 nm to 150 nm, from 100 nm to 150 nm, from 50 nm to 140 nm, from 50 nm to 130 nm, from 50 nm to 120 nm, from 50 nm to 110 nm, from 50 nm to 100 nm, from 50 nm to 90 nm, from 50 nm to 80 nm, from 40 nm to 80 nm, from 30 nm to 70 nm, from 30 nm to 60 nm, or from 10 nm to 50 nm. In some embodiments, the absorbance width at half absorbance maximum of the nanoparticle is less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, less than 40 nm, or less than 30 nm.

In some embodiments, the narrow-band absorption Pdots can have a secondary absorption peak. In certain embodiments, the secondary absorption peak is distinct from the main absorption peak (i.e., the absorbance curves do not overlap significantly). The secondary absorption peak can have an decreased wavelength value compared to the main absorption peak (i.e., the secondary peak wavelength is shorter than the main absorption peak). In certain embodiments, the main absorption peak is the absorption peak with the highest absorbance in the region from 380 nm to 1200 nm. In some embodiments, the secondary absorption peak is in the ultraviolet region. In specific embodiments, the secondary absorption peak has a wavelength value of shorter than 350 nm. In other specific embodiments, the secondary absorption peak has a wavelength value of longer than 380 nm. For example, when the absorbing monomeric units are copolymerized with other absorbing units to produce narrow-band absorption Pdots, the final Pdots can have a secondary peak because of incomplete absorption by the absorbing monomeric unit. In some embodiments, the narrow-band absorbing Pdots can also have a secondary peak in the composite Pdot chemically cross-linked with fluorescent dyes (e.g., fluorescent polymers and/or fluorescent small molecules), metal complexes, etc. Besides the narrow absorption peak, the secondary peak in the Pdots can be less than 30% of the maximum intensity of the main narrow-band absorption. In some embodiments, the secondary peak in the Pdots is less than 25% of the maximum intensity of the main narrow-band absorption. In some embodiments, the secondary peak in the Pdots is less than 20% of the maximum intensity of the main narrow-band absorption. In some embodiments, the secondary peak in the Pdots is less than 15% of the maximum intensity of the main narrow-band absorption. In some embodiments, the secondary peak in the Pdots is less than 10% of the maximum intensity of the main narrow-band absorption. In some embodiments, the secondary peak in the Pdots is less than 5% of the maximum intensity of the main narrow-band absorption, or less.

In certain embodiments, the emission qualities of the polymer dots can be manipulated. The emission wavelength of the polymer dots can vary from ultraviolet to the near infrared region. The chromophoric polymer dot includes at least one chromophoric polymer. As provided herein, the chemical composition and structure of the polymer can be tuned to obtain small bandwidth (FWHM) of the Pdot emission. Other species such as narrow-band emissive units, metal complexes or inorganic materials can be blended or chemically cross linked within the chromophoric polymer dots to obtain small bandwidth (FWHM) of the Pdot emission. In some embodiments, the FWHM is less than about 100 nm. In some embodiments, the FWHM is less than about 90 nm. In some embodiments, the FWHM is less than about 80 nm. In some embodiments, the FWHM is less than about 70 nm. In some embodiments, the FWHM is less than about 65 nm. In some embodiments, the FWHM is less than about 60 nm. In some embodiments, the FWHM is less than about 55 nm. In some embodiments, the FWHM is less than about 50 nm. In some embodiments, the FWHM is less than about 45 nm. In some embodiments, the FWHM is less than about 40 nm. In some embodiments, the FWHM is less than about 35 nm. In some embodiments, the FWHM is less than about 30 nm. In some embodiments, the FWHM is less than about 25 nm. In certain embodiments, the FWHM is less than about 24 nm, 23 nm, 22 nm, 21 nm, 20 nm, 19 nm, 18 nm, 17 nm, 16 nm, 15 nm, 14 nm, 13 nm, 12 nm, 11 nm, 10 nm, or less. In some embodiments, the FWHM of the polymer dots described herein can range between about 5 nm to about 70 nm, from about 10 nm to about 60 nm, from about 20 nm to about 50 nm, or from about 30 nm to about 45 nm.

In certain embodiments, the chemical composition and structure of the polymer in the polymer dots can affect the fluorescence lifetime of the narrow-band absorption Pdots. The fluorescence lifetime can vary from 10 ps to 1 ms. In some embodiments, the fluorescence lifetime varies from 10 ps to 100 ps. In some embodiments, the fluorescence lifetime varies from 100 ps to 1 ns. In some embodiments, the fluorescence lifetime varies from 1 ns to 10 ns. In some embodiments, the fluorescence lifetime varies from 10 ns to 100 ns. In some embodiments, the fluorescence lifetime varies from 100 ns to 1 ps. In some embodiments, the fluorescence lifetime varies from 1 ps to 10 ps. In some embodiments, the fluorescence lifetime varies from 10 ps to 100 ps. In some embodiments, the fluorescence lifetime varies from 100 ps to 1 ms.

In certain embodiments, the narrow-band absorption Pdots can be characterized by their stability. The optical properties (e.g. absorption spectrum, absorption bandwidth, absorption peak, emission spectrum, emission band width, fluorescence quantum yield, fluorescence lifetime, side peaks, brightness at the particular wavelength or emission intensity at a particular wavelength) can be stable for over 1 day, or 1 week, or 2 weeks, or 1 month, or 2 months, or 3 months, or 6 months, or 1 year, or longer. The stable fluorescence quantum yield means that the fluorescence quantum yield of the emission does not change by more than 5%, or 10%, or 20%, or 50%, or higher. The stable absorption spectrum means that the width of the main peak doesn't change by more than 5%, 10%, or 20%. The stable emission spectrum means that the width of the main peak doesn't change by more than 5%, 10%, or 20%.

In some embodiments, the narrow-band absorbing nanoparticle has a hydrodynamic diameter of less than 1000 nm, less than 900 nm, less than 800 nm, less than 700 nm, less than 600 nm, less than 500 nm, less than 400 nm, less than 300 nm, less than 200 nm, less than 150 nm, less than 100 nm, less than 90 nm, less than 80 nm, less than 70 nm, less than 60 nm, less than 50 nm, less than 40 nm, less than 30 nm, less than 20 nm, or less than 10 nm as measured by dynamic light scattering. In some aspects, the narrow-band absorbing nanoparticle has a critical dimension of greater than 3 nm and less than 1000 nm, greater than 10 nm and less than 1000 nm, greater than 20 nm and less than 1000 nm, greater than 30 nm and less than 1000 nm, greater than 40 nm and less than 1000 nm, greater than 50 nm and less than 1000 nm, greater than 3 nm and less than 100 nm, greater than 3 nm and less than 90 nm, greater than 3 nm and less than 80 nm, greater than 3 nm and less than 70 nm, greater than 3 nm and less than 60 nm, greater than 3 nm and less than 50 nm, greater than 3 nm and less than 40 nm, greater than 3 nm and less than 30 nm, greater than 3 nm and less than 20 nm, or greater than 3 nm and less than 10 nm.

In some embodiments, the narrow-band absorbing nanoparticle has a quantum yield of greater than 5%, greater than 10%, greater than 15%, greater than 20%, greater than 25%, greater than 30%, greater than 35%, greater than 40%, greater than 50%, greater than 55%, greater than 60%, greater than 65%, greater than 70%, greater than 75%, greater than 80%, greater than 85%, greater than 90%, or greater than 95%. In certain embodiments, the narrow-band absorbing nanoparticle has a quantum yield of from 0.10 to 1.00, from 0.10 to 0.90, from 0.10 to 0.75, from 0.10 to 0.50, from 0.25 to 1.00, from 0.25 to 0.90, from 0.25 to 0.75, from 0.25 to 0.50, from 0.50 to 1.00, from 0.50 to 0.90, or from 0.50 to 0.75. In some embodiments, the narrow-band absorbing nanoparticle has a quantum yield of greater than 0.1, greater than 0.2, greater than 0.3, greater than 0.4, greater than 0.5, greater than 0.6, greater than 0.7, greater than 0.8, or greater than 0.9. In certain embodiments, the quantum yield is measured from 400 nm to 900 nm.

In certain embodiments, a low mass percentage of the absorbing monomeric unit in the narrow-band absorbing nanoparticle is beneficial. In some embodiments, the absorbing monomeric unit is less than 50% of the total mass of the nanoparticle, less than 40% of the total mass of the nanoparticle, less than 30% of the total mass of the nanoparticle, less than 25% of the total mass of the nanoparticle, less than 20% of the total mass of the nanoparticle, less than 15% of the total mass of the nanoparticle, less than 14% of the total mass of the nanoparticle, less than 13% of the total mass of the nanoparticle, less than 12% of the total mass of the nanoparticle, less than 11% of the total mass of the nanoparticle, less than 10% of the total mass of the nanoparticle, less than 9% of the total mass of the nanoparticle, less than 8% of the total mass of the nanoparticle, less than 7% of the total mass of the nanoparticle, less than 6% of the total mass of the nanoparticle, less than 5% of the total mass of the nanoparticle, less than 4% of the total mass of the nanoparticle, less than 3% of the total mass of the nanoparticle, less than 2% of the total mass of the nanoparticle, or less than 1% of the total mass of the nanoparticle.

In other embodiments, a high mass percentage of the absorbing monomeric unit in the narrow-band absorbing nanoparticle is beneficial. In some embodiments, the absorbing monomeric unit is greater than 1% of the total mass of the nanoparticle, greater than 2% of the total mass of the nanoparticle, greater than 3% of the total mass of the nanoparticle, greater than 4% of the total mass of the nanoparticle, greater than 5% of the total mass of the nanoparticle, greater than 6% of the total mass of the nanoparticle, greater than 7% of the total mass of the nanoparticle, greater than 8% of the total mass of the nanoparticle, greater than 9% of the total mass of the nanoparticle, greater than 10% of the total mass of the nanoparticle, greater than 11% of the total mass of the nanoparticle, greater than 12% of the total mass of the nanoparticle, greater than 13% of the total mass of the nanoparticle, greater than 14% of the total mass of the nanoparticle, greater than 15% of the total mass of the nanoparticle, greater than 20% of the total mass of the nanoparticle, greater than 25% of the total mass of the nanoparticle, greater than 30% of the total mass of the nanoparticle, greater than 40% of the total mass of the nanoparticle, greater than 50% of the total mass of the nanoparticle, greater than 60% of the total mass of the nanoparticle, or greater than 70% of the total mass of the nanoparticle.

In some embodiments, the emitting monomeric units emit luminescent light following absorption of energy, which excites electrons within the monomeric unit, and results in the emission of a photon of light. In certain embodiments, the energy comes from intrachain or interchain energy transfer. For example, the absorbing monomeric unit can be excited by an external emission (e.g., a laser beam); the excited absorbing monomeric unit can then transfer energy intra-chain to general monomeric units, inter-chain to general monomeric units, intra-chain to emitting monomeric units, and/or inter-chain to emitting monomeric units. The general monomeric units can further transfer energy intra-chain or inter-chain to emitting monomeric units. In certain embodiments, the energy transfer includes FRET. In some embodiments, the energy transfer includes inter-chain energy transfer. In certain embodiments, the energy transfer includes through-bond energy transfer.

In some embodiments, it is beneficial to have a low ratio of the number of absorbing monomeric units in the narrow-band absorbing monomeric unit in comparison to the number of emitting monomeric units. Without being limited to a particular theory or concept, a high number of emitting monomeric units can provide increased brightness, and can allow for better signal identification or interpretation (e.g., if an absorbing monomeric unit and/or general monomeric units are particularly efficient at absorption and/or energy transfer, a plethora of emitting monomeric units can provide for several distinct luminescent signals, or a stronger individual signal). As a non-limiting example, a narrow-band absorbing monomeric unit including 3 absorbing monomeric units and 15 emitting monomeric units has a ratio of the absorbing monomeric unit to the emitting monomeric unit of 1:5. In certain embodiments, the narrow-band absorbing nanoparticle has a ratio of the absorbing monomeric unit to the emitting monomeric unit of less than 1:1, less than 1:2, less than 1:3, less than 1:4, less than 1:5, less than 1:6, less than 1:7, less than 1:8, less than 1:9, less than 1:10, less than 1:11, less than 1:12, less than 1:13, less than 1:14, less than 1:15, less than 1:16, less than 1:17, less than 1:18, less than 1:19, less than 1:20, less than 1:25, less than 1:30, less than 1:35, less than 1:40, less than 1:50, less than 1:60, less than 1:70, less than 1:80, less than 1:90, or less than 1:100.

In other embodiments, it is beneficial to have a high ratio of the number of absorbing monomeric units in the narrow-band absorbing monomeric unit in comparison to the number of emitting monomeric units. Without being limited to a particular theory or concept, a high number of absorbing monomeric units can improve brightness by increasing absorption cross section and can allow for better signal identification or interpretation (e.g., if an absorbing monomeric unit and/or general monomeric units are not efficient at absorption and/or energy transfer, a plethora of absorbing monomeric units can improve luminescent signal by increasing the number of excitation points within the nanoparticle). As a non-limiting example, a narrow-band absorbing monomeric unit including 15 absorbing monomeric units and 3 emitting monomeric units has a ratio of the absorbing monomeric unit to the emitting monomeric unit of 5:1. In certain embodiments, the narrow-band absorbing nanoparticle has a ratio of the absorbing monomeric unit to the emitting monomeric unit of greater than 1:1, greater than 2:1, greater than 3:1, greater than 4:1, greater than 5:1, greater than 6:1, greater than 7:1, greater than 8:1, greater than 9:1, greater than 10:1, greater than 11:1, greater than 12:1, greater than 13:1, greater than 14:1, greater than 15:1, greater than 16:1, greater than 17:1, greater than 18:1, greater than 19:1, greater than 20:1, greater than 25:1, greater than 30:1, greater than 35:1, greater than 40:1, greater than 50:1, greater than 60:1, greater than 70:1, greater than 80:1, greater than 90:1, or greater than 100:1.

In certain embodiments, the narrow-band absorbing nanoparticle emits a bright signal, the brightness of which can be calculated as the product of quantum yield and absorption cross-section. In some embodiments, the narrow-band absorbing nanoparticle has a brightness of greater than 1.0×10⁻¹⁶ cm², greater than 1.0×10⁻¹⁵ cm², greater than 1.0×10⁻¹⁴ cm², greater than 1.0×10⁻¹³ cm², greater than 1.0×10⁻¹² cm², greater than 1.0×10⁻¹¹ cm², greater than 1.0×10⁻¹⁰ cm², greater than 1.0×10⁻⁹ cm², greater than 1.0×10⁻⁸ cm², greater than 1.0×10⁻⁷ cm², greater than 1.0×10⁻⁶ cm², greater than 1.0×10⁻⁵ cm², or greater than 1.0×10⁻⁴ cm².

In some embodiments, the narrow-band absorbing nanoparticle has a brightness of greater than 1.0×10⁻¹³ cm², greater than 2.0×10⁻¹³ cm², greater than 3.0×10⁻¹³ cm², greater than 4.0×10⁻¹³ cm², greater than 5.0×10⁻¹³ cm², greater than 6.0×10⁻¹³ cm², greater than 7.0×10⁻¹³ cm², greater than 8.0×10⁻¹³ cm², greater than 9.0×10⁻¹³ cm², greater than 1.0×10⁻¹² cm², greater than 2.0×10⁻¹² cm², greater than 3.0×10⁻¹² cm², greater than 4.0×10⁻¹² cm², greater than 5.0×10⁻¹² cm², greater than 6.0×10⁻¹² cm², greater than 7.0×10⁻¹² cm², greater than 8.0×10⁻¹² cm², greater than 9.0×10⁻¹² cm², greater than 1.0×10⁻¹¹ cm², greater than 2.0×10⁻¹¹ cm², greater than 3.0×10⁻¹¹ cm², greater than 4.0×10⁻¹¹ cm², greater than 5.0×10⁻¹¹ cm², greater than 6.0×10⁻¹¹ cm², greater than 7.0×10⁻¹¹ cm², greater than 8.0×10⁻¹¹ cm², or greater than 9.0×10⁻¹¹ cm². In some embodiments, the narrow-band absorbing nanoparticle has a brightness from 1.0×10⁻¹⁴ cm² to 1.0×10⁻¹³ cm². In some embodiments, the narrow-band absorbing nanoparticle has a brightness from 1.0×10⁻¹³ cm² to 1.0×10⁻¹² cm². In some embodiments, the narrow-band absorbing nanoparticle has a brightness from 1.0×10⁻¹² cm² to 1.0×10⁻¹¹ cm².

In some embodiments, the narrow-band absorbing nanoparticles have a high brightness, calculated as the product of emission quantum yield and absorption cross-section (i.e., brightness=Φ_{PL}×σ). In some embodiments, the brightness is greater than 1.0×10⁻¹ cm², greater than 1.0×10⁻¹⁴ cm², greater than 1.0×10⁻¹³ cm², greater than 1.0×10⁻¹² cm², greater than 1.0×10⁻¹¹ cm², greater than 1.0×10⁻¹⁰ cm², or greater than 1.0×10⁻⁹ cm². In certain embodiments, the brightness is from 1.0×10⁻¹⁵ cm² to 1.0×10⁻⁹ cm². In certain embodiments, the brightness is from 1.0×10⁻¹⁴ cm² to 1.0×10⁻¹⁰ cm². In certain embodiments, the brightness is from 1.0×10⁻¹⁴ cm² to 1.0×10⁻¹¹ cm². In certain embodiments, the brightness is from 1.0×10⁻¹⁴ cm² to 1.0×10⁻¹² cm². In certain embodiments, the brightness is from 1.0×10⁻¹³ cm² to 1.0×10⁻¹² cm². In certain embodiments, the brightness is from 1.0×10⁻¹⁵ cm² to 1.0×10⁻¹⁴ cm². In certain embodiments, the brightness is from 1.0×10⁻¹⁴ cm² to 1.0×10⁻¹³ cm². In certain embodiments, the brightness is from 1.0×10⁻¹³ cm² to 1.0×10⁻¹² cm². In certain embodiments, the brightness is from 1.0×10⁻¹² cm² to 1.0×10⁻¹¹ cm². For example, a polymer nanoparticle can have a brightness of 2.0×10⁻¹³ cm².

In specific embodiments, the narrow-band absorbing nanoparticle includes at least one characteristic selected from each of (a), (b), and (c):

(a) an absorbance width of less than 200 nm, less than 190 nm, less than 180 nm, less than 170 nm, less than 160 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, or less than 40 nm at 10% (or at 15%, in some embodiments) of the absorbance maximum;

(b) a quantum yield of greater than 5%, greater than 10%, greater than 15%, greater than 20%, greater than 25%, greater than 30%, greater than 35%, greater than 40%, greater than 50%, greater than 55%, greater than 60%, greater than 65%, greater than 70%, greater than 75%, greater than 80%, greater than 85%, greater than 90%, or greater than 95%; and

(c) a brightness of greater than 1.0×10⁻¹⁶ cm², greater than 1.0×10⁻¹⁵ cm² greater than 1.0×10⁻¹⁴ cm², greater than 1.0×10⁻¹³ cm², greater than 1.0×10⁻¹² cm², greater than 1.0×10⁻¹¹ cm², greater than 1.0×10⁻¹⁰ cm², greater than 1.0×10⁻⁹ cm², greater than 1.0×10⁻⁸ cm², greater than 1.0×10⁻⁷ cm², greater than 1.0×10⁻⁶ cm², greater than 1.0×10⁻⁵ cm², or greater than 1.0×10⁻⁴ cm².

In some embodiments, the emitting polymers (i.e., polymers including emitting monomeric units) can exhibit broad-band emission in a good solvent, such as some hydrophobic polymers in tetrahydrofuran solution. However, after forming these polymers into Pdot nanoparticles in water, the nanoparticles exhibit narrow-band emission. In a good solvent, hydrophobic semiconducting polymers typically adopt an extended rod-like conformation, and the inter-chain energy transfer is not efficient. When the polymers are densely packed into a compact nanoparticle, because intra-particle energy transfer and inter-chain energy transfer are much more efficient in the nanoparticle form, therefore the resulting Pdots have narrow-band emission.

In some embodiments, the emitting polymers (i.e. polymers including emitting monomeric units) can have narrow emissions in a good solvent, such as some hydrophobic polymer in toluene solution. After forming these polymers into nanoparticles in water using nanoprecipitation, however, the Pdots exhibit broad emissions because of the complex backbone folding behaviors, disordered morphologies and chain aggregation. The Pdots can be prepared using a miniemulsion method, which can maintain the narrow emission from the polymer.

In certain embodiments, the narrow-band absorbing nanoparticles have a high energy transfer efficiency. In some embodiments, the energy transfer efficiency can be estimated, as disclosed further herein. In some embodiments, the estimated energy transfer efficiency from an absorbing polymer to an emitting polymer is greater than 99%, greater than 98%, greater than 97%, greater than 96%, greater than 95%, greater than 94%, greater than 93%, greater than 92%, greater than 91%, greater than 90%, greater than 89%, greater than 88%, greater than 87%, greater than 86%, greater than 85%, greater than 84%, greater than 83%, greater than 82%, greater than 81%, greater than 80%, greater than 75%, greater than 70%, greater than 65%, greater than 60%, greater than 55%, or greater than 50%. In some embodiments, the estimated energy transfer efficiency from an absorbing monomeric unit to an emitting monomeric unit is greater than 99%, greater than 98%, greater than 97%, greater than 96%, greater than 95%, greater than 94%, greater than 93%, greater than 92%, greater than 91%, greater than 90%, greater than 89%, greater than 88%, greater than 87%, greater than 86%, greater than 85%, greater than 84%, greater than 83%, greater than 82%, greater than 81%, greater than 80%, greater than 75%, greater than 70%, greater than 65%, greater than 60%, greater than 55%, or greater than 50%.

In some embodiments, the narrow-band absorbing nanoparticles have a high molar attenuation coefficient (i.e., molar extinction coefficient, molar absorptivity). The molar attenuation coefficient is a measurement of how strongly the nanoparticles attenuate light at a given wavelength. In certain embodiments, the molar attenuation coefficient is measured at 380 nm, at 405 nm, at 450 nm, at 488 nm, at 532 nm, at 561 nm, at 633 nm, at 640 nm, at 655 nm, at 700 nm, at 750 nm, at 800 nm, at 900 nm, at 980 nm, or at 1064 nm. In some embodiments, the molar attenuation coefficient is measured at a value from 380 nm to 1200 nm. In certain embodiments, the molar attenuation coefficient can be greater than 1.0×10⁵ M⁻¹ cm⁻¹, greater than 1.0×10⁶ M⁻¹ cm⁻¹, greater than 1.0×10⁷ M⁻¹ cm⁻¹, greater than 1.0×10⁸ M⁻¹ cm⁻¹, greater than 1.0×10⁹ M⁻¹ cm⁻¹, or greater than 1.0×10¹⁰ M⁻¹ cm⁻¹. In some embodiments, the molar attenuation coefficient can be from 1.0×10⁵ M⁻¹ cm⁻¹ to 1.0×10⁶ M⁻¹ cm⁻¹. In some embodiments, the molar attenuation coefficient can be from 1.0×10⁶ M⁻¹ cm⁻¹ to 1.0×10⁷ M⁻¹ cm⁻¹. In some embodiments, the molar attenuation coefficient can be from 1.0×10⁷ M⁻¹ cm⁻¹ to 1.0×10⁸ M⁻¹ cm⁻¹. In some embodiments, the molar attenuation coefficient can be from 1.0×10⁸ M⁻¹ cm⁻¹ to 1.0×10⁹ M⁻¹ cm⁻¹. As a non-limiting example, the molar attenuation coefficient of a polymer nanoparticle can be measured at 532 nm, and provide a value of 2.0×10⁸ M⁻¹ cm⁻¹.

In certain embodiments, the narrow-band absorbing nanoparticles have a high cross-section absorbance (also referred to herein as the “absorption cross-section”). The cross-section absorbance can be represented by “a”. In certain embodiments, the cross-section absorbance is measured at 380 nm, at 405 nm, at 450 nm, at 488 nm, at 532 nm, at 561 nm, at 633 nm, at 640 nm, at 655 nm, at 700 nm, at 750 nm, at 800 nm, at 900 nm, at 980 nm, or at 1064 nm. In some embodiments, the absorption cross-section is greater than 1.0×10⁻¹⁵ cm², greater than 1.0×10⁻¹⁴ cm², greater than 1.0×10⁻¹³ cm², greater than 1.0×10⁻¹² cm², greater than 1.0×10⁻¹¹ cm², or greater than 1.0×10⁻¹⁰ cm². In some embodiments, the absorption cross-section is from 1.0×10⁻¹⁵ cm² to 1.0×10⁻¹⁴ cm². In some embodiments, the absorption cross-section is from 1.0×10⁻¹⁴ cm² to 1.0×10⁻¹³ cm². In some embodiments, the absorption cross-section is from 1.0×10⁻¹³ cm² to 1.0×10⁻¹² cm². In some embodiments, the absorption cross-section is from 1.0×10⁻¹² cm² to 1.0×10⁻¹¹ cm². In some embodiments, the absorption cross-section is from 1.0×10⁻¹¹ cm² to 1.0×10⁻¹⁰ cm². In certain embodiments, the absorption cross-section is greater than 5.0×10⁻¹⁴ cm², greater than 1.0×10⁻¹³ cm², greater than 2.0×10⁻¹³ cm², greater than 3.0×10⁻¹³ cm², greater than 4.0×10⁻¹³ cm², greater than 5.0×10⁻¹³ cm², greater than 6.0×10⁻¹³ cm², greater than 7.0×10⁻¹³ cm², greater than 8.0×10⁻¹³ cm², greater than 9.0×10⁻¹³ cm², greater than 1.0×10⁻¹² cm², greater than 2.0×10⁻¹² cm², greater than 3.0×10⁻¹² cm², greater than 4.0×10⁻¹² cm², greater than 5.0×10⁻¹² cm², greater than 6.0×10⁻¹² cm², greater than 7.0×10⁻¹² cm², greater than 8.0×10⁻¹² cm², greater than 9.0×10⁻¹² cm², greater than 1.0×10⁻¹¹ cm², greater than 2.0×10⁻¹¹ cm², greater than 3.0×10⁻¹¹ cm², greater than 4.0×10⁻¹ cm², greater than 5.0×10⁻¹¹ cm², greater than 6.0×10⁻¹¹ cm², greater than 7.0×10⁻¹¹ cm², greater than 8.0×10⁻¹¹ cm², or greater than 9.0×10⁻¹¹ cm². As a non-limiting example, the absorption cross-section of a nanoparticle can be measured at 532 (“σ_{532 nm}”), and has a value of 1.0×10⁻¹² cm².

In certain embodiments, the narrow-band absorbing nanoparticle has a high brightness per volume of the nanoparticle. Brightness per volume can be calculated by dividing the brightness value by the volume of the nanoparticle (i.e., brightness per volume=(Φ_{PL}×σ)/V). In some embodiments, the brightness per volume is greater than 3,000 cm⁻¹, greater than 4,000 cm⁻¹, greater than 5,000 cm⁻¹, greater than 6,000 cm⁻¹, greater than 7,000 cm⁻¹, greater than 8,000 cm⁻¹, greater than 9,000 cm⁻¹, 6, greater than 10,000 cm⁻¹, greater than 11,000 cm⁻¹, greater than 12,000 cm⁻¹, greater than 13,000 cm⁻¹, greater than 14,000 cm⁻¹, greater than 15,000 cm⁻¹, greater than 16,000 cm⁻¹, greater than 17,000 cm⁻¹, greater than 18,000 cm⁻¹, greater than 19,000 cm⁻¹, greater than 20,000 cm⁻¹, greater than 25,000 cm⁻¹, greater than 30,000 cm⁻¹, greater than 35,000 cm⁻¹, greater than 40,000 cm⁻¹, greater than 45,000 cm⁻¹, greater than 50,000 cm⁻¹, greater than 60,000 cm¹, greater than 70,000 cm⁻¹, greater than 80,000 cm⁻¹, greater than 90,000 cm⁻¹, greater than 100,000 cm⁻¹, greater than 250,000 cm⁻¹, greater than 500,000 cm⁻¹, or greater than 1,000,000 cm⁻¹. In certain embodiments, the brightness per volume of a nanoparticle is from 5,000 cm⁻¹, to 100,000 cm⁻¹. In certain embodiments, the brightness per volume of a nanoparticle is from 10,000 cm⁻¹ to 90,000 cm⁻¹. In certain embodiments, the brightness per volume of a nanoparticle is from 20,000 cm⁻¹ to 80,000 cm⁻¹. In certain embodiments, the brightness per volume of a nanoparticle is from 30,000 cm⁻¹ to 70,000 cm⁻¹.In certain embodiments, the brightness per volume of a nanoparticle is from 30,000 cm⁻¹ to 60,000 cm⁻¹. In certain embodiments, the brightness per volume of a nanoparticle is from 30,000 cm⁻¹ to 50,000 cm⁻¹. For example, a polymer nanoparticle can have a brightness per volume of 40,000 cm⁻¹.

### Compositions of Narrow-Band Absorption Polymer Dots

As described further herein, the present disclosure includes a wide variety of polymer nanoparticles that exhibit narrow-band absorbing properties, and additionally exhibit emission properties. The polymer nanoparticles can include an absorbing polymer, an emitting polymer, an absorbing and emitting polymer, or any combination thereof. As described further herein, the variety of polymer dots of the present disclosure can include polymers that have an emissive unit (e.g., an emitting monomeric unit and/or an emitting unit). For example, the present disclosure can include a heteropolymer including an emitting monomeric unit, such as a BODIPY, a BODIPY derivative, a squaraine, a squaraine derivative, or any combination thereof. The present disclosure can include a heteropolymer including an emitting unit, such as a metal complex and/or metal complex derivative monomeric unit, a porphyrin and/or porphyrin derivative monomeric unit, a phthalocyanine and/or phthalocyanine derivative monomeric unit, a lanthanide complex and/or lanthanide complex derivative monomeric unit, a perylene and/or perylene derivative monomeric unit, a cyanine and/or cyanine derivative monomeric unit, a rhodamine and/or rhodamine derivative monomeric unit, a coumarin and/or coumarin derivative monomeric unit, and/or a xanthene and/or xanthene derivative monomeric unit. An emitting unit can be, e.g., an emitting monomeric unit or a fluorescent nanoparticle embedded in or attached to the polymer dot. The fluorescent nanoparticle can be, e.g., a quantum dot. An emitting unit can also include a polymer or fluorescent dye molecule that gives an emission in a polymer dot of the present disclosure.

As described further herein, the present disclosure includes a wide variety of polymer dots that exhibit absorption properties. As described further herein, the variety of polymer dots of the present disclosure can include polymers that have an absorption unit (e.g., an absorbing monomeric unit and/or an absorbing unit). For example, the present disclosure can include a heteropolymer including an absorbing monomeric unit, such as a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof. The present disclosure can include a heteropolymer including an absorbing unit, such as a metal complex and/or metal complex derivative monomeric unit, a porphyrin and/or porphyrin derivative monomeric unit, a phthalocyanine and/or phthalocyanine derivative monomeric unit, a perylene and/or perylene derivative monomeric unit, a cyanine and/or cyanine derivative monomeric unit, a rhodamine and/or rhodamine derivative monomeric unit, a coumarin and/or coumarin derivative monomeric unit, and/or a xanthene and/or xanthene derivative monomeric unit. An absorption unit can also include a polymer or fluorescent dye molecule that gives an absorption in a polymer dot of the present disclosure. In certain embodiments, the absorbing monomeric unit is a narrow-band absorbing monomeric unit.

The absorbing monomeric units can be integrated into a heteropolymer with other general monomeric units that can, e.g., act as energy donors. For example, the general monomeric units can include an absorption spectrum that is tuned to substantially overlap the emission spectrum of a narrow-band absorbing monomeric unit, thereby acting as an energy acceptor for the narrow-band absorbing monomeric unit. As another example, the general monomeric units can include an emission spectrum that is tuned to substantially overlap the absorption spectrum of an emitting monomeric unit, thereby acting as an energy donor for the emitting monomeric unit. The energy transfer, e.g., can occur along the backbone of a polymer (e.g., intrachain) or between multiple polymer backbones (e.g., interchain). In some embodiments, absorbing units can be attached (e.g., covalently attached) to a polymer backbone or sidechain of the polymer. For example, the absorbing unit can be attached to a general monomeric unit that can include an absorption spectrum that is tuned to substantially overlap the emission spectrum of a narrow-band absorbing unit, thereby acting as an energy acceptor for the narrow-band absorbing unit.

In some embodiments, the absorbing monomeric units can be integrated into a heteropolymer with energy transfer monomeric units. In certain embodiments, the narrow-band absorbing nanoparticle includes an energy transfer monomeric unit. Energy transfer monomeric units can have a large Stokes shift (i.e., the difference between the band maximum of the absorption peak and the emission peak. In certain embodiments, the energy transfer monomeric units have a Stokes shift of greater than 30 nm, greater than 40 nm, greater than 50 nm, greater than 60 nm, greater than 70 nm, greater than 80 nm, greater than 90 nm, greater than 100 nm, greater than 110 nm, greater than 120 nm, greater than 130 nm, greater than 140 nm, greater than 150 nm, greater than 175 nm, greater than 200 nm, greater than 225 nm, greater than 250 nm, greater than 275 nm, greater than 300 nm, greater than 320 nm, greater than 350 nm, greater than 375 nm, or greater than 400 nm. In some embodiments, the energy transfer monomeric units have a Stokes shift from 20 nm to 250 nm, from 30 nm to 200 nm, from 30 nm to 175 nm, from 30 nm to 150 nm, from 30 nm to 140 nm, from 30 nm to 130 nm, from 30 nm to 120 nm, from 30 nm to 110 nm, from 30 nm to 100 nm, from 40 nm to 200 nm, from 40 nm to 175 nm, from 40 nm to 150 nm, from 40 nm to 140 nm, from 40 nm to 130 nm, from 40 nm to 120 nm, from 40 nm to 110 nm, from 40 nm to 100 nm, from 50 nm to 200 nm, from 50 nm to 175 nm, from 50 nm to 150 nm, from 50 nm to 140 nm, from 50 nm to 130 nm, from 50 nm to 120 nm, from 50 nm to 110 nm, or from 50 nm to 100 nm. In specific embodiments, the energy transfer monomeric units can be general monomeric units as described herein.

The general monomeric units can include a wide variety of structures that are further described herein (e.g., G1, G2, G2′). In some embodiments, the general monomeric units can include, e.g., fluorene, a fluorene derivative, a phenyl vinylene, a phenyl vinylene derivative, a phenylene, a phenylene derivative, a benzothiazole, a benzothiazole derivative, a thiophene, a thiophene derivative, a carbazole fluorene, and/or a carbazole fluorene derivative. As also described herein, the various polymers used in the polymer dots can be combined in a variety of ways. For example, the polymers of the present disclosure can be chemically crosslinked and/or physically blended in the polymer dots. The polymers described herein can further include at least one functional group for, e.g., conjugation reactions, such as for bioconjugation reactions to antibodies or other biomolecules further described herein. The present disclosure further includes compositions including the polymer dots described herein. The compositions of the present disclosure can include, e.g., polymer dots described herein suspended in a solvent (e.g., an aqueous solution).

In some embodiments, the narrow-band absorption polymer dots include at least one narrow-band absorbing polymer. The narrow-band absorbing polymer can be a homopolymer or a heteropolymer (e.g., a copolymer). The narrow-band absorbing polymers can have broad-band absorptions in solvents. However, the final Pdots made from the narrow-band absorbing polymers have narrow-band absorptions.

In certain embodiments, the polymer dots can include luminescent semiconducting polymer with delocalized pi-electrons. The term “semiconducting polymer” is recognized in the art. Conventional luminescent semiconducting polymers include, but are not limited to fluorene polymers, phenylene vinylene polymers, phenylene polymers, benzothiadiazole polymers, thiophene polymers, carbazole polymers and related copolymers. While conventional semiconducting polymers typically have broad-band absorptions, narrow-band absorbing polymers include chemical units such as narrow-band absorbing monomeric units so that the final Pdots give narrow-band absorptions.

In some embodiments, the narrow-band absorbing polymers for making Pdots include narrow-band absorbing monomeric units. The narrow-band absorbing polymer dots can also include other monomeric units that are broad-band absorbing. The narrow-band absorbing monomeric units can be energy acceptors and other monomeric units can be energy donors. The narrow-band absorbing monomeric units can be energy donors and other monomeric units can be energy acceptors. For example, polymer dots of the present disclosure can include condensed polymer nanoparticles that have intrachain energy transfer between, e.g., a narrow-band absorbing monomeric unit and one or more general monomeric units on the same polymer chain. The polymer dots can also have interchain energy transfer in which a condensed polymer nanoparticle can include two or more polymer chains physically blended and/or chemically crosslinked together. For interchain energy transfer, one of the chains can include a narrow-band absorbing monomeric unit and another chain can include one or more general monomeric units that can act as an energy acceptor to the narrow band absorbing monomeric unit, which is an energy donor. Some polymer dots can include both intrachain and interchain energy transfer. In some instances, the combination of intrachain and interchain energy transfer can increase the quantum yield of the polymer dots. In some embodiments, the narrow-band absorbing Pdots are narrow band absorbing without relying on the formation of any defined secondary structures.

The compounds of the present disclosure can be prepared in a variety of ways known to one skilled in the art of organic synthesis. The compounds of the present disclosure can be synthesized using the methods as hereinafter described below, together with synthetic methods known in the art of synthetic organic chemistry or variations thereon as appreciated by those skilled in the art.

The compounds of this disclosure can be prepared from readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry; or by chromatography such as high performance liquid chromatography (HPLC) or thin layer chromatography. The compounds obtained by the reactions can be purified by any suitable method known in the art. For example, chromatography (medium pressure) on a suitable adsorbent (e.g., silica gel, alumina and the like) HPLC, or preparative thin layer chromatography; distillation; sublimation, trituration, or recrystallization.

Preparation of compounds can involve the protection and deprotection of various chemical groups. The need for protection and deprotection, and the selection of appropriate protecting groups can be readily determined by one skilled in the art. The chemistry of protecting groups can be found, for example, in Wuts and Greene, Greene's Protective Groups in Organic Synthesis, 4^{th}Ed., John Wiley & Sons: New York, 2006, which is incorporated herein by reference in its entirety.

The reactions of the processes described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the reaction step, suitable solvent(s) for that particular reaction step can be selected. Appropriate solvents include water, alkanes (such as pentanes, hexanes, heptanes, cyclohexane, etc., or a mixture thereof), aromatic solvents (such as benzene, toluene, xylene, etc.), alcohols (such as methanol, ethanol, isopropanol, etc.), ethers (such as dialkylethers, methyl tert-butyl ether (MTBE), tetrahydrofuran (THF), dioxane, etc.), esters (such as ethyl acetate, butyl acetate, etc.), halogenated solvents (such as dichloromethane (DCM), chloroform, dichloroethane, tetrachloroethane), dimethylformamide (DMF), dimethylsulfoxide (DMSO), acetone, acetonitrile (ACN), hexamethylphosphoramide (HMPA) and N-methylpyrrolidone (NMP). Such solvents can be used in either their wet or anhydrous forms.

Resolution of racemic mixtures of compounds can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art.

The compounds of the disclosure can be prepared, for example, using the reaction pathways and techniques describe in this disclosure, including the figures.

As will be appreciated by one of ordinary skill in the art, the various chemical terms defined herein can be used for describing chemical structures of the polymers and monomeric units of the present disclosure. For example, a variety of the monomeric unit derivatives (e.g., BODIPY derivatives, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof) can include a variety of the chemical substituents and groups described herein. For example, in some embodiments, derivatives of the various monomeric units can be substituted with hydrogen, deuterium, alkyl, alkyl-aryl, aryl, alkoxy-aryl, N-dialkyl-4-phenyl, N-diphenyl-4-phenyl, N-dialkoxyphenyl-4-phenyl, amino, sulfide, aldehyde, ester, ether, acid, and/or hydroxyl.

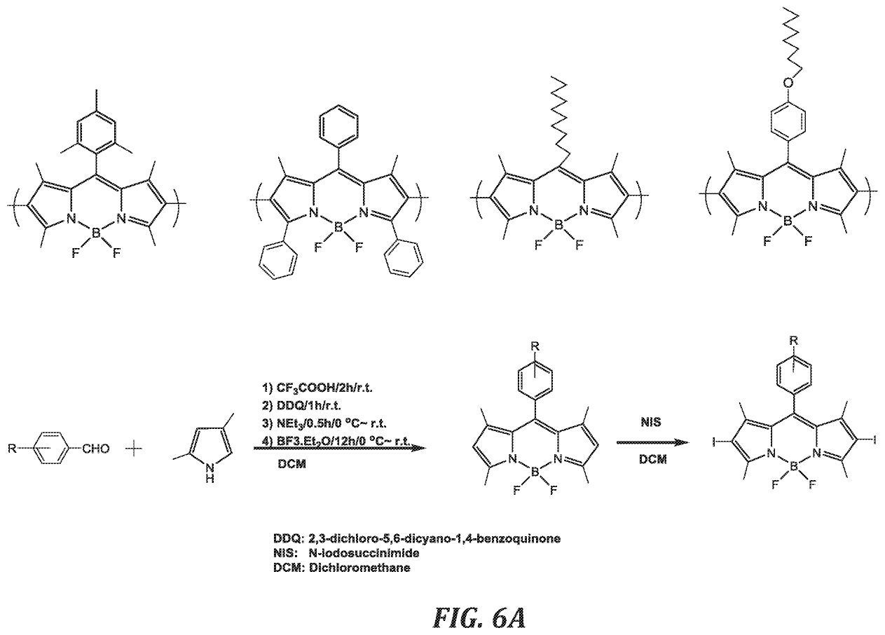

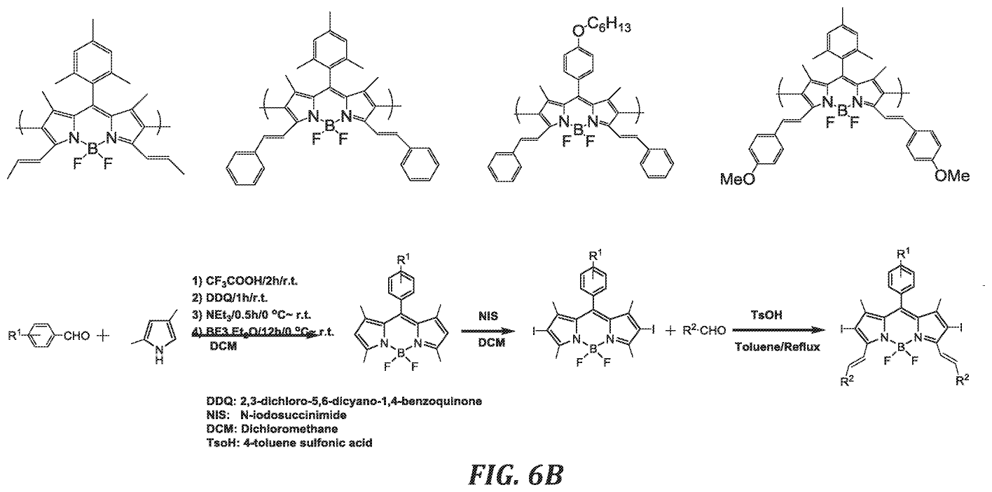

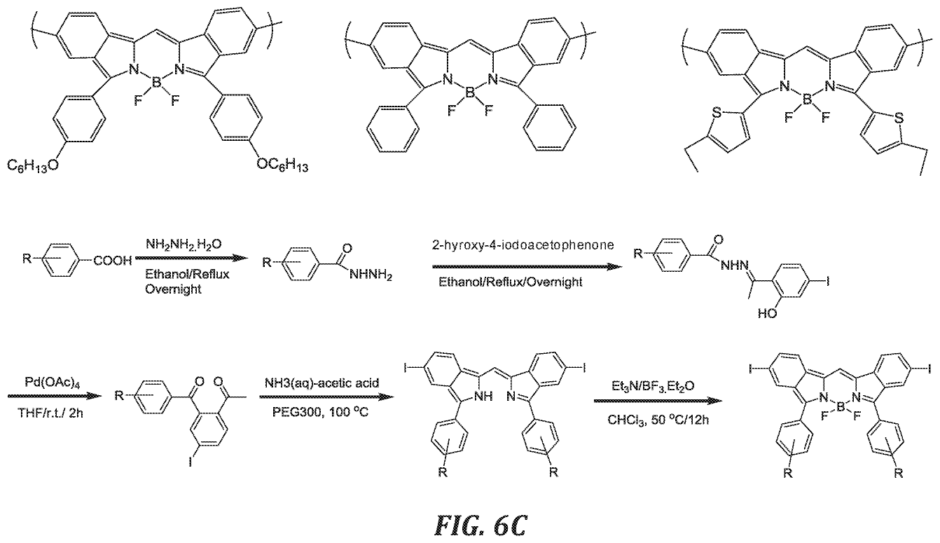

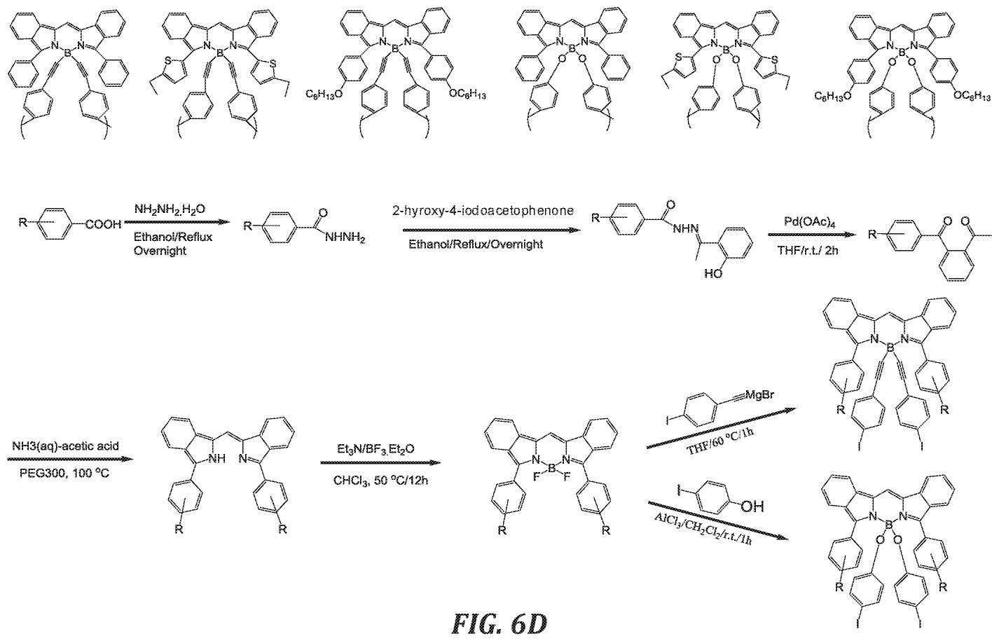

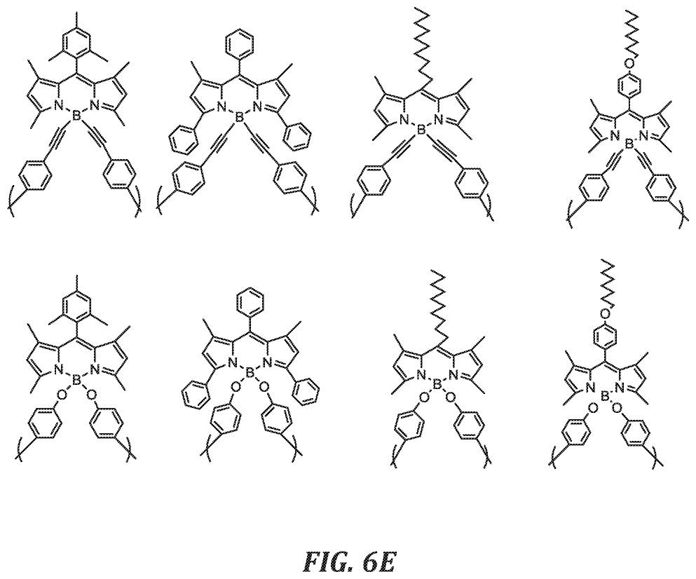

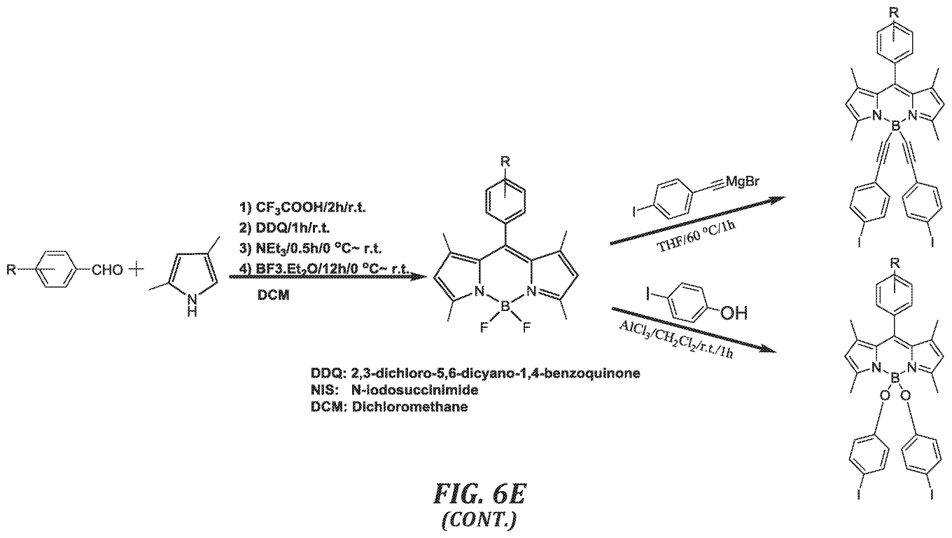

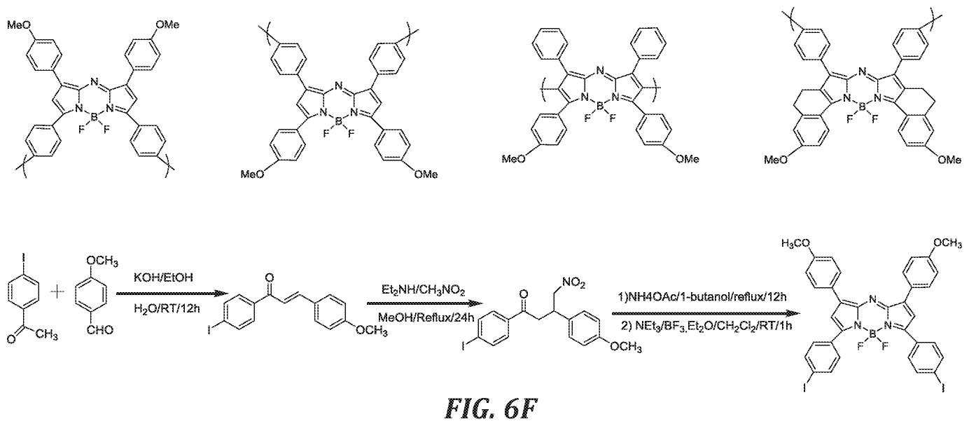

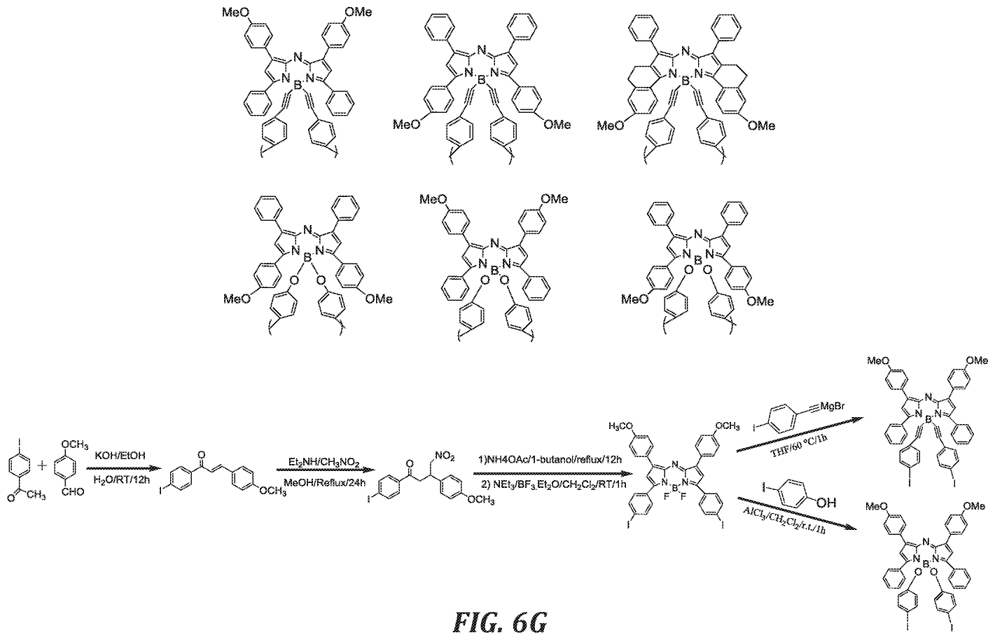

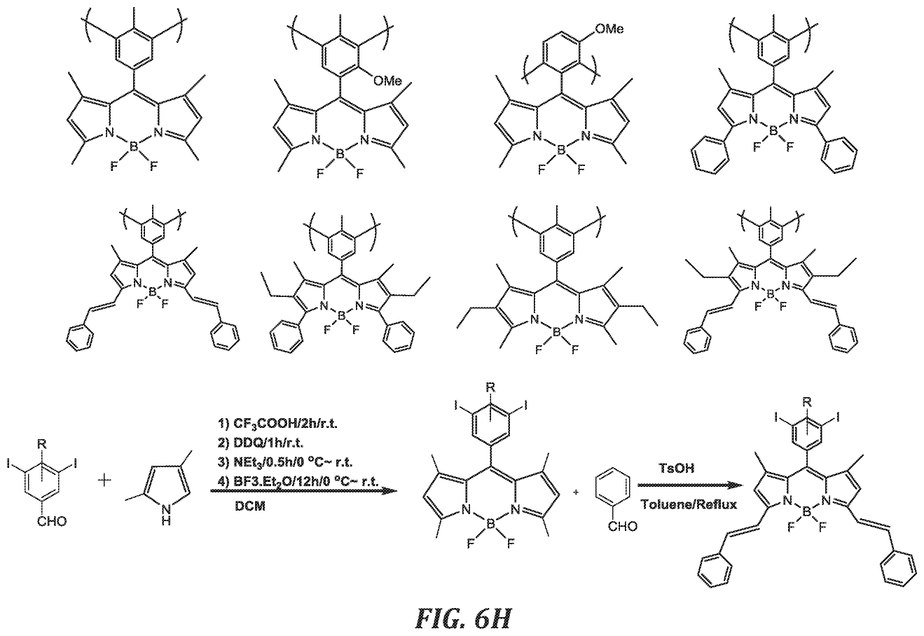

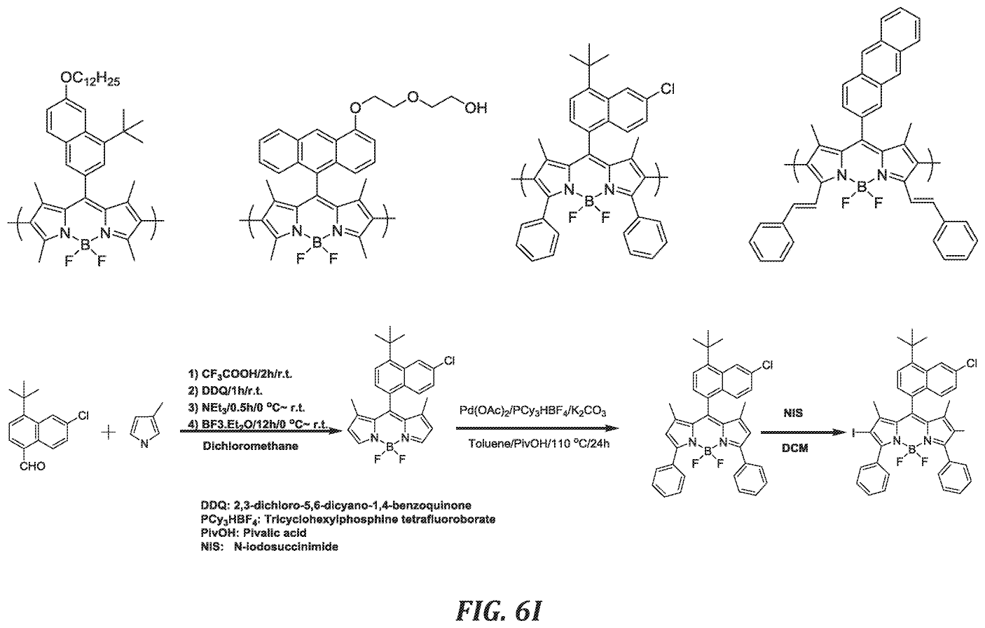

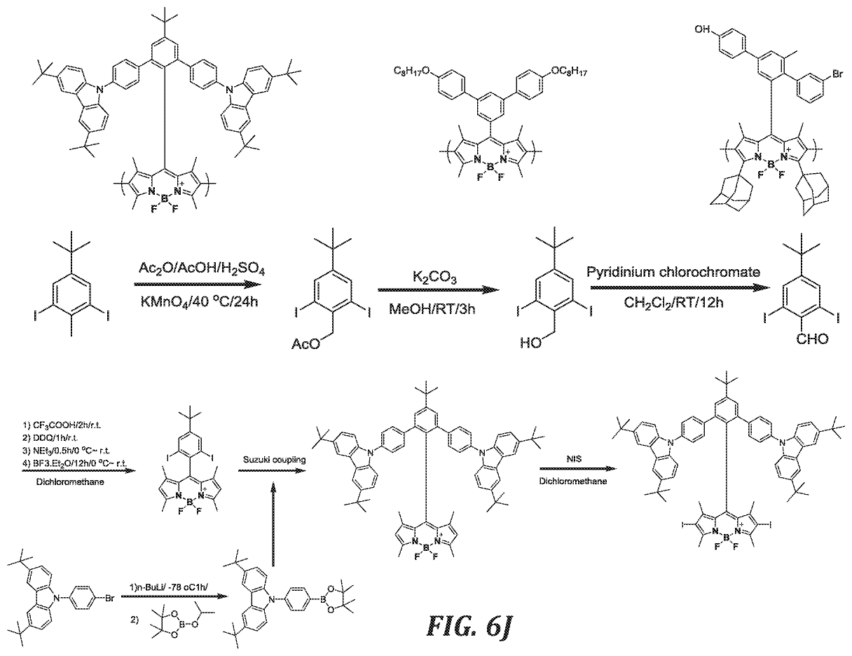

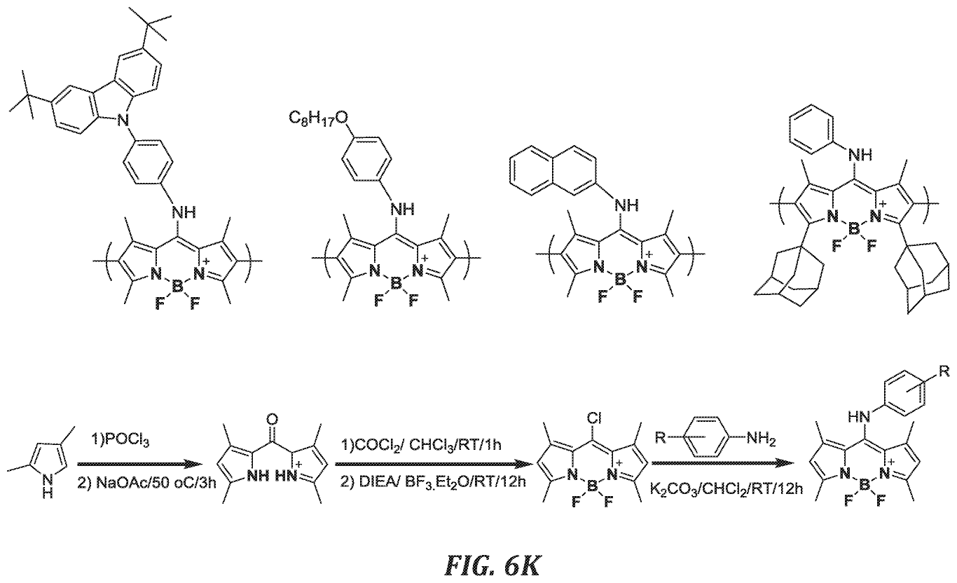

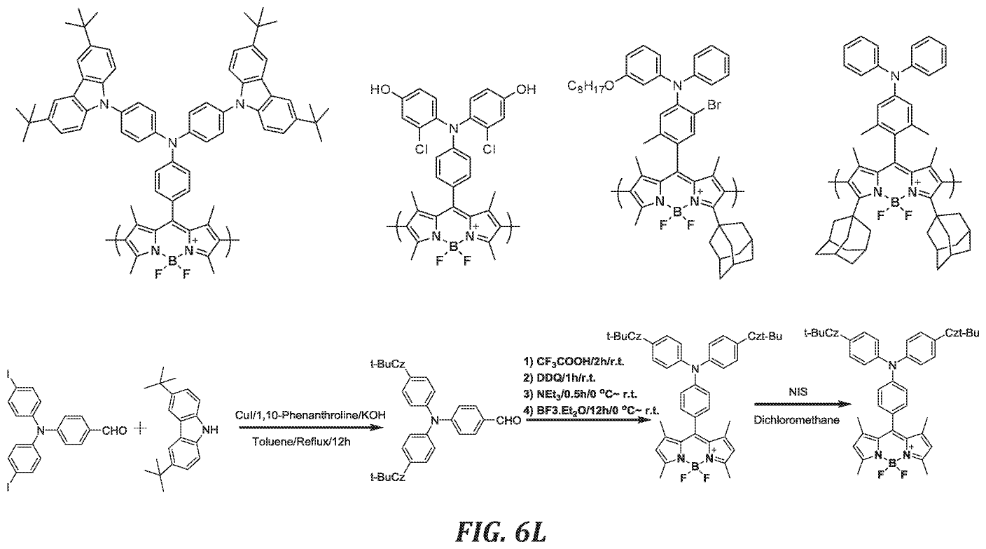

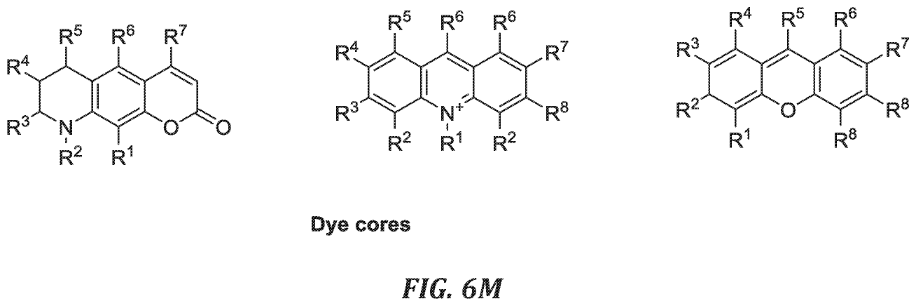

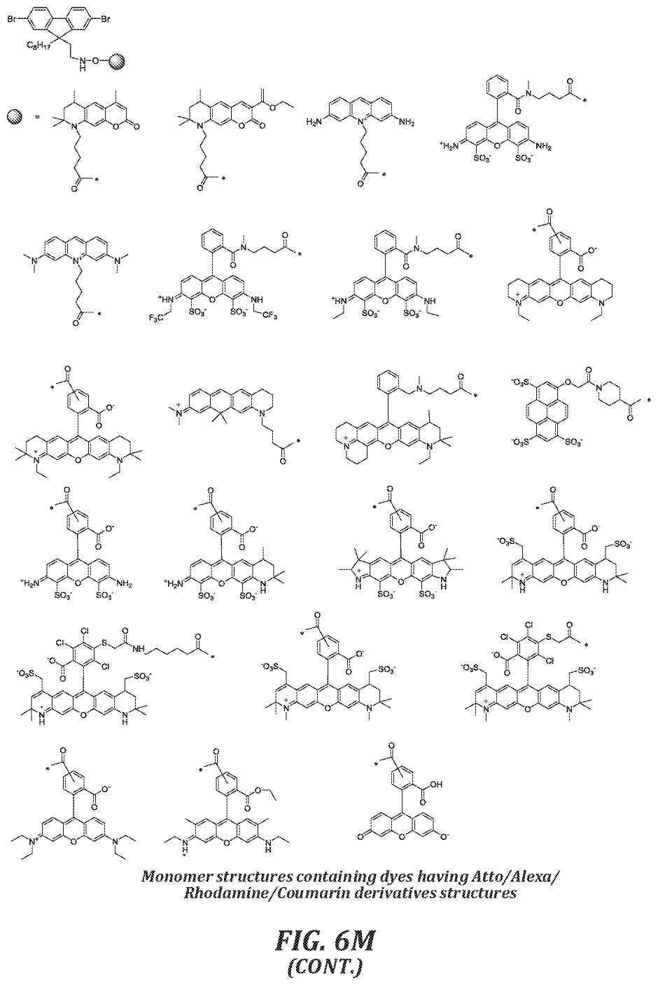

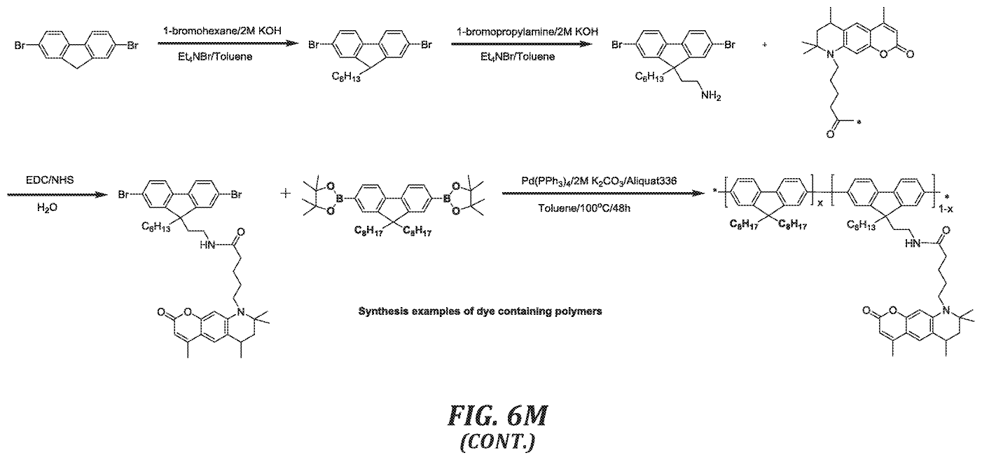

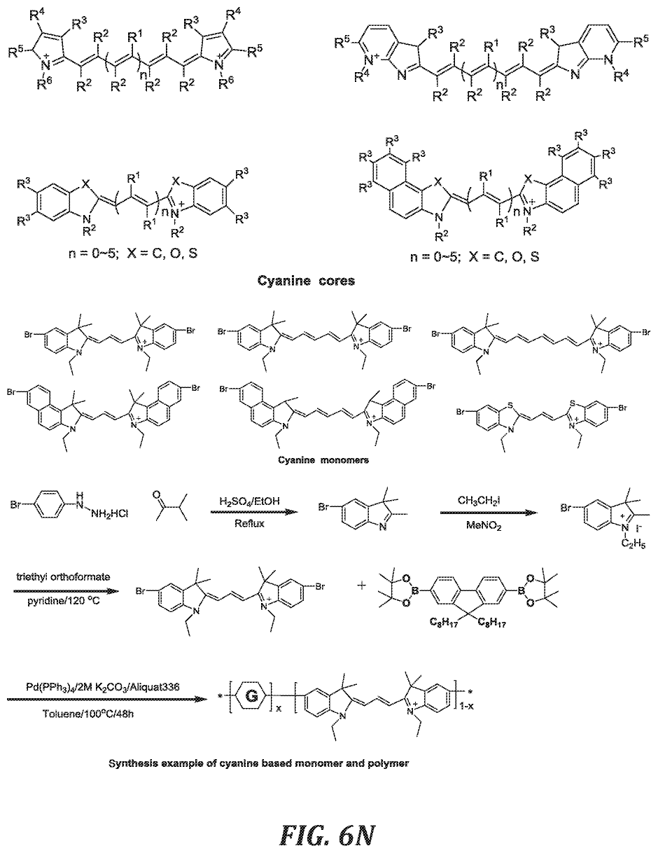

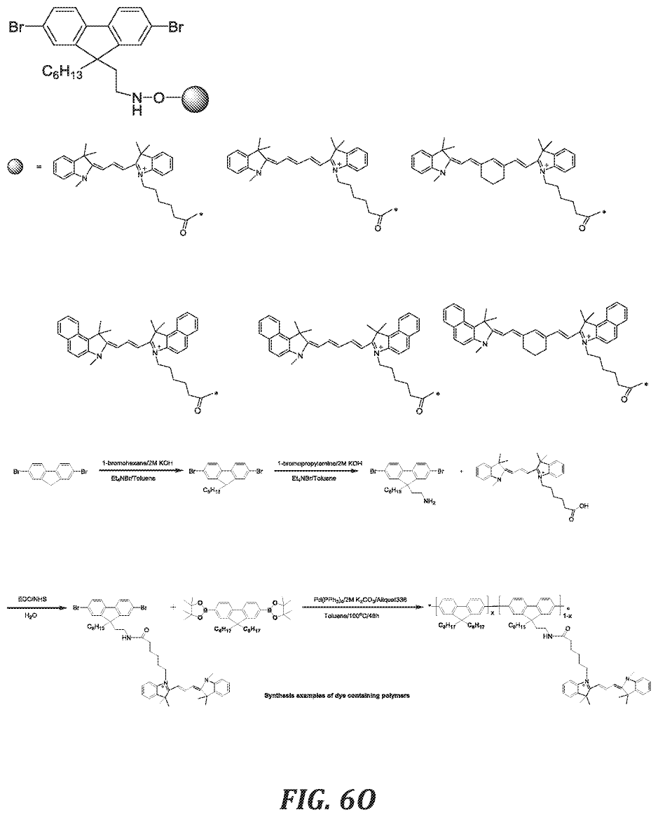

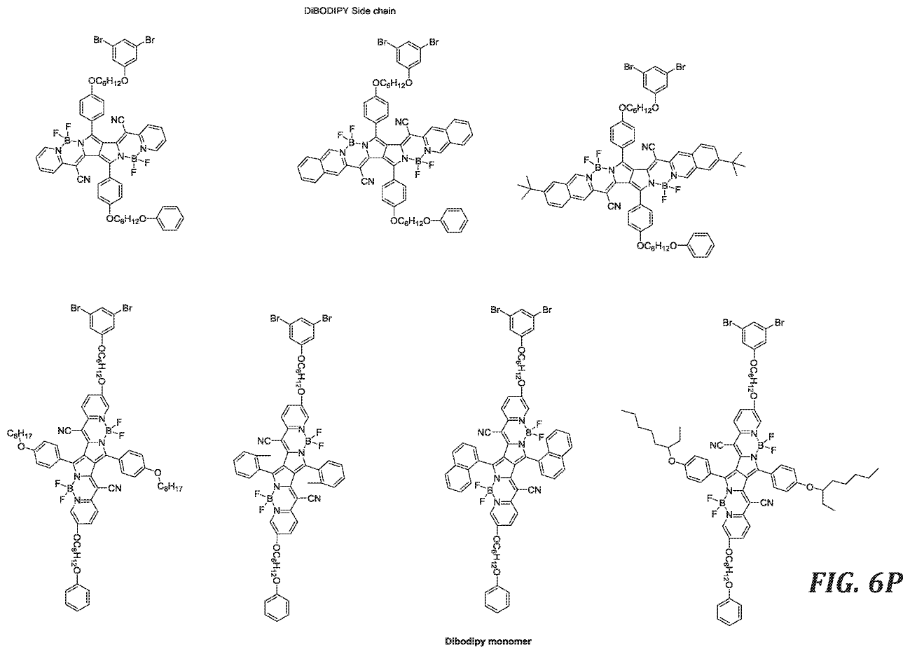

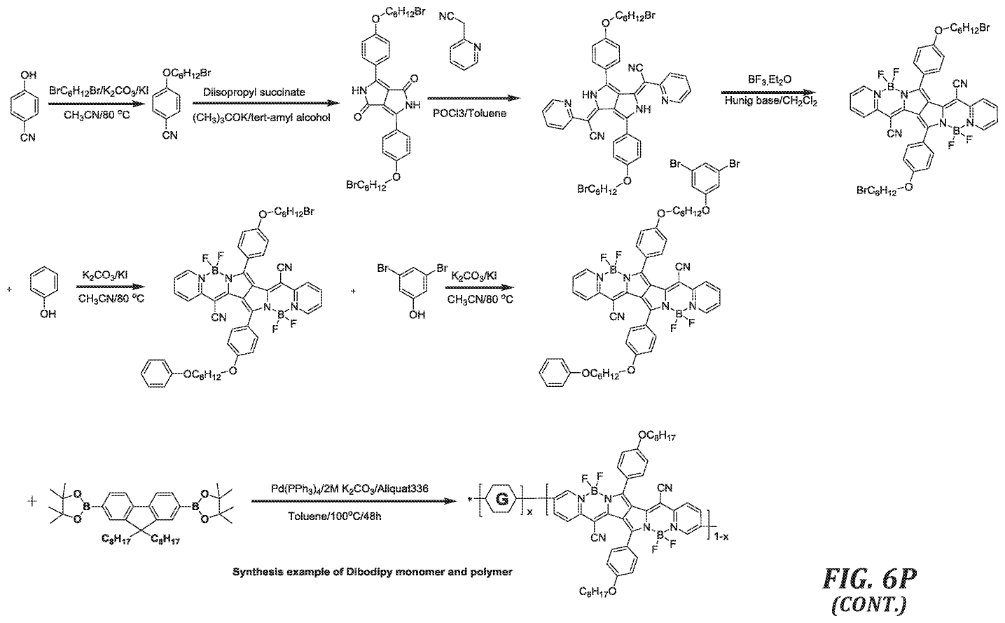

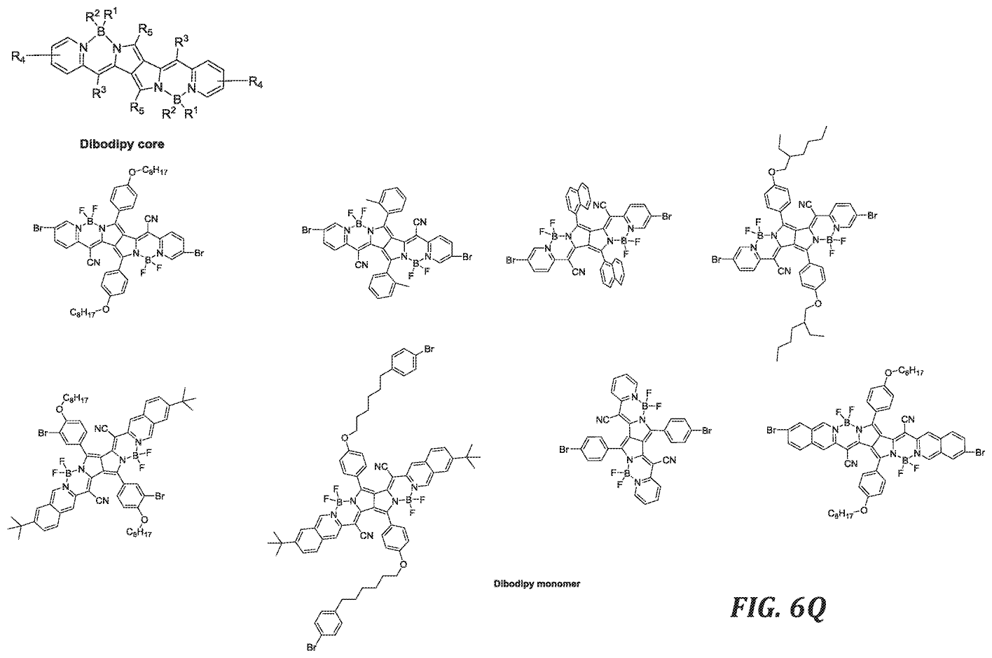

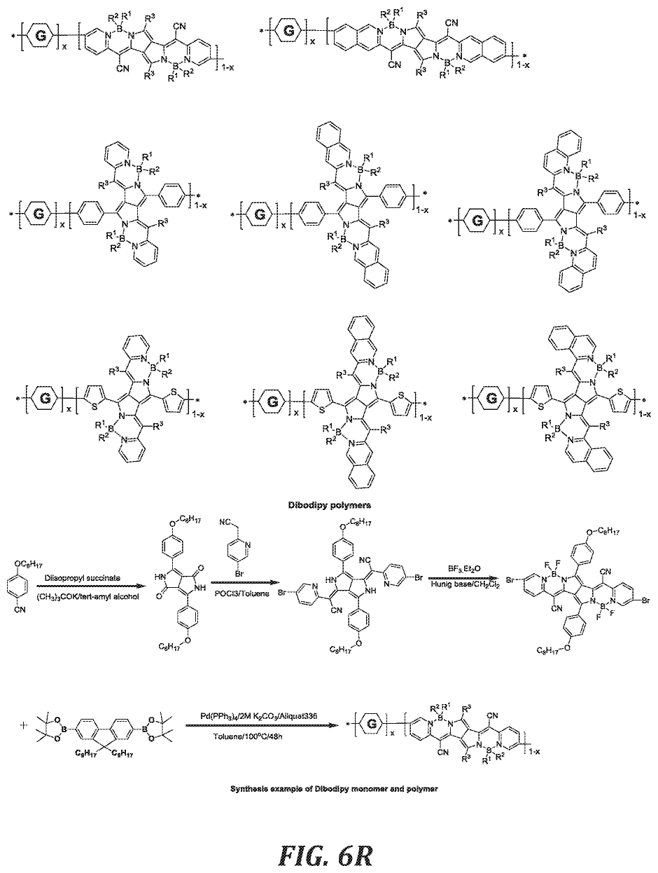

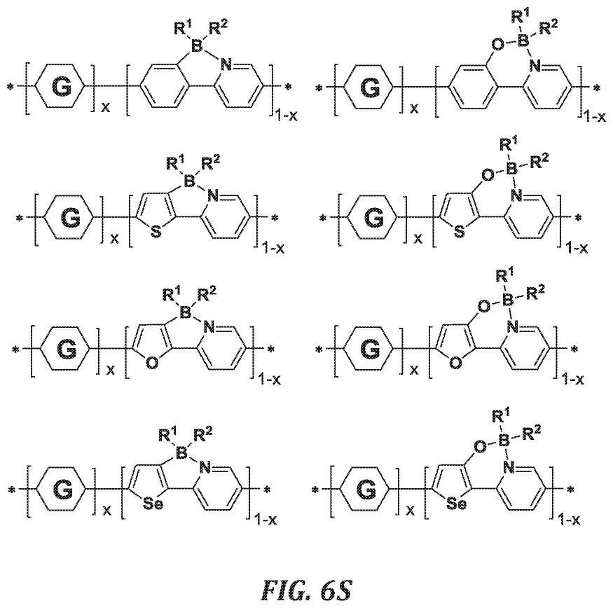

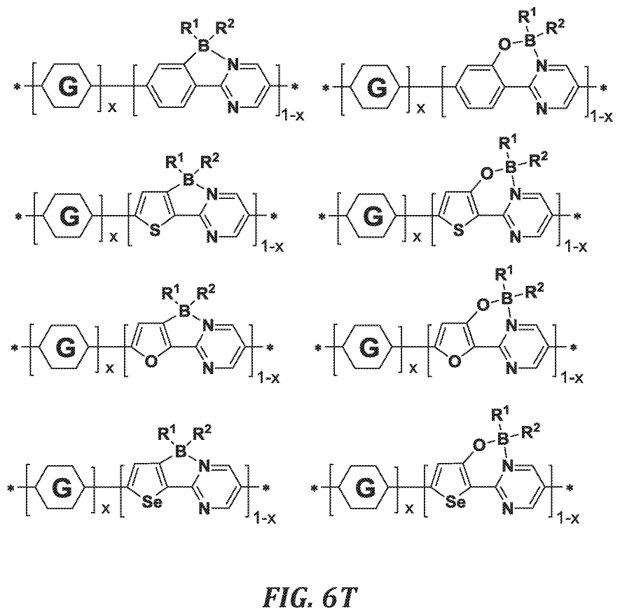

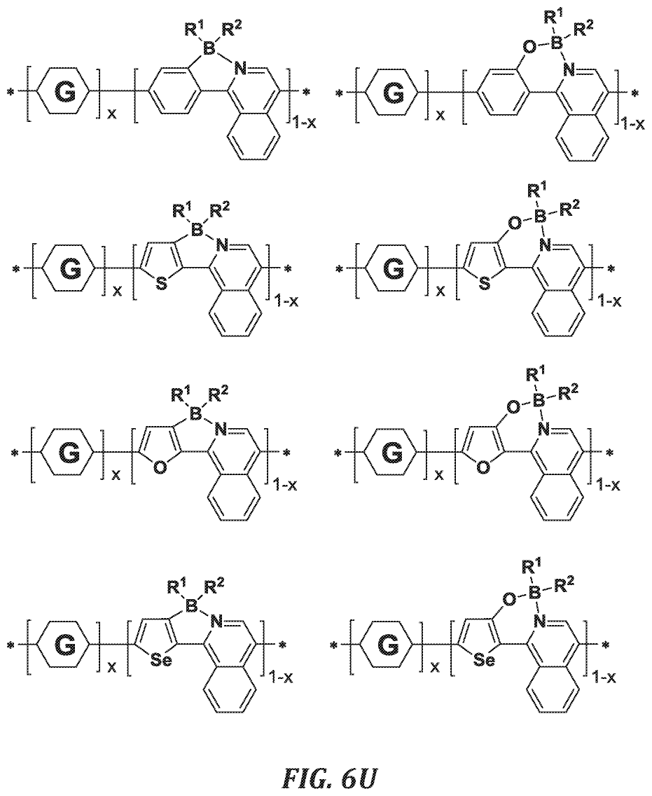

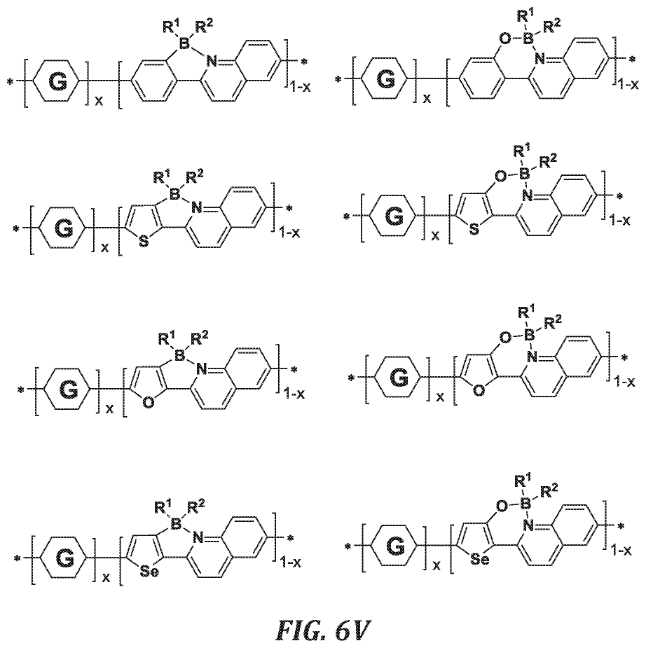

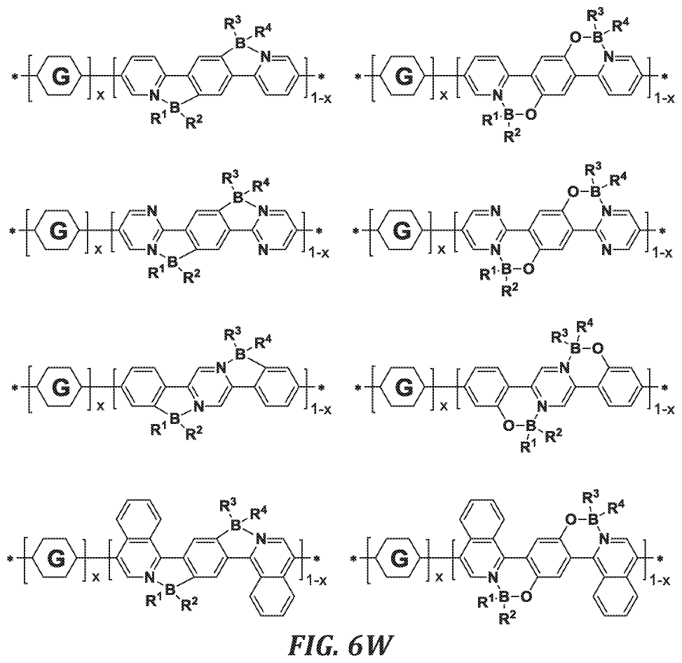

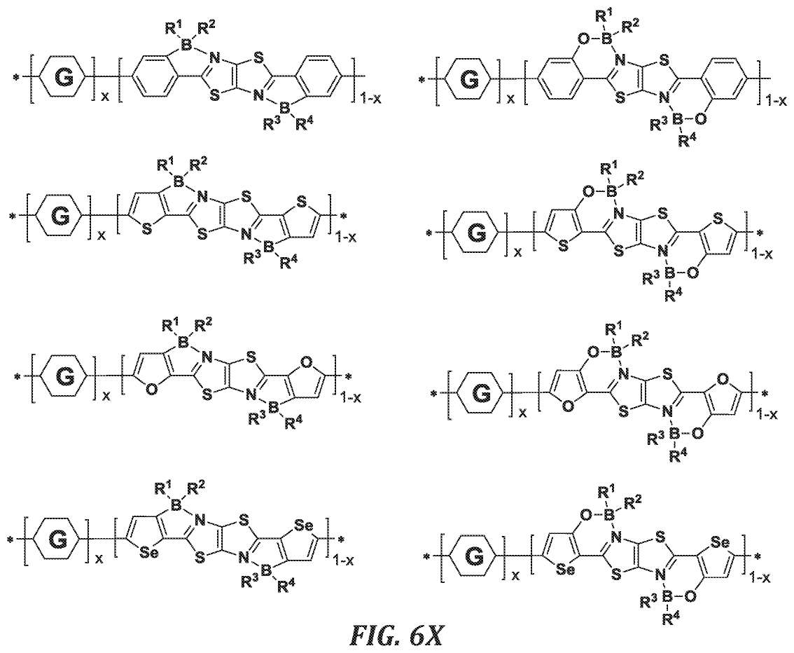

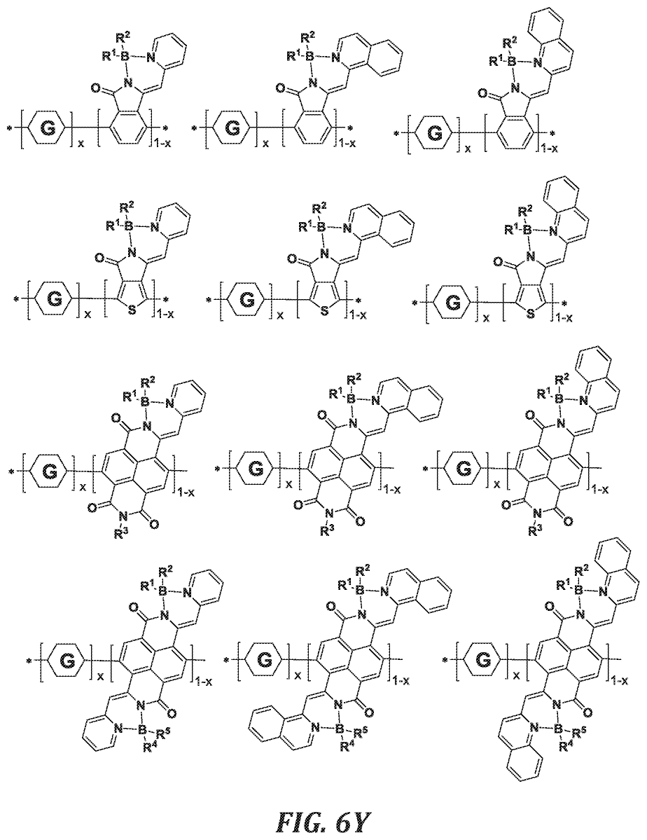

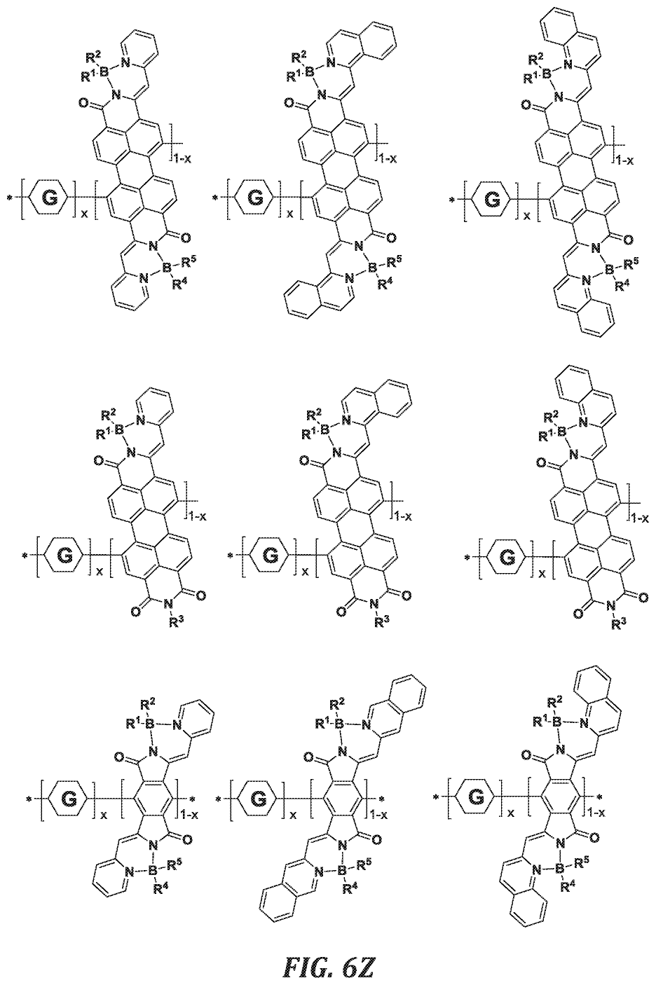

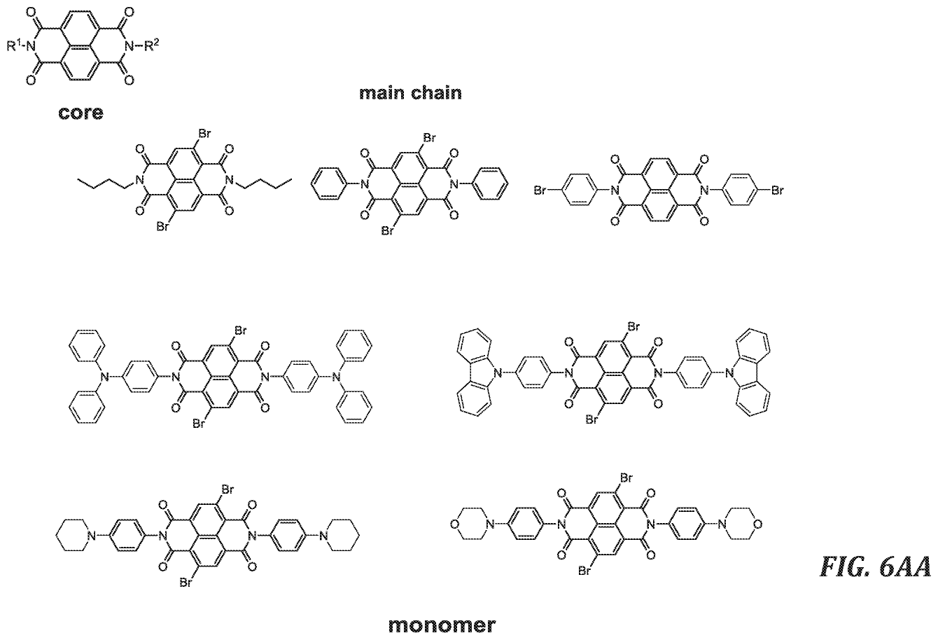

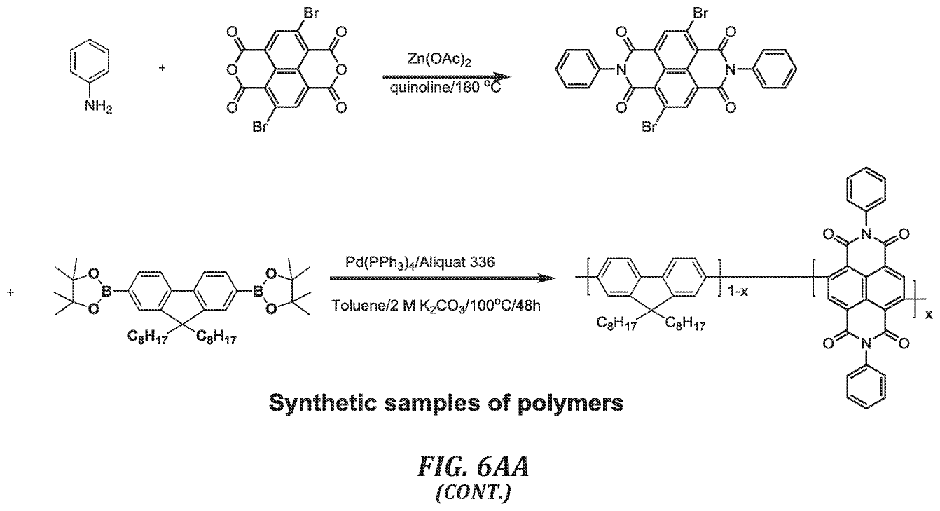

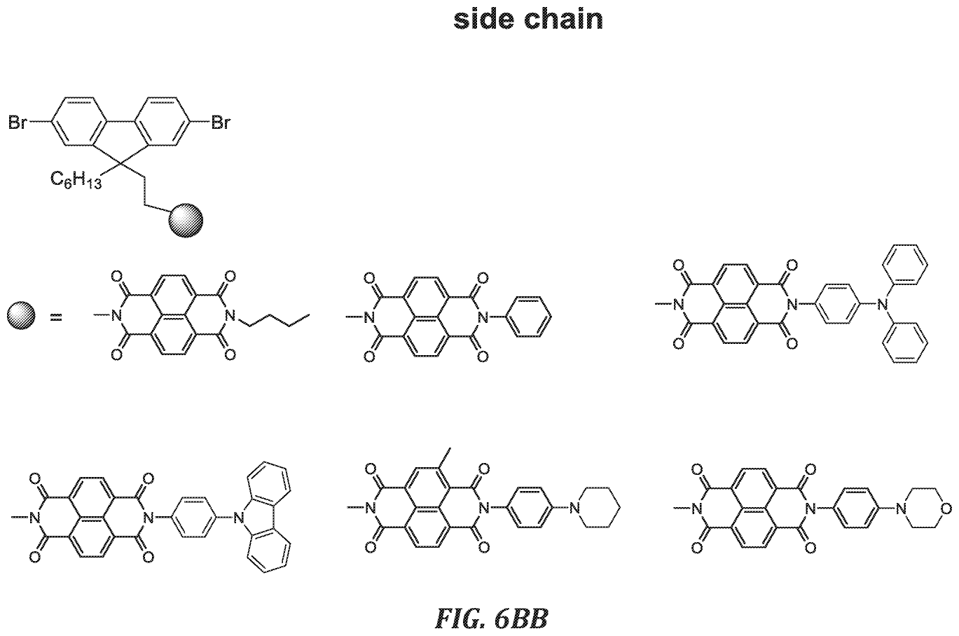

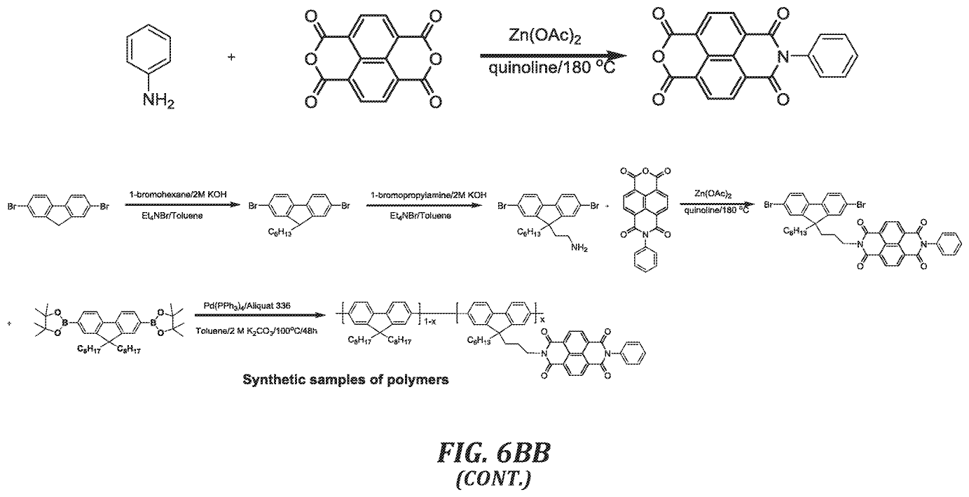

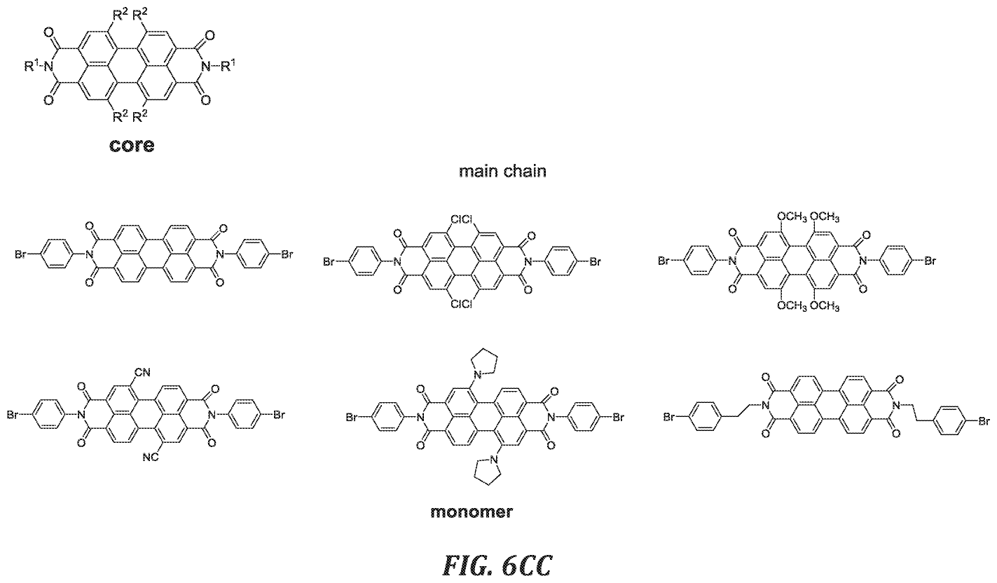

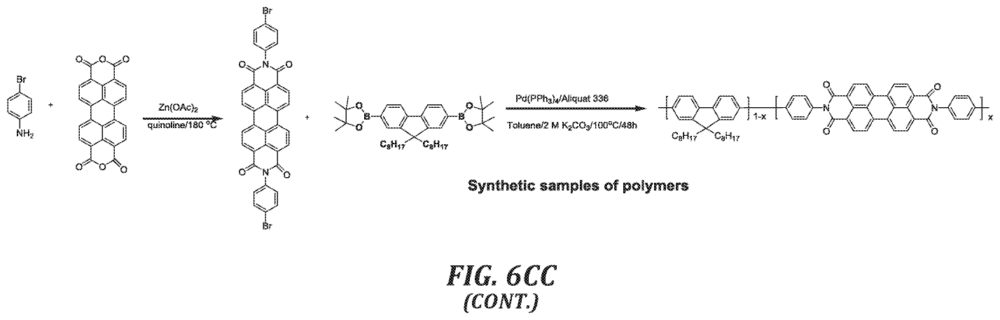

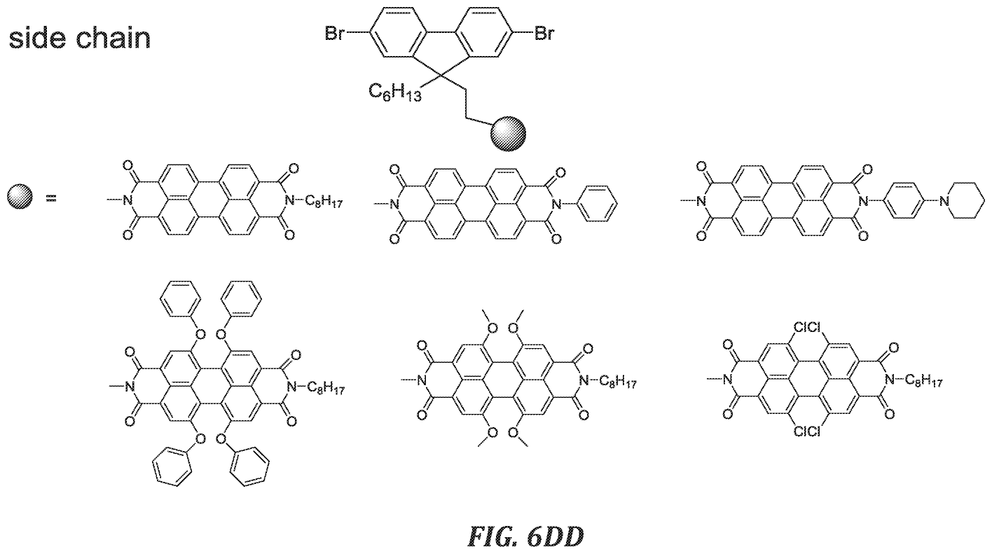

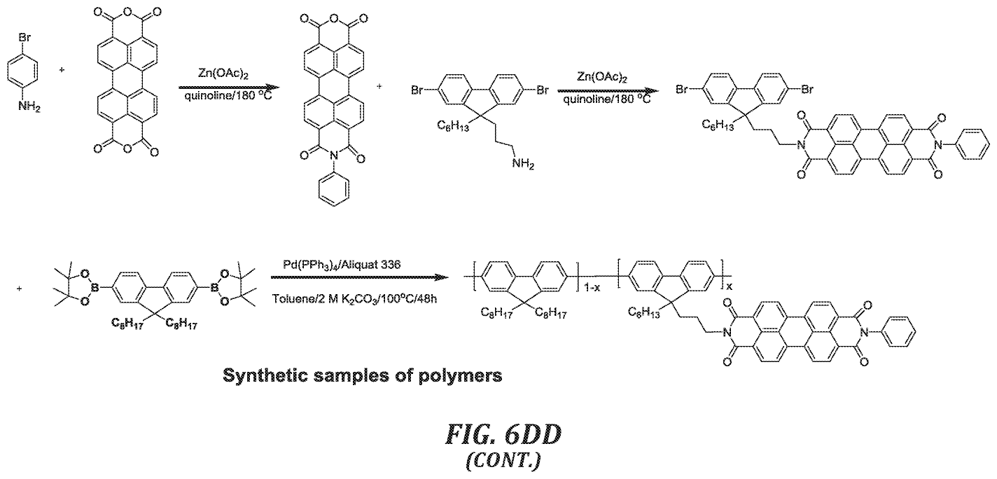

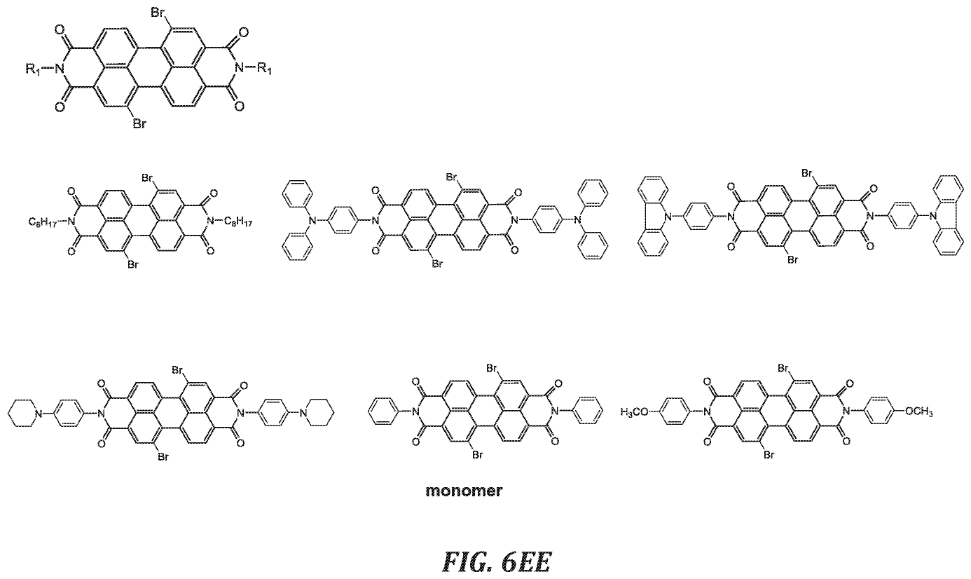

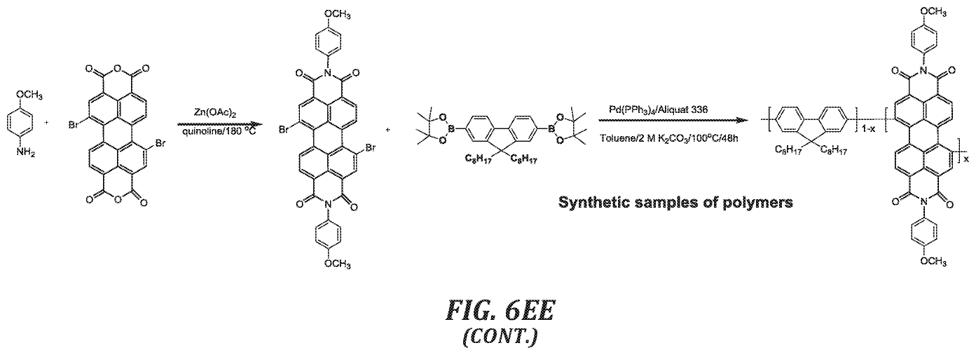

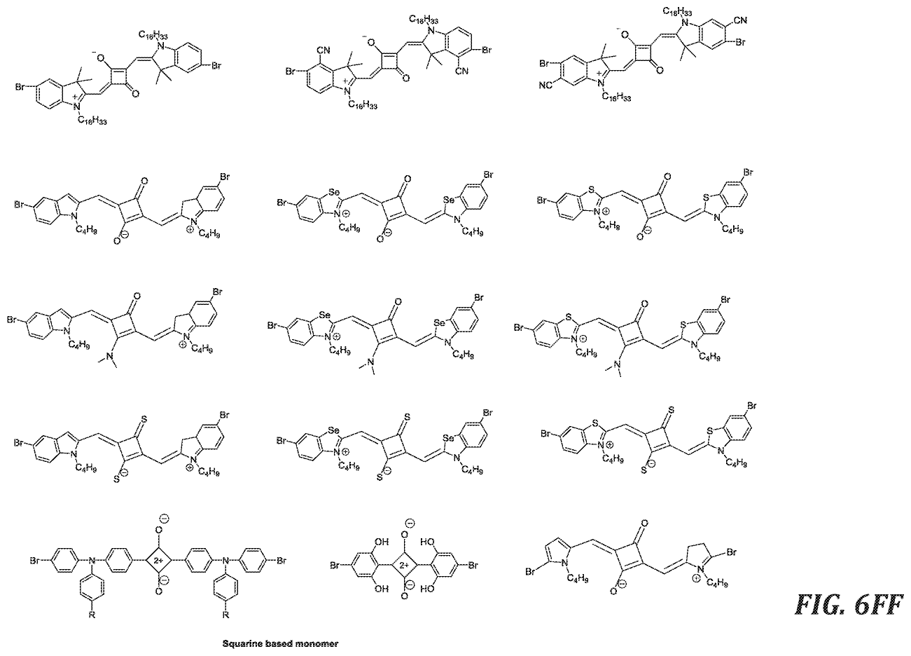

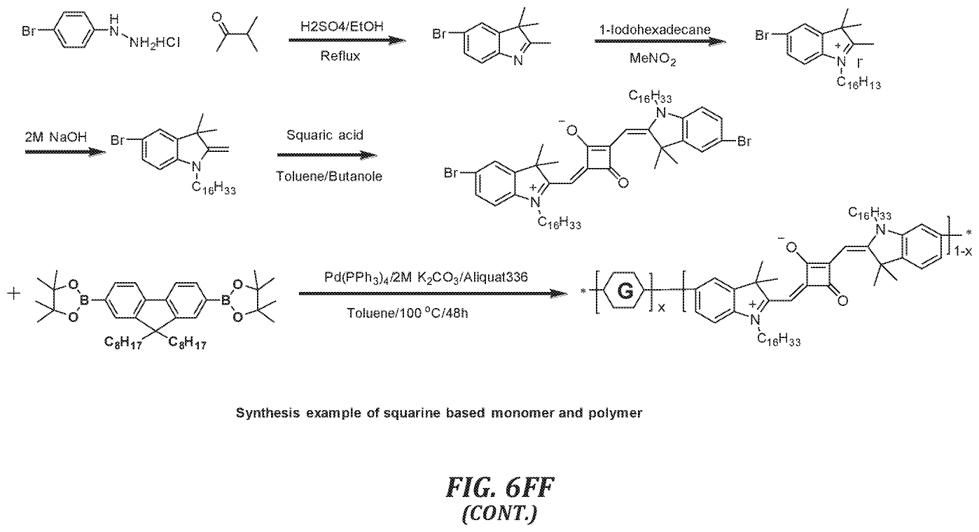

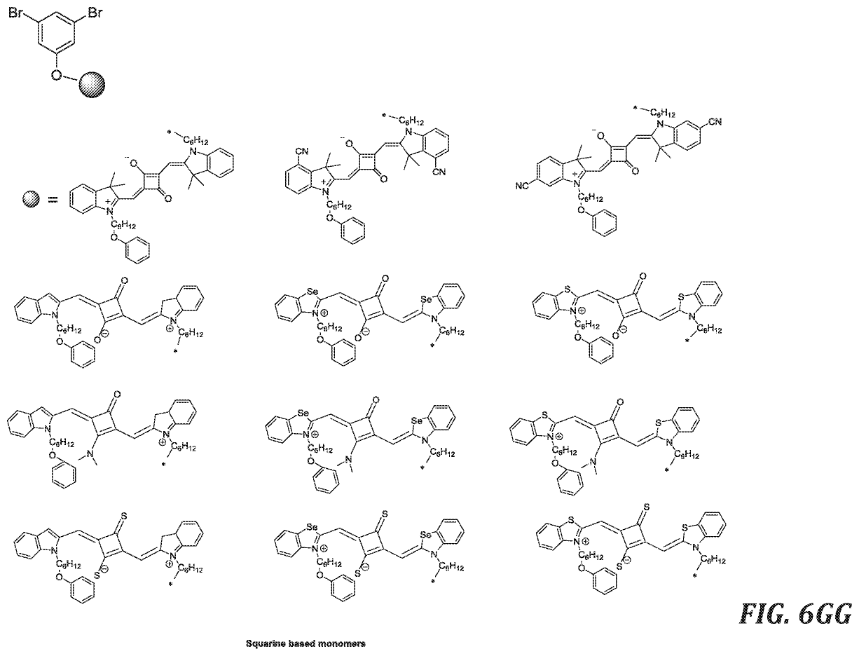

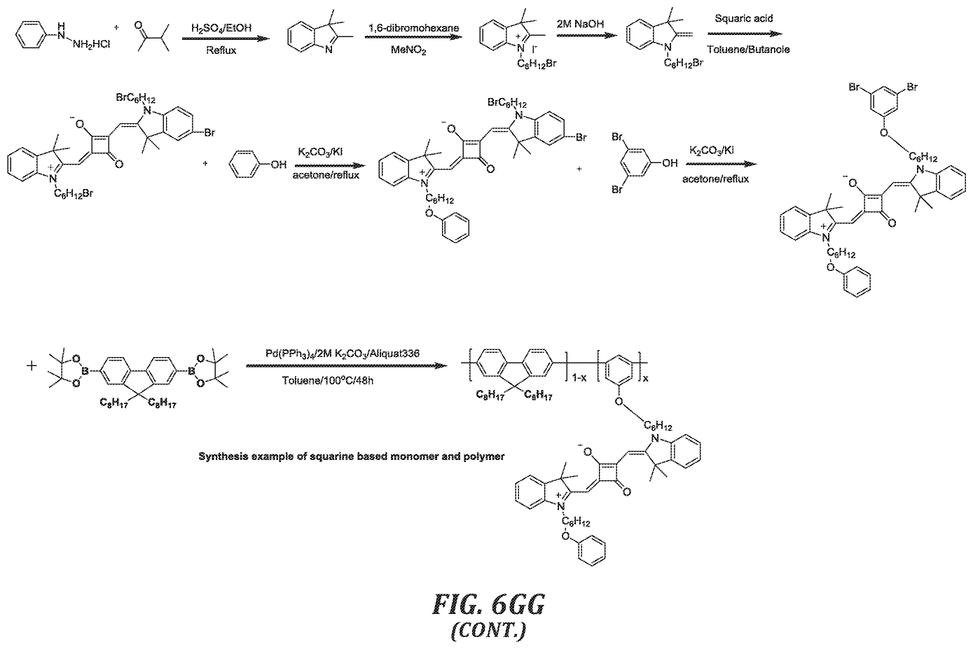

BODIPY and a variety of BODIPY derivatives can be used for the present disclosure. BODIPY and BODIPY derivatives can be polymerized to form polymers (e.g., homopolymers or heteropolymers) and/or can be attached (e.g., covalently attached) to a polymer backbone, sidechain and/or terminus. BODIPY monomeric units and their derivatives include but are not limited to their alkyl derivatives, aryl derivatives, alkyne derivatives, aromatic derivatives, alkoxide derivatives, aza derivatives, BODIPY extended systems and other BODIPY derivatives. In some embodiments, the polymer dots of the present disclosure can include a polymer that includes an absorbing monomeric unit (e.g., a narrow-band absorbing monomeric unit) and/or an emitting monomeric unit having the formula:

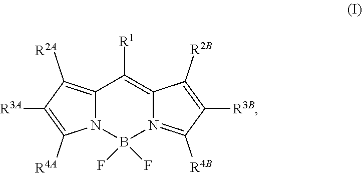

wherein each of variables R¹, R^{2A}, R^{2B}, R^{3A}, R^{3B}, R^{4A} and R^{4B}, or two variables on adjacent atoms (e.g., R^{2A} and R^{3A}, R^{3A} and R^{4A}, R^{2B} and R^{3B}, R^{3B} and R^{4B}) together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, and 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl, and 7-diphenylaminyl-9,9-dialkyl-substituted fluorenyl; alkyl-substituted carbazolyl can include N-alkyl-substituted carbazolyl, 6-alkyl-substituted carbazolyl, and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl, and 4′,4″-alkyl-substituted triphenylaminyl; alkyl-substituted thiophenyl can include 2-alkylthiophenyl, 3-alkylthiophenyl, and 4-alkylthiophenyl; other substituted phenyl can include N-dialkyl-4-phenyl, N-diphenyl-4-phenyl, and N-dialkoxyphenyl-4-phenyl. In some embodiments, each of variables R, R^{2A}, R^{2B}, R^{3A}, R^{3B}, R^{4A} and R^{4B}, or two variables on adjacent atoms (e.g., R^{2A} and R^{3A}, R^{3A} and R^{4A}, R^{2B} and R^{3B}, R^{3B} and R^{4B}) together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R^{2A}, R^{2B}, R^{3A}, R^{3B}, R^{4A}, R^{4B}, or any combination thereof. FIG. 6A shows examples of monomeric units that, e.g., can be integrated with the polymer by attachment to R^{3A} and R^{3B} groups.

In some embodiments, the polymer dots of the present disclosure can include a polymer that includes an absorbing monomeric unit (e.g., a narrow-band absorbing monomeric unit) and/or an emitting monomeric unit having the formula:

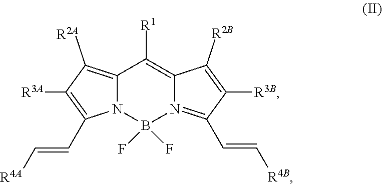

wherein each of R¹, R^{2A}, R^{2B}, R^{3A}, R^{3B}, R^{4A} and R^{4B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. In some embodiments, each of R¹, R^{2A}, R^{2B}, R^{3A}, R^{3B}, R^{4A} and R^{4B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R^{2A}, R^{2B}, R^{3A}, R^{3B}, R^{4A}, R^{4B}, or any combination thereof. The monomeric unit can, for example, integrate with the backbone of the polymer by attachment to the R^{3A} and R^{3B} groups. FIG. 6B shows examples of monomeric units that, e.g., can be integrated with the polymer by attachment to R^{3A} and R^{3B} groups.

In some embodiments, the polymer dots of the present disclosure can include a polymer that includes an absorbing monomeric unit (e.g., a narrow-band absorbing monomeric unit) and/or an emitting monomeric unit having the formula:

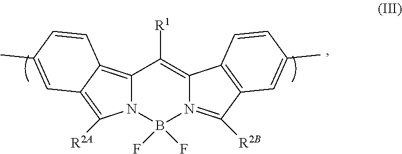

wherein each of R¹, R^{2A} and R^{2B} is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R^{2A} and R^{2B} is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety), e.g., to R¹, R^{2A}, R^{2B}, or any combination thereof. The parentheses indicate points of attachment of the monomeric unit to the backbone of the polymer. FIG. 6C shows examples of monomeric units that, e.g., can be integrated with the polymer (e.g., copolymerized in the polymer).

In some embodiments, the polymer dots of the present disclosure can include a polymer that includes an absorbing monomeric unit (e.g., a narrow-band absorbing monomeric unit) and/or an emitting monomeric unit having the formula:

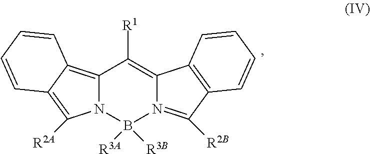

wherein each of R¹, R^{2A}, R^{2B}, R^{3A}, and R^{3B} is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R^{2A}, R^{2B}, R^{3A}, and R^{3B} is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R^{2A}, R^{2B}, R^{3A}, and R^{3B} or any combination thereof. FIG. 6D shows examples of monomeric units that, e.g., can be integrated with the polymer by attachment to R^{3A} and R^{3B} groups.

In some embodiments, the polymer dots of the present disclosure can include a polymer that includes an absorbing monomeric unit (e.g., a narrow-band absorbing monomeric unit) and/or an emitting monomeric unit having the formula:

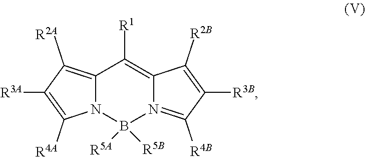

wherein each of R¹, R^{2A}, R^{2B}, R^{3A}, R^{3B}, R^{4A}, R^{4B}, R^{5A}, and R^{5B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R^{2A}, R^{2B}, R^{3A}, R^{3B}, R^{4A}, R^{4B}, R^{5A}, and R^{5B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., copolymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R^{2A}, R^{2B}, R^{3A}, R^{3B}, R^{4A}, R^{4B}, R^{5A}, R^{5B}, or any combination thereof.

In certain embodiments, the narrow-band monomeric units can be integrated into the backbone by attachment to the R^{5A} and R^{5B} groups. FIG. 6E shows examples of monomeric units that, e.g., can be integrated with the polymer by attachment to R^{5A} and R^{5B} groups.

In some embodiments, the polymer dots of the present disclosure can include a polymer that includes an absorbing monomeric unit (e.g., a narrow-band absorbing monomeric unit) and/or an emitting monomeric unit having the formula:

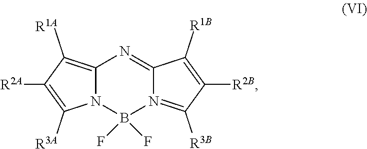

wherein each of R^{1A}, R^{1B}, R^{2A}, R^{2B}, R^{3A} and R^{3B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R^{1A}, R^{1B}, R^{2A}, R^{2B}, R^{3A} and R^{3B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R^{1A}, R^{1B}, R^{2A}, R^{2B}, R^{3A}, R^{3B}, or any combination thereof. FIG. 6F shows examples of monomeric units that, e.g., can be integrated with the polymer by attachment to R^{1A}, R^{1B}, R^{2A}, R^{2B}, R^{3A} or R^{3B} groups.

In some embodiments, the polymer dots of the present disclosure can include a polymer that includes an absorbing monomeric unit (e.g., a narrow-band absorbing monomeric unit) and/or an emitting monomeric unit having the formula:

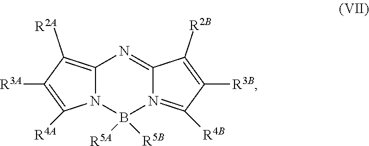

wherein each of R^{2A}, R^{2B}, R^{3A}, R^{3B}, R^{4A}, R^{4B}, R^{5A} and R^{5B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R^{2A}, R^{2B}, R^{3A}, R^{3B}, R^{4A}, R^{4B}R^{5A} and R^{5B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R^{2A}, R^{2B}, R^{3A}, R^{3B}, R^{4A}, R^{4B}, R^{5A}, R^{5B}, or any combination thereof. FIG. 6G shows examples of monomeric units that, e.g., can be integrated with the polymer by attachment to R^{5A} and R^{5B} groups.

In some embodiments, the polymer dots of the present disclosure can include a polymer that includes an absorbing monomeric unit (e.g., a narrow-band absorbing monomeric unit) and/or an emitting monomeric unit having the formula:

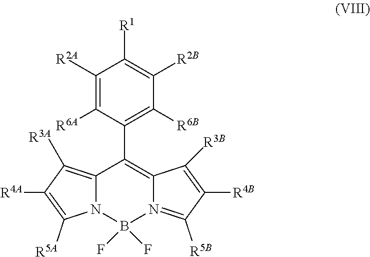

wherein each of R¹, R^{2A}, R^{2B}, R^{3A}, R^{3B}, R^{4A} and R^{4B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl, and wherein each of R^{5A}, R^{5B}, R^{6A} and R^{6B} are independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, hydroxyl, cyano, nitro, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R^{2A}, R^{2B}, R^{3A}, R^{3B}, R^{4A} and R^{4B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., copolymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R^{2A}, R^{2B}, R^{3A}, R^{3B}, R^{4A}, R^{4B}, R^{5A}, R^{5B}, R^{6A}, R^{6B} or any combination thereof. FIG. 6H shows examples of monomeric units that, e.g., can be integrated with the polymer by attachment to R^{2A}, R^{2B}, R^{6A} or R^{6B} groups.

In some embodiments, the polymer dots of the present disclosure can include a polymer that includes an absorbing monomeric unit (e.g., a narrow-band absorbing monomeric unit) and/or an emitting monomeric unit having the formula:

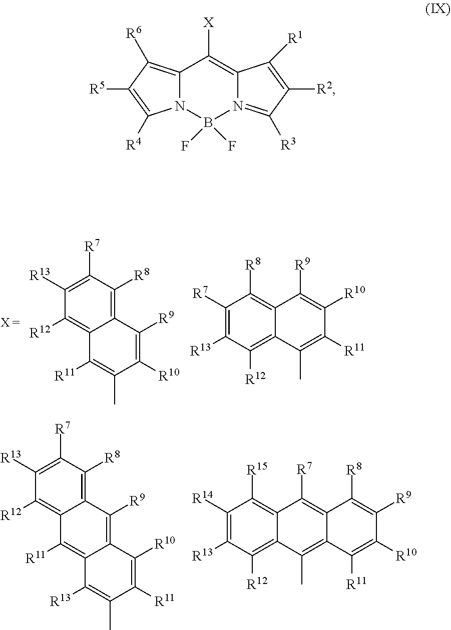

wherein X represents aryl group and its derivatives, each of R¹, R², R³, R⁴, R⁵, R⁶, R⁷, R⁸, R⁹, R¹⁰, R¹¹, R¹², R¹³, R¹⁴ and R¹⁵, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R², R³, R⁴, R⁵, R₆, R⁷, R⁸, R⁹, R¹⁰, R¹¹, R¹², R¹³, R¹⁴ and R¹⁵, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. When X represents naphthalene and its derivatives, the absorbing monomeric unit, the emitting monomeric unit, or a combination of both the absorbing monomeric unit and the emitting monomeric unit can be integrated into a backbone (e.g., polymerized in the polymer) and/or covalently attached to the backbone, a terminus, or a sidechain of the polymer) of the polymer through at least one attachment (or an attachment via a linker moiety) to R⁷, R⁸, R⁹, R¹⁰, R¹¹, R¹² or any combination thereof. When X represents anthracene and its derivatives, the absorbing monomeric unit, the emitting monomeric unit, or a combination of both the absorbing monomeric unit and the emitting monomeric unit can be integrated into a backbone of the polymer and/or covalently attached to the backbone, a terminus, or a sidechain of the polymer through at least one attachment (or an attachment via a linker moiety) to R⁷, R⁸, R⁹, R¹⁰, R¹¹, R¹², R³, R¹⁴, R⁵ or any combination thereof. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², R³, R⁴, R⁵, R⁶, R⁷, R⁸, R⁹, R¹⁰, R¹¹, R¹², R¹³, R¹⁴, R¹⁵ or any combination thereof. FIG. 6I shows examples of monomeric units that, e.g., can be integrated with the polymer by attachment to R² or R⁵ groups

In some embodiments, the polymer dots of the present disclosure can include a polymer that includes an absorbing monomeric unit (e.g., a narrow-band absorbing monomeric unit) and/or an emitting monomeric unit having the formula:

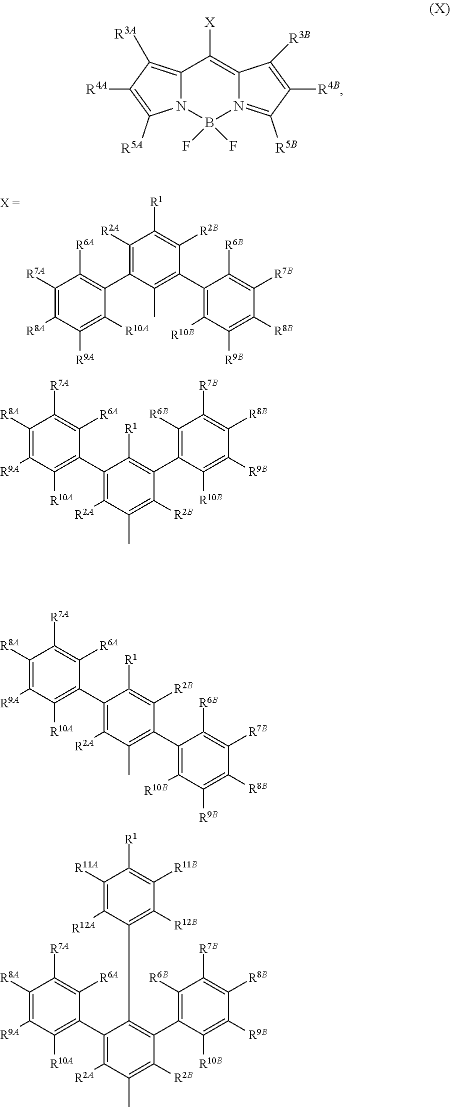

wherein X represents aryl groups and their derivatives, each of R¹, R^{2A}, R^{2B}, R^{3A}, R^{3B}, R^{4A}, R^{4B}, R^{5A}, R^{5B}, R^{6A}, R^{6B}, R^{7A}, R^{7B}, R^{8A}, R^{8B}, R^{9A}, R^{9B}, R^{10A}, R^{10B}, R^{11A}, R^{11B}, R^{12A}, and R^{12B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R^{2A}, R^{2B}, R^{3A}, R^{3B}, R^{4A}, R^{4B}, R^{5A}, R^{5B}, R^{6A}, R^{6B}, R^{7A}, R^{7B}, R^{8A}, R^{8B}, R^{9A}, R^{9B}, R^{10A}, R^{10B}, R^{11A}, R^{11B}, R^{12A}, and R^{12B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R^{2A}, R^{2B}, R^{3A}, R^{3B}, R^{4A}, R^{4B}, R^{5A}, R^{5B}, R^{6A}, R^{6B}, R^{7A}, R^{7B}, R^{8A}, R^{8B}, R^{9A}, R^{9B}, R^{10A}, R^{10B}, R^{11A}, R^{11B}, R^{12A}, R^{12B}, or any combination thereof. FIG. 6J shows examples of monomeric units that, e.g., can be integrated with the polymer by attachment to RA or RB groups.

In some embodiments, the polymer dots of the present disclosure can include a polymer that includes an absorbing monomeric unit (e.g., a narrow-band absorbing monomeric unit) and/or an emitting monomeric unit having the formula:

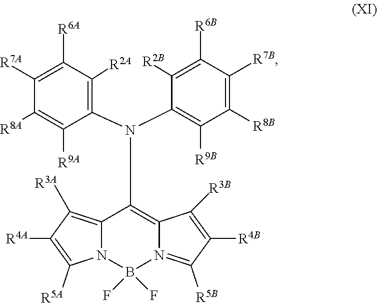

wherein each of R^{2A}, R^{2B}, R^{3A}, R^{3B}, R^{4A}, R^{4B}, R^{5A}, R^{5B}, R^{6A}, R^{6B}, R^{7A}, R^{7B}, R^{8A}, R^{8B}, R^{9A}, and R^{9B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R^{2A}, R^{2B}, R^{3A}, R^{3B}, R^{4A}, R^{4B}, R^{5A}, R^{5B}, R^{6A}, R^{6B}, R^{7A}, R^{7B}, R^{8A}, R^{8B}, R^{9A}, and R^{9B}, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R^{2A}, R^{2B}, R^{3A}, R^{3B}, R^{4A}, R^{4B}, R^{5A}, R^{5B}, R^{6A}, R^{6B}, R^{7A}, R^{7B}, R^{8A}, R^{8B}, R^{9A}, R^{9B}, or any combination thereof. FIG. 6K shows examples of monomeric units that, e.g., can be integrated with the polymer by attachment to R^{4A} or R^{4B} groups.

In some embodiments, the polymer dots of the present disclosure can include a polymer that includes an absorbing monomeric unit (e.g., a narrow-band absorbing monomeric unit) and/or an emitting monomeric unit having the formula:

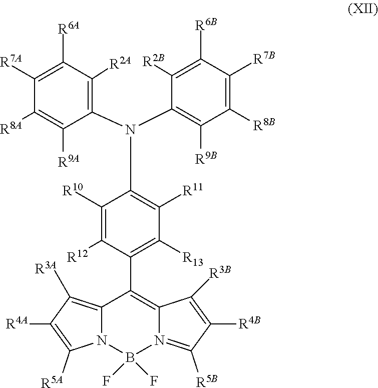

wherein each of R^{2A}, R^{2B}, R^{3A}, R^{3B}, R^{4A}, R^{4B}, R^{5A}, R^{5B}, R^{6A}, R^{6B}, R^{7A}, R^{7B}, R^{8A}, R^{8B}, R^{9A}, R^{9B}, R¹⁰, R¹¹, R¹², and R¹³, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R^{2A}, R^{2B}, R^{3A}, R^{3B}, R^{4A}, R^{4B}, R^{5A}, R^{5B}, R^{6A}, R^{6B}, R^{7A}, R^{7B}, R^{8A}, R^{8B}, R^{9A}, R^{9B}, R¹⁰, R¹¹, R¹², and R¹³, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R^{2A}, R^{2B}, R^{3A}, R^{3B}, R^{4A}, R^{4B}, R^{5A}, R^{5B}, R^{6A}, R^{6B}, R^{7A}, R^{7B}, R^{8A}, R^{8B}, R^{9A}, R^{9B}, R¹⁰, R¹¹, R¹², R¹³, or any combination thereof. FIG. 6L shows examples of monomeric units that, e.g., can be integrated with the polymer by attachment to R^{4A} or R^{4B} groups.

In some embodiments, the polymer dots of the present disclosure can include a polymer that includes an absorbing monomeric unit (e.g., a narrow-band absorbing monomeric unit) and/or an emitting monomeric unit having the formula:

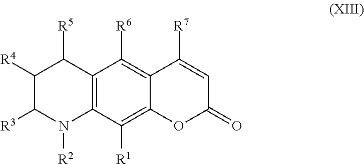

wherein each of R¹, R², R³, R⁴, R⁵, R⁶, and R⁷, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R², R³, R⁴, R⁵, R⁶, and R⁷, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², R³, R⁴, R⁵, R⁶, R⁷, or any combination thereof. FIG. 6M shows examples of monomeric units that, e.g., can be integrated with the polymer by attachment to the R², for example, via a linker moiety.

In some embodiments, the polymer dots of the present disclosure can include a polymer that includes an absorbing monomeric unit (e.g., a narrow-band absorbing monomeric unit) and/or an emitting monomeric unit having the formula:

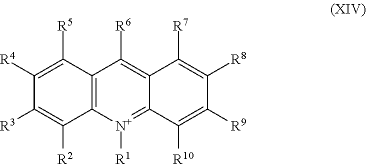

wherein each of R¹, R², R³, R⁴, R⁵, R⁶, R⁷, R⁸, R⁹, and R¹⁰, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R², R³, R⁴, R⁵, R⁶, R⁷, R⁸, R⁹, and R¹⁰, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², R³, R⁴, R⁵, R⁶, R⁷, R⁸, R⁹, R¹⁰, or any combination thereof. FIG. 6M shows examples of monomeric units that, e.g., can be integrated with the polymer by attachment to the R¹, for example, via a linker moiety.

In some embodiments, the polymer dots of the present disclosure can include a polymer that includes an absorbing monomeric unit (e.g., a narrow-band absorbing monomeric unit) and/or an emitting monomeric unit having the formula:

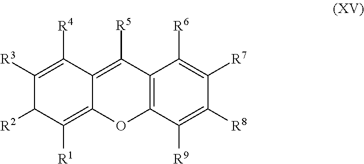

wherein each of R¹, R², R³, R⁴, R⁵, R⁶, R⁷, R⁸, and R⁹, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R², R³, R⁴, R⁵, R⁶, R⁷, R⁸, and R⁹, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², R³, R⁴, R⁵, R⁶, R⁷, R⁸, R⁹, or any combination thereof. FIG. 6M shows examples of monomeric units that, e.g., can be integrated with the polymer by attachment to the R⁵, for example, via a linker moiety.

In some embodiments, the polymer dots of the present disclosure can include a polymer that includes an absorbing monomeric unit (e.g., a narrow-band absorbing monomeric unit) and/or an emitting monomeric unit having the formula:

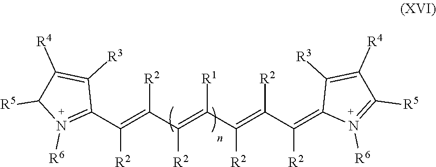

wherein each of R¹, R², R³, R⁴, R⁵, and R⁶, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R², R³, R⁴, R⁵, and R⁶, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², R³, R⁴, R⁵, R⁶, or any combination thereof. FIGS. 6N and 6O show examples of monomeric units that, e.g., can be integrated with the polymer by attachment to R³, R⁴, or R⁵ for example, through a linker moiety.

In some embodiments, the polymer dots of the present disclosure can include a polymer that includes an absorbing monomeric unit (e.g., a narrow-band absorbing monomeric unit) and/or an emitting monomeric unit having the formula:

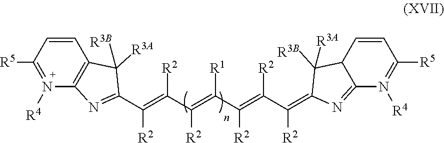

wherein each of R¹, R², R^{3A}, R^{3B}, R⁴, and R⁵, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R², R^{3A}, R^{3B}, R⁴, and R⁵, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², R^{3A}, R^{3B}, R⁴, R⁵, or any combination thereof. FIGS. 6N and 6O show examples of monomeric units that, e.g., can be integrated with the polymer by attachment to R^{3A}, R^{3B}, R⁴, or R⁵ for example, through a linker moiety.

In some embodiments, the polymer dots of the present disclosure can include a polymer that includes an absorbing monomeric unit (e.g., a narrow-band absorbing monomeric unit) and/or an emitting monomeric unit having the formula:

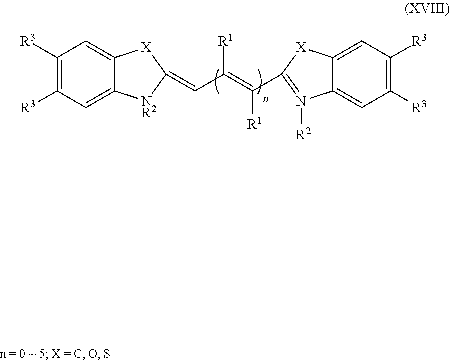

wherein each of R¹, R², and R³, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R², and R³, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², R³, or any combination thereof. FIGS. 6N and 6O show examples of monomeric units that, e.g., can be integrated with the polymer by attachment to R² and R³, for example, through a linker moiety.

In some embodiments, the polymer dots of the present disclosure can include a polymer that includes an absorbing monomeric unit (e.g., a narrow-band absorbing monomeric unit) and/or an emitting monomeric unit having the formula:

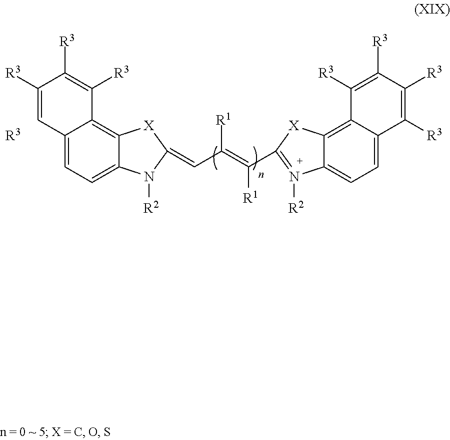

wherein each of R¹, R², or R³, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R², or R³, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², R³, or any combination thereof. FIGS. 6N and 6O show examples of monomeric units that, e.g., can be integrated with the polymer by attachment to R² or R³, for example, through a linker moiety.

In some embodiments, the polymer dots of the present disclosure can include a polymer that includes an absorbing monomeric unit (e.g., a narrow-band absorbing monomeric unit) and/or an emitting monomeric unit having the formula:

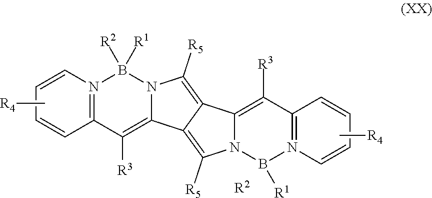

wherein each of R¹, R², R³, R⁴, and R⁵, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R², R³, R⁴, and R⁵, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², R³, R⁴, R⁵, or any combination thereof. FIGS. 6P, 6Q, and 6R show examples of monomeric units that, e.g., can be integrated with the polymer by attachment to R⁴ or R⁵ groups, via, for example, a linker group.

In some embodiments, the polymer dots of the present disclosure can include a polymer that includes an absorbing monomeric unit (e.g., a narrow-band absorbing monomeric unit) and/or an emitting monomeric unit having the formula:

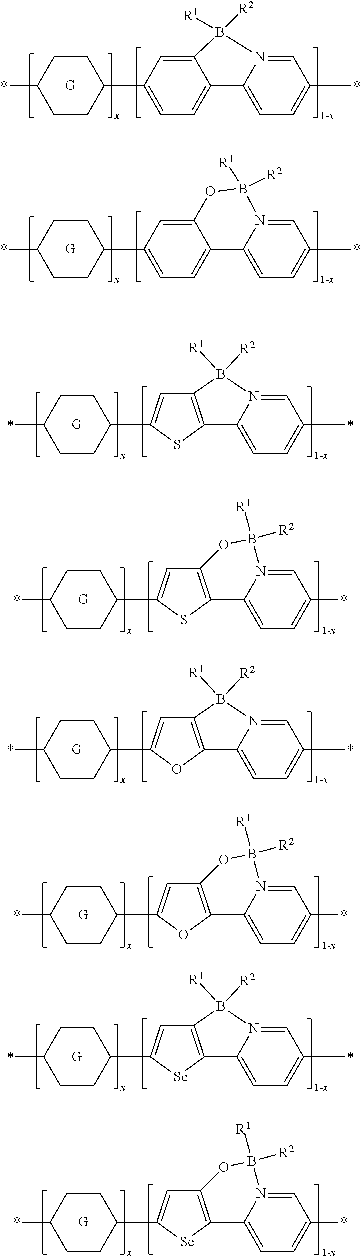

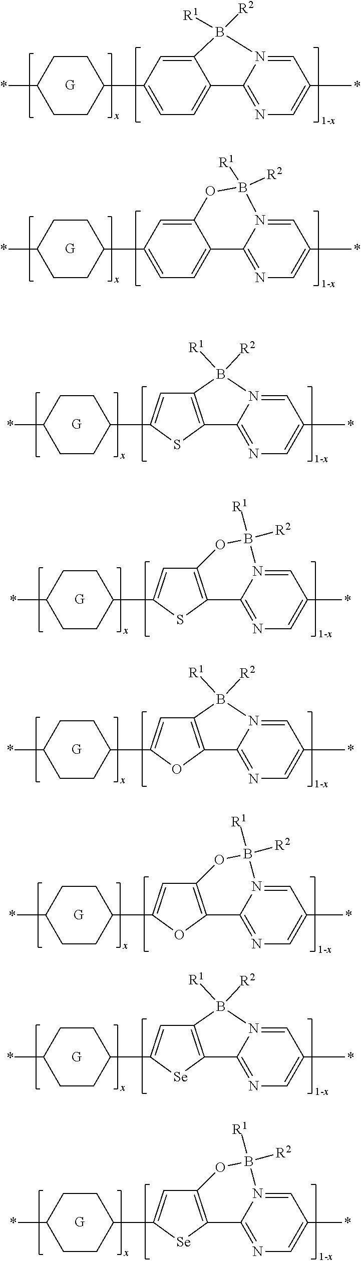

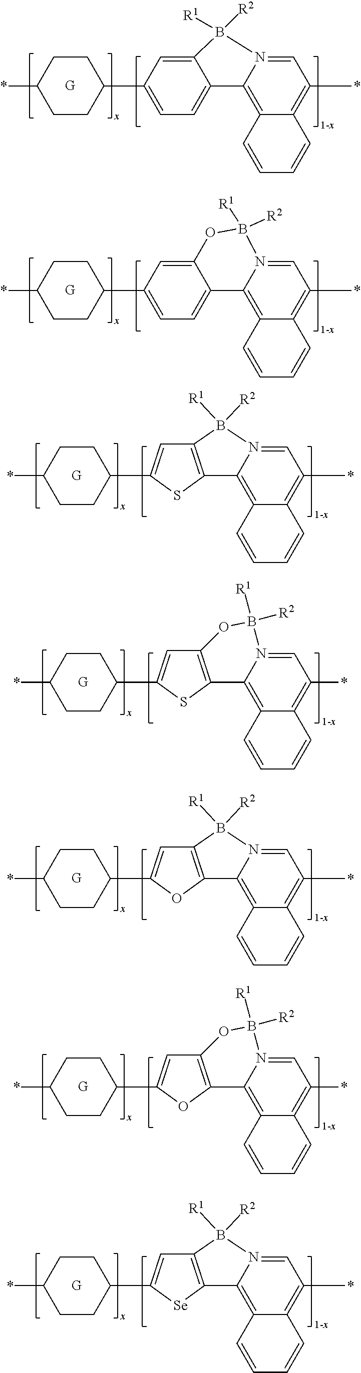

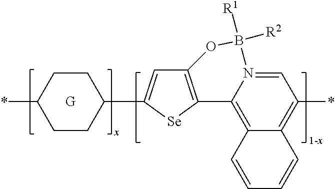

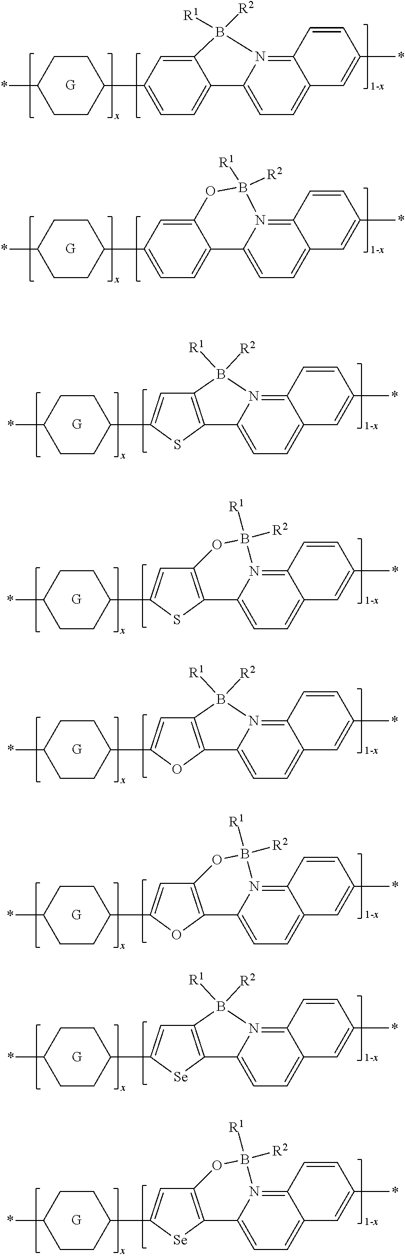

wherein each of R¹ and R² is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹ and R² is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², or any combination thereof. FIGS. 6S, 6T, 6U, and 6V show examples of polymers including absorbing monomeric units.

In some embodiments, the polymer dots of the present disclosure can include a polymer that includes an absorbing monomeric unit (e.g., a narrow-band absorbing monomeric unit) and/or an emitting monomeric unit having the formula:

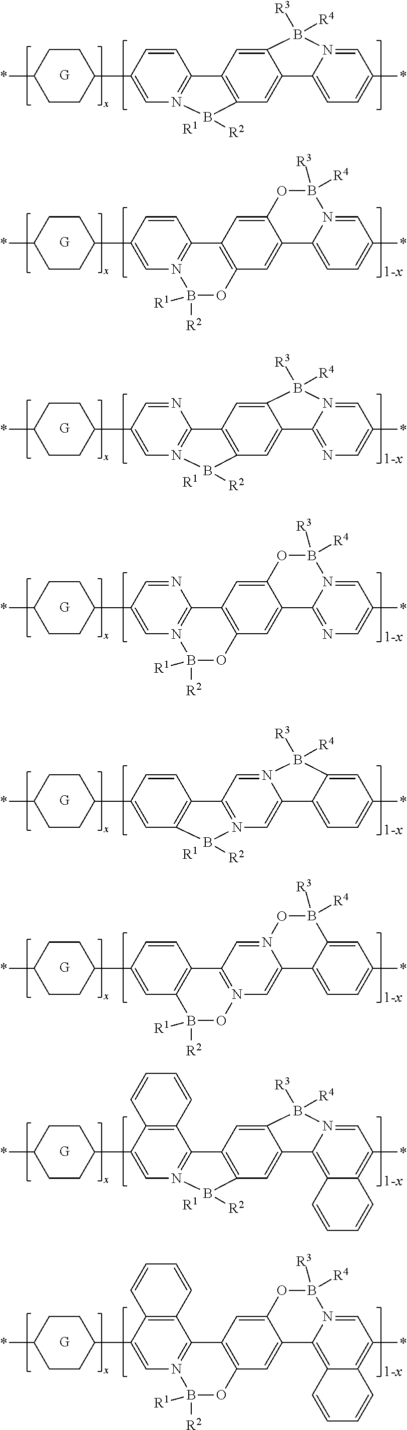

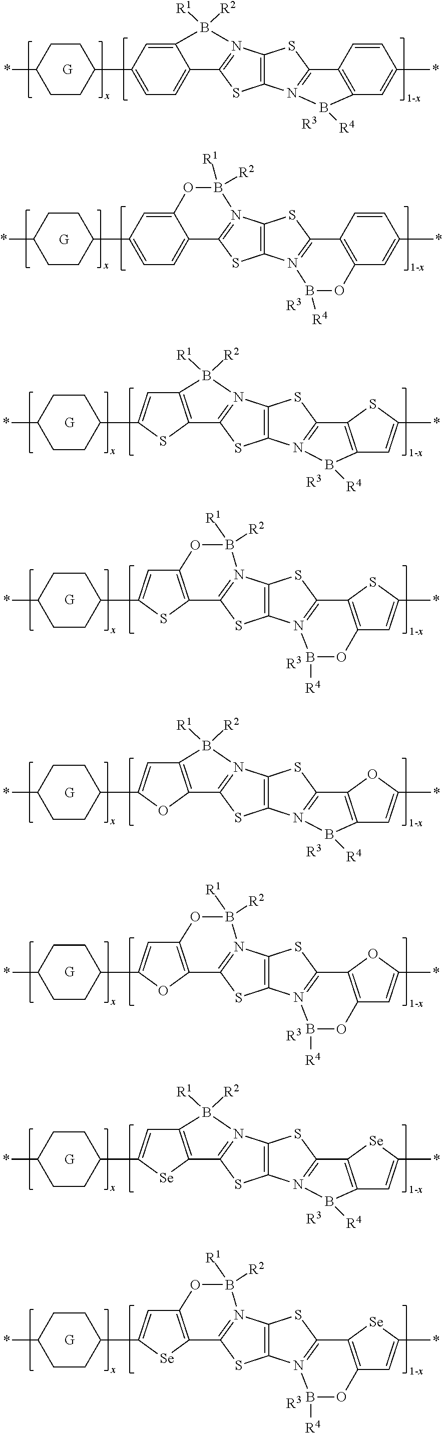

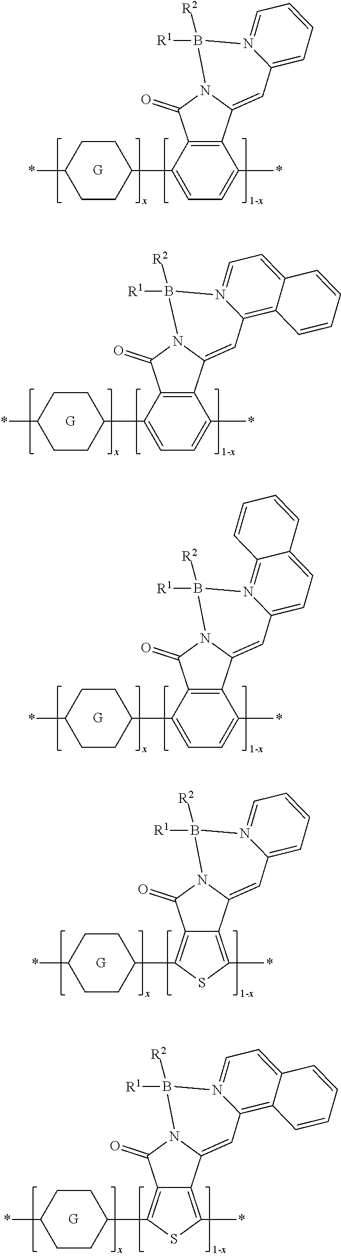

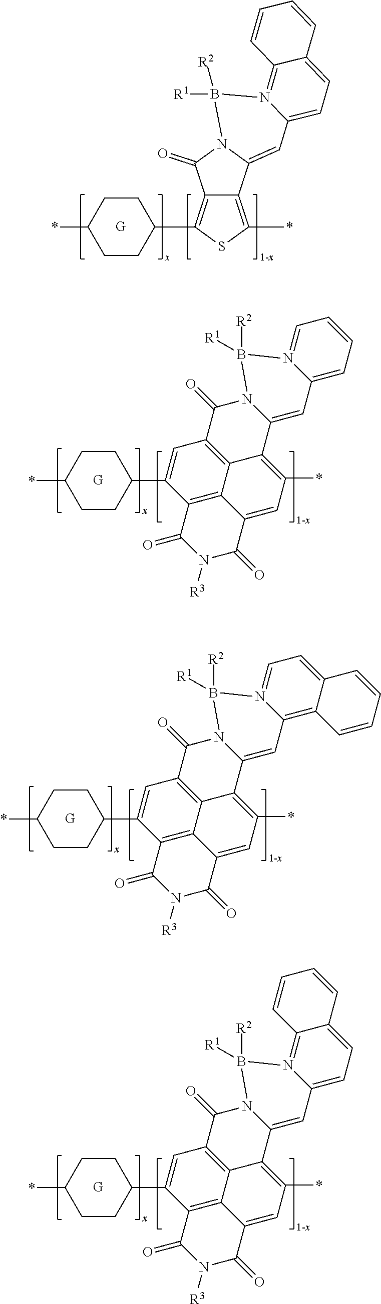

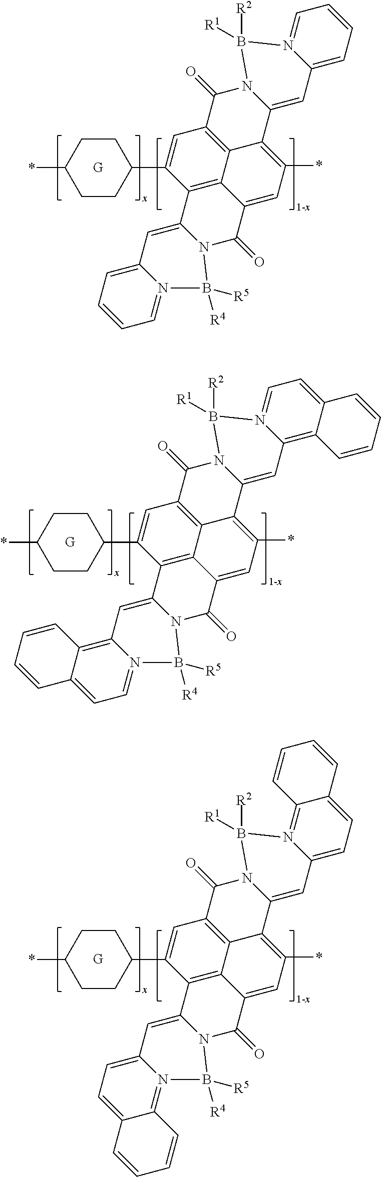

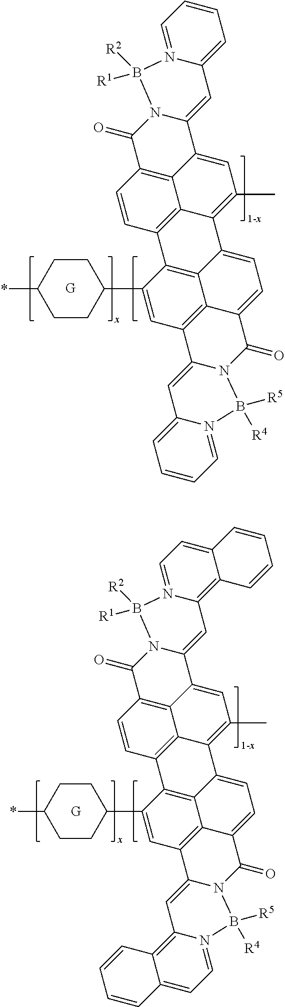

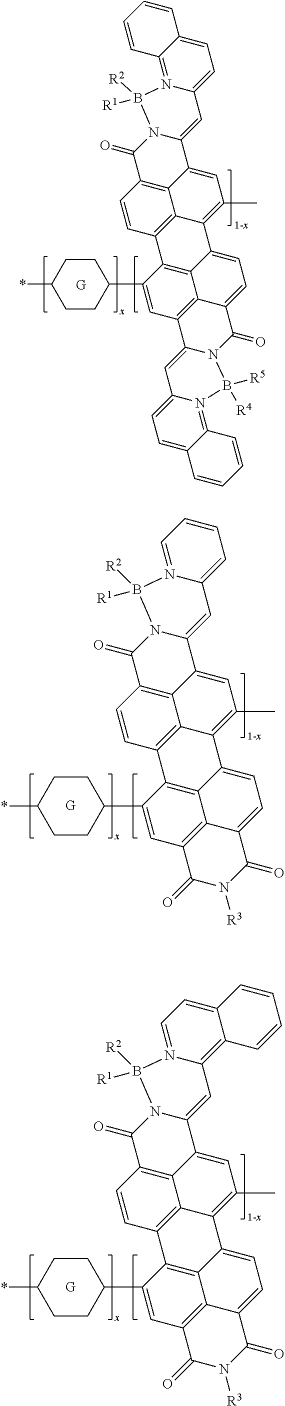

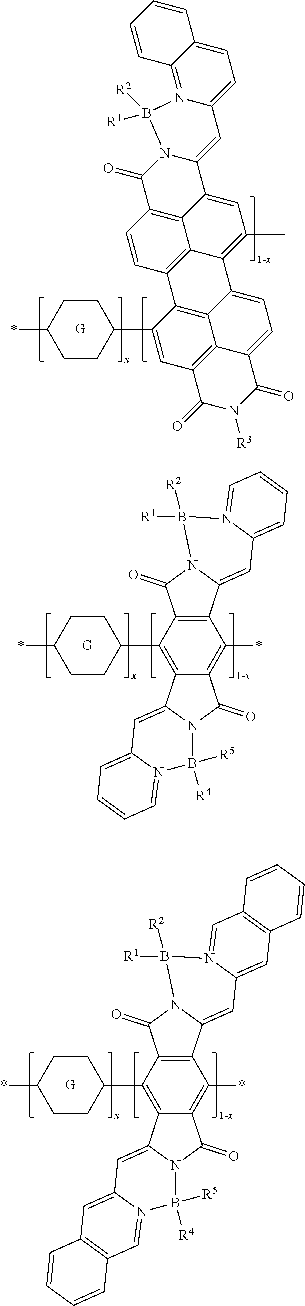

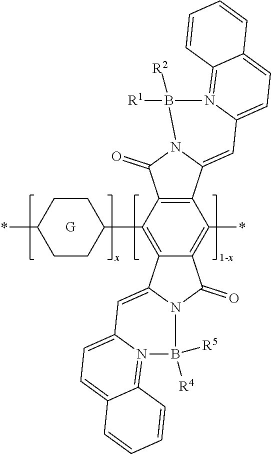

wherein each of R¹, R², R³, R⁴, R⁵, when present, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R², R³, R⁴, R⁵, when present, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², R³, R⁴, R⁵, or any combination thereof. FIGS. 6W-6Z show examples of polymers including absorbing monomeric units.

In some embodiments, the polymer dots of the present disclosure can include a polymer that includes an absorbing monomeric unit (e.g., a narrow-band absorbing monomeric unit) and/or an emitting monomeric unit having the formula:

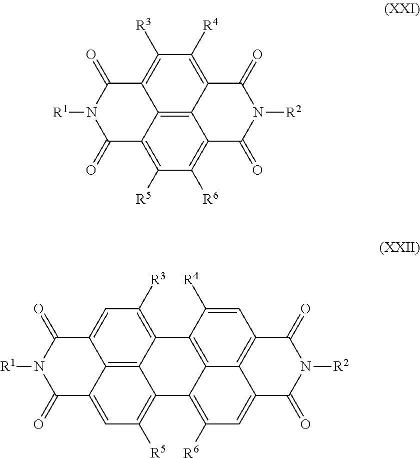

wherein each of R¹, R², R³, R⁴, R⁵, and R⁶, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ether and its derivatives, ester and its derivatives, alkyl ketone, alkyl ester, aryl ester, alkynyl, alkyl amine, fluoroalkyl, fluoroaryl, and polyalkalene (e.g., methoxyethoxyethoxy, ethoxyethoxy, and —(OCH₂CH₂)ₙOH, n=1-50), phenyl, alkyl-(alkoxy-, aryl-, fluoroalkyl-, fluoroaryl-)substituted phenyl, pyridyl, alkyl-(alkoxy-, aryl-, fluoroalkyl-, fluoroaryl-)substituted pyridyl, bipyridyl, alkyl-(alkoxy-, aryl-, fluoroalkyl-, fluoroaryl-)substituted bipyridyl tripyridyl, alkyl-(alkoxy-, aryl-, fluoroalkyl-, fluoroaryl-)substituted tripyridyl, furyl, alkyl-(alkoxy-, aryl-, fluoroalkyl-, fluoroaryl-)substituted furyl, thienyl, alkyl-(alkoxy-, aryl-, fluoroalkyl-, fluoroaryl-)substituted thienyl, pyrrolyl, alkyl-(alkoxy-, aryl-, fluoroalkyl-, fluoroaryl-)substituted pyrrolyl, pyrazolyl, alkyl-(alkoxy-, aryl-, fluoroalkyl-, fluoroaryl-)substituted pyrazolyl, oxazolyl, alkyl-(alkoxy-, aryl-, fluoroalkyl-, fluoroaryl-)substituted oxazolyl, thiazolyl, alkyl-(alkoxy-, aryl-, fluoroalkyl-, fluoroaryl-)substituted thiazolyl, imidazolyl, alkyl-(alkoxy-, aryl-, fluoroalkyl-, fluoroaryl-)substituted imidazolyl, pyrazinyl, alkyl-(alkoxy-, aryl-, fluoroalkyl-, fluoroaryl-)substituted pyrazinyl, benzoxazolyl, alkyl-(alkoxy-, aryl-, fluoroalkyl-, fluoroaryl-)substituted benzoxazolyl, benzothiadiazolyl, alkyl-(alkoxy-, aryl-, fluoroalkyl-, fluoroaryl-)substituted benzothiadiazolyl, fluorenyl, alkyl-(alkoxy-, aryl-, fluoroalkyl-, fluoroaryl-)substituted fluorenyl, triphenylaminyl-substituted fluorenyl, diphenylaminyl-substituted fluorenyl, carbazole, alkyl-(alkoxy-, aryl-fluoroalkyl-, fluoroaryl-)substituted carbazole, carbazolyl, alkyl-substituted carbazolyl, alkyl-substituted triphenylaminyl and alkyl-substituted thiophenyl. As exemplary embodiments, substituents can include alkyl-aryl-substituted carbazole (e.g., 3,6-di-tert-butyl-9-phenyl-9H-carbazole), alkyl substituted phenyl can include 2-alkylphenyl, 3-alkylphenyl, 4-alkylphenyl, 2,4-dialkylphenyl, 3,5-dialkylphenyl, 3,4-dialkylphenyl; alkyl-substituted fluorenyl can include 9, 9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl; alkyl-substituted carbazolyl can include N-alkyl-substituted carbazolyl, 6-alkyl-substituted carbazolyl and 7-alkyl-substituted carbazolyl; alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-alkyl-substituted triphenylaminyl; alkyl-substituted thiophenyl can include 2-alkylthiophenyl, 3-alkylthiophenyl, and 4-alkylthiophenyl, N-dialkyl-4-phenyl, N-diphenyl-4-phenyl, and N-dialkoxyphenyl-4-phenyl. In some embodiments, each of R¹, R², R³, R⁴, R⁵, and R⁶, or two variables on adjacent atoms together with the atoms (e.g., carbons) to which they are attached, when applicable, is independently selected from the group consisting of, but not limited to, hydrogen (H), deuterium (D), halogen, direct or branched alkyl, heteroalkyl, cycloalkyl, heterocycloalkyl, cycloalkylene, heterocycloalkylene, cycloalkenyl, heterocycloalkenyl, alkoxy, aryl, alkaryl (or aralkyl) heteroaryl, aryloxy, hydroxyl, acyl, cyano, nitro, azide, carboxyl, amino, sulfide, ester, and alkynyl. The absorbing monomeric unit, the emitting monomeric unit, or a combination of both the absorbing monomeric unit and the emitting monomeric unit can be integrated into a backbone of the polymer (e.g., polymerized in the polymer) and/or covalently attached to the backbone, a terminus, or a sidechain of the polymer. For example, the absorbing monomeric unit and/or emitting monomeric unit can be covalently attached to the polymer through at least one attachment (or an attachment via a linker moiety) to R¹, R², R³, R⁴, R⁵, and R⁶, or any combination thereof. FIGS. 6AA-6EE shows examples of monomeric units that, e.g., can be integrated with the polymer by attachment to R¹, R³ or R⁶ groups.

In some embodiments, the polymer dots of the present disclosure can include a polymer that includes an absorbing monomeric unit (e.g., a narrow-band absorbing monomeric unit) and/or an emitting monomeric unit derived from a squaraine derivative monomer shown in FIGS. 6FF and 6GG. Exemplary syntheses of a polymer containing a squaraine derivative monomeric unit is also shown in FIGS. 6FF and 6GG.

In some embodiments, the absorbing monomeric units of the present disclosure can be incorporated into the backbone of a conventional semiconducting polymer to obtain narrow-band absorbing polymers. In this embodiment, the narrow-band absorbing monomeric units can be copolymerized with other monomeric units such as fluorene monomeric unit, phenylene vinylene monomeric unit, phenylene monomeric unit, benzothiadiazole monomeric unit, thiophene monomeric unit, carbazole monomeric unit, or any other monomeric units to form narrow-band absorbing polymers. In some embodiments, the absorbing monomeric units can be chemically linked to the side chains of the conventional semiconducting polymer to obtain narrow-band absorbing polymers. In some embodiments, the semiconducting polymer is luminescent. In this embodiment, conventional luminescent semiconducting polymers include, but are not limited to fluorene polymers, phenylene vinylene polymers, phenylene polymers, benzothiadiazole polymers, thiophene polymers, carbazole fluorene polymers and their copolymers, and any other conventional semiconducting polymers.

In some embodiments, a semiconducting polymer is a broad-band semiconducting polymer. The concentration of the absorbing monomeric units relative to broad-band semiconducting polymers can be adjusted to maximize the emission and fluorescence performance of the Pdots, such as narrow emission FWHM, high fluorescence quantum yield, desirable fluorescence lifetime, etc. In some embodiments, the narrow-band absorbing nanoparticle further includes metal complexes and/or their derivatives. Metal complexes and their derivatives include but are not limited to their alkyl derivatives, aryl derivatives, alkyne derivatives, aromatic derivatives, alkoxide derivatives, aza derivatives, their extended systems, and analogues. The absorbing polymers, emitting polymers, and/or absorbing and emitting polymers can also include any other monomeric units. The metals can be any metal such as Na, Li, Zn, Mg, Fe, Mn, Co, Ni, Cu, In, Si, Ga, Al, Pt, Pd, Ru, Rh, Re, Os, Ir, Ag, Au and so on.

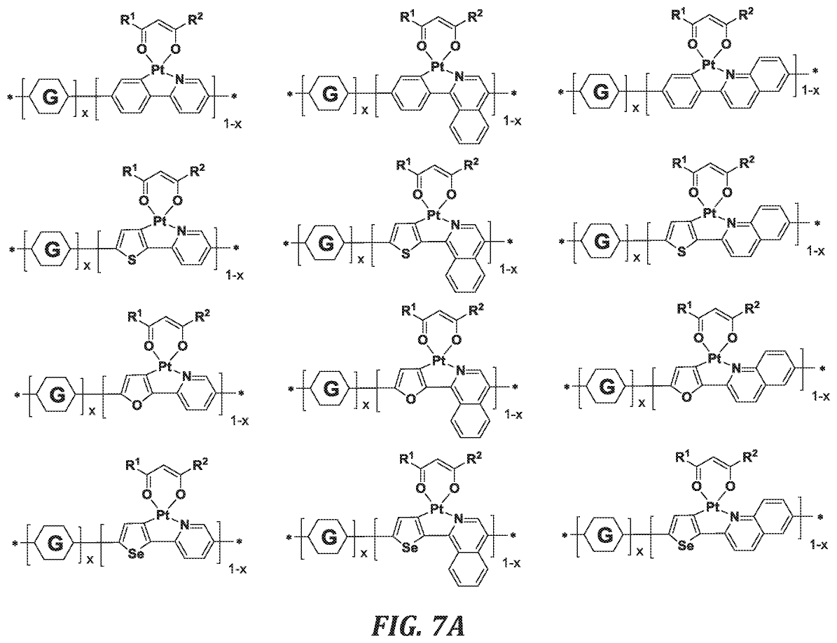

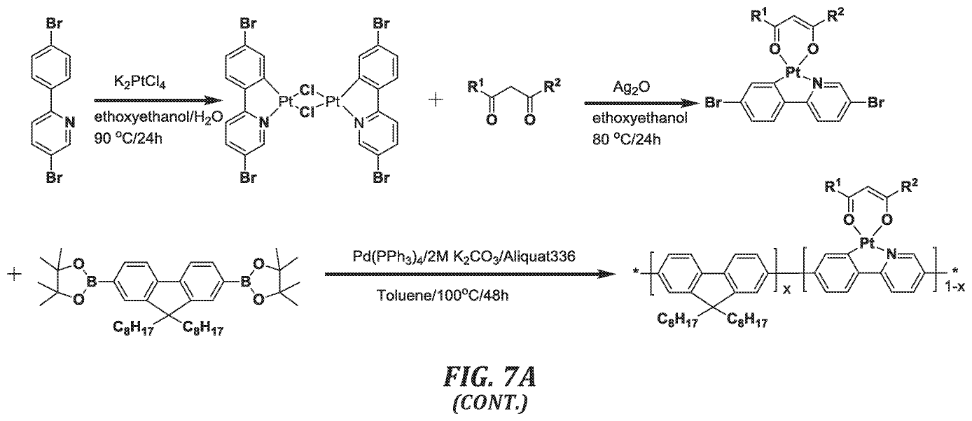

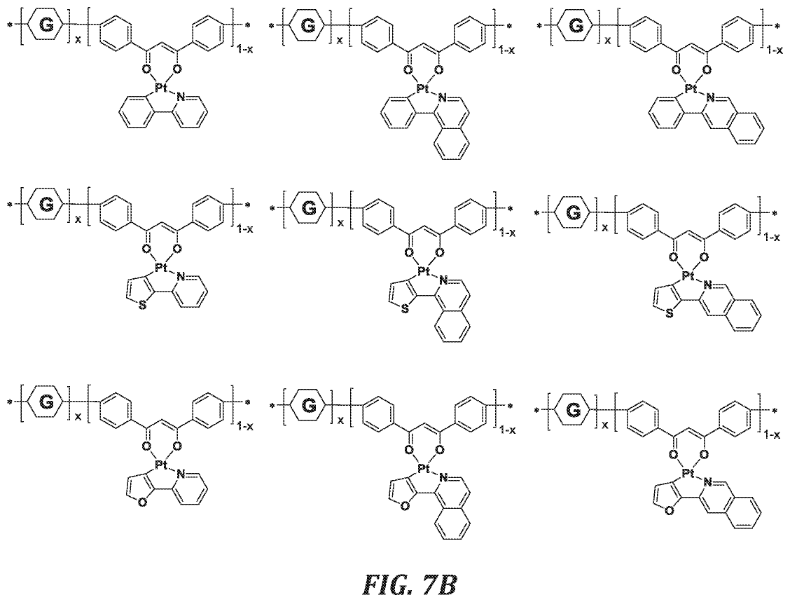

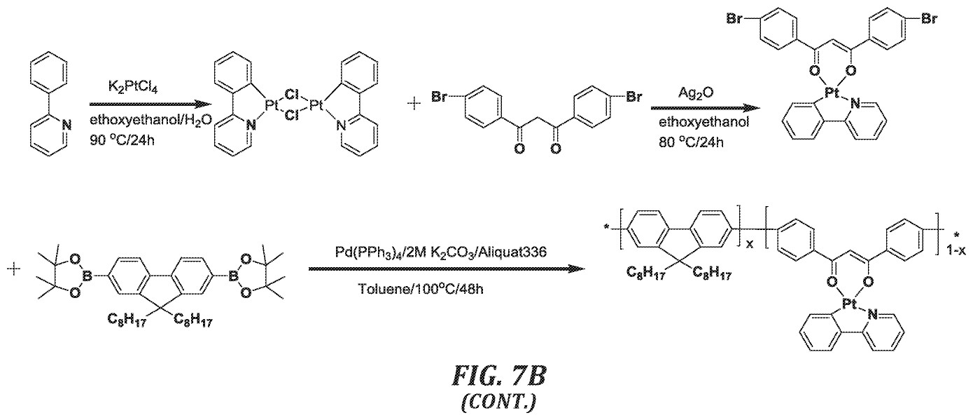

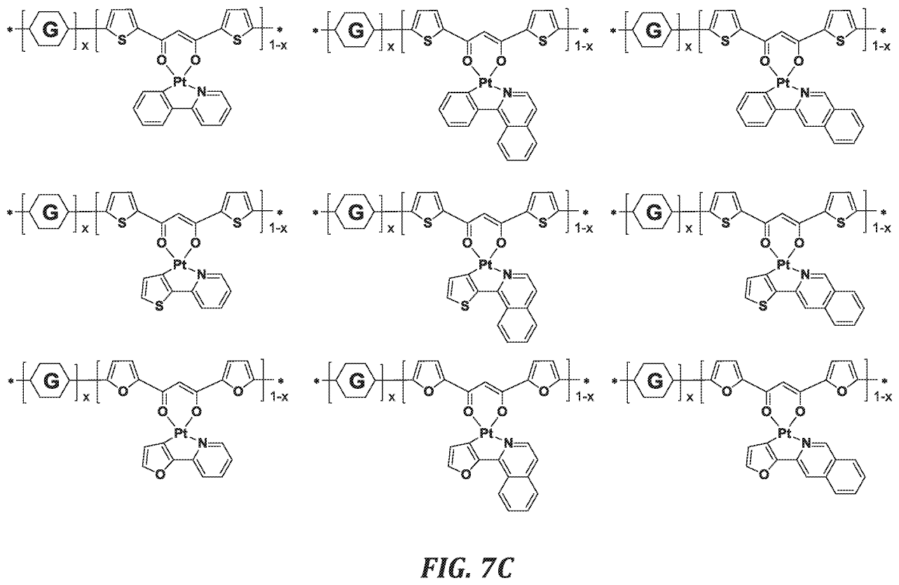

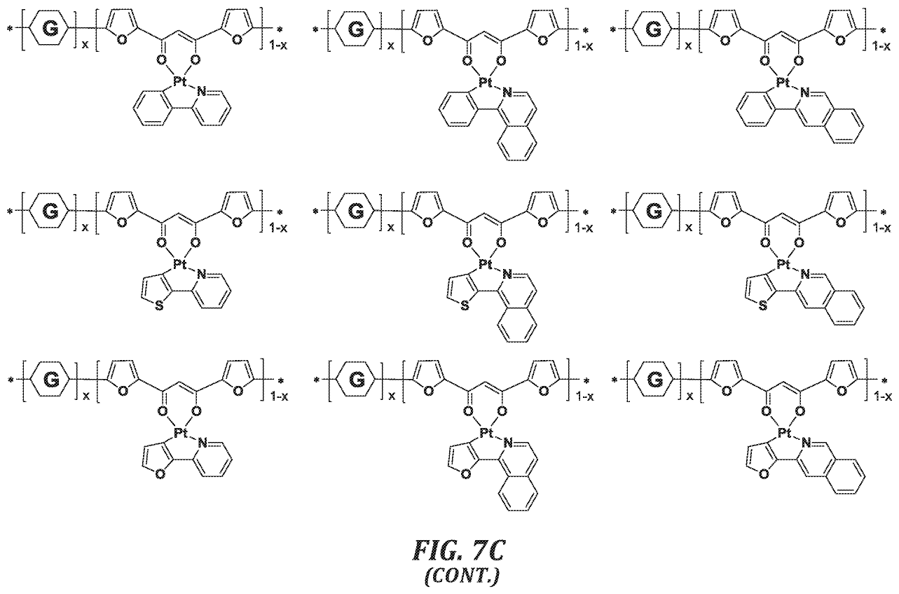

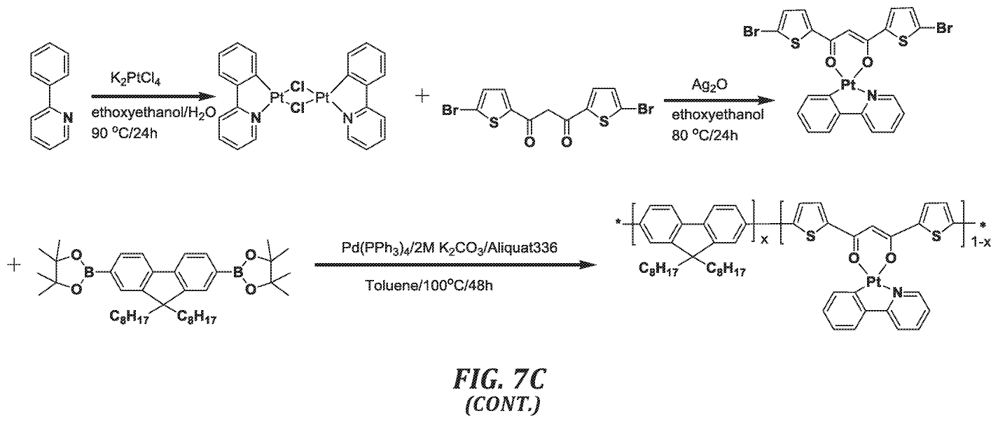

Examples of metal complexes and metal complex derivatives are shown in FIG. 7A, FIG. 7B, and FIG. 7C. Metal complexes and metal complex derivatives can be polymerized to form polymers (e.g., homopolymers or heteropolymers) and/or can be attached (e.g., covalently attached) to a polymer backbone, sidechain and/or terminus. As shown in FIG. 7A, the metal complexes of the present disclosure include derivatives of the metal complexes. The metal complex monomeric units shown in FIG. 7A can include the compounds as shown, wherein R¹ and R² are independently selected from the group consisting of, but not limited to, phenyl, alkyl-substituted phenyl, alkyl-substituted fluorenyl, diphenyl-substituted fluorenyl, triphenylaminyl-substituted fluorenyl, diphenylaminyl-substituted fluorenyl, alkyl-substituted carbazolyl, alkyl-substituted triphenylaminyl and alkyl-substituted thiophenyl. Alkyl substituted phenyl can include 2-alkylphenyl, 3-alkylphenyl, 4-alkylphenyl, 2,4-dialkylphenyl, 3,5-dialkylphenyl, and 3,4-dialkylphenyl. Alkyl-substituted fluorenyl can include 9,9-dialkyl-substituted fluorenyl, 7-alkyl-9,9-dialkyl-substituted fluorenyl, 6-alkyl-9,9-dialkyl-substituted fluorenyl, 7-triphenylaminyl-9,9-dialkyl-substituted fluorenyl and 7-diphenylaminyl-9,9-dialkyl-substituted fluorenyl. Alkyl-substituted carbazolyl can include N-alkyl-substituted carbazolyl, 6-alkyl-substituted carbazolyl and 7-alkyl-substituted carbazolyl. Alkyl-substituted triphenylaminyl can include 4′-alkyl-substituted triphenylaminyl, 3′-alkyl-substituted triphenylaminyl, 3′,4′-dialkyl-substituted triphenylaminyl and 4′,4″-dialkyl-substituted triphenylaminyl. Alkyl-substituted thiophenyl can include 2-alkylthiophenyl, 3-alkylthiophenyl, and 4-alkylthiophenyl. The alkyl substituents can include CₙH₂ₙ₊₁, or CₙF₂₊₁ or —CH₂CH₂[OCH₂CH₂]ₙ—OCH₃, wherein n is 1 to 20. In some embodiments, n can be between 1 to 50 or higher. As will be further understood by one of ordinary skill in the art, the general monomeric unit (G) and the narrow band metal complex monomeric units are present in the polymer at a ratio where G is present as x and the narrow band monomeric unit is present as 1−x. For example, G can be present at 90% or x=0.9 and the narrow band monomeric unit is present at 10% or 1−x=0.1. FIGS. 7B and 7C show additional example monomeric units for use in the present disclosure.

In some embodiments, the absorbing polymers, emitting polymers, and/or absorbing and emitting polymers for making nanoparticles include porphyrin, metalloporphyrin, and their derivatives as monomeric units. Porphyrin, metalloporphyrin, and their derivatives can be polymerized to form polymers (e.g., homopolymers or heteropolymers) and/or can be attached (e.g., covalently attached) to a polymer backbone, sidechain and/or terminus. Porphyrin, metalloporphyrin, and their derivatives include but are not limited to their alkyl derivatives, aryl derivatives, alkyne derivatives, aromatic derivatives, alkoxide derivatives, aza derivatives, their extended systems and analogues. The metals in the metalloporphyrins can be any metal such as Na, Li, Zn, Mg, Fe, Mn, Co, Ni, Cu, In, Si, Ga, Al, Pt, Pd, Ru, Rh, Re, Os, Ir, Ag, Au and so on. The narrow-band absorbing polymers can also include any other monomeric units.

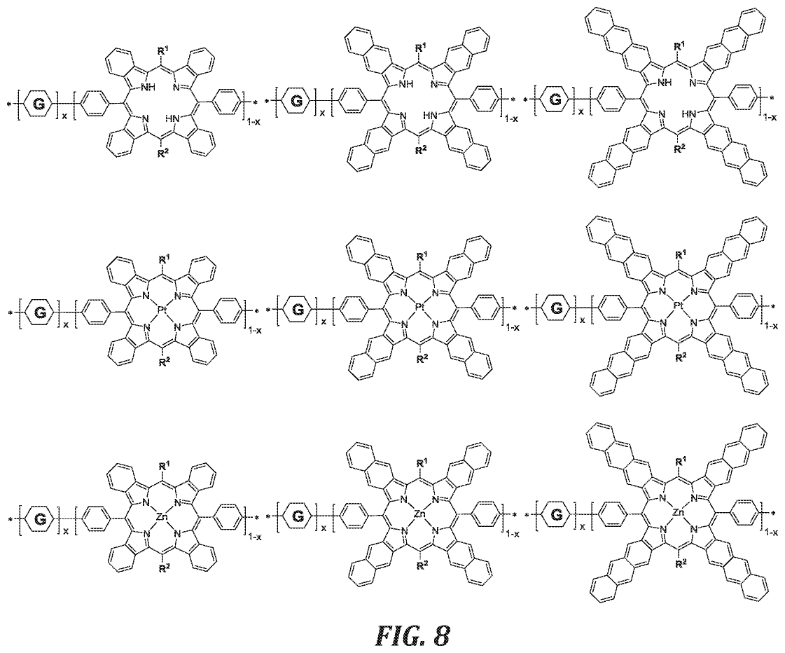

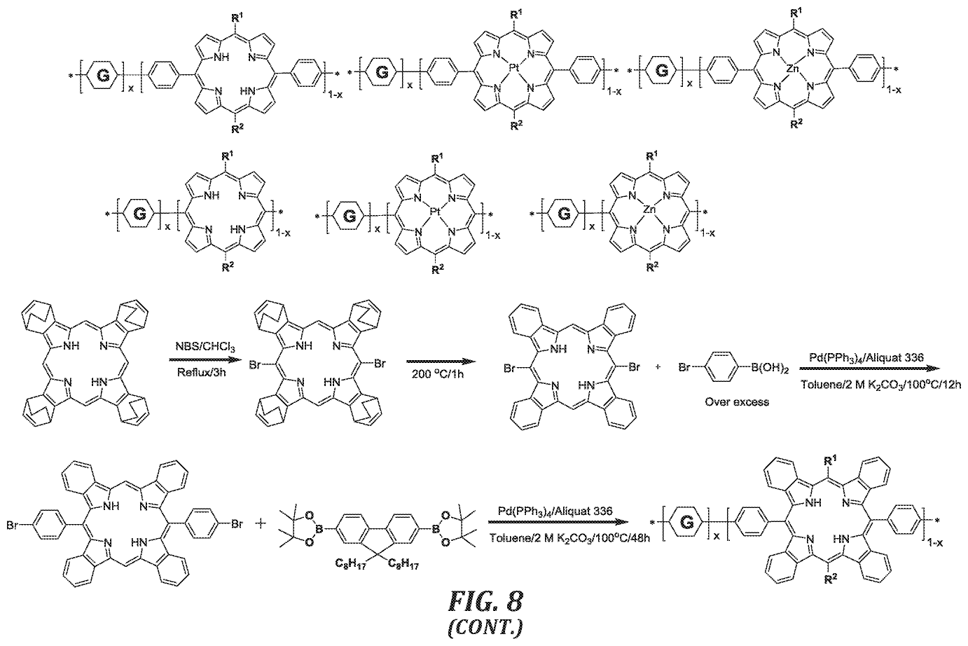

FIG. 8 shows example porphyrin and porphyrin derivatives for use in the present disclosure. As shown in FIG. 8, the porphyrin derivatives can complex, e.g., with Pt and Zn. Also, R¹ and R² can be independently selected from the group consisting of, but not limited to, phenyl, alkyl-substituted phenyl, alkyl-substituted fluorenyl, alkyl-substituted carbazolyl, alkyl-substituted triphenylaminyl, alkyl-substituted thiophenyl, fluorine (F), cyano (CN) and trifluoro (CF₃). Alkyl substituted phenyl can include 2-alkylphenyl, 3-alkylphenyl, 4-alkylphenyl, 2,4-dialkylphenyl, 3,5-dialkylphenyl, and 3,4-dialkylphenyl. Alkyl-substituted fluorenyl can include 9,9-dialkyl-substituted fluorenyl, 7-alkyl-9,9-dialkyl-substituted fluorenyl and 6-alkyl-9,9-dialkyl-substituted fluorenyl. Alkyl-substituted carbazolyl can include N-alkyl-substituted carbazolyl, 6-alkyl-substituted carbazolyl and 7-alkyl-substituted carbazolyl. Alkyl-substituted thiophenyl can include 2-alkylthiophenyl, 3-alkylthiophenyl, and 4-alkylthiophenyl. The alkyl substituents can include CₙH₂ₙ₊₁, or CₙF₂ₙ₊₁ or —CH₂CH₂[OCH₂CH₂]ₙ—OCH₃, wherein n is 1 to 20. In some embodiments, n can be between 1 to 50 or higher. The monomeric units can be integrated into a backbone of the polymer (e.g., by copolymerizing in the polymer) and/or attached by covalent attachment to the backbone, a terminus, or a sidechain of the polymer through at least one attachment (or an attachment via a linker moiety) to R¹, R², or any combination thereof. Alternatively, as shown in FIG. 8, the monomeric units described herein can be integrated with the polymer by attachment as shown by brackets.

In some embodiments, the narrow-band absorbing nanoparticle can also include emissive polymer, physically mixed or chemically cross-linked with other components including, e.g. inorganic luminescent materials, to tune emission color, improve quantum yield and photostability, and the like.

In certain embodiments, the narrow-band absorbing nanoparticle further includes a matrix polymer. In some embodiments, the matrix polymer is a non-semiconducting polymer. In some embodiments, the matrix polymer is a semiconducting polymer. In some embodiments, the matrix polymer is both semiconducting and non-semiconducting (e.g., the matrix polymer can have semiconducting segments as well as non-semiconducting segments). In some embodiments, the matrix polymer is an amphiphilic polymer. In specific embodiments, the matrix polymer includes a poly((meth)acrylic acid)-based copolymer, a polydiene-based copolymer, a poly(ethylene oxide)-based copolymer, a polyisobutylene-based copolymer, a polystyrene-based copolymer, a polysiloxane-based copolymer, a poly(ferrocenyldimethylsilane)-based copolymer, a poly(2-vinyl naphthalene)-based copolymer, a poly (vinyl pyridine and N-methyl vinyl pyridinium iodide)-based copolymer, a poly(vinyl pyrrolidone)-based copolymer, a polyacrylamide-based copolymer, a poly(meth)acrylate-based copolymer, a polyphenylene-based copolymer, a polyethylene-based copolymer, a poly(ethylene glycol)-based copolymer, a polylactide-based copolymer, a polyurethane-based copolymer, or any combination thereof. In certain embodiments, the matrix polymer is polystyrene-graft-poly(ethylene oxide).

In some embodiments, the matrix polymer is functionalized, and can be referred to as a “functionalization polymer.” A functionalization polymer includes functional groups which can be used for, e.g., bioconjugation. Exemplary functional groups include without limitation alkyne, strained alkyne, azide, diene, alkene, cyclooctyne, haloformyl, hydroxyl, aldehyde, alkenyl, alkynyl, anhydride, carboxamide, amines, amides, azo compound, carbonate, carboxylate, carboxyl, cyanates, ester, haloalkane, imine, isocyanates, nitrile, nitro, phosphino, phosphate, phosphate, pyridyl, sulfonyl, sulfonic acid, sulfoxide, and thiol groups, or any combination thereof.

In some embodiments, the narrow-band absorbing nanoparticle is bioconjugated to a biomolecule. In some embodiments, the biomolecule is conjugated to the absorbing polymer, the emitting polymer, the absorbing and emitting polymer, the matrix polymer, or any combination thereof. In certain embodiments, the attachment (i.e., conjugation) of the biomolecule to the nanoparticle (“bioconjugation”) includes a covalent bond. In some embodiments, the absorbing polymer, the emitting polymer, the absorbing and emitting polymer, the matrix polymer, or any combination thereof include at least one functional group suitable for conjugation. In certain embodiments, a functional group includes a hydrophilic functional group that is hydrophilic in nature and is attached to the polymer (e.g., on the side chain). In some aspects, hydrophilic functional groups include carboxylic acid or salts thereof, amino, mercapto, azido, aldehyde, ester, hydroxyl, carbonyl, sulfate, sulfonate, phosphate, cyanate, succinimidyl ester, substituted derivatives thereof. In certain embodiments, hydrophilic functional groups include carboxylic acid or salts thereof, amino, mercapto, azido, aldehyde, ester, hydroxyl, carbonyl, sulfate, phosphate, cyanate, succinimidyl ester, and substituted derivatives thereof. In certain embodiments, the hydrophilic functional groups are suitable for bioconjugation. In some aspects, the hydrophilic functional groups are suitable for bioconjugation and also stable in aqueous solution (e.g., the groups do not hydrolyze). Such functional groups can be found by one of ordinary skill in the art, for example in Bioconjugate Techniques (Academic Press, New York, 1996 or later versions) the content of which is herein incorporated by reference in its entirety for all purposes. Some hydrophilic functional groups suitable for bioconjugation include carboxylic acid or salts thereof, amino, mercapto, azido, aldehyde, ester, hydroxyl, carbonyl, phosphate, cyanate, succinimidyl ester, and substituted derivatives thereof. In some aspects, hydrophilic functional groups suitable for conjugation include carboxylic acid or salts thereof, amino groups, mercapto, succinimidyl ester, and hydroxyl. A non-limiting list of hydrophilic functional group pairs is provided below in Table 1.

TABLE 1
Exemplary hydrophilic functional group
pairs for conjugation chemistry.

| Functional Groups | Reacts With |
|---|---|
| Ketone and aldehyde groups | Amino, hydrazido and aminooxy |
| Imide | Amino, hydrazido and aminooxy |
| Cyano | Hydroxy |
| Alkylating agents (such as haloalkyl | Thiol, amino, hydrazido, |
| groups and maleimido derivatives) | aminooxy |
| Carboxyl groups (including activated | Amino, hydroxyl, hydrazido, |
| carboxyl groups) | aminooxy |

In some embodiments, the functional group includes a hydrophobic functional group that is attached to the polymer (e.g., on a hydrophobic side chain). In some embodiments, hydrophobic functional groups generally include, but are not limited to, alkynes, alkenes, and substituted alkyl derivatives that are suitable for conjugation. Some of the hydrophobic functional groups are chemically modified to form hydrophilic functional groups used for bioconjugation. In certain embodiments, hydrophobic functional groups attached to a polymer are suitable for bioconjugation. For example, in some aspects, the hydrophobic functional groups include without limitation those used for click chemistry, such as alkyne, strained alkyne, azide, diene, alkene, cyclooctyne, and phosphine groups. In some aspects, these hydrophobic functional groups are, e.g., used for bioconjugation reactions that covalently couple the narrow-band absorbing nanoparticles to a biologically relevant molecule (e.g., an antibody).

### Bioconjugated Narrow-Band Absorbing Nanoparticles

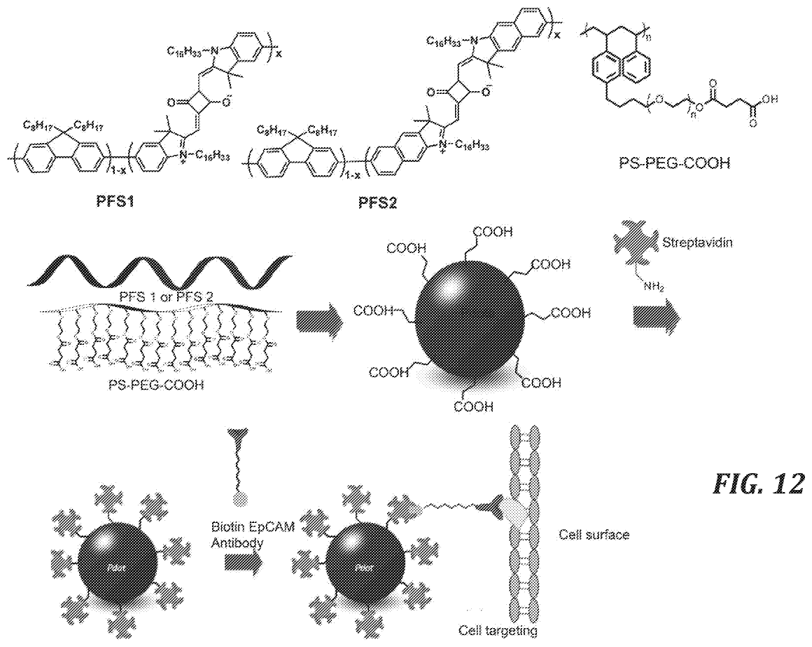

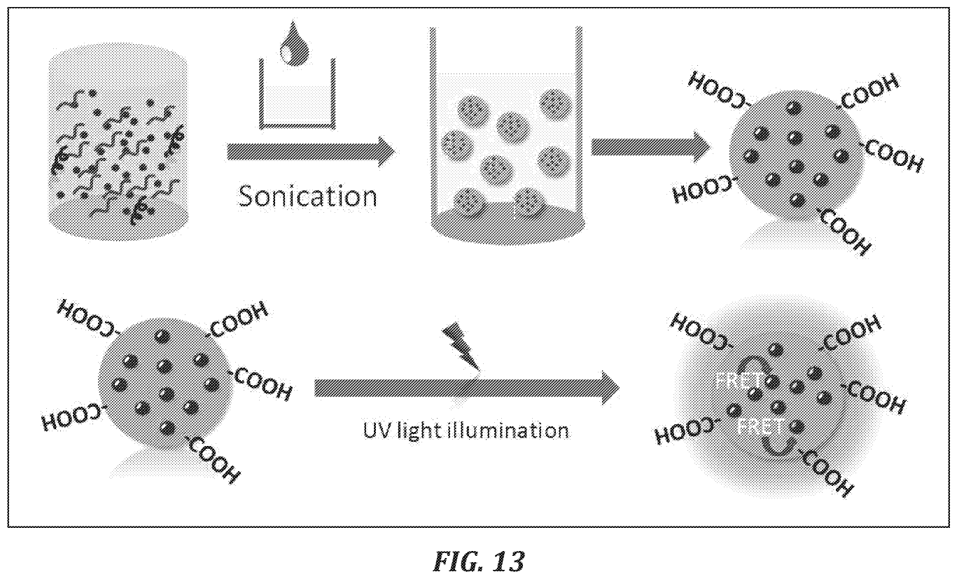

In certain embodiments, a polymer nanoparticle can be attached to a biomolecule using biotinylation and/or activated bioconjugation (FIG. 12). This attachment can be referred to as “bioconjugation” wherein the biomolecule is conjugated (bioconjugated) to the polymer nanoparticle. For example, a polymer nanoparticle including a plurality of carboxylic acid functional groups can undergo coupling in the presence of EDC as a bioconjugation agent (i.e., activates the bioconjugation) and a biomolecule including a primary amine. The biomolecule can further undergo biotinylation, e.g., with a biotinylated antibody. The biotinylated construct can then bind to a selected target, e.g., a cell surface.

As described herein, some of the functional groups are “suitable for bioconjugation,” which is used to refer to a functional group that is covalently bonded to a biomolecule, such as an antibody, protein, nucleic acid, streptavidin, or other molecule of biological relevance. Such functional groups can be found by one of ordinary skill in the art, for example in Bioconjugate Techniques (Academic Press, New York, 1996 or later versions) the content of which is herein incorporated by reference in its entirety for all purposes. In some aspects, functional groups suitable for bioconjugation include functional groups that are capable of being conjugated to a biomolecule under a variety of conditions, such as, e.g., in polar or non-polar solvents. In certain embodiments, functional groups suitable for bioconjugation include functional groups that are conjugated to a biomolecule in an aqueous solution. In some aspects, functional groups suitable for bioconjugation can include functional groups that are conjugated to a biomolecule in an aqueous solution in which the biomolecule retains its biological activity (e.g., monoclonal binding specificity for an antibody). In certain embodiments, functional groups suitable for bioconjugation can include functional groups that are covalently bonded to a biomolecule. For example, typical covalent bonding attachments of functional groups to biomolecules can include, e.g., a carboxyl functional group reacting with an amine on a biomolecule to form an amide bond, a sulfhydryl functional group reacting with a sulfhydryl group on a biomolecule to form a cysteine bond, or an amino functional group reacting with a carboxyl group on a biomolecule to form an amide bond. In some aspects, the specific reactions of bioconjugation can include the functional group pairs in Table 1.

In some embodiments, the biomolecule includes a biomarker, an antibody, an antigen, a cell, a nucleic acid, an enzyme, a substrate for an enzyme, a protein, a lipid, a carbohydrate, or any combination thereof. In some embodiments, the biomolecule includes streptavidin, a protein, an antibody, a nucleic acid molecule, a lipid, a peptide, an aptamer, a drug, or any combination thereof. In specific embodiments, the biomolecule includes a protein, a nucleic acid molecule, a lipid, a peptide, a carbohydrate, or any combination thereof. In certain embodiments, the biomolecule includes an aptamer, a drug, an antibody, an enzyme, a nucleic acid, or any combination thereof. In specific embodiments, the biomolecule includes streptavidin. In certain embodiments, the biomolecule includes a cell.

In certain embodiments, the term “biomolecule” describes a synthetic or naturally occurring protein, glycoprotein, peptide, amino acid, metabolite, drug, toxin, nucleic acid, nucleotide, carbohydrate, sugar, lipid, fatty acid and the like. Desirably, the biomolecule is attached to the functional group of the narrow-band absorbing nanoparticle via a covalent bond. For example, if the functional group of the nanoparticle is a carboxyl group, a protein biomolecule can be directly attached to the nanoparticle by cross-linking the carboxyl group with an amine group of the protein molecule. In some embodiments, each narrow-band absorbing polymer nanoparticle can have only one biomolecule attached. In some embodiments, each narrow-band absorbing nanoparticle can have two biomolecules attached. The two biomolecules can be the same or different. In some embodiments, each narrow-band absorbing nanoparticle can have three or more biomolecules attached. The three or more biomolecules can be the same or different. In some embodiments, the biomolecular conjugation does not change substantively the absorptive and/or emissive properties of the narrow-band absorbing nanoparticles. For example, the bioconjugation does not broaden the absorption spectra, does not reduce fluorescence quantum yield, does not change the photostability etc.

In some aspects, the narrow-band absorbing nanoparticles are modified with a functional group and/or biomolecular conjugates for a variety of applications, including but not limited to flow cytometry, fluorescence activated sorting, immunofluorescence, immunohistochemistry, fluorescence multiplexing, single molecule imaging, single particle tracking, protein folding, protein rotational dynamics, DNA and gene analysis, protein analysis, metabolite analysis, lipid analysis, FRET based sensors, high throughput screening, cellular imaging, in vivo imaging, bioorthogonal labeling, click reactions, fluorescence-based biological assays such as immunoassays and enzyme-based assays, fluorescence microscopy, and a variety of fluorescence techniques in biological assays and measurements.

In some embodiments, the emitting monomeric unit includes a chromophoric unit. In some embodiments, the emitting monomeric unit emits luminescent light. In certain embodiments, the emitting monomeric unit emits fluorescent light. In some embodiments, the emitting monomeric unit includes a benzene, a benzene derivative, a fluorene, a fluorene derivative, a benzothiadiazole, a benzothiadiazole derivative, a thiophene, a thiophene derivative, a BODIPY, a BODIPY derivative, a porphyrin, a porphyrin derivative, a perylene, a perylene derivative, a squaraine, a squaraine derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, or any combination thereof. In particular embodiments, the emitting monomeric unit includes BODIPY, a BODIPY derivative, squaraine, a squaraine derivative, or any combination thereof. In specific embodiments, the emitting monomeric unit includes BODIPY or a BODIPY derivative. In some embodiments, the emitting monomeric unit includes squaraine or a squaraine derivative.

As described further herein, the present disclosure includes a wide variety of polymer dots that exhibit narrow band absorption properties (e.g., an absorbance width of less than 150 nm at 10% (or in some embodiments, at 15%) of the absorbance maximum). As described further herein, the variety of polymer dots of the present disclosure can include polymers that have a narrow band absorption unit (e.g., a narrow band absorbing monomeric unit, and/or a narrow band absorbing unit). For example, the present disclosure can include a homopolymer or heteropolymer including a narrow band absorbing monomeric unit, such as BODIPY, a BODIPY derivative monomeric unit, or any combination thereof. For example, the homopolymer or heteropolymer can include a narrow band absorbing monomeric unit that includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof.

### Methods of Making Narrow-Band Absorbing Nanoparticles

A variety of polymerization reactions can be used for synthesis of the polymers described herein. For example, semiconducting polymers including homo-polymer and multi-component copolymer or heteropolymer can be synthesized by using a variety of different reactions. Non-limiting examples of reactions for synthesizing semiconducting polymers include the Heck, Mcmurray and Knoevenagel, Wittig, Homer, Suzuki-Miyaura, Sonogashira, Yamamoto, Stille coupling reaction, and so on. Other polymerization strategies such as electropolymerization, oxidative polymerization can also be employed to make semiconducting polymers. Furthermore, microwave-assisted polymerization takes less time and often can give higher molecular weight and yield. The monomeric units and any of the substituents on the monomeric units (such as the substituents described herein) can also be made using standard synthesis methods generally well known in the art.

In some embodiments, narrow-band absorbing nanoparticle can be prepared by using the solvent mixing method. The solvent mixing method involves quickly mixing a solution of the polymer(s) in a good solvent (such as tetrahydrofuran) with a miscible solvent (such as water) to fold the polymer(s) into nanoparticle form, and nanoparticles can be obtained after removal of the good solvent. In some embodiments, the narrow-band absorbing polymer dots can also be prepared by an emulsion or miniemulsion method, based on shearing a mixture including two immiscible liquid phases (such as water and another immiscible organic solvent) with the presence of a surfactant.

In some embodiments, the present disclosure can include methods of making a nanoparticle. The methods can include providing a solvent solution including an absorbing polymer, an emitting polymer, and/or an absorbing and emitting polymer, the polymer being in an elongated coil for; and mixing the solvent solution including the polymer(s) with a miscible solvent to form a condensed polymer (nanoparticle). In another aspect, the present disclosure can include a method of making a nanoparticle that includes providing a solvent solution including an absorbing polymer, an emitting polymer, and/or an absorbing and emitting polymer, the polymer being in an elongated coil form; and mixing the solvent solution including the polymer(s) with an immiscible solvent to form a condensed polymer (nanoparticle).

In some embodiments, nanoparticles can be made as polymer nanoparticles that have intrachain energy transfer between, e.g., an absorbing monomeric unit and one or more general monomeric units and/or emitting monomeric units on the same polymer chain. The present disclosure can further include methods of making polymer dots by physically blending and/or chemically crosslinking two or more polymer chains together.

For example, the polymer dots can have interchain energy transfer in which a polymer nanoparticle can include two or more polymer chains physically blended and/or chemically crosslinked together. For interchain energy transfer, one of the chains can include an absorbing monomeric unit and another chain can include an emitting monomeric unit. In certain embodiments, the present disclosure provides for methods of making polymer dots by physically blending and/or chemically crosslinking an absorbing polymer and an emitting polymer, as described herein. Some of the polymer dots can be made to have both intrachain and interchain energy transfer. In some instances, the combination of intrachain and interchain energy transfer can increase the quantum yield of the polymer dots. In certain embodiments, the final Pdots can exhibit narrow-band absorption.

The present disclosure provides, in certain embodiments, methods of making the nanoparticles described herein. In some embodiments, the present disclosure provides a method of making nanoparticles, the method including: (i) providing a solution including a polymer, the polymer including an absorbing monomeric unit (the absorbing monomeric unit including a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof), and an emitting monomeric unit; and (ii) collapsing the polymer to form the nanoparticles. In certain embodiments, the nanoparticles have an absorbance width of less than 150 nm at 10% (or in some embodiments, at 15%) of the absorbance maximum. In some embodiments, the polymer has a backbone including the absorbing monomeric unit, has a side chain including the absorbing monomeric unit (e.g., the absorbing monomeric unit is an absorbing unit that is cross-linked to the polymer), has a terminus including the absorbing monomeric unit, or any combination thereof.

The present disclosure provides, in some embodiments, a method of making nanoparticles, the method including: (i) providing a solution including a first polymer (the first polymer including an absorbing monomeric unit) and a second polymer (the second polymer including an emitting monomeric unit); and (ii) collapsing the first polymer and the second polymer to form the nanoparticles, wherein the nanoparticles have an absorbance width of less than 150 nm at 10% (or in some embodiments, 15%) of the absorbance maximum. In certain embodiments, the absorbing monomeric unit includes a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof. In some embodiments, the first polymer has a backbone including the absorbing monomeric unit, has a side chain including the absorbing monomeric unit (e.g., the absorbing monomeric unit is an absorbing unit that is cross-linked to the polymer), has a terminus including the absorbing monomeric unit, or any combination thereof. In some embodiments, the second polymer has a backbone including the emitting monomeric unit, has a side chain including the emitting monomeric unit (e.g., the emitting monomeric unit is an emitting unit that is cross-linked to the polymer), has a terminus including the absorbing monomeric unit, or any combination thereof.

The polymer including an emitting monomeric unit and an absorbing polymer can be referred to as an “emitting and absorbing polymer,” the details of which are disclosed further herein. The polymer including an emitting monomeric unit can be referred to as an “emitting polymer,” the details of which are disclosed further herein. The polymer including an absorbing monomeric unit can be referred to as an “absorbing polymer,” the details of which are disclosed further herein.

Collapsing polymers to form a nanoparticle can include, without limitation, methods relying on precipitation, methods relying on the formation of emulsions (e.g., mini or micro emulsion), and methods relying on condensation. In a preferred embodiment, a narrow-band absorbing nanoparticles are formed by nanoprecipitation. The nanoprecipitation method involves the introduction of a solution of a polymer in a good solvent into a poor solvent, where the solubility collapses the polymer into a nanoparticle form. In specific embodiments, the poor solvent can be an aqueous solution. Collapsed polymer(s) refers to polymer(s) that have been collapsed into stable sub-micron sized particles. As a non-limiting example, a solution including the absorbing polymer, the emitting polymer, the emitting and absorbing polymer, or any combination thereof can include a non-protic solvent. Some or all of the non-protic solvent can be introduced (e.g., by injecting) to a solution including a protic solvent, thereby collapsing the polymer(s) into nanoparticles. In specific embodiments, the protic solvent is water (i.e., an aqueous solution).

In a specific embodiment, the method for preparing a narrow-band absorbing nanoparticle includes the steps of (i) preparing a mixture including an absorbing polymer, an emitting polymer, and a non-protic solvent; (ii) introducing all or a portion of the mixture into a solution including a protic solvent, thereby collapsing the absorbing polymer and emitting polymer into a nanoparticle; and (iii) removing the aprotic solvent from the mixture formed in step (ii), thereby forming a suspension of nanoparticles. In specific embodiments, the protic solvent is water (i.e., an aqueous solution).

In another embodiment, the method for preparing a narrow-band absorbing nanoparticle includes the steps of (i) preparing a mixture including an absorbing and emitting polymer, and a non-protic solvent; (ii) introducing all or a portion of the mixture into a solution including a protic solvent, thereby collapsing the absorbing and emitting polymer into a nanoparticle; and (iii) removing the aprotic solvent from the mixture formed in step (ii), thereby forming a suspension of nanoparticles. In specific embodiments, the protic solvent is water (i.e., an aqueous solution).

In specific embodiments, the collapsing step includes combining the solution including the absorbing polymer, the emitting polymer, and/or the absorbing and emitting polymer with an aqueous liquid.

In certain embodiments, the solution including the absorbing polymer, the emitting polymer, and/or the absorbing and emitting polymer includes a small percentage of the absorbing monomeric unit by weight. In some embodiments, the solution includes 15% or less of the absorbing monomeric unit by weight, 14% or less of the absorbing monomeric unit by weight, 13% or less of the absorbing monomeric unit by weight, 12% or less of the absorbing monomeric unit by weight, 11% or less of the absorbing monomeric unit by weight, 10% or less of the absorbing monomeric unit by weight, 9% or less of the absorbing monomeric unit by weight, 8% or less of the absorbing monomeric unit by weight, 7% or less of the absorbing monomeric unit by weight, 6% or less of the absorbing monomeric unit by weight, 5% or less of the absorbing monomeric unit by weight, 4% or less of the absorbing monomeric unit by weight, 3% or less of the absorbing monomeric unit by weight, 2% or less of the absorbing monomeric unit by weight, or 1% or less of the absorbing monomeric unit by weight.

In some embodiments, the solution including the absorbing polymer, the emitting polymer, and/or the absorbing and emitting polymer includes a large percentage of the absorbing monomeric unit by weight. In some embodiments, the solution includes 1% or more of the absorbing monomeric unit by weight, 2% or more of the absorbing monomeric unit by weight, 3% or more of the absorbing monomeric unit by weight, 4% or more of the absorbing monomeric unit by weight, 5% or more of the absorbing monomeric unit by weight, 6% or more of the absorbing monomeric unit by weight, 7% or more of the absorbing monomeric unit by weight, 8% or more of the absorbing monomeric unit by weight, 9% or more of the absorbing monomeric unit by weight, 1⁰% or more of the absorbing monomeric unit by weight, 11% or more of the absorbing monomeric unit by weight, 12% or more of the absorbing monomeric unit by weight, 13% or more of the absorbing monomeric unit by weight, 14% or more of the absorbing monomeric unit by weight, 15% or more of the absorbing monomeric unit by weight, 20% or more of the absorbing monomeric unit by weight, 25% or more of the absorbing monomeric unit by weight, 30% or more of the absorbing monomeric unit by weight, 35% or more of the absorbing monomeric unit by weight, or 40% or more of the absorbing monomeric unit by weight.

As disclosed herein, the narrow-band absorbing nanoparticles can have various beneficial optical properties. In certain embodiments, the nanoparticles can have a quantum yield of greater than 5%, greater than 10%, greater than 15%, greater than 20%, greater than 25%, greater than 30/a, greater than 35%, greater than 40%, greater than 45%, or greater than 50%.

In some embodiments, the narrow-band absorbing nanoparticles are prepared by precipitation. This technique involves the rapid addition (e.g., facilitated by sonication or vigorous stirring) of a dilute polymer solution (e.g., absorbing polymer, emitting polymer, and/or absorbing and emitting polymer dissolved in an organic solvent) into an excess volume of non-solvent (but miscible with the organic solvent), such as water or other physiologically relevant aqueous solution. For example, in some embodiments, the polymer(s) is first dissolved into an organic solvent where the solubility is good (good solvent), such as THF (tetrahydrofuran), after which the dissolved polymer(s) in THF is added to an excess volume of water or aqueous buffer solution, which is a poor solvent for the hydrophobic polymer(s), but which is miscible with the good solvent (THF). The resulting mixture is sonicated or vigorously stirred to assist the formation of polymer dots, then the organic solvent is removed to leave behind well dispersed nanoparticles. In using this procedure, the polymer(s) must be sufficiently hydrophobic to dissolve in the organic solvent.

In some aspects, the nanoparticles are formed by other methods, including but not limited to various methods based on emulsions (e.g., mini or micro emulsion) or precipitations or condensations. Other polymers having hydrophobic functional groups can also be employed, in which the hydrophobic functional groups do not affect the collapse and stability of the narrow-band absorbing nanoparticle. The hydrophobic functional groups on the surface of the nanoparticles can then be converted to hydrophilic functional groups (e.g., by post-functionalization) for bioconjugation or directly link the hydrophobic functional groups to biomolecules. This latter approach can work particularly well using functional groups that are both hydrophobic and clickable (i.e., chemical reactions that fall within the framework of click chemistry), including but not limited to alkyne, strained alkyne, azide, diene, alkene, cyclooctyne, and phosphine groups.

### Methods of Using Narrow-Band Absorbing Nanoparticles

The present disclosure provides, in at least one embodiment, a method of analyzing a biological molecule (“biomolecule”), the method including optically detecting the presence or absence of the biomolecule, wherein the biomolecule is attached to a nanoparticle as disclosed herein. In some embodiments, the attachment of the nanoparticle to the biomolecule includes a covalent bond, an ionic bond, or any combination thereof. In certain embodiments, the detecting includes using a detector. In certain embodiments, the detecting includes multiplex detection. Specific embodiments of multiplex detection can be found in international application PCT/US2012/071767, which is incorporated herein by reference.

In some embodiments, the detector includes an imaging device. In specific embodiments, the detector is selected from the group consisting of a camera, an electron multiplier, a charge-coupled device (CCD) image sensor, a photomultiplier tube (PMT), an avalanche photodiode (APD), a single-photon avalanche diode (SPAD), and a complementary metal oxide semiconductor (CMOS) image sensor; or includes a photo, electro, acoustical, or magnetic detector; or wherein the detector incorporates fluorescence microscopy imaging.

In some embodiments, the method further includes performing an assay. In certain embodiments, the assay is a digital assay. In specific embodiments, the assay includes fluorescence activated sorting. In specific embodiments, the assay includes flow cytometry. In certain embodiments, the assay includes RNA extraction (with or without amplification), cDNA synthesis (reverse transcription), gene microarrays, DNA extraction, Polymerase Chain Reaction (PCR) (single, nested, quantitative real-time, or linker-adapter), isothermal nucleic acid amplification, DNA-methylation analysis, cell culturing, comparative genomic hybridization (CGH) studies, electrophoresis, Southern blot analysis, enzyme-linked immunosorbent assay (ELISA), digital nucleic acid assay, digital protein assay, assays to determine the microRNA and siRNA contents, assays to determine the DNA/RNA content, assays to determine lipid contents, assays to determine protein contents, assays to determine carbohydrate contents, functional cell assays, or any combination thereof.

In some embodiments, the method includes amplifying the biomolecule to produce an amplified product. In certain embodiments, a moiety associated with the biomolecule is amplified to produce an amplified product. In specific embodiments, the amplifying includes performing polymerase chain reaction (PCR), isothermal nucleic acid amplification, rolling circle amplification (RCA), nucleic acid sequence based amplification (NASBA), loop-mediated amplification (LAMP), strand-displacement amplification (SDA), or any combination thereof.

In some embodiments, a plurality of biomolecules is analyzed. In some embodiments, a portion of the plurality of biomolecules is associated with a nanoparticle as disclosed herein. The biomolecules may be attached to a nanoparticle as disclosed herein (e.g., covalently and/or ionically bonded). In some embodiments, all the biomolecules are associated with a nanoparticle as disclosed herein.

The present disclosure further provides methods of using the narrow-band absorbing polymer dots described herein. For example, the present disclosure provides methods of luminescence-based detection using the narrow-band absorbing polymer dots as a novel class of luminescent probe and their bioconjugates for a variety of applications, including but not limited to flow cytometry, fluorescence activated sorting, immunofluorescence, immunohistochemistry, fluorescence multiplexing, single molecule imaging, single particle tracking, protein folding, protein rotational dynamics, DNA and gene analysis, protein analysis, metabolite analysis, lipid analysis, FRET based sensors, high throughput screening, cell detection, bacteria detection, virus detection, biomarker detection, cellular imaging, in vivo imaging, fluorescence microscopy, bioorthogonal labeling, click reactions, fluorescence-based biological assays such as immunoassays and enzyme-based assays, and a variety of fluorescence techniques in biological assays and measurements. In some embodiments, the nanoparticles disclosed herein can be used for methods that involve digital assays. In certain aspects, the nanoparticles disclosed herein can be used for methods of detection that involve multiplexing over a variety of wavelength ranges. In some embodiments, the nanoparticles disclosed herein can be used for methods that involve exposing the nanoparticle to a variety of wavelength emission ranges.

In some aspects, the narrow-band absorbing nanoparticles are modified with a functional group and/or biomolecular conjugates for a variety of applications, including but not limited to flow cytometry, fluorescence activated sorting, immunofluorescence, immunohistochemistry, fluorescence multiplexing, single molecule imaging, single particle tracking, protein folding, protein rotational dynamics, DNA and gene analysis, protein analysis, metabolite analysis, lipid analysis, FRET based sensors, high throughput screening, cellular imaging, in vivo imaging, fluorescence microscopy, bioorthogonal labeling, click reactions, fluorescence-based biological assays such as immunoassays and enzyme-based assays, and a variety of fluorescence techniques in biological assays and measurements.

In one aspect, the present disclosure provides methods for imaging polymer dots that include administering a population of polymer dots described herein to a subject and exciting at least one polymer dot in the population of polymer dots, e.g., with an imaging system. The method can further include detecting a signal from at least one excited polymer dot in the population of polymer dots. As described further herein, the polymer dots can be administered in a composition.

In another aspect, the present disclosure includes a method of multiplex excitation and/or detection with a polymer dot. The method can include exciting the nanoparticle (i.e., transferring energy to the nanoparticle by, as a non-limiting example, exposing an absorbing monomeric unit within or on the nanoparticle to a source of radiation), and can further include detecting the polymer dot with a detector system, wherein the polymer dot includes an absorbing monomeric unit and an emitting monomeric unit. In some embodiments, the nanoparticle is excited by a source of radiation, such as a laser beam. In certain embodiments, the source of radiation has an emission wavelength range of less than 200 nm, less than 150 nm, less than 140 nm, less than 130 nm, less than 120 nm, less than 110 nm, less than 100 nm, less than 90 nm, less than 80 nm, less than 70 nm, less than 60 nm, less than 50 nm, less than 40 nm, less than 30 nm, or less than 20 nm.

As described further herein, the polymer dots of the present disclosure can include, e.g., a homopolymer or heteropolymer including an absorbing monomeric unit, such as an absorbing monomeric unit including a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof. In some aspects of the present disclosure, a system for optically marking and sorting cells with narrow-band absorbing nanoparticles is provided. In certain aspects, the system includes a plurality of biomolecules (e.g., cells) optionally attached to a substrate, a source of electromagnetic radiation (e.g., a light source), one or more processors operably coupled to the source, and a sorting device. (e.g., a flow based analysis or sorting device, or imaging based analysis device). Each of the plurality of biomolecules includes an attachment to at least one narrow-band absorbing nanoparticle (also referred to herein as an “optical marker”), as discussed herein. In certain embodiments, the optical marker includes the nanoparticle as disclosed herein, which, when excited by a wavelength of light, can induce luminescent emission. In various aspects, the sorting device is configured to sort the plurality of biomolecules when detached from a substrate based on the optical state of an optical marker, e.g., to separate cells of the subset from cells not of the subset. For example, in some aspects, the cell sorting is performed based on the emission intensity of the optical marker of each cell at a particular excitation wavelength.

In some aspects, the source of electromagnetic radiation includes a laser, a lamp (e.g., a mercury lamp, halogen lamp, metal halide lamp, or other suitable lamp), an LED, or any combination thereof. In some aspects, the peak wavelength emitted by the light source of is between about 350 nm and about 450 nm, about 400 nm and about 500 nm, about 450 nm and about 550 nm, about 500 nm and about 600 nm, about 550 nm and about 650 nm, about 600 nm and about 700 nm, about 650 nm and about 750 nm, about 700 nm and about 800 nm, about 750 nm and about 850 nm, about 800 nm and about 900 nm, about 850 nm and about 950 nm, or about 900 nm and about 1000 nm. In some aspects, two or more light sources having distinct peak wavelengths can be used. In some aspects, light emitted by the light source is spectrally filtered by a light filtering apparatus. In some aspects, the light filtering apparatus includes a filter, e.g., a bandpass filter that only allows light wavelengths falling within a certain range to pass through it towards the cells. In some aspects, the light filtering apparatus includes a multichroic mirror that can separate light into distinct spectral components, such that it only allows light wavelengths falling within a certain range to be directed towards the biomolecule. In some aspects, the longest wavelength that passes through a light filtering apparatus is less than 400 nm, less than 500 nm, less than 600 nm, less than 700 nm, less than 800 nm, less than 900 nm, or less than 1000 nm. In some aspects, the shortest wavelength that passes through a light filtering apparatus is more than 300 nm, more than 400 nm, more than 500 nm, more than 600 nm, more than 700 nm, more than 800 nm, or more than 900 nm.

In some aspects of the present disclosure, the system also includes an imaging device, such as a microscope (e.g., a confocal microscope, spinning disk microscope, multi-photon microscope, planar illumination microscope, Bessel beam microscope, differential interference contrast microscope, phase contrast microscope, epifluorescent microscope, or any combination thereof). Optionally, the source of electromagnetic radiation is a component of the imaging device, e.g., provides illumination for imaging. In certain aspects, the imaging device is used to obtain image data of the plurality of cells, e.g., when attached to the substrate. Optionally, the image data is used as a basis for selecting the subset of biomolecules to be optically marked. In some aspects, this process occurs manually, e.g., a user views the image data and input instructions to select and optically mark the subset. In other aspects, this process occurs automatically, e.g., the one or more processors analyze the image data, such as by using computer vision or image analysis algorithms, and select the cells to be marked without requiring user input. In alternative aspects, the selection and marking procedure is semi-automated, e.g., involving some user input and some automatic processing.

In some aspects, a system configured for optical encoding and sorting of biomolecules is provided. In certain aspects, the system includes a plurality of biomolecules, a source of electromagnetic radiation (e.g., a light source), one or more processors operably coupled to the source, and an analysis or sorting device (e.g., a flow based analysis or sorting device, or imaging based analysis device). Each of the plurality of biomolecules includes a first optical marker that is convertible from a first optical state to a second optical state upon application of a first light energy, and a second optical marker that is convertible from a third optical state to a fourth optical state upon application of a second light energy. As a non-limiting example, a biomolecule may be attached to a first narrow-band absorbing nanoparticle that, following application of a first light energy within its narrow absorbance spectrum, results in luminescence of a first signal (i.e., converts from a first optical state to the second optical state), and a second narrow-band absorbing nanoparticle that, following application of a second light energy within its narrow absorbance spectrum, results in excitation from its ground state (i.e., the third optical state) to luminescence of a second signal (i.e., converts to a fourth optical state upon application of a second light energy). In various aspects, the second light energy is different from the first light energy (e.g., has a different wavelength). In various aspects, the second light energy has the same wavelength as the first light energy but has a different light intensity. In various aspects, the first optical marker has different optical properties than the second optical marker (e.g., different emission spectra, different absorption spectra). In some aspects, the one or more processors are configured to cause the source to selectively apply the first light energy to a first subset of the biomolecules and the second light energy to a second subset of the biomolecules. In certain aspects, the first and second subsets are different from each other, so as to produce cells with differing combinations of optical states that, for example, represent different optical absorptions and emissions. Optionally, the analysis or sorting device can be used to analyze or sort the biomolecules according to the different optical encodings.

In some aspects, a system configured for optical encoding and single-biomolecule dispensing of biomolecules is provided. In certain aspects, the system includes a plurality of biomolecules attached to a substrate, a source of electromagnetic radiation (e.g., a light source), one or more processors operably coupled to the source, and a single-biomolecule dispensing system (e.g. into holders such as microwells or droplets). Each of the plurality of biomolecules includes a first optical marker that is convertible from a first optical state to a second optical state upon application of a first light energy, and a second optical marker that is convertible from a third optical state to a fourth optical state upon application of a second light energy. In various aspects, the second light energy is different from the first light energy (e.g., has a different wavelength). In various aspects, the second light energy has the same wavelength as the first light energy but has a different light intensity. Different light intensity can be achieved via either adjusting the power of the light source or adjusting the duration of illumination with a given power of the light source or a combination of the two. In various aspects, the first optical marker has different optical properties than the second optical marker (e.g., different emission spectra, different absorption spectra). In some aspects, the one or more processors are configured to cause the source to selectively apply the first light energy to a first subset of the biomarkers and the second light energy to a second subset of the biomarkers. In certain aspects, the first and second subsets are different from each other, so as to produce biomarkers with differing combinations of optical states that, for example, represent different optical encodings. The single-biomarker dispensing device can be used to analyze or dispense individual biomarkers, and the identity or characteristics of each biomarker is decoded optically (e.g. by fluorescence imaging or flow-based optical interrogation) according to the different optical encodings. Single-biomarker analysis can include imaging, PCR, isothermal nucleic acid amplification, RNA-seq, genotyping, sequencing, genetic analysis, ELISA, digital nucleic acid assay, digital protein, assays, functional studies, -omics analysis (e.g. metabolomics, genomics, lipidomics, proteomics), or biomarker culture.

In some aspects, the systems described herein include a computer including one or more processors and a memory device with executable instructions stored thereon. In some aspects, the computer is used to perform the methods described herein. In various aspects, a computer can be used to implement any of the systems or methods illustrated and described above. In some aspect, a computer includes a processor that communicates with a number of peripheral subsystems via a bus subsystem. These peripheral subsystems can include a storage subsystem, including a memory subsystem and a file storage subsystem, user interface input devices, user interface output devices, and a network interface subsystem.

In some aspects, a bus subsystem provides a mechanism for enabling the various components and subsystems of the computer to communicate with each other as intended. The bus subsystem can include a single bus or multiple busses.

In some aspects, a network interface subsystem provides an interface to other computers and networks. The network interface subsystem can serve as an interface for receiving data from and transmitting data to other systems from a computer. For example, a network interface subsystem can enable a computer to connect to the Internet and facilitate communications using the Internet.

In some aspect, the computer includes user interface input devices such as a keyboard, pointing devices such as a mouse, trackball, touchpad, or graphics tablet, a scanner, a barcode scanner, a touch screen incorporated into the display, audio input devices such as voice recognition systems, microphones, and other types of input devices. In general, use of the term “input device” is intended to include all possible types of devices and mechanisms for inputting information to a computer.

In some aspect, the computer includes user interface output devices such as a display subsystem, a printer, a fax machine, or non-visual displays such as audio output devices, etc. The display subsystem can be a flat-panel device such as a liquid crystal display (LCD), or a projection device. In general, use of the term “output device” is intended to include all possible types of devices and mechanisms for outputting information from a computer.

In some aspects, the computer includes a storage subsystem that provides a computer-readable storage medium for storing the basic programming and data constructs. In some aspects, the storage subsystem stores software (programs, code modules, instructions) that when executed by a processor provides the functionality of the methods and systems described herein. These software modules or instructions can be executed by one or more processors. A storage subsystem can also provide a repository for storing data used in accordance with the present disclosure. The storage subsystem can include a memory subsystem and a file/disk storage subsystem.

In some aspects, the computer includes a memory subsystem that can include a number of memories including a main random access memory (RAM) for storage of instructions and data during program execution and a read only memory (ROM) in which fixed instructions are stored. A file storage subsystem provides a non-transitory persistent (non-volatile) storage for program and data files, and can include a hard disk drive, a floppy disk drive along with associated removable media, a Compact Disk Read Only Memory (CD-ROM) drive, an optical drive, a flash drive, removable media cartridges, and other like storage media.

The computer can be of various types including a personal computer, a portable computer, a workstation, a network computer, a mainframe, a kiosk, a server or any other data processing system. Due to the ever-changing nature of computers and networks, the description of computer contained herein is intended only as a specific example for purposes of illustrating the aspect of the computer. Many other configurations having more or fewer components than the system described herein are possible.

The specific dimensions of any of the apparatuses, devices, systems, and components thereof, of the present disclosure can be readily varied depending upon the intended application, as will be apparent to those of skill in the art in view of the disclosure herein. Moreover, it is understood that the examples and aspects described herein are for illustrative purposes only and that various modifications or changes in light thereof may be suggested to persons skilled in the art and are included within the spirit and purview of this application and scope of the appended claims. Numerous different combinations of aspects described herein are possible, and such combinations are considered part of the present disclosure.

In certain embodiments, the methods provided herein may further be coupled to an assay protocol following biological nanoparticle (i.e., a nanoparticle attached to a biomolecule) sorting or collection. Non-limiting examples of assays that may be coupled to the methods provided herein include nucleic-acid based methods such as RNA extraction (with or without amplification), cDNA synthesis (reverse transcription), gene microarrays, DNA extraction, Polymerase Chain Reactions (PCR) (single, nested, quantitative real-time, or linker-adapter), isothermal nucleic acid amplification, or DNA-methylation analysis; cytometric methods such as fluorescence in situ hybridization (FISH), laser capture microdissection, flow cytometry, fluorescence activated sorting (e.g., fluorescence activated cell sorting, FACS), cell culturing, or comparative genomic hybridization (CGH) studies; chemical assay methods such as electrophoresis, Southern blot analysis or enzyme-linked immunosorbent assay (ELISA); digital nucleic acid assay, digital protein assay, assays to determine the microRNA and siRNA contents; assays to determine the DNA/RNA content; assays to determine lipid contents; assays to determine carbohydrate contents; assays to determine metabolite contents; assays to determine protein contents; and functional cell assays (e.g. apoptotic assays, cell migration assays, cell proliferation assays, cell differentiation assays, etc.), and the like.

In some aspects of the present disclosure, the device also includes an imaging device, such as a microscope (e.g., a confocal microscope, spinning disk microscope, multi-photon microscope, planar illumination microscope, Bessel beam microscope, differential interference contrast microscope, phase contrast microscope, epifluorescent microscope, transmission electron microscope, or any combination thereof). Optionally, the source of interrogating is a component of the imaging device, e.g., provides illumination for imaging. In certain aspects, the imaging device is used to obtain image data of the biological nanoparticles, e.g., when captured by a coating. Optionally, the image data is used as a basis for assigning a biological nanoparticle identification. In some aspects, this process occurs manually, e.g., a user views the image data and input instructions to assign an identifier based on, e.g., detectable agents (i.e., the narrow-band absorbing nanoparticles disclosed herein) associated with a biomolecule. In other aspects, this process occurs automatically, e.g., the device includes one or more processors to analyze the image data, such as by using computer vision or image analysis algorithms, and assign a value to the biological nanoparticles without requiring user input. In alternative aspects, the assigning is semi-automated, e.g., involving some user input and some automatic processing.

In some embodiments, at least some of the plurality of biological nanoparticles are captured with a coating and are imaged. In certain embodiments, the imaging includes fluorescence microscopy. In specific embodiments, the fluorescence microscopy is super-resolution imaging. In certain embodiments, the imaging includes atomic force microscopy. In some embodiments, the imaging includes transmission electron microscopy. In certain embodiments, the imaging includes photographic capture. In some embodiments, the imaging includes real-time monitoring and/or video capture.

In yet another embodiment, the methods provided herein may further be coupled to flow cytometry, for example, to further partition or isolate biological nanoparticles present in a fluid sample. In one embodiment, a channel of the device used for the methods provided herein may be in fluidic communication with a flow cytometer. In certain embodiments, the coupling of device and flow cytometry allows for selected biological nanoparticles to be further examined or serially sorted to further enrich a population of biological nanoparticles and/or biomolecules of interest. In certain embodiments of the methods provided herein, this configuration allows for upstream gross-sorting of biological nanoparticles and/or biomolecules and only directs biological nanoparticles and/or biomolecules including a desired size value, or biological nanoparticles associated with a particular detectable agent, into downstream processes such as flow cytometry, in order to decrease time, cost, and/or labor.

As used herein A and/or B encompasses one or more of A or B, and combinations thereof such as A and B.

All features discussed in connection with any aspect or aspect herein can be readily adapted for use in other aspects and aspects herein. The use of different terms or reference numerals for similar features in different aspects does not necessarily imply differences other than those expressly set forth. Accordingly, the present disclosure is intended to be described solely by reference to the appended claims, and not limited to the aspects disclosed herein.

Unless otherwise specified, the presently described methods and processes can be performed in any order. For example, a method describing steps (a), (b), and (c) can be performed with step (a) first, followed by step (b), and then step (c). Or, the method can be performed in a different order such as, for example, with step (b) first followed by step (c) and then step (a). Furthermore, those steps can be performed simultaneously or separately unless otherwise specified with particularity.

The particulars shown herein are by way of example and for purposes of illustrative discussion of the preferred aspects of the present disclosure only and are presented in the cause of providing what is believed to be the most useful and readily understood description of the principles and conceptual aspects of various aspects of the disclosure. In this regard, no attempt is made to show structural details of the disclosure in more detail than is necessary for the fundamental understanding of the disclosure, the description taken with the drawings and/or examples making apparent to those skilled in the art how the several forms of the disclosure may be embodied in practice.

While preferred aspects of the present disclosure have been shown and described herein, it is to be understood that the disclosure is not limited to the particular aspects of the disclosure described, as variations of the particular aspects can be made and still fall within the scope of the appended claims. It is also to be understood that the terminology employed is for the purpose of describing particular aspects of the disclosure, and is not intended to be limiting. Instead, the scope of the present disclosure is established by the appended claims.

Where a range of values is provided, it is understood that each intervening value, to the tenth of the unit of the lower limit unless the context clearly dictates otherwise, between the upper and lower limit of that range, and any other stated or intervening value in that stated range, is encompassed within the disclosure provided herein. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure provided herein.

All features discussed in connection with an aspect or aspect herein can be readily adapted for use in other aspects and aspects herein. The use of different terms or reference numerals for similar features in different aspects does not necessarily imply differences other than those expressly set forth. Accordingly, the present disclosure is intended to be described solely by reference to the appended claims, and not limited to the aspects disclosed herein.

### EXAMPLES

The specific dimensions of any of the apparatuses, devices, systems, and components thereof, of the present disclosure can be readily varied depending upon the intended application, as will be apparent to those of skill in the art in view of the disclosure herein. Moreover, it is understood that the examples and aspects described herein are for illustrative purposes only and that various modifications or changes in light thereof can be suggested to persons skilled in the art and are included within the spirit and purview of this application and scope of the appended claims. Numerous different combinations of aspects described herein are possible, and such combinations are considered part of the present disclosure. In addition, all features discussed in connection with any one aspect herein can be readily adapted for use in other aspects herein. The use of different terms or reference numerals for similar features in different aspects does not necessarily imply differences other than those expressly set forth. Accordingly, the present disclosure is intended to be described solely by reference to the appended claims, and not limited to the aspects disclosed herein.

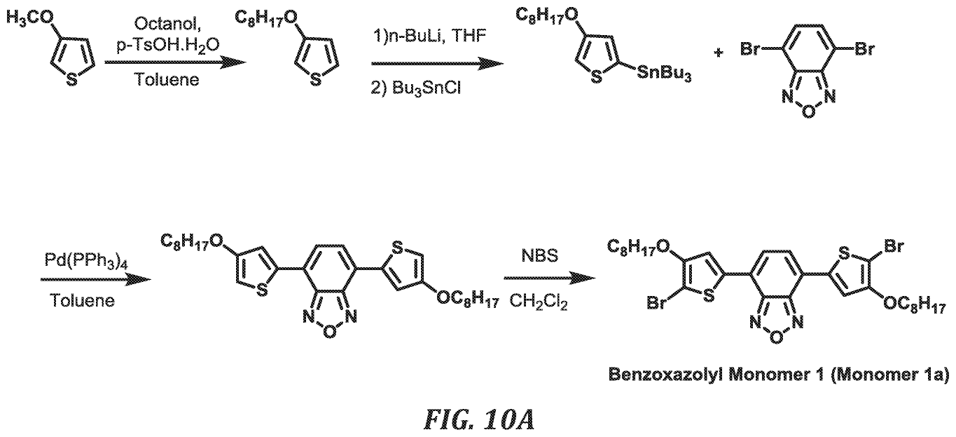

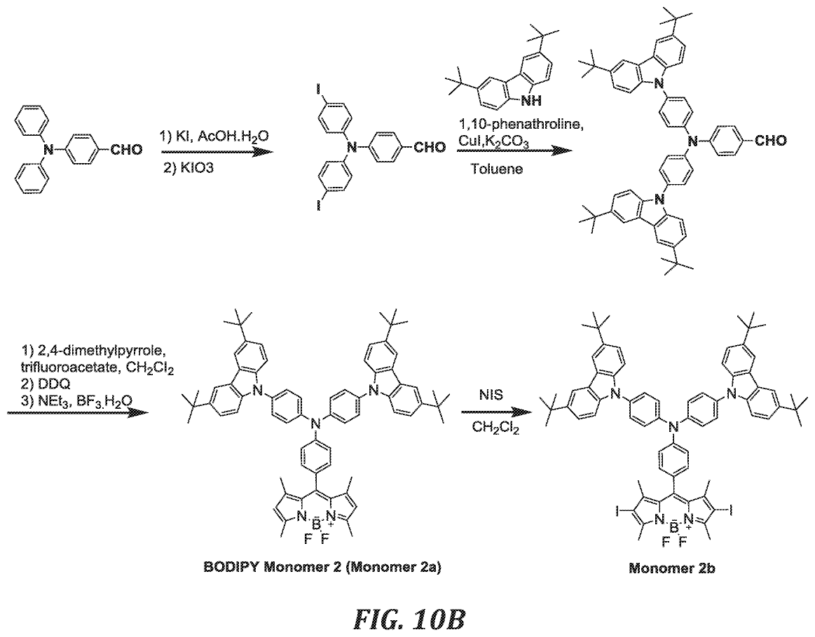

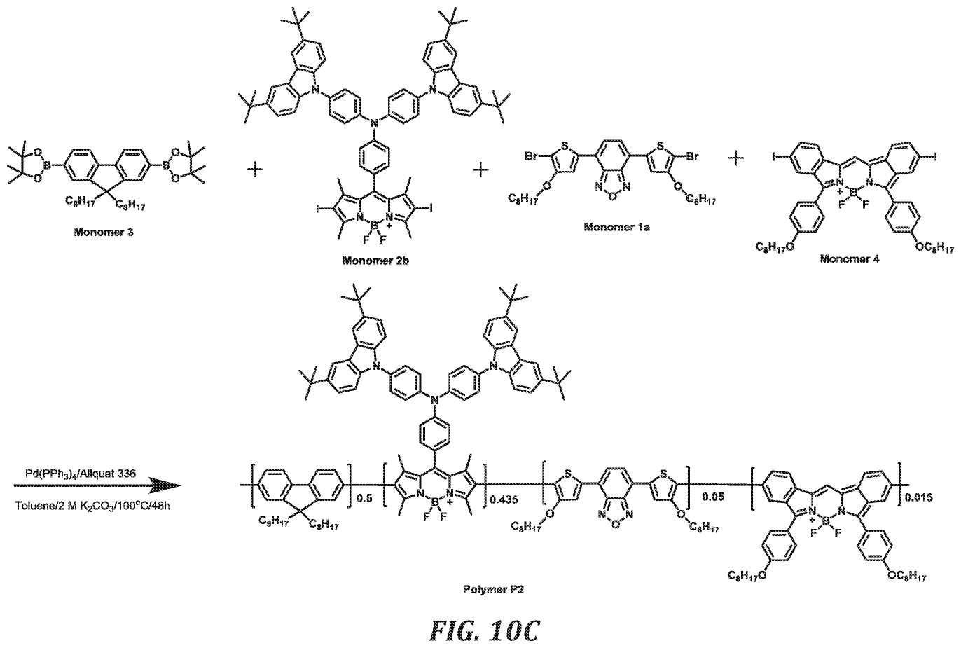

### Example 1. Synthesis of Narrow-Band Absorbing Polymer P2 (FIG. 10)

This Example describes the synthesis of monomers (i.e., benzooxdiazolyl-based Monomer 1 (FIG. 10A) and BODIPY-based Monomer 2 (FIG. 10B)), as well as the synthesis of narrow-band absorbing copolymer Polymer P2 (FIG. 10C).

### Synthesis of Benzoxazolyl Monomer 1 (Monomer 1a) (FIG. 10A)

A mixture of 3-methoxythiophene (32.7 mmol, 3.4 g), octanol (25 mL), p-toluenesulfonic acid monohydrate (1.0 g), and toluene (75 mL) was refluxed and stirred overnight. The solution was cooled and washed thrice with water, dried over Na₂SO₄, and filtered. The filtrate was concentrated and purified by silica gel column chromatography to afford 3-(octyloxy)thiophene (5.7 g, yield: 81.7%).

3-(Octyloxy)thiophene (10.0 mmol, 2.0 g) was dissolved in degassed anhydrous THF (30 mL). The solution was cooled to −78° C., then 4.5 mL n-BuLi (2.5 mol L⁻¹) was added dropwise to the solution, which was stirred for 1 hour. Tributyltin chloride (13.5 mmol, 3.4 mL) was injected to the solution, which was stirred overnight. To the THF solution was added 200 mL hexane, and the organic solution was washed thrice with aqueous saturated sodium bicarbonate, dried over Na₂SO₄, and filtered. The filtrate was concentrated to afford tributyl(4-(octyloxy)thiophen-2-yl)stannane, which was used directly without further purification.

The obtained tributyl(4-(octyloxy)thiophen-2-yl)stannane, 4,7-dibromobenzooxadiazole (4.0 mmol, 1.1 g), and Pd(PPh₃)₄ (0.1 g) were dissolved in toluene (50 mL) and stirred at 100° C. for 24 hours. The solution was cooled, then 30 mL saturated aqueous KF solution was added, and the mixture was stirred vigorously for 3 hours to remove residual stannane derivatives. The solution was washed thrice with water, then the organic layer was dried over Na₂SO₄ and filtered. The filtrate was concentrated purified by silica gel column chromatography to afford 4,7-bis(4-(octyloxy)thiophen-2-yl)benzo[c][1,2,5]oxadiazole as a yellow solid (1.0 g, yield: 46.1%).

4,7-bis(4-(octyloxy)thiophen-2-yl)benzo[c][1,2,5]oxadiazole (1.0 mmol, 0.54 g) and N-bromosuccinimide (2.2 mmol, 0.39 g) were dissolved in degassed anhydrous dichloromethane (30 mL) and stirred at room temperature in darkness for 12 hours. The resulting solution was purified using flash silica gel column chromatography, recrystallized in dichloromethane and ethanol, then filtered to afford Benzoxazolyl Monomer 1 (Monomer 1a) as a dark red solid (0.42 g, yield: 60.7%).

### Syntheses of BODIPY Monomer 2 (Monomer 2a) and Monomer 2b (FIG. 10B)

4-(diphenylamino)benzaldehyde (10.0 mmol, 2.73 g) and KI (22.0 mmol, 3.65 g) were dissolved in a mixture of acetic acid (24 mL) and H₂O (2.4 mL) under N₂. The mixture was warmed and stirred to afford a yellow transparent solution, then KIO₃ (22.0 mmol, 4.71 g) was added over four portions. The reaction mixture was warmed to reflux and stirred for 1 h. The mixture was cooled to room temperature, and to the mixture was added distilled water, precipitating a dark yellow solid. The mixture was filtered, and the collected solids were purified by silica gel column chromatography to afford 4-(bis(4-iodophenyl)amino)benzaldehyde as an orange solid (4.62 g, yield: 88.9%).

4-(bis(4-iodophenyl)amino)benzaldehyde (8.6 mmol, 4.47 g), 3,6-di-tert-butyl-9H-carbazole (19.0 mmol, 5.31 g), CuI (3.4 mmol, 0.64 g), 1,10-phenanthroline (7.5 mmol, 1.34 g), and K₂CO₃ (23.2 mmol, 3.2 g) were mixed in DMF (50 mL), and the mixture was heated to 160° C. under nitrogen for 24 h. After cooling to room temperature, the reaction mixture was poured into water (100 mL). The precipitate was filtered and dried, then purified by silica gel column chromatography to afford 4-(bis(4-(3,6-di-tert-butyl-9H-carbazol-9-yl)phenyl)amino)benzaldehyde as a white solid (6.4 g, Yield: 90.1%).

4-(bis(4-(3,6-di-tert-butyl-9H-carbazol-9-yl)phenyl)amino)benzaldehyde (4.8 mmol, 4.0 g), 2,4-dimethylpyrrole (13.4 mmol, 1.27 g), and trifluoroacetate (0.3 mL) were dissolved in degassed dichloromethane (500 mL), and the reaction was stirred for 3 hours. 2,3-Dichloro-5,6-dicyano-1,4-benzoquinone (DDQ) (4.8 mmol, 1.1 g) was added over four portions, then the solution was stirred for 1 hour. The solution was cooled to 4° C., trimethylamine (Me₃N) (10 mL) was injected, and then BF₃.H₂O (14 mL) was added dropwise. After stirred overnight, the resulting solution was washed thrice with saturated aqueous K₂CO₃, dried over Na₂SO₄, and filtered. The filtrate was concentrated and purified by silica gel column chromatography to afford BODIPY Monomer 2 (Monomer 2a) as a red solid (1.95 g, yield: 38.8%).

BODIPY Monomer 2 (1.1 mmol, 1.2 g), N-iodosuccinimide (2.4 mmol, 0.59 g), and degassed dichloromethane (50 mL) were combined and stirred at room temperature in darkness for 12 hours. The resulting solution was concentrated and purified by silica gel column chromatography to afford Monomer 2b as a red product (0.96 g, yield: 67.2%).

### Synthesis of Polymer P2 (FIG. 10C)

A mixture of Monomer 1a (0.01 mmol, 7.0 mg), Monomer 2b (0.087 mmol, 112.9 mg), (9,9-Dioctylfluorene-2,7-diboronic acid bis(1,3-propanediol) ester (Monomer 3, 0.1 mmol, 55.8 mg), Monomer 4 (0.003 mmol, 2.9 mg), Aliquat 336 (1 drop), Pd(PPh₃)₄ (5 mg, 0.005 mmol), 2 M aqueous K₂CO₃ (2 mL), and toluene (6 mL) was degassed 5 times under nitrogen gas. The resulting mixture was stirred at 100° C. for 48 h to afford Polymer P2. The polymer was then end-capped via the addition of 0.1 M phenylboronic acid (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped Polymer P2 with a yield of 75%.

Polymer P2 is a narrow-band absorbing polymer that has a backbone including a BODIPY-based absorbing monomeric unit (Monomer 2b). The polymer backbone also includes a BODIPY-based emitting monomeric unit (Monomer 4), an energy transfer monomeric unit (Monomer 1a), and a general monomeric unit (Monomer 3) that interacts with the BODIPY-based absorbing monomeric unit (Monomer 2b) and which together create a narrow-band absorbing polymer.

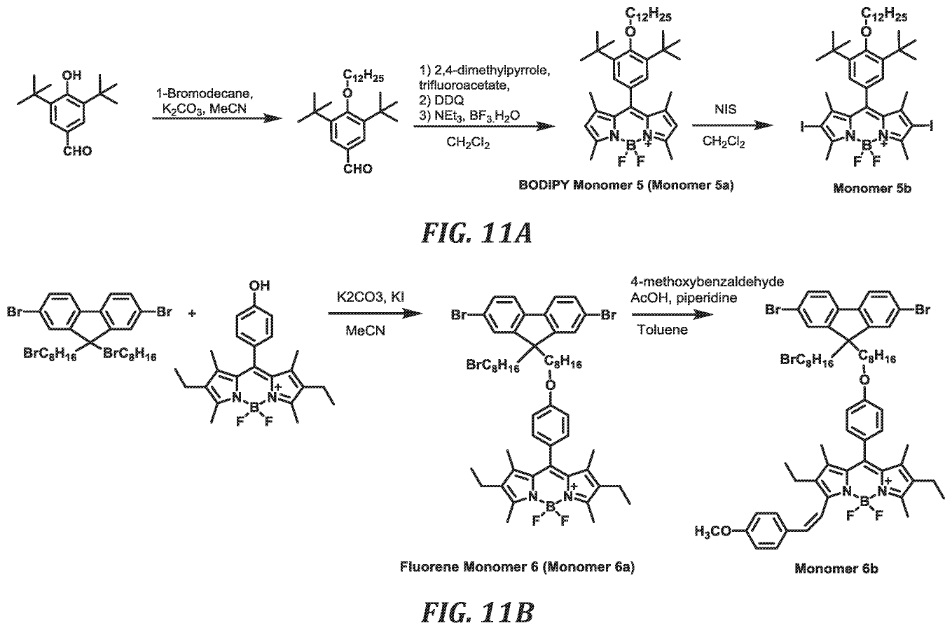

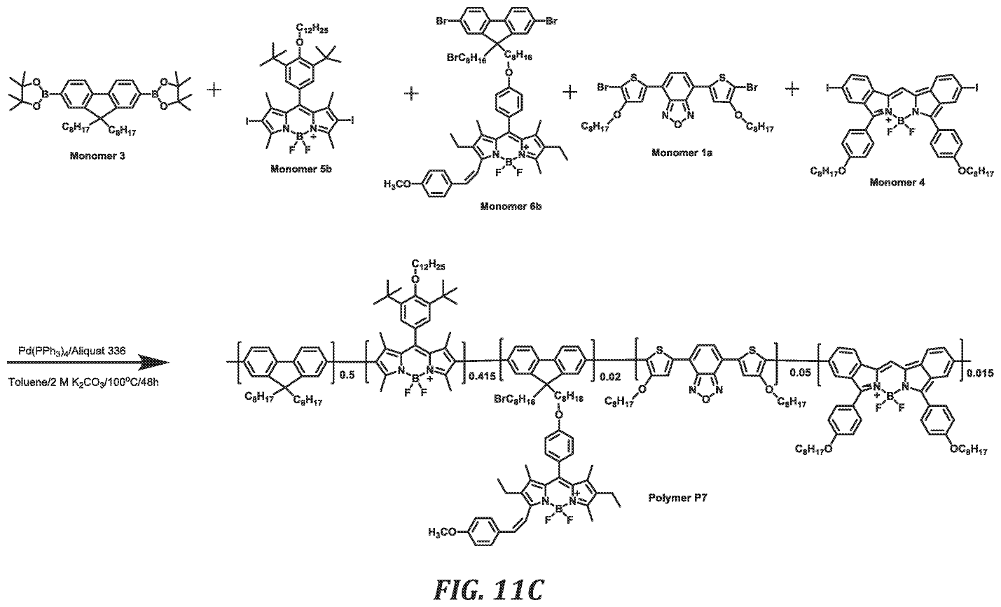

### Example 2. Synthesis of Narrow-Band Absorbing Polymer P7 (FIG. 11)

This Example describes the synthesis of monomers (i.e., BODIPY-based Monomer 5 (FIG. 11A)), including monomers cross-linked with absorbing units (i.e., fluorene-based Monomer 6 (FIG. 11B), including a BODIPY monomer as an absorbing unit), and the synthesis of narrow-band absorbing copolymer Polymer P7 (FIG. 11C).

### Syntheses of BODIPY Monomer 5 (Monomer 5a) and Monomer 5b (FIG. 11A)

3,5-di-tert-butyl-4-hydroxybenzaldehyde (50 mmol, 11.7 g), 1-bromododecane (100 mmol, 24.9 g), and K₂CO₃ (200 mmol, 27.0 g) were dissolved in degassed acetonitrile (250 mL) and stirred at 90° C. for 24 hours. The reaction was cooled, and the mixture was filtered. The filtrate was concentrated purified by silica gel column chromatography to afford 3,5-di-tert-butyl-4-(dodecyloxy)benzaldehyde as a white solid product (12.3 g, yield: 61.1%).

3,5-di-tert-butyl-4-(dodecyloxy)benzaldehyde (7.3 mmol, 2.52 g), 2,4-dimethylpyrrole (17.4 mmol, 1.66 g), and trifluoroacetate (0.3 mL) were combined in degassed dichloromethane (500 mL), and the mixture was stirred for 3 hours. 2,3-Dichloro-5,6-dicyano-1,4-benzoquinone (DDQ) (7.3 mmol, 1.67 g) was added over four portions, and the mixture was stirred for 1 hour. After cooling the mixture to 4° C., trimethylamine (Me₃N) (15 mL) was injected, then BF₃.H₂O (20 mL) was added dropwise. The mixture was stirred overnight, and the resulting solution was washed thrice with saturated aqueous K₂CO₃, dried over Na₂SO₄, and filtered. The filtrate was concentrated and purified by silica gel column chromatography to afford BODIPY Monomer 5 (Monomer 5a) as a red solid (1.91 g, yield: 42.0%).

Monomer 5a (1.0 mmol, 0.62 g) and N-iodosuccinimide (2.4 mmol, 0.54 g) were added to degassed dichloromethane (30 mL), and the mixture was stirred at room temperature in darkness for 12 hours. The resulting mixture was concentrated and purified by silica gel column chromatography to afford Monomer 5b as a deep red product (0.69 g, yield: 78.8%).

### Syntheses of Fluorene Monomer 6 (Monomer 6a) and Monomer 6b (FIG. 11B)

2,7-dibromo-9,9-bis(8-bromooctyl)-9H-fluorene (2.0 mmol, 1.4 g), (T-4)-[4-[(4-Ethyl-3,5-dimethyl-1H-pyrrol-2-yl-κN)(4-ethyl-3,5-dimethyl-2H-pyrrol-2-ylidene-κN)methyl]phenolato]difluoroboron, (5.0 mmol, 1.98 g), K₂CO₃ (20.0 mmol, 2.7 g), and KI (2.0 mmol, 0.33 g) were added to degassed acetone (100 mL) and stirred at 90° C. for 12 hours. After cooling, the mixture was filtered and the filtrate was concentrated and purified by silica gel column chromatography to afford Fluorene Monomer 6 (Monomer 6a) as a red solid (0.79 g, yield: 38.7%).

A mixture of Monomer 6a (0.5 mmol, 0.51 g), 4-methoxybenzaldehyde (2.0 mmol, 0.27 g), acetic acid (2 mL), and piperidine (2 mL) in toluene (20 mL) was heated to reflux under N₂ for 6 hours. After cooling, the mixture was washed thrice with water. The organic layer was dried over Na₂SO₄, and filtered. The filtrate was concentrated and purified by silica gel column chromatography to afford Monomer 6b as a red solid product (0.12 g, yield: 21.1%).

### Synthesis of Polymer P7 (FIG. 11C)

A mixture of Monomer 1a (0.01 mmol, 7.0 mg), Monomer 5b (0.083 mmol, 72.4 mg), Monomer 3 (0.1 mmol, 55.8 mg), Monomer 4 (0.003 mmol, 2.9 mg), Monomer 6b (0.004 mmol, 4.5 mg), Aliquat 336 (1 drop), Pd(PPh₃)₄ (5 mg, 0.005 mmol), 2 M aqueous K₂CO₃ (2 mL), and toluene (6 mL) was combined and degassed 5 times under nitrogen gas. The resulting mixture was stirred at 100° C. for 48 h to afford Polymer P7. The polymer was then end-capped via the addition of 0.1 M phenylboronic acid (1 mL) and bromobenzene (1 mL). The reaction mixture was cooled, then poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped Polymer P2 with a yield of 67%.

Polymer P7 is a narrow-band absorbing polymer that has a backbone including a BODIPY-based absorbing monomeric unit (Monomer 5b), a monomeric unit including an absorbing unit cross-linked to the polymer backbone (Monomer 6b), an emitting monomeric unit (Monomer 4), an energy transfer monomeric unit (Monomer 1a), and a general monomeric unit (Monomer 3) that interacts with the BODIPY-based absorbing monomeric unit (Monomer 5b) and which together create a narrow-band absorbing polymer.

### Example 3. General Procedure for Preparing Polymer Nanoparticles

This Example describes a general nanoprecipitation method that can be used to produce narrow-band absorbing nanoparticles as described herein.

Generally, the narrow-band absorbing polymers were first dissolved in THF to make a 1.0 g L⁻¹ stock solution. The stock polymer solution was diluted with copolymers of interest (e.g., PS-PEG-COOH) to produce a 10 mL THF solution having a total polymer concentration of 0.1 g L⁻¹. Generally, the copolymer solution included 0.08 g L⁻¹ of the narrow-band absorbing polymers and 0.02 g L⁻¹ of PS-PEG-COOH copolymer. A 5 mL aliquot of the copolymer solution mixture was quickly injected into 10 mL of Milli-Q water under sonication. THF was removed by blowing nitrogen gas into solution at 70° C. for about 30 minutes. The obtained polymer nanoparticles were in aqueous solution, which was sonicated for 1-2 minutes and filtered through a 0.2-μm cellulose membrane filter to remove any aggregates, and to obtain a˜0.05 mg mL⁻¹ Pdot solution.

### Example 4. Photophysical Properties of Polymers and Polymer Nanoparticles

This Example describes the photophysical properties of polymer and polymer nanoparticles corresponding to polymer PL.

The polymer P1 was dissolved in THF. The dissolved polymer had a number average molecular mass (Mₙ) of 36.9 KDa, and a polydispersity index (PDI) of 2.3.

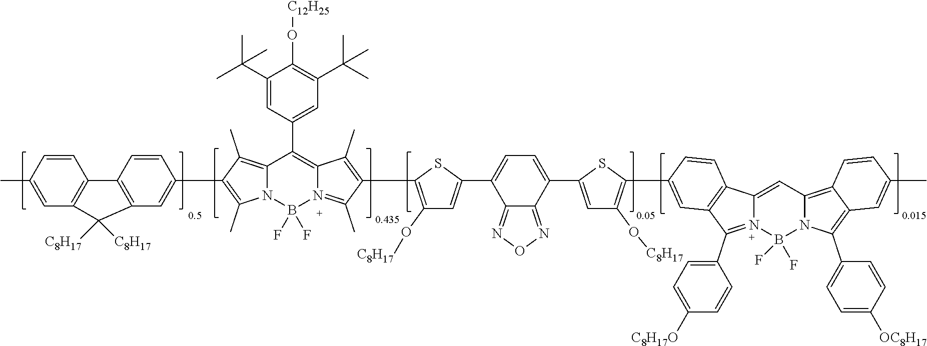

Polymer P1

The polymer solution was injected into aqueous solution to form nanoparticles via nanoprecipitation. Table 2 shows the photophysical properties of P1 dissolved in THF solution and in a collapsed nanoparticle state. The resulting polymer dots had an average hydrodynamic diameter of 23.8 nm.

TABLE 2
Photophysical properties of P1

| State | λ_{abs} (nm) | λ_{PL} (nm) | Φ_{PL} (%) |
|---|---|---|---|
| Solution in THF | 558 | 693 | 57.5 |
| Polymer dot | 551 | 717 | 15.8 |

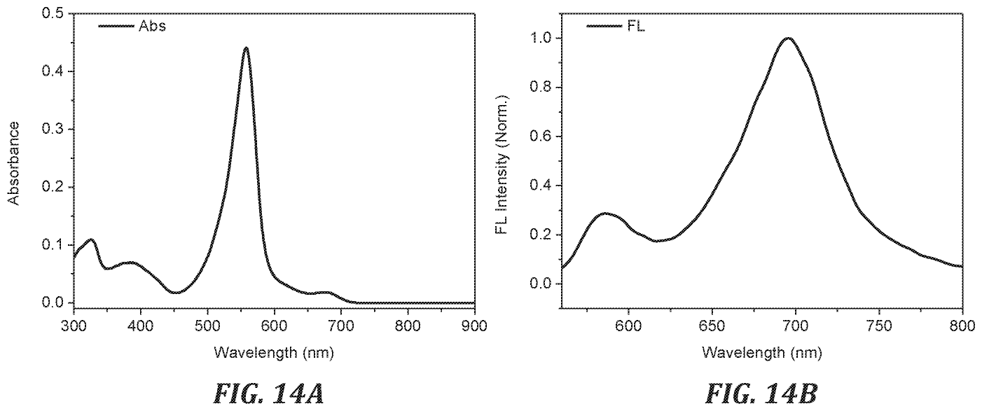

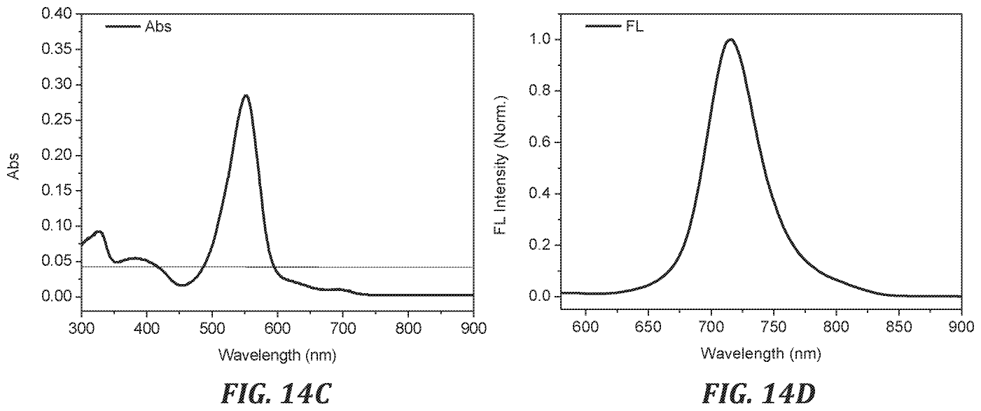

The absorbance and emission spectra of the polymer were measured (FIG. 14). While in solution, the polymer P1 had an absorbance wavelength maximum (λ_{abs}) of 558 nm (FIG. 14A), while the polymer dot had λ_{abs}=551 nm (FIG. 14C). The polymer dot of P1 was narrow-band absorbing, having an absorbance width at 15% maximum of 108 nm. The horizontal line of FIG. 14C represents a value of 15% of the absorbance maximum. In THF solution, the polymer P1 had a photoluminescence wavelength maximum (λ_{PL}) of 693 nm (FIG. 14B), while the polymer dot had a red-shifted emission with λ_{PL}=717 nm (FIG. 14D). The quantum yield decreased from 57.5% for the main emission peak in THF solution to 15.8% when in Pdot state.

### Example 5. Photophysical Properties of Polymers and Polymer Nanoparticles

This Example describes the photophysical properties of polymer and polymer nanoparticles corresponding to polymer P2.

The polymer P2 was dissolved in THF. The dissolved polymer had Mₙ=21.6 KDa and PDI=1.9.

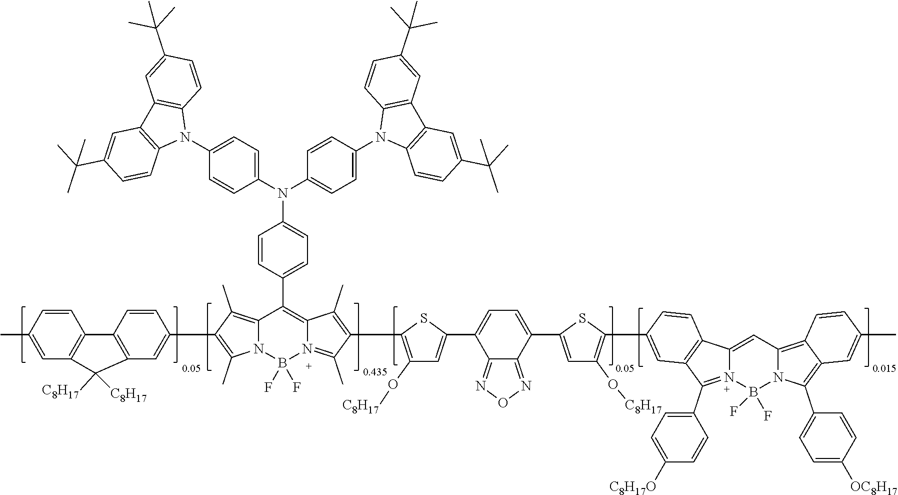

Polymer 2

The polymer solution was injected into aqueous solution to form nanoparticles via nanoprecipitation. Table 3 shows the photophysical properties of P2 dissolved in THF solution and in a collapsed nanoparticle state. The resulting polymer dots had an average hydrodynamic diameter of 33.8 nm.

TABLE 3
Photophysical properties of P2

| State | λ_{abs} (nm) | λ_{PL} (nm) | Φ_{PL} (%) |
|---|---|---|---|
| Solution in THF | 559 | 695 | 47.5 |
| Polymer dot | 558 | 715 | 13.7 |

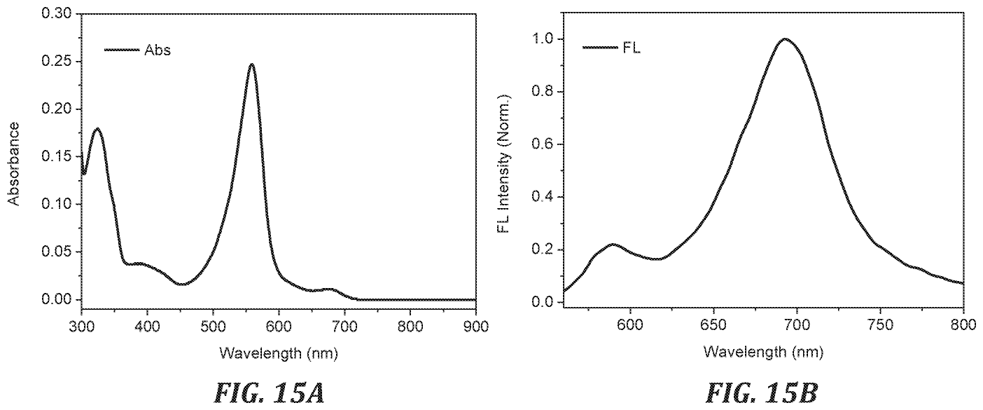

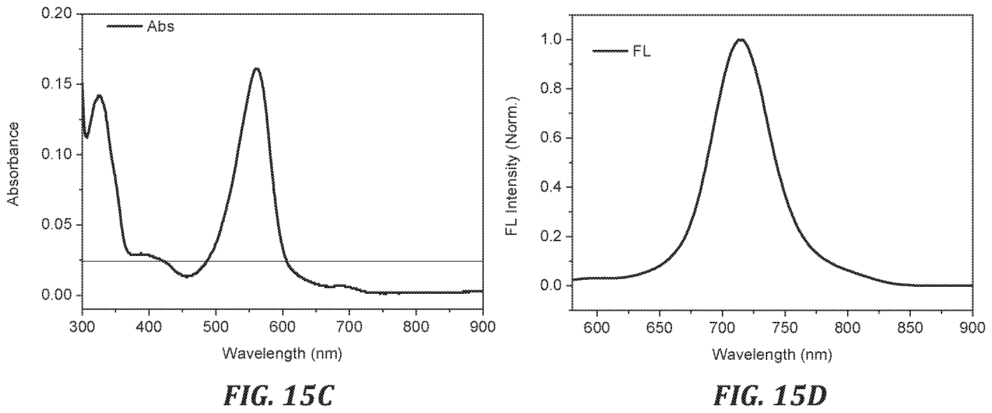

The absorbance and emission spectra of the polymer were measured (FIG. 15). While in solution, the polymer P2 had an λ_{abs}=559 nm (FIG. 15A), while the polymer dot had λ_{abs}=558 nm (FIG. 15C). The polymer dot of P2 was narrow-band absorbing, having an absorbance width at 15% maximum of 120 nm. The horizontal line of FIG. 15C represents a value of 15% of the absorbance maximum. In THF solution, the polymer P2 had λ_{PL}=695 nm (FIG. 15B), while the polymer dot had a red-shifted emission with λ_{PL}=715 nm (FIG. 15D). The quantum yield decreased from 47.5% for the main emission peak in THF solution to 13.7% when in Pdot state.

### Example 6. Photophysical Properties of Polymers and Polymer Nanoparticles

This Example describes the photophysical properties of polymer and polymer nanoparticles corresponding to polymer P3.

The polymer P3 was dissolved in THF. The dissolved polymer had Mₙ=17.6 KDa and PDI=2.1.

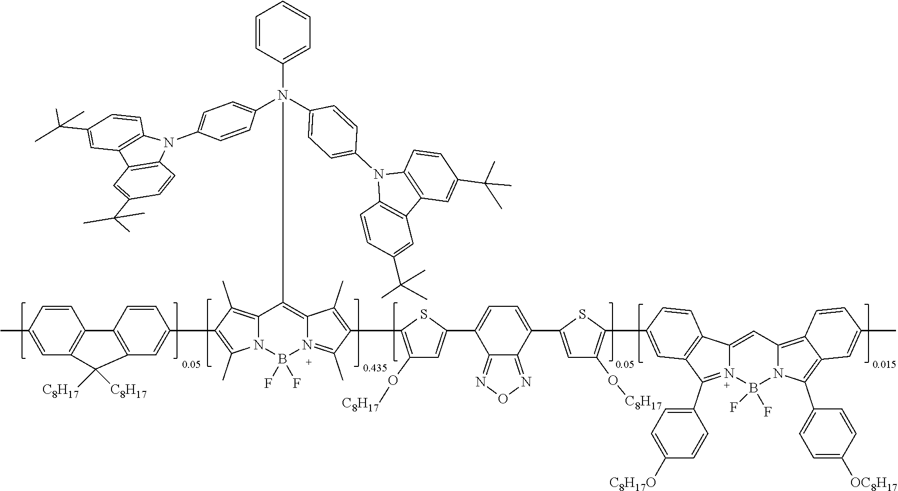

Polymer P3

The polymer solution was injected into aqueous solution to form nanoparticles via nanoprecipitation. Table 4 shows the photophysical properties of polymer P3 dissolved in THF solution and in a collapsed nanoparticle state. The resulting polymer dots had an average hydrodynamic diameter of 27.9 nm.

TABLE 4
Photophysical properties of P3

| State | λ_{abs} (nm) | λ_{PL} (nm) | Φ_{PL} (%) |
|---|---|---|---|
| Solution in THF | 572 | 696 | 58.0 |
| Polymer dot | 569 | 712 | 20.2 |

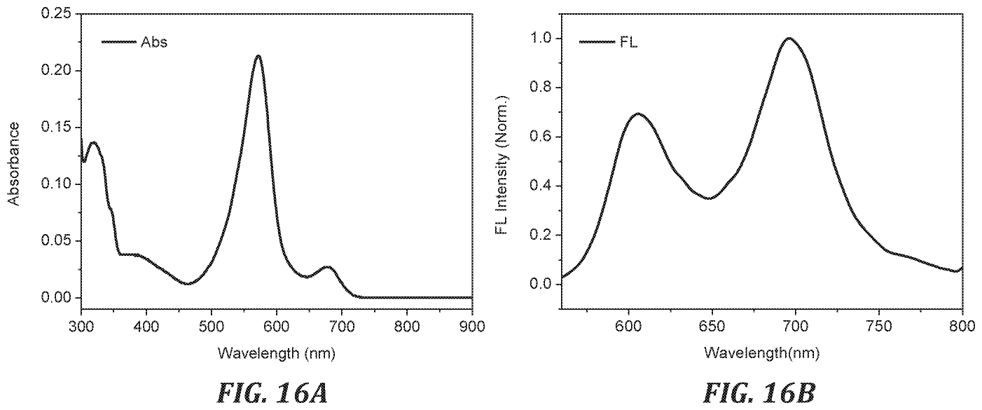

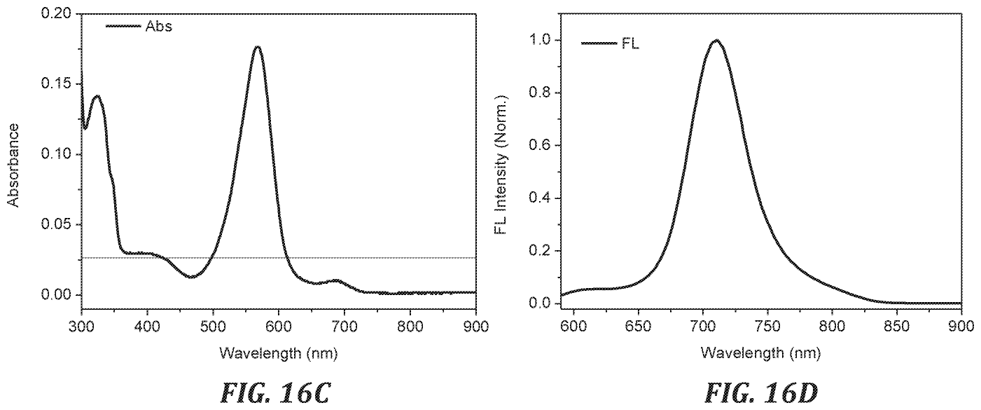

The absorbance and emission spectra of the polymer were measured (FIG. 16). While in solution, the polymer P3 had an λ_{abs}=572 nm (FIG. 16A), while the polymer dot had λ_{abs}=569 nm (FIG. 16C). The polymer dot of P3 was narrow-band absorbing, having an absorbance width at 15% maximum of 105 nm. The horizontal line of FIG. 16C represents a value of 15% of the absorbance maximum. In THF solution, the polymer P3 had Δ_{PL}=696 nm (FIG. 16B), while the polymer dot had a red-shifted emission with Δ_{PL}=712 nm (FIG. 16D). The quantum yield decreased from 58.0% for the main emission peak in THF solution to 20.2% when in Pdot state.

### Example 7. Photophysical Properties of Polymers and Polymer Nanoparticles

This Example describes the photophysical properties of polymer and polymer nanoparticles corresponding to polymer P4.

The polymer P4 was dissolved in THF. The dissolved polymer had Mₙ=29.3 KDa and PDI=2.5.

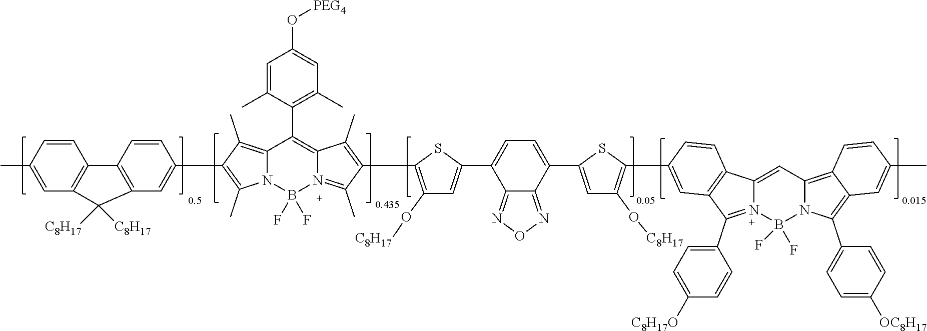

Polymer P4

The polymer solution was injected into aqueous solution to form nanoparticles via nanoprecipitation. Table 5 shows the photophysical properties of P4 dissolved in THF solution and in a collapsed nanoparticle state. The resulting polymer dots had an average hydrodynamic diameter of 18.9 nm.

TABLE 5
Photophysical properties of P4

| State | λ_{abs} (nm) | λ_{PL} (nm) | Φ_{PL} (%) |
|---|---|---|---|
| Solution in THF | 561 | 695 | 58.3 |
| Polymer dot | 554 | 714 | 10.0 |

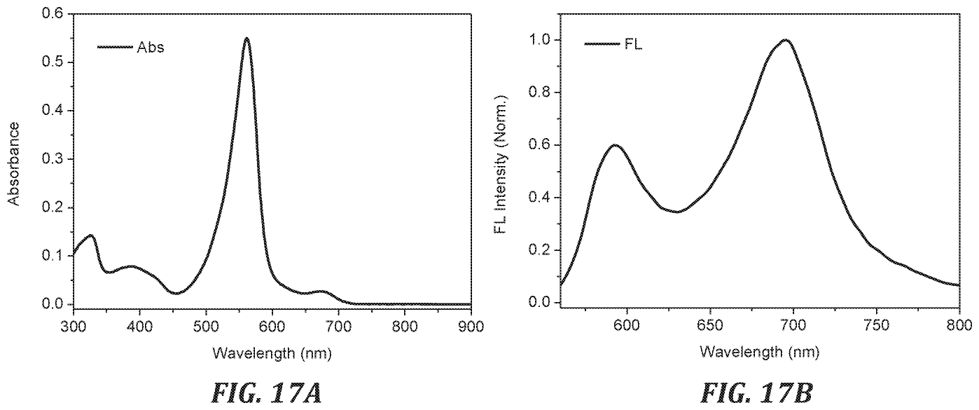

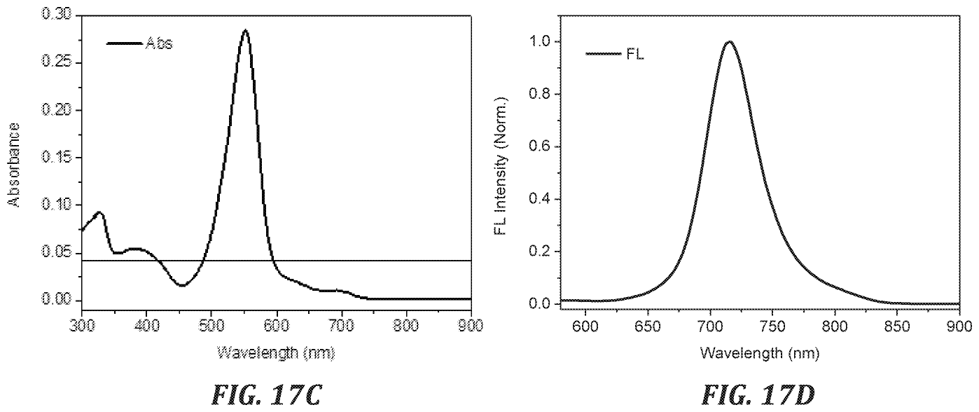

The absorbance and emission spectra of the polymer were measured (FIG. 17). While in solution, the polymer P4 had an λ_{abs}=561 nm (FIG. 17A), while the polymer dot had λ_{abs}=554 nm (FIG. 17C). The polymer dot of P4 was narrow-band absorbing, having an absorbance width at 15% maximum of 108 nm. The horizontal line of FIG. 17C represents a value of 15% of the absorbance maximum. In THF solution, the polymer P4 had Δ_{PL}=695 nm (FIG. 17B), while the polymer dot had a red-shifted emission with Δ_{PL}=714 nm (FIG. 17D). The quantum yield decreased from 58.3% for the main emission peak in THF solution to 10.0% when in Pdot state.

### Example 8. Photophysical Properties of Polymers and Polymer Nanoparticles

This Example describes the photophysical properties of polymer and polymer nanoparticles corresponding to polymer P5.

The polymer P5 was dissolved in THF. The dissolved polymer had Mₙ=23.8 KDa and PDI=3.1.

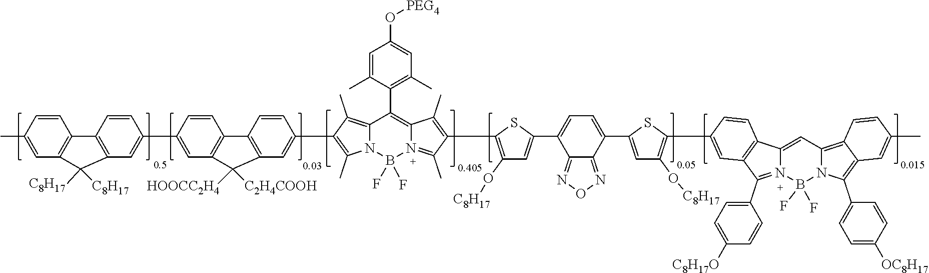

Polymer P5

The polymer solution was injected into aqueous solution to form nanoparticles via nanoprecipitation. Table 6 shows the photophysical properties of P5 dissolved in THF solution and in a collapsed nanoparticle state. The resulting polymer dots had an average hydrodynamic diameter of 20.8 nm.

TABLE 6
Photophysical properties of P5

| State | λ_{abs} (nm) | λ_{PL} (nm) | Φ_{PL} (%) |
|---|---|---|---|
| Solution in THF | 561 | 694 | 59.6 |
| Polymer dot | 553 | 714 | 12.6 |

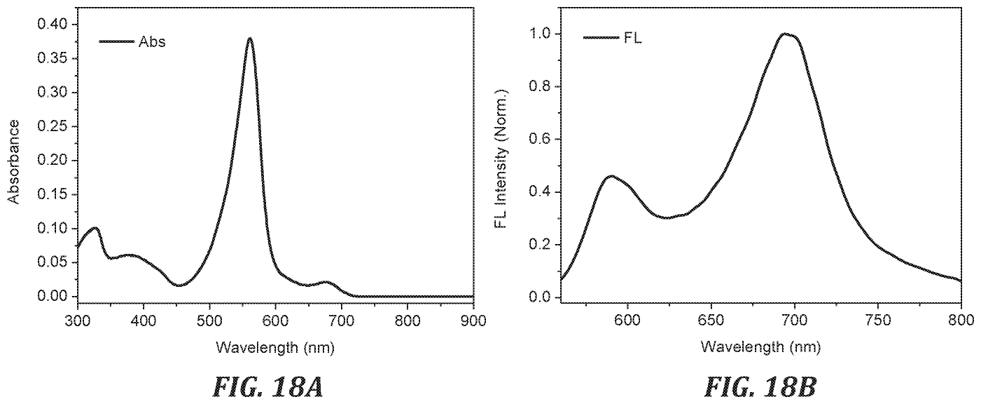

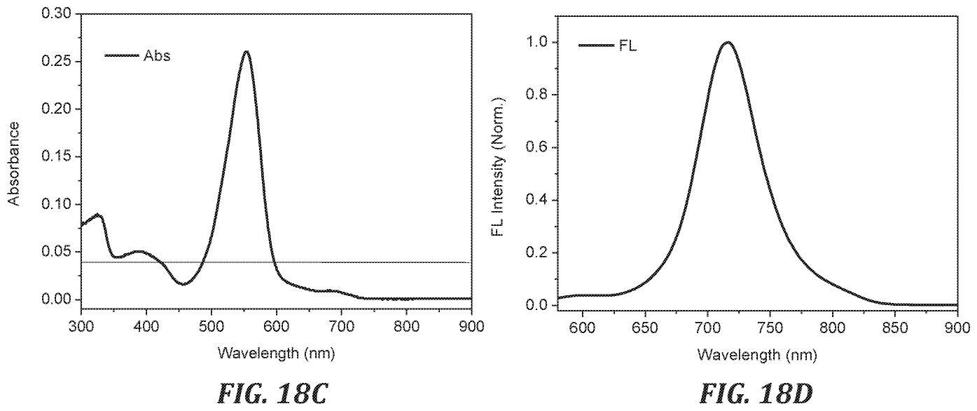

The absorbance and emission spectra of the polymer were measured (FIG. 18). While in solution, the polymer P5 had an λ_{abs}=561 nm (FIG. 18A), while the polymer dot had λ_{abs}=553 nm (FIG. 18C). The polymer dot of P5 was narrow-band absorbing, having an absorbance width at 15% maximum of 110 nm. The horizontal line of FIG. 18C represents a value of 15% of the absorbance maximum. In THF solution, the polymer P5 had Δ_{PL}=694 nm (FIG. 18B), while the polymer dot had a red-shifted emission with Δ_{PL}=714 nm (FIG. 18D). The quantum yield decreased from 59.6% for the main emission peak in THF solution to 12.6% when in Pdot state.

### Example 9. Photophysical Properties of Polymers and Polymer Nanoparticles

This Example describes the photophysical properties of polymer and polymer nanoparticles corresponding to polymer P6.

The polymer P6 was dissolved in THF. The dissolved polymer had Mₙ=14.3 KDa and PDI=1.7.

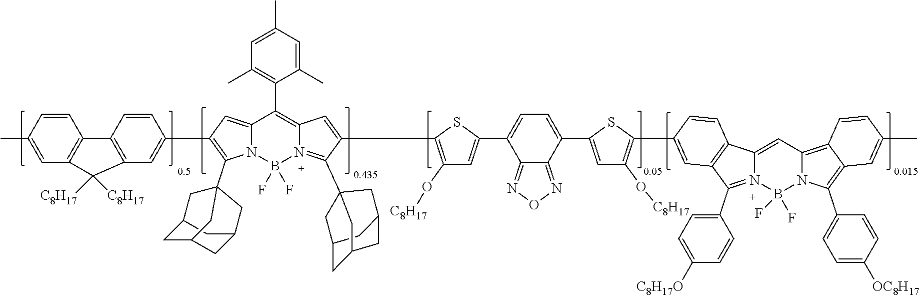

Polymer P6

The polymer solution was injected into aqueous solution to form nanoparticles via nanoprecipitation. Table 7 shows the photophysical properties of P6 dissolved in THF solution and in a collapsed nanoparticle state. The resulting polymer dots had an average hydrodynamic diameter of 22.6 nm.

TABLE 7
Photophysical properties of P6

| State | λ_{abs} (nm) | λ_{PL} (nm) | Φ_{PL} (%) |
|---|---|---|---|
| Solution in THF | 550 | 590/694 | 47.3 |
| Polymer dot | 545 | 714 | 12.7 |

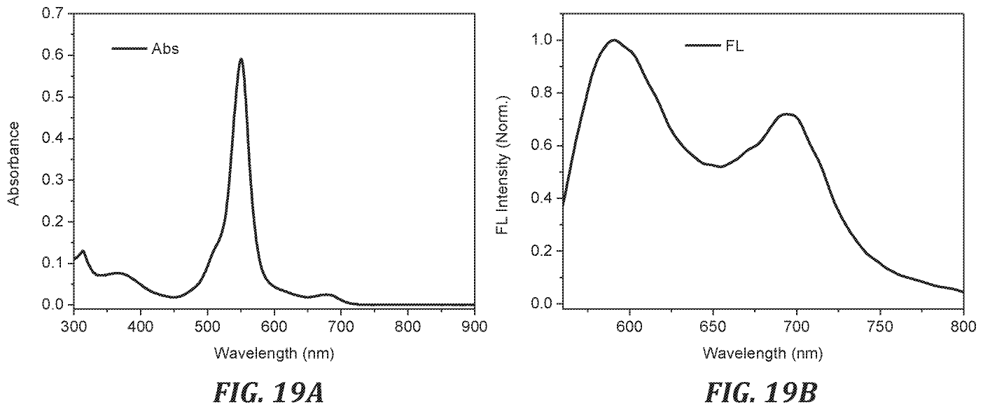

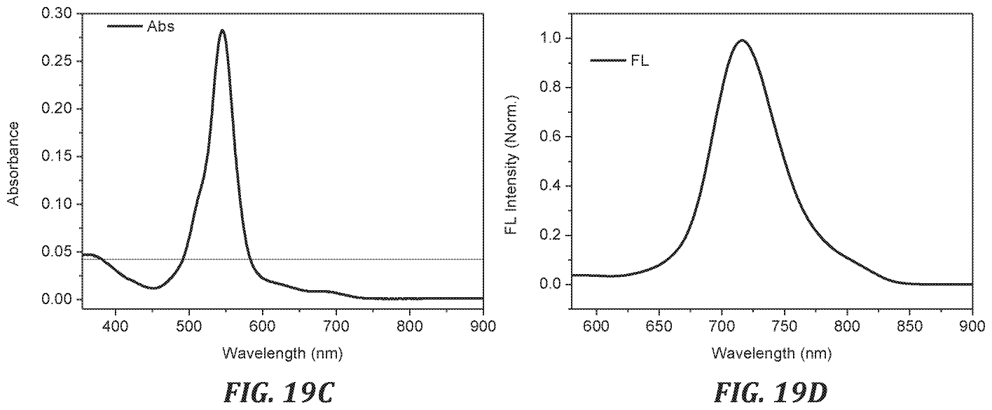

The absorbance and emission spectra of the polymer were measured (FIG. 19). In solution, the polymer P6 had an λ_{abs}=550 nm (FIG. 19A), while the polymer dot had λ_{abs}=545 nm (FIG. 19C). The polymer dot of P6 was narrow-band absorbing, having an absorbance width at 15% maximum of 95 nm. The horizontal line of FIG. 19C represents a value of 15% of the absorbance maximum. In THF solution, the polymer P6 had λ_{PL} values of 590 nm and 694 nm (FIG. 19B), while the polymer dot had a red-shifted emission with a single λ_{PL}=714 nm (FIG. 19D). The quantum yield decreased from 47.3% for the main emission peak in THF solution to 12.7% when in Pdot state.

### Example 10. Photophysical Properties of Polymers and Polymer Nanoparticles

This Example describes the photophysical properties of polymer and polymer nanoparticles corresponding to polymer P7.

The polymer P7 was dissolved in THF. The dissolved polymer had Mₙ=22.1 KDa and PDI=1.6.

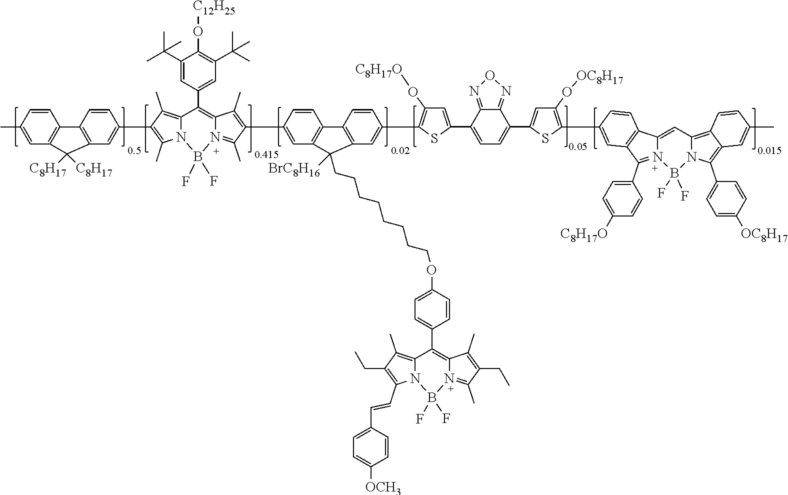

Polymer P7

The polymer solution was injected into aqueous solution to form nanoparticles via nanoprecipitation. Table 8 shows the photophysical properties of P7 dissolved in THF solution and in a collapsed nanoparticle state. The resulting polymer dots had an average hydrodynamic diameter of 27.6 nm.

TABLE 8
Photophysical properties of P7

| State | λ_{abs} (nm) | λ_{PL} (nm) | Φ_{PL} (%) |
|---|---|---|---|
| Solution in THF | 558 | 598/694 | 47.1 |
| Polymer dot | 551 | 714 | 15.9 |

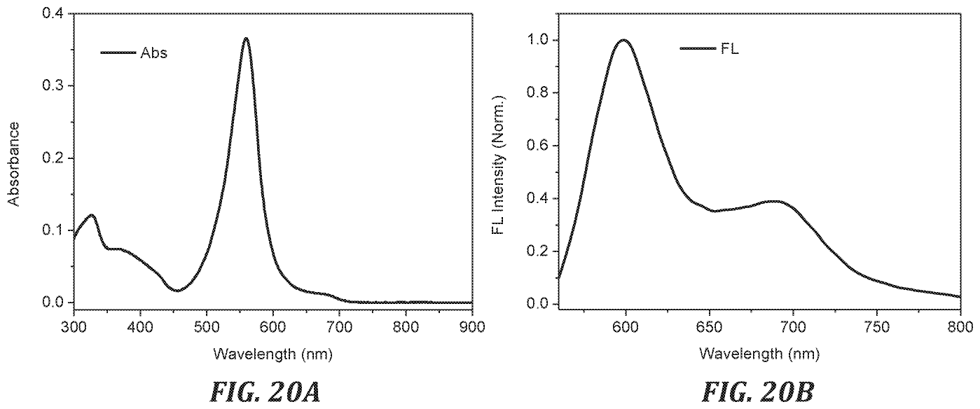

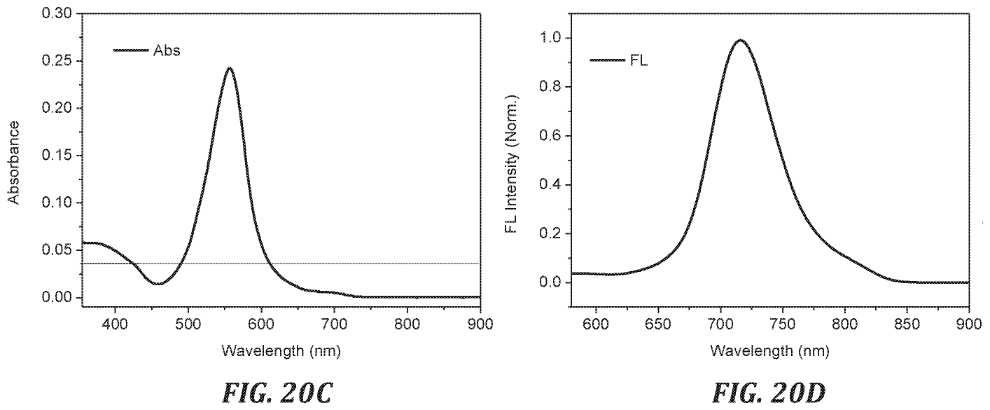

The absorbance and emission spectra of the polymer were measured (FIG. 20). While in solution, the polymer P7 had an λ_{abs}=558 nm (FIG. 20A), while the polymer dot had λ_{abs}=551 nm (FIG. 20C). The polymer dot of P7 was narrow-band absorbing, having an absorbance width at 15% maximum of 121 nm. The horizontal line of FIG. 20C represents a value of 15% of the absorbance maximum. In THF solution, the polymer P7 had λ_{PL} values of 598 nm and 694 nm (FIG. 20B), while the polymer dot had a red-shifted emission with a single λ_{PL}=714 nm (FIG. 20D). The quantum yield decreased from 47.1% for the main emission peak in THF solution to 15.9% when in Pdot state.

### Example 11. Photophysical Properties of Blended Nanoparticles

This Example describes the photophysical results of collapsing emissive polymers including a blend of polymer P8 and polymer P9 into nanoparticles.

A mixture of polymer P8 and polymer P9 having a weight ratio of 4:1 of P8 to P9 was dissolved in THF. The polymer solution was injected into aqueous solution to form nanoparticles via nanoprecipitation. The resulting polymer dots had an average hydrodynamic diameter of 29.2 nm and a quantum yield of 40.1%. The polymer dots included a blend of 80 wt % P8 and 20 wt % P9.

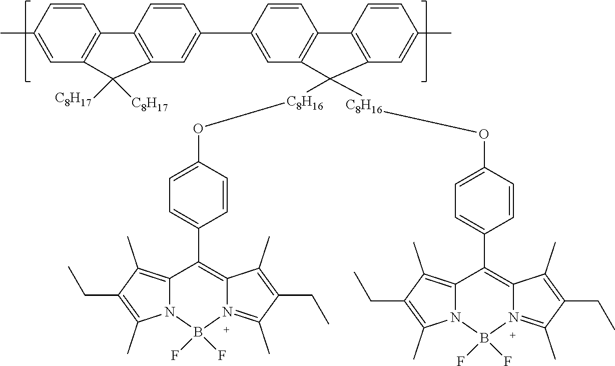

Polymer P8 (“PFGBDP”)

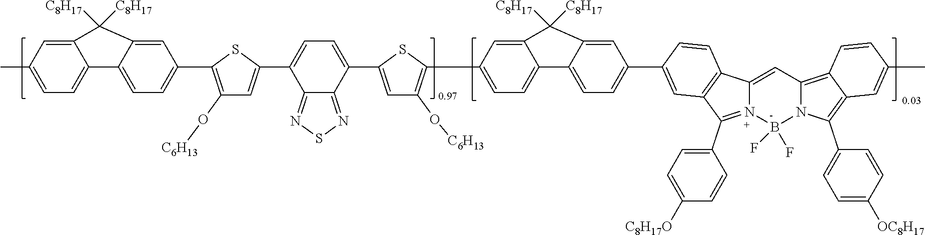

Polymer P9 (“PFDHTBT-BDP720”)

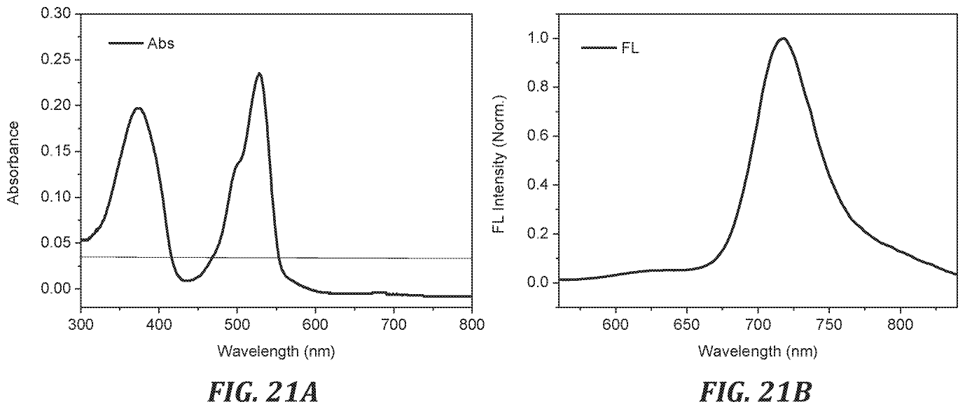

The absorbance and emission spectra of the blended polymer dots were measured (FIG. 21). The polymer dots had λ_{abs}=528 nm (FIG. 21A). The blended polymer dots of 80 wt % P8 and 20 wt % P9 were narrow-band absorbing, having absorbance width at 15% maximum of 85 nm. The horizontal line of FIG. 21A represents a value of 15% of the absorbance maximum. The polymer dot had an emission with a single λ_{PL}=721 nm (FIG. 21B).

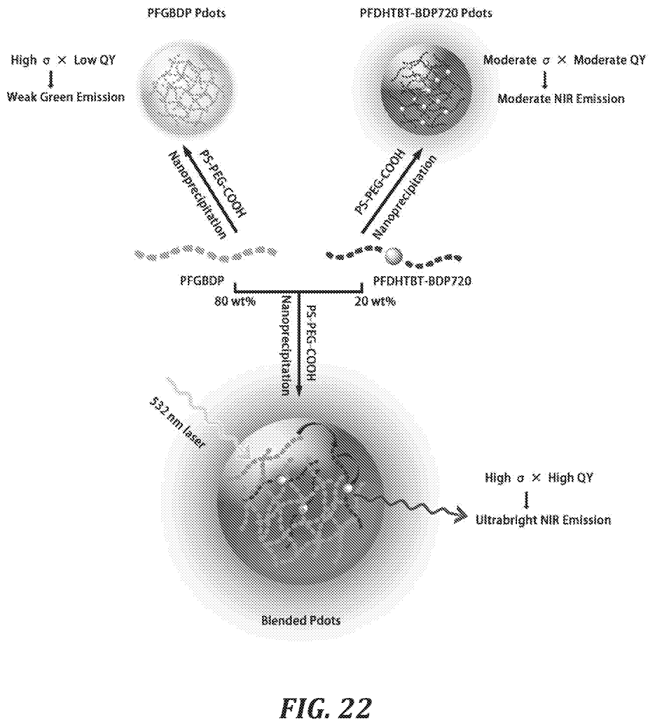

Blended nanoparticles can provide enhanced optical properties that are beneficial when compared to non-blended nanoparticles of each polymer type (FIG. 22). Polymer nanoparticles were formed via nanoprecipitation using polymer P8 (“PFGBDP,” including PFO monomeric units and PFO monomeric units with a BODIPY unit attached as a side chain) and PS-PEG-COOH, polymer P9 (“PFDHTBT-BDP720,” including poly[((9,9-dioctyl)-fluorene)-alt-(4,7-di-2-hexyl-thienyl-2,1,3-benzothiadiazole)](“PFDHTBT”) and BODIPY monomeric unit, “BDP720”) and PS-PEG-COOH, or a blend of P8, P9, and PS-PEG-COOH. A depiction of the P8 nanoparticle, the P9 nanoparticle, and the blended nanoparticle including 80 wt % P8 (“PFGBDP”) and 20 wt % P9 (“PFDHTBT-BDP720”) is depicted in FIG. 22. Blended nanoparticles were also formed using P8, P9, and poly(9.9-dioctyl-2,7-fluorene (PFO). The photophysical properties of the collapsed nanoparticles were measured (Table 9).

TABLE 9
Photophysical properties of P8, P9, and Blended Nanoparticles

| | λ_{abs} | λ_{PL} | | Φ_{PL} |
|---|---|---|---|---|
| Nanoparticle | (nm) | (nm) | A_{532 nm} | (%) |
| PFGBDP (P8) | 528 | 548 | 0.272 | 0.3 |
| 80 wt % P8 + 20 wt % PFO | 528 | 547 | 0.210 | 0.6 |
| PFDHTBT-BDP720 (P9) | 520 | 724 | 0.102 | 17.7 |
| 80 wt % PFO + 20 wt % P9 | 528 | 720 | 0.019 | 44.0 |
| 80 wt % P8 + 20 wt % P9 | 528 | 721 | 0.225 | 40.2 |

While long-wavelength excitable nanoparticles were formed that emit signal in the near-infrared region, low quantum yield can be observed due to, e.g., fluorescence self-quenching in the solid-like nanoparticle state (see: nanoparticle of P8, above).

Nanoparticle brightness is proportional to the product of quantum yield (Φ_{PL}) and absorption cross-section. Accordingly, nanoparticles with a relatively high quantum yield that is achieved at the cost of a lower absorption cross-section may not provide substantially beneficial brightness, due to reduced absorbing ability. In previously-developed nanoparticles, a trade-off between quantum yield and absorption cross-section limits brightness improvement. As provided by FIG. 22 and Table 9, P8 nanoparticles had a high cross-section absorbance (0.272 at 532 nm), but a low quantum yield (0.3%), providing a weak green emission. In contrast, P9 nanoparticles had a moderately high quantum yield (17.7%), but a poorer cross-section absorbance (0.102 at 532 nm), providing a moderately near-infrared emission. The blended nanoparticle including 80 wt % P8 and 20 wt % P9 had a high quantum yield (40.2%), as well as a high cross-section absorbance (0.225), combining to provide an ultra-bright near-infrared emission.

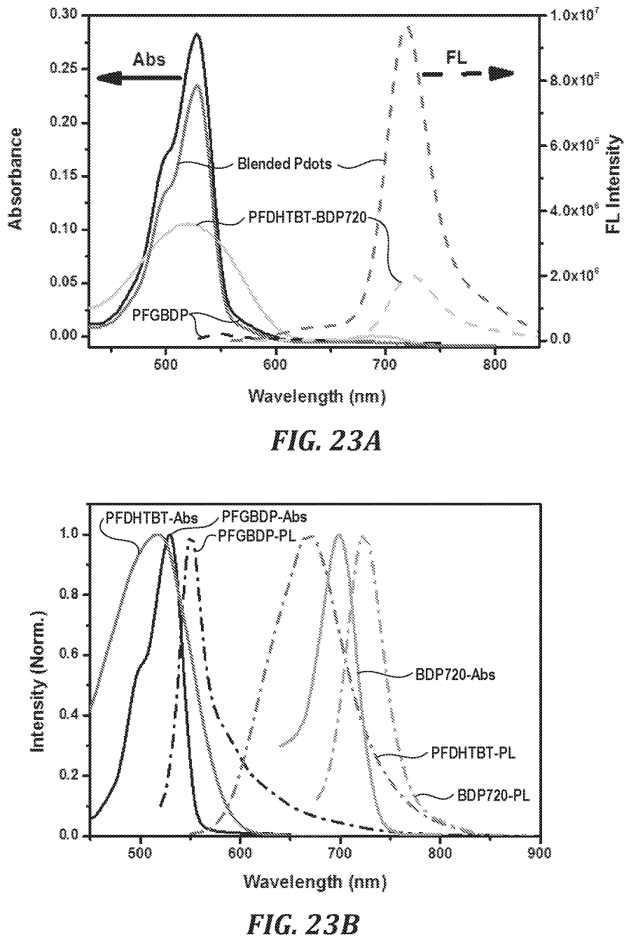

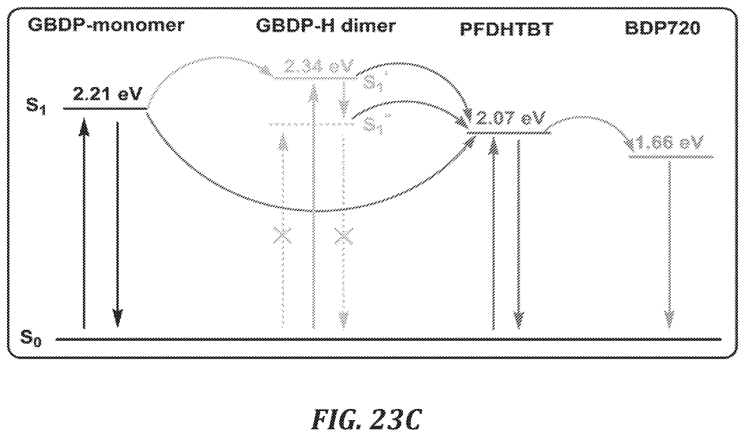

The green boron-dipyrromethene (GBDP) absorbing unit is attached as a side-chain to PFO to form polymer P8. PFGBDP has a strong absorbance at 532 nm (FIG. 23A). The boron-dipyrromethene unit can act as an energy donor, but while P8 has a quantum yield of 85% in diluted THF solution, its poor nanoparticle state (quantum yield of 0.3%), is in part due to the formation of H-aggregation dimers or other similar aggregates. GBDP aggregates can form in nanoparticles due to parallel plane-to-plane stacking, resulting in quenched fluorescence. In addition, the overlap between P8 emission and the near-infrared dye absorption spectra was poor, causing inefficient FRET. By providing a blend of polymers, an efficient cascade of energy transfer exists from GBDP through PFDHTBT to BDP720 emitting monomeric unit (FIG. 23C). There is also good spectral overlap between PFGBDPPFDHTBT and PFDHTBT/BDP720 (FIG. 23B).

As shown in FIG. 23C, when PFDHTBT is not present, excited GBDP monomer and dimer (S₁′) can rapidly fall into the dimer's lower energy level (S₁″), which can be dipole-dipole forbidden for radiative emission. To compete with the non-emissive GBDP dimer, PFDHTBT was provided to maximize capture of energy from excited GBDP monomers and dimers via FRET. A high content of PFDHTBT can result in small average distance between GBDP and PFDHTBT, allowing for short-range energy transfer via electron exchange coupling, or via orbital overlap between donor and acceptor electronic densities, so that even in GBDP H-dimer in S₁″ state can transfer its energy to PFDHTBT. By incorporating BDP720 emitting monomeric unit into the PFDHTBT backbone, efficient energy transfer can occur via a cascade from GBDP (absorbing unit) to PFDHTBT, and from PFDHTBT to BDP720 (emitting monomeric unit). The BDP720 content of the nanoparticles are low, so its incorporation into the backbone of PFDHTBT can facilitate FRET; covalent conjugation of the donor and acceptor also can enable through-bond energy transfer.

The self-quenching of PFDHTBT and BDP720 can be restricted due to their low concentration in the blended nanoparticles. In addition to the efficient cascade energy transfer, the high quantum yield and absorbance of the blended polymer dots of P8 and P9 can be attributed to suppressed self-quenching. While nanoparticles including P9 alone showed low quantum yield (17.7%, Table 9), when the polymer was dispersed into a PFO nanoparticle host, the quantum yield improved to 44.0%. The PFO host polymer by itself has no absorption at 532 nm, indicating the improved quantum yield is due to suppressed self-quenching.

The estimated energy-transfer efficiency (Φ_{ET}) of the absorbing polymer (PFGBDP) to the emitting polymer (PFDHTBT-BDP720) can be calculated using the blended nanoparticles including PFO, because PFO by itself has no absorption at 532 nm. Accordingly, PFGBDP and PFDHTBT-BDP720 in the blended “80 wt % P8+20 wt % P9” nanoparticles can behave similarly as in the corresponding PFO-blended systems. Therefore, the energy transfer efficiency can be calculated as follows:

$${Φ}_{total} = [ \frac{{A}_{1}}{{A}_{1} + {A}_{2}} \times ( 1 - {Φ}_{1} ) \times {Φ}_{ET} + \frac{{A}_{2}}{{A}_{1} + {A}_{2}} ] \times {Φ}_{2}$$

wherein Φₜₒₜₐₗ is the quantum yield of the “80 wt % P8+20 wt % P9” blended nanoparticle, Φ₁ is the quantum yield of the nanoparticle including 80 wt % P8+20 wt % PFO, Φ₂ is the quantum yield of the nanoparticle including 80 wt % PFO+20 wt % P9, A₁ is the measured absorbance at 532 nm of the nanoparticle including 80 wt % P8+20 wt % PFO, A₂ is the measured absorbance at 532 nm of the nanoparticle including 80 wt % PFO+20 wt % P9, and (D_{E} is the estimated energy transfer efficiency. Using the measured values provided by Table 9, Φₜₒₜₐₗ=40.2%, Φ₁=0.6%, Φ₂=44.0%, A₁=0.210, and A₂=0.019. Accordingly, Φ_{ET} is calculated, and Φ_{ET}=91.1%.

An important criterion for evaluating the photophysical properties of luminescent nanoparticles is single molecule or single particle brightness. Theoretical brightness is proportional to the product of the absorbance cross-section and quantum yield. Nanoparticles including PFDHTBT-BDP720, as well as the blended nanoparticles were compared with PEGylated near-infrared emitting quantum dot Qdot 705 (Table 10). Nanoparticles were excited by a 532 nm laser; values of molar attenuation (ε₅₃₂) are provided.

TABLE 10
Photophysical properties of P9, P8-P9 blended nanoparticle, and Qdot 705

| | λ_{abs} | λ_{PL} | ε_{532 nm} | σ_{532 nm} | Φ_{PL} | Φ_{PL} × σ | (Φ_{PL} × σ)/V |
|---|---|---|---|---|---|---|---|
| Nanoparticle | (nm) | (nm) | (M⁻¹ cm⁻¹) | (cm²) | (%) | (cm²) | (cm⁻¹) |
| Qdot 705 | <300 | 707 | 2.1 × 10⁶ | 8.0 × 10⁻¹⁵ | 82.0 | 6.5 × 10⁻¹⁵ | 3089.9 |
| Polymer P9 | 520 | 724 | 1.5 × 10⁸ | 5.7 × 10⁻¹³ | 17.7 | 1.0 × 10⁻¹³ | 8236.2 |
| 80 wt % P8 + | 528 | 721 | 3.5 × 10⁸ | 1.3 × 10⁻¹² | 40.2 | 5.2 × 10⁻¹³ | 40068.6 |
| 20 wt % P9 | | | | | | | |

Theoretical brightness was calculated as the product of quantum yield and cross-section absorbance (Φ_{PL}×σ), and brightness per volume was similarly calculated (Φ_{PL}×σ)N). Brightness per volume of Qdot 705 was based on an average quantum dot diameter of 15.9 nm as measured by dynamic light scattering. The blended nanoparticles have approximately 5.2-fold greater single-particle brightness when compared to the nanoparticles including only polymer P9. While the quantum dot Qdot 705 has a high quantum yield of 82%, the blended nanoparticle is approximately 80 times brighter when excited by a 532 nm laser. This shows the importance of molar attenuation and absorption cross-section in determining overall brightness of nanoparticles. Single-particle brightness is also sensitive to particle size, so brightness per volume basis was calculated for the nanoparticles. The P9 nanoparticles were 2.7-fold brighter, and the blended 80 wt % P8+20 wt % P9 nanoparticles were 13.0-fold brighter than Qdot 705 per volume, when normalized to the size of water-soluble Qdot 705.

### Example 12. Synthesis of Cyanine Dye-Based Monomer and Related Polymer

This example describes the synthesis of cyanine dye based monomer and narrow-band absorbing copolymer Polymer P8.

Synthesis of Cyanine Dye Based Monomer

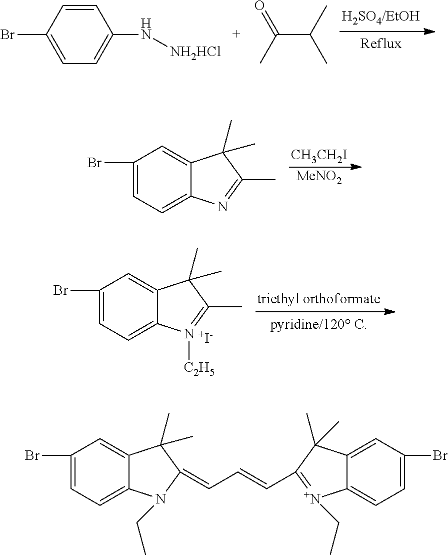

A mixture of 4-bromophenylhydrazine 1 (4.46 g, 20 mmol), isopropylmethylketone 2 (3.44 g, 40 mmol), EtOH (80 mL) and concentrated H₂SO₄ (1.86 g, 40 mmol) was heated under reflux for overnight. After cooling, the mixture was diluted with CH₂Cl₂ (100 mL) and was washed with 10% NaHCO₃ (100 ml) twice and water (100 mL) twice, then dried over Magnesium Sulfate and filtered. The solution was then passed through a short column quickly, and evaporated under reduced pressure to get 4.25 g product as reddish oil. (Yield: 90%).

A mixture of 5-Bromo-2,3,3-trimethylindolenine 3 (900 mg, 3.78 mmol), iodoethane (1.6 g, 4.45 mmol) and nitromethane (5 mL) was refluxed for overnight. After cooling and concentrating the mixture under reduced pressure, diethyl ether (25 mL) was added. The solution was cooled to 4° C. for 1 h, and the precipitate was collected, then washed with diethyl ether (50 mL) and dried. The yellow solid, 1.1 g, was obtained (Yield: 70%).]

A solution of indolium (1.0 mmol, 1.0 equiv) and triethyl orthoformate (296 mg, 2.0 mmol, 4.0 equiv) in dry pyridine (1 mL) was heated at reflux for 16 h under argon. The reaction mixture was cooled to room temperature, pyridine was removed in vacuum, and the residue was purified by column chromatography to obtain the cyanine-based monomer as a purple solid 0.16 g. (Yield: 59%).

Synthesis of Polymer P8

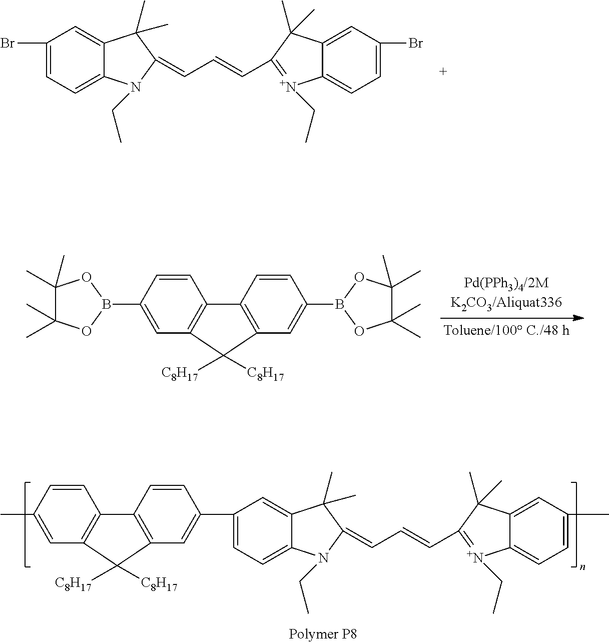

A mixture of cyanine-based monomer (0.1 mmol, 54.3 mg), (9,9-Dioctylfluorene-2,7-diboronic acid bis(1,3-propanediol) ester (0.1 mmol, 55.8 mg), Aliquat 336 (1 drop), Pd(PPh₃)₄ (5 mg, 0.005 mmol), 2 M aqueous K₂CO₃ (2 mL), and toluene (6 mL) was degassed 5 times under nitrogen gas. The resulting mixture was stirred at 100° C. for 48 h to afford Polymer P8. The polymer was then end-capped via the addition of 0.1 M phenylboronic acid (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped Polymer P2 with a yield of 75%.

### Example 13. Synthesis of Squaraine Dye-Based Monomer and Related Polymer

This example describes the synthesis of squaraine dye based monomer and narrow-band absorbing copolymer Polymer P9.

Synthesis of Squaraine Dye Based Monomer

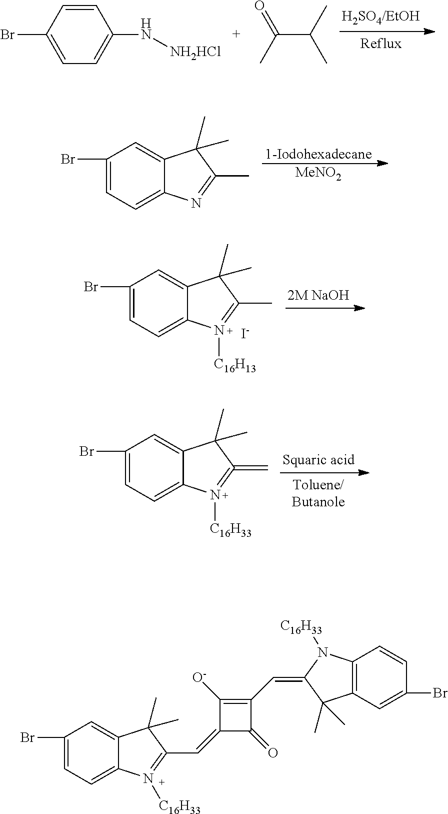

A mixture of 4-bromophenylhydrazine 1 (4.46 g, 20 mmol), isopropylmethylketone 2 (3.44 g, 40 mmol), EtOH (80 mL) and concentrated H₂SO₄ (1.86 g, 40 mmol) was heated under reflux for overnight. After cooling, the mixture was diluted with CH₂Cl₂ (100 mL) and was washed with 10% NaHCO₃ (100 ml) twice and water (100 mL) twice, then dried over Magnesium Sulfate and filtered. The solution was then passed through a short column quickly, and evaporated under reduced pressure to get 4.25 g product as reddish oil. (Yield: 90%).

A mixture of 5-Bromo-2,3,3-trimethylindolenine 3 (900 mg, 3.78 mmol), 1-iodohexadecane (1.6 g, 4.45 mmol) and nitromethane (5 mL) was refluxed for overnight. After cooling and concentrating the mixture under reduced pressure, diethyl ether (25 mL) was added. The solution was cooled to 4° C. for 1 h, and the precipitate was collected, then washed with diethyl ether (50 mL) and dried. The yellow solid, 1.1 g, was obtained (Yield: 70%).]

5-Bromo-1-hexadecyl-2,3,3-trimethyl-3H-indolium Iodide 4 (2.92 g, 3.26 mmol) was suspended in 2N NaOH aqueous solution (50 mL) and diethyl ether (50 mL), stirred for 30 minutes, extracted with diethyl ether and water, then dried and evaporated under vacuum. The product was yellowish oil, 1.84 g (Yield: 98%).

A mixture of 3,4-dihydroxy-3-cyclobutene-1,2-dione 4 (105 mg, 0.9 mmol) and 5-Bromo-1-hexadecyl-3,3-dimethyl-2-methylene-2,3-dihydroindole (840 mg, 1.84 mmol) in toluene/butanol (1:1, 15 mL) was refluxed overnight with a Dean-stark trap. After cooling to room temperature, the solvent was removed under vacuum. The residue was purified by silica gel with chromatography, and the product was obtained as a dark green solid, 500 mg (Yield: 50%).

Synthesis of Polymer P9

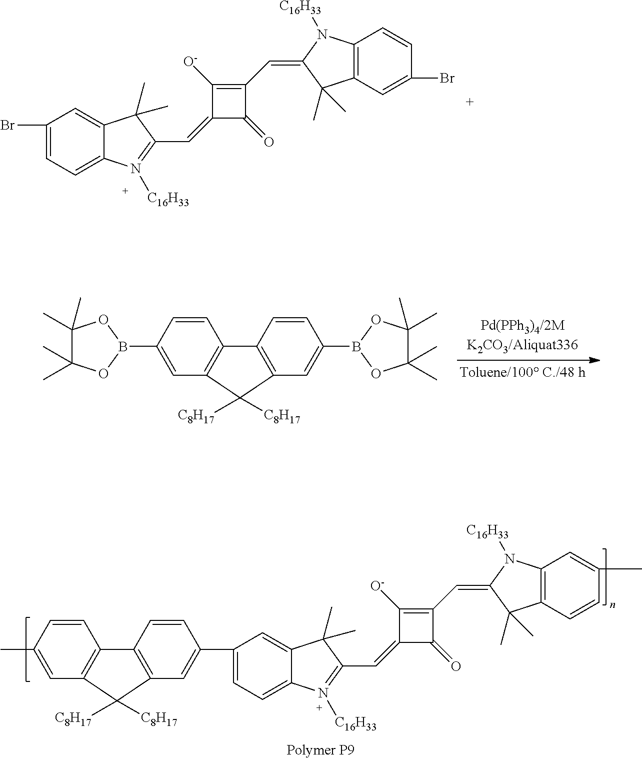

A mixture of squaraine-based monomer (0.1 mmol, 100.3 mg), (9,9-Dioctylfluorene-2,7-diboronic acid bis(1,3-propanediol) ester (0.1 mmol, 55.8 mg), Aliquat 336 (1 drop), Pd(PPh₃)₄ (5 mg, 0.005 mmol), 2 M aqueous K₂CO₃ (2 mL), and toluene (6 mL) was degassed 5 times under nitrogen gas. The resulting mixture was stirred at 100° C. for 48 h to afford polymer P9. The polymer was then end-capped via the addition of 0.1 M phenylboronic acid (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped polymer P9 with a yield of 68%.

### Example 14. Synthesis of diBODIPY-Based Monomer and Related Polymer

This example describes the synthesis of diBODIPY-based monomer and narrow-band absorbing copolymer Polymer P10.

Synthesis of diBODIPY Based Monomer

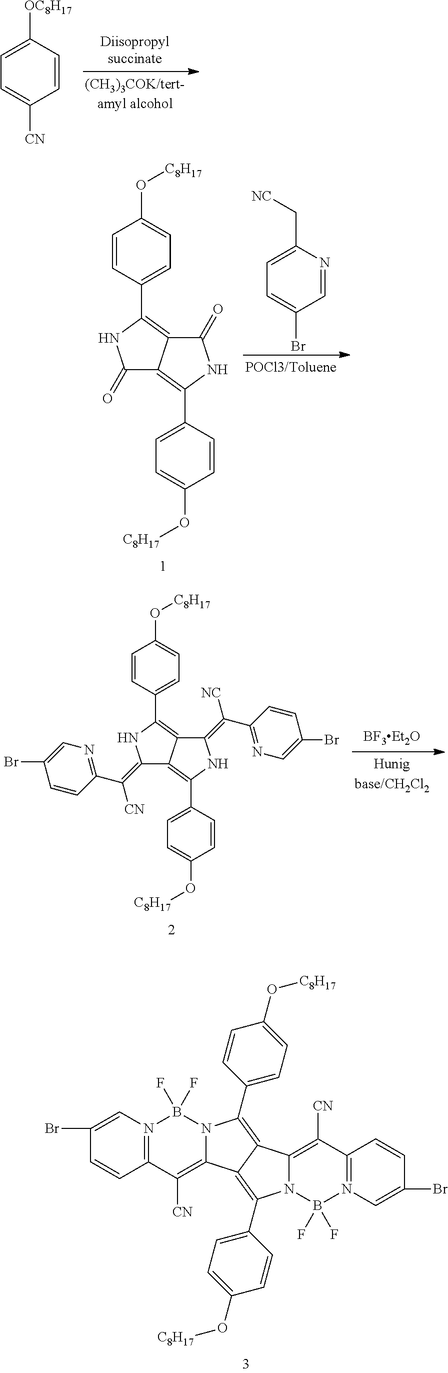

Potassium tert-butoxide (1.68 g, 15 mmol) was added to 2-methyl-2-butanol (15 mL), and the mixture was heated to reflux. When the base had dissolved, 4-(octyloxy)benzonitrile (2.3 g, 10 mmol) was added in one portion. Then diisopropyl succinate (1.01 g, 5 mmol) was added over 3 h with a dropping funnel. After heating for another 3 h at 110° C., the mixture was cooled and slowly added to a mixture of 100 mL of ethanol with 2 mL of concentrated hydrochloric acid. The red precipitate was collected by filtration and washed with ethanol. The solid was digested in boiling ethanol, collected by filtration and washed with ethanol. This procedure was repeated until the filtrate was clear. Drying in vacuum yielded an orange solid 1.6 g (Yield, 30%).

Compound 1 (0.54 g, 1 mmol) and 2-cyanomethylpyridine (0.295 g, 2.5 mmol) were heated to reflux in absolute toluene (20 mL) under nitrogen. Phosphoryl chloride (0.75 mL, 8 mmol) was then added. The reaction was monitored by TLC. As soon as 2 was used up, the reaction mixture was cooled, quenched with water, and alkalized with sodium bicarbonate solution. Water was separated and extracted with chloroform. The combined organic layer was dried over anhydrous sodium sulfate. After filtration, the volatile substances were removed under reduced pressure. The crude product was purified by column chromatography on silica to give compound 2 as a bluish green solid 0.16 g (Yield, 17%).

Compound 2 (108 mg, 0.12 mmol) and N,N-diisopropylethylamine (1.2 mL, 7.2 mmol) were dissolved in DCM (12 mL). Trifluoroborane etherate (1.2 mL, 9.6 mmol) was added and the mixture was stirred at room temperature for 2 h. The reaction mixture was washed with water and dried over anhydrous sodium sulfate. After removing the solvent, the crude product was purified by column chromatography with dichloromethane as eluent to give compound 3 as a green solid 91.4 mg (Yield, 91%).

Synthesis of Polymer P10

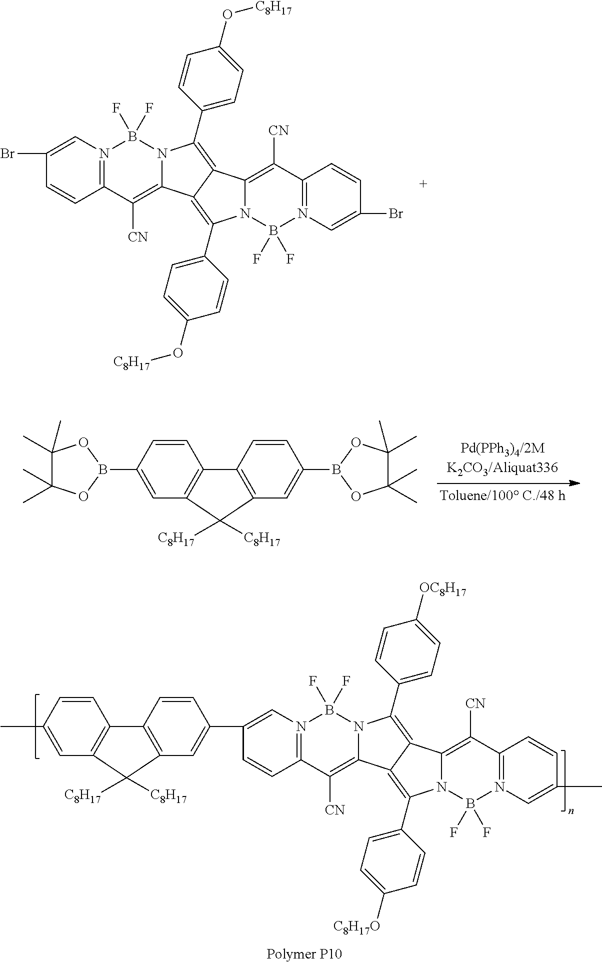

A mixture of diBODIPY-based monomer (0.1 mmol, 99.8 mg), (9,9-Dioctylfluorene-2,7-diboronic acid bis(1,3-propanediol) ester (0.1 mmol, 55.8 mg), Aliquat 336 (1 drop), Pd(PPh₃)₄ (5 mg, 0.005 mmol), 2 M aqueous K₂CO₃ (2 mL), and toluene (6 mL) was degassed 5 times under nitrogen gas. The resulting mixture was stirred at 100° C. for 48 h to afford polymer P10. The polymer was then end-capped via the addition of 0.1 M phenylboronic acid (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped polymer P10 with a yield of 63%.

### Example 15. Synthesis of Naphthalene Diimide-Based Monomer and Related Polymer

This example describes the synthesis of Naphthalene Diimide and narrow-band absorbing copolymer Polymer P11.

Synthesis of Naphthalene Diimide Based Monomer

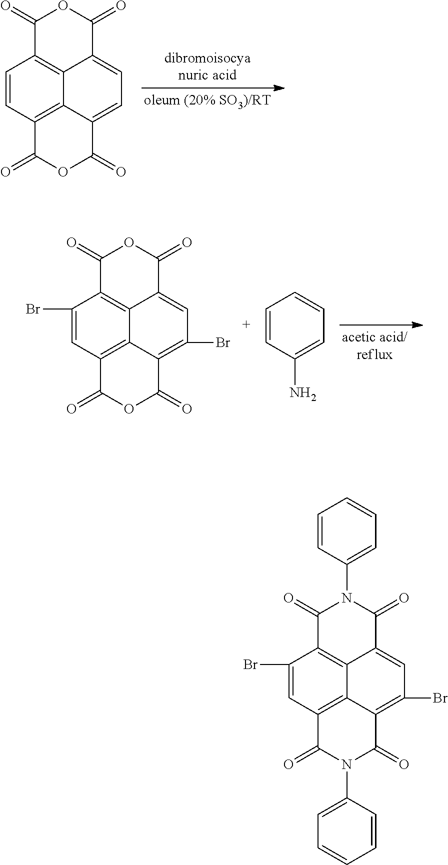

A solution of dibromoisocyanuric acid (2.86 g, 10.0 mmol) in oleum (20% SO3, 50 mL) was added at room temperature to a solution of naphthalene dianhydride 10 (2.68 g, 10.0 mmol) in oleum (20% SO3, 100 mL) over a period of 4 h. The resulting mixture was stirred at room temperature for 1 h and then cautiously poured onto ice (500 g) to give a bright yellow precipitate. Water (1.5 L) was added, and the mixture was allowed to stand for 3 h. The yellow solid was collected on a Buchner funnel, washed with dilute HCl, and dried to obtain 3.41 g crude product, which was used without further purification (Yield, 80%).

To a stirred suspension of dibromide anhydride (2.1 g, 5 mmol) in glacial acetic acid (10 mL per mmol dianhydride) was slowly added 8 equiv of the aniline (3.7 g, 40 mmol) at room temperature. After being heated to reflux for 10 min, the reaction mixture was cooled to room temperature. The resulting colorless to slightly brown precipitate was collected on a Buchner funnel and purified by recrystallization with glacial acetic acid to get 1.73 g product (Yield, 60%).

Synthesis of Polymer P11

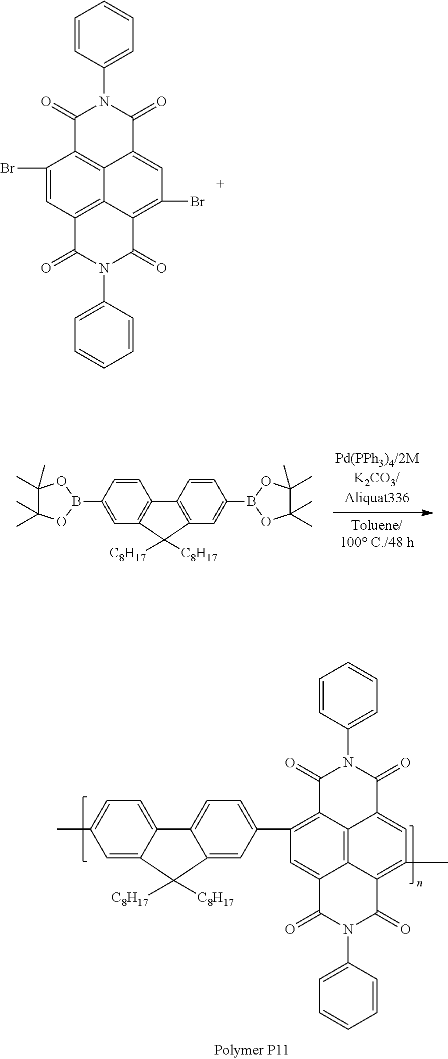

A mixture of naphthalene diimide-based monomer (0.1 mmol, 57.6 mg), (9,9-Dioctylfluorene-2,7-diboronic acid bis(1,3-propanediol) ester (0.1 mmol, 55.8 mg), Aliquat 336 (1 drop), Pd(PPh₃)₄ (5 mg, 0.005 mmol), 2 M aqueous K₂CO₃ (2 mL), and toluene (6 mL) was degassed 5 times under nitrogen gas. The resulting mixture was stirred at 100° C. for 48 h to afford polymer P11. The polymer was then end-capped via the addition of 0.1 M phenylboronic acid (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped polymer P11 with a yield of 72%.

### Example 16. Synthesis of Perylene Diimide-Based Monomer and Related Polymer

This example describes the synthesis of Perylene Diimide and narrow-band absorbing copolymer Polymer P12.

Synthesis of perylene Diimide Based Monomer

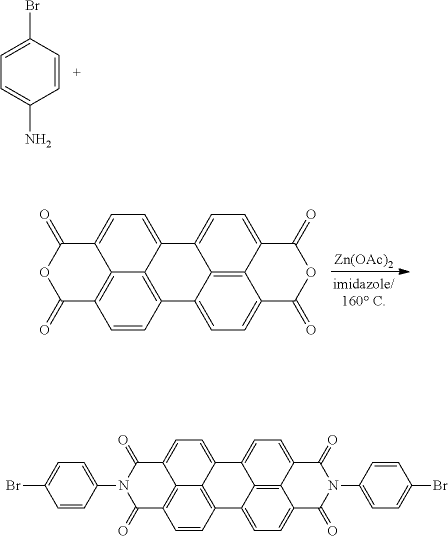 [SMILES: Nc1ccc(Br)cc1 ; O=C1OC(=O)c2ccc3c4ccc5c6c(ccc(c7ccc1c2c73)c64)C(=O)OC5=O ; O=C1c2ccc3c4ccc5c6c(ccc(c7ccc(c2c37)C(=O)N1c1ccc(Br)cc1)c64)C(=O)N(c1ccc(Br)cc1)C5=O ; "Zn(OAc)2"]

A mixture of 3,4,9,10-perylene tetracarboxylic dianhydride (5 g, 12.7 mmol), 4-bromobenzenamine (5.4 g 32 mmol) of, 100 g of imidazole and (1.0 g, 4.56 mmol) of zinc acetate were heated at 100° C. for 2 h. The mixture was heated at 160° C. for 20 h under an argon atmosphere. Then the mixture was cooled to room temperature and acidified with 500 mL of 2N hydrochloric acid. The precipitate was collected by filtration and washed with copious amounts of water and methanol to remove impurities. The precipitate was finally dried under vacuum at 100° C. to obtain 5.7 g product (Yield, 64%)

Synthesis of Polymer P12

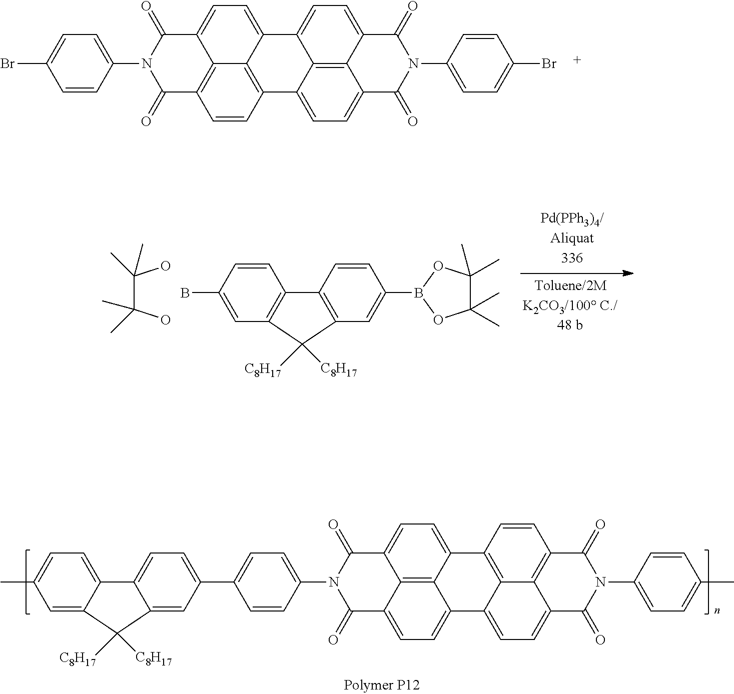

A mixture of perylene diimide-based monomer (0.1 mmol, 70 mg), (9,9-Dioctylfluorene-2,7-diboronic acid bis(1,3-propanediol) ester (0.1 mmol, 55.8 mg), Aliquat 336 (1 drop), Pd(PPh₃)₄ (5 mg, 0.005 mmol), 2 M aqueous K₂CO₃ (2 mL), and toluene (6 mL) was degassed 5 times under nitrogen gas. The resulting mixture was stirred at 100° C. for 48 h to afford polymer P12. The polymer was then end-capped via the addition of 0.1 M phenylboronic acid (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped polymer P12 with a yield of 81%.

### Example 17. Synthesis of Perylene Diimide-Based Monomer and Related Polymer

This example describes the synthesis of perylene diimide and narrow-band absorbing copolymer Polymer P13.

Synthesis of perylene Diimide Based Monomer

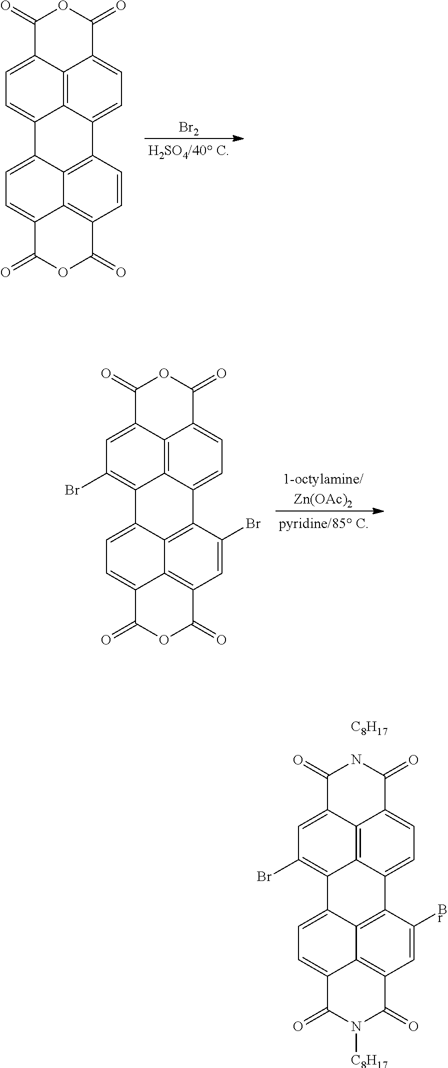

Perylene-3,4,9,10-tetracarboxylic acid dianhydride (5.00 g, 12.7 mmol, 1 equiv.) was suspended in conc. sulfuric acid (150 mL) and stirred 1 h at room temperature. Iodine (0.26 g, 1.0 mmol, 0.08 equiv.) was added, and the mixture was warmed up to 85° C. over 45 min. Finally, bromine (3.92 mL, 12.2 g, 76.5 mmol, 6 equiv.) was added, and the mixture was stirred overnight at 95° C. After cooling, the intensive red raw product was precipitated by the addition of water. The residue was washed with water until the wash solution had a neutral pH value and then dried to obtain a rust-colored 6.91 g product (Yield, 99%)

Dibromoperylene-3,4,9,10-Tetracarboxylic Acid Dianhydride (1.00 g, 1.8 mmol) and zinc acetate dihydrate (200 mg, 0.9 mmol) were suspended in pyridine (200 mL) and warmed up to 85° C. After reaching this temperature, 1-octylamine (3.0 mL, 2.36 g, 18 mmol) in pyridine (30 mL) was added dropwise over 3 h, and the mixture was stirred for another 12 h. The solvent was evaporated, and the dark red residue was purified by column chromatography to obtain a dark red solid 0.97 g (Yield, 70%).

Synthesis of Polymer P13

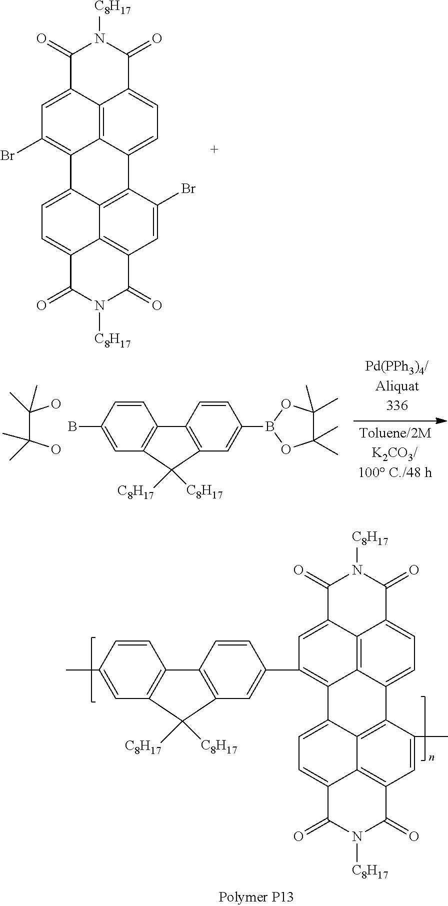

A mixture of perylene diimide-based monomer (0.1 mmol, 77.2 mg), (9,9-Dioctylfluorene-2,7-diboronic acid bis(1,3-propanediol) ester (0.1 mmol, 55.8 mg), Aliquat 336 (1 drop), Pd(PPh₃)₄ (5 mg, 0.005 mmol), 2 M aqueous K₂CO₃ (2 mL), and toluene (6 mL) was degassed 5 times under nitrogen gas. The resulting mixture was stirred at 100° C. for 48 h to afford polymer P13. The polymer was then end-capped via the addition of 0.1 M phenylboronic acid (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped polymer P13 with a yield of 74%.

### Example 18. Synthesis of Cyanine Side Chain-Containing Monomer and Related Polymer

Dyes can be grafted to the side chains of polymers in a variety of ways, including but not limited to alkyl chain, ether, amide, ester bonds. A variety of polymerization reactions can be used for synthesis of the polymers described herein, including Heck, Mcmurray and Knoevenagel, Wittig, Homer, Suzuki-Miyaura, Sonogashira, Yamamoto, Stille coupling reaction, etc., as described above.

This example describes the synthesis of side chain cyanine dye based monomer and narrow-band absorbing copolymer Polymer P14.

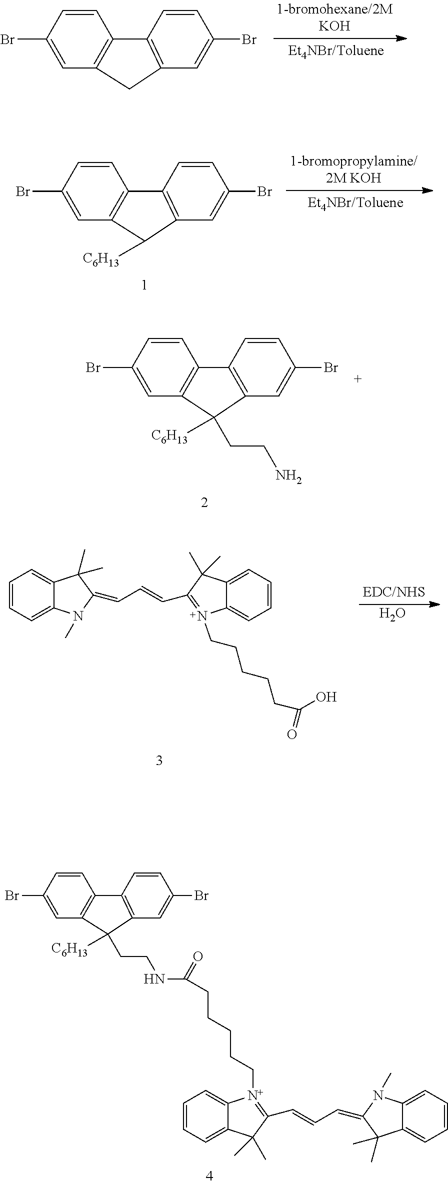

2,7-dibromo-fluorene (9.7 g, 30 mmol) and tetraethyl ammonium bromide (0.6 g, 3 mmol) was dissolved in degassed mixture toluene (120 mL) and 1 N NaOH (80 mL) and stirred under 80° C. for 30 min. After that, 1-bromohexane (5.9 g, 36 mmol) in 50 mL toluene was dropwise added in 2 hours and then continue to react for overnight. After cooling down, the organic layer was acidified using 0.1 M HCl and washed with water for three times, filtered and dried with Na₂SO4. After evaporating the solvent under vacuum, the crude product was purified by column chromatography to give 3.7 g white solid product of compound 1 (Yield, 31%).

A mixture of 2,7-dibromo-9-hexylfluorene (2.0 g, 5 mmol), 3-bromopropylamine hydrobromide (1.0 g, 6 mmol), DMSO (10 mL), KOH (0.8 g, 15 mmol) and (n-C₄Hg)₄NBr (0.16 g, 0.05 mmol) was stirred at 35° C. overnight. After workup, the mixture was poured into water and extracted with CH₂Cl₂. The organic layer was washed with water and then dried with Na₂SO₄. After filtration and removal of the solvent, the residue was purified by column chromatography to afford 1.6 g product as a colorless viscous liquid (Yield, 67%).

1-Ethyl-3-(3-dimethylaminopropyl)carbodiimide (EDC) hydrochloride (1.9 g, 10 mmol) soluble in anhydrous CH₂Cl₂ was added to anhydrous THF solution containing compound 3 (0.46 g, 1 mmol) and N-Hydroxysuccinimide (NHS, 0.23 g, 2 mmol) and then stirred at room temperature 2 hours. Then, compound 2 (0.9 g, 2 mmol) was added to the solution and reacted for additional 24 hours. After workup, 200 mL CH₂Cl₂ was added and washed with di-water for three times. After dried and evaporated the solvent, the crude product was purified by column chromatography to afford 0.65 g purple solid (Yield, 73%)

Synthesis of Polymer P13

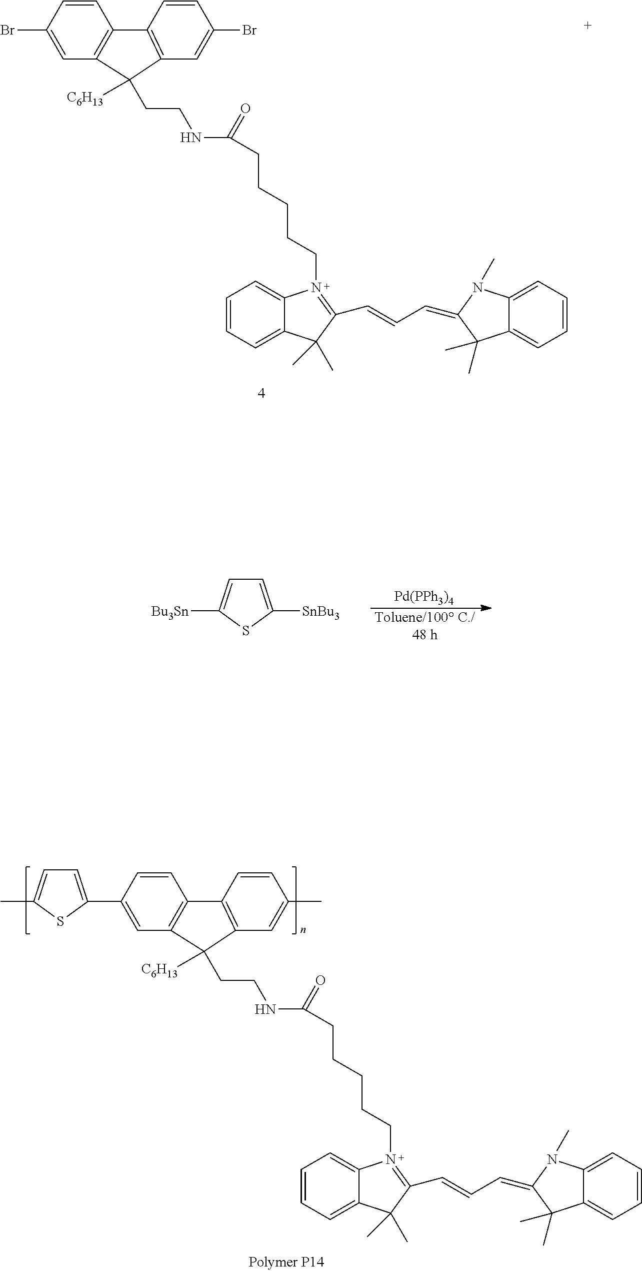

A mixture of cyanine-based monomer (0.1 mmol, 89.0 mg), 2,5-bis(tributylstannyl)thiophene (0.1 mmol, 66.2 mg), Pd(PPh₃)₄ (5 mg, 0.005 mmol), and toluene (6 mL) was degassed 5 times under nitrogen gas. The resulting mixture was stirred at 100° C. for 48 h to afford polymer P14. The polymer was then end-capped via the addition of 0.1 M tributyl(thiophen-2-yl)stannane (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped polymer P14 with a yield of 57%.

### Example 19. Synthesis of Squaraine Dye Side Chain-Containing Monomer and Related Polymer

This example describes the synthesis of side chain squaraine dye based monomer and narrow-band absorbing copolymer Polymer P15.

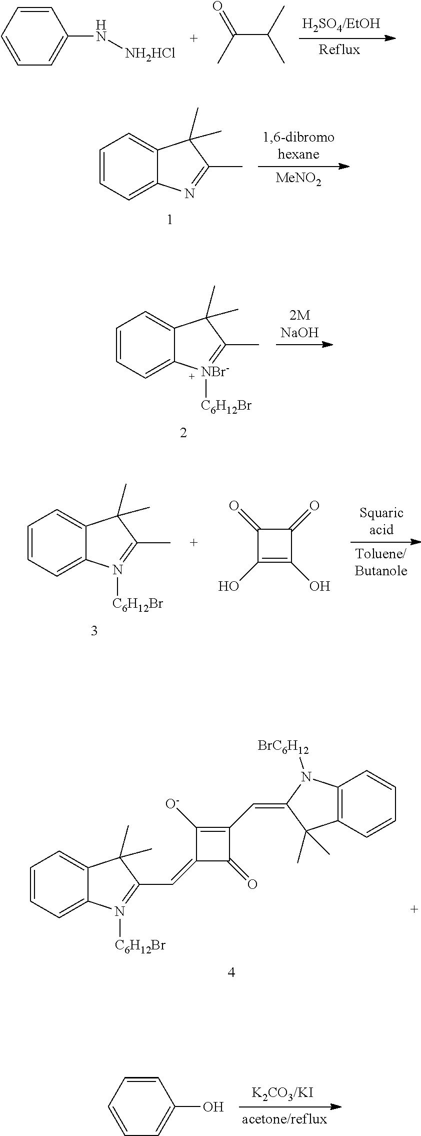

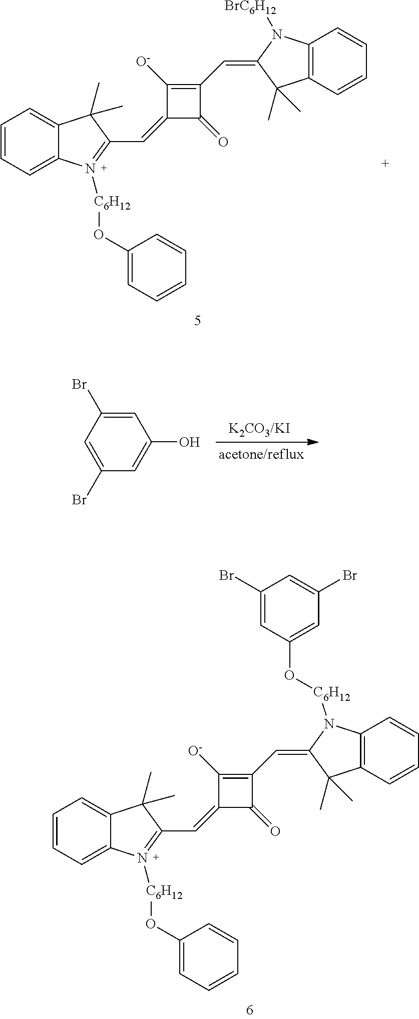

A mixture of 4-bromophenylhydrazine 1 (2.9 g, 20 mmol), isopropylmethylketone 2 (3.44 g, 40 mmol), EtOH (80 mL) and concentrated H₂SO₄ (1.86 g, 40 mmol) was heated under reflux for overnight. After cooling, the mixture was diluted with CH₂Cl₂ (100 mL) and was washed with 10% NaHCO₃ (100 ml) twice and water (100 mL) twice, then dried over Magnesium Sulfate and filtered. The solution was then passed through a short column quickly, and evaporated under reduced pressure to get 2.9 g product as reddish oil. (Yield: 90%).

A mixture of 2,3,3-trimethylindolenine 1 (1.6 g, 10 mmol), 1,6-dibromohexane (24.2 g, 100 mmol) and nitromethane (150 mL) was refluxed for overnight. After cooling and concentrating the mixture under reduced pressure, diethyl ether (100 mL) was added and sonicated. The solution was cooled to 4° C. for 1 h, and the precipitate was collected, then washed with diethyl ether and dried to obtain 2.5 g yellow solid compound 2 (Yield: 62%).

Compound 2 (1.6 g, 4 mmol) was suspended in 2N NaOH aqueous solution (50 mL) and diethyl ether (50 mL), stirred for 30 minutes, extracted with diethyl ether and water, then dried and evaporated under vacuum. The compound 3 product was yellowish oil, 1.22 g (Yield: 95%).

A mixture of 3,4-dihydroxy-3-cyclobutene-1,2-dione (0.16 g, 1.4 mmol) and compound 3 (0.96 g, 3 mmol) in toluene/butanol (1:1, 30 mL) was refluxed overnight with a Dean-stark trap. After cooling to room temperature, the solvent was removed under vacuum. The residue was purified by silica gel with chromatography, and the product was obtained compound 4 as a dark green solid, 0.73 mg (Yield: 72%).

A mixture of compound 4 (0.58 g, 0.8 mmol) and phenol (0.96 g, 0.8 mmol), K₂CO₃ (0.7 g, 5 mmol and KI (83 mg, 0.5 mmol) in acetone (30 mL) was refluxed overnight. After cooling to room temperature, the mixture was filtered and washed with DCM, and then the organic solvent was removed under vacuum. The residue was purified by silica gel with chromatography, and the product compound 5 was obtained as a dark green solid, 0.22 g (Yield: 38%).

A mixture of compound 5 (0.22 g, 0.3 mmol) and 3,5-dibromophenol (0.75 g, 3 mmol), K₂CO₃ (0.7 g, 5 mmol and KI (83 mg, 0.5 mmol) in acetone (30 mL) was refluxed overnight. After cooling to room temperature, the mixture was filtered and washed with DCM, and then the organic solvent was removed under vacuum. The residue was purified by silica gel with chromatography, and the monomer compound 6 was obtained as a dark green solid, 0.24 g (Yield: 90%/).

Synthesis of Polymer P15

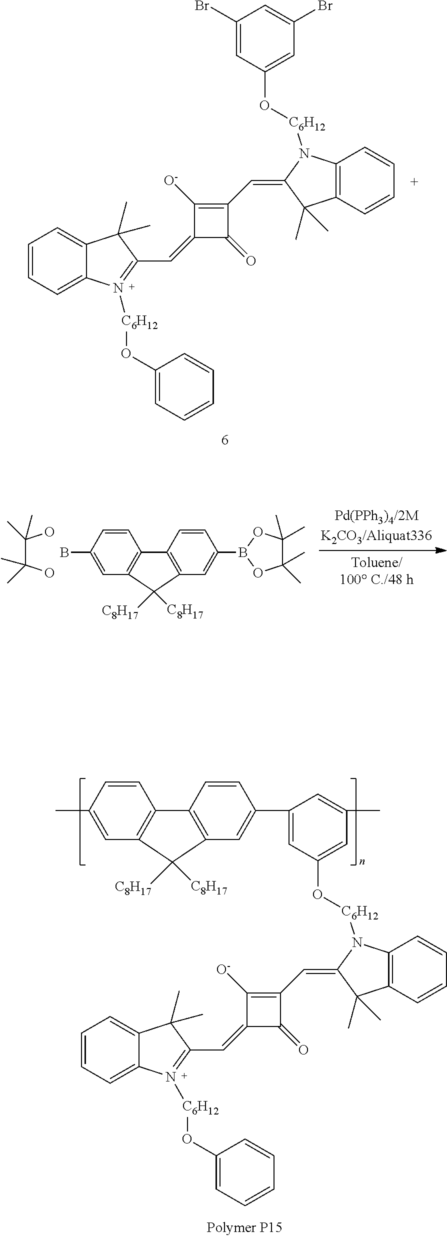

A mixture of squaraine-based monomer (0.1 mmol, 90.7 mg), (9,9-Dioctylfluorene-2,7-diboronic acid bis(1,3-propanediol) ester (0.1 mmol, 55.8 mg), Aliquat 336 (1 drop), Pd(PPh₃)₄ (5 mg, 0.005 mmol), 2 M aqueous K₂CO₃ (2 mL), and toluene (6 mL) was degassed 5 times under nitrogen gas. The resulting mixture was stirred at 100° C. for 48 h to afford polymer P15. The polymer was then end-capped via the addition of 0.1 M phenylboronic acid (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped polymer P15 with a yield of 62%.

### Example 20. Synthesis of diBODIPY Side Chain-Containing Monomer and Related Polymer

This example describes the synthesis of side chain diBODIPY dye based monomer and narrow-band absorbing copolymer Polymer P16.

Synthesis of Side Chain diBODIPY Dye Based Monomer

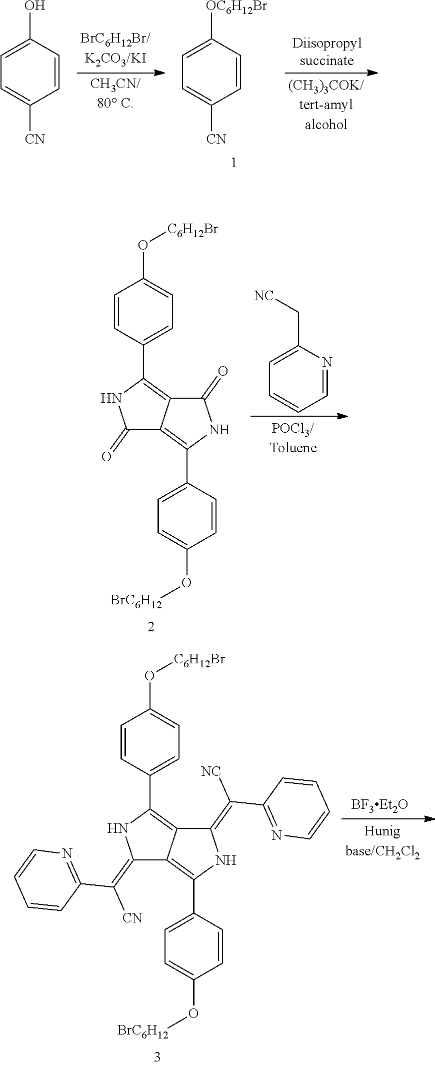

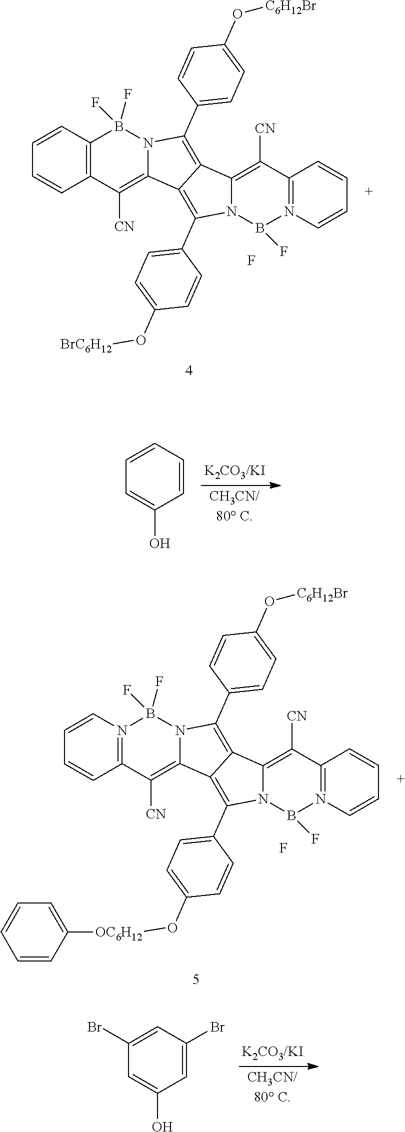

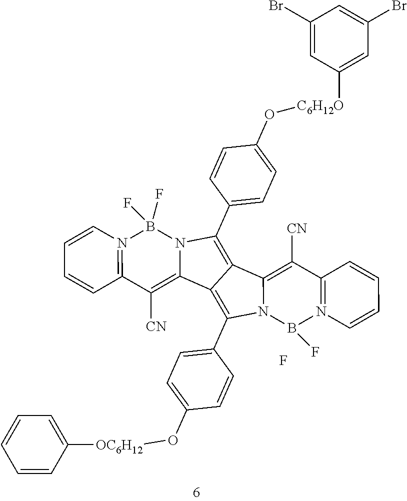

A mixture of 4-hydroxybenzonitrile (2.4 g, 20 mmol) and 1,8-dibromohexane, (48.4 g, 200 mmol), K₂CO₃ (5.6 g, 40 mmol and KI (0.33 g, 2 mmol) in acetonitrile (200 mL) was refluxed overnight. After cooling to room temperature, the mixture was filtered and washed with DCM, and then the organic solvent was removed under vacuum. The residue was purified by silica gel with chromatography, and the product compound 1 was obtained as a white solid, 5.1 g (Yield: 90%).

Potassium tert-butoxide (3.4 g, 30 mmol) was added to 2-methyl-2-butanol (30 mL), and the mixture was heated to reflux. When the base had dissolved, 4-(6-bromohexyloxy)benzonitrile (5.0 g, 18 mmol) was added in one portion. Then diisopropyl succinate (2.0 g, 10 mmol) was added over 3 h with a dropping funnel. After heating for another 3 h at 110° C., the mixture was cooled and slowly added to a mixture of 200 mL of ethanol with 4 mL of concentrated hydrochloric acid. The red precipitate was collected by filtration and washed with ethanol. The solid was digested in boiling ethanol, collected by filtration and washed with ethanol. This procedure was repeated until the filtrate was clear. Drying in vacuum yielded an orange solid 2.9 g compound 2 (Yield, 25%).

Compound 2 (2.6 g, 4 mmol) and 2-cyanomethylpyridine (1.2 g, 10 mmol) were heated to reflux in absolute toluene (80 mL) under nitrogen. Phosphoryl chloride (3 mL, 32 mmol) was then added. The reaction was monitored by TLC. As soon as 2 was used up, the reaction mixture was cooled, quenched with water, and alkalized with sodium bicarbonate solution. Water was separated and extracted with chloroform. The combined organic layer was dried over anhydrous sodium sulfate. After filtration, the volatile substances were removed under reduced pressure. The crude product was purified by column chromatography on silica to give compound 3 as a bluish green solid 0.54 g (Yield, 16%).

Compound 3 (508 mg, 0.6 mmol) and N,N-diisopropylethylamine (2.5 mL, 15 mmol) were dissolved in DCM (50 mL). Trifluoroborane etherate (2.5 mL, 20 mmol) was added and the mixture was stirred at room temperature for 2 h. The reaction mixture was washed with water and dried over anhydrous sodium sulfate. After removing the solvent, the crude product was purified by column chromatography with dichloromethane as eluent to give compound 4 as a green solid 537 mg (Yield, 95%).

A mixture of compound 4 (0.47 g, 0.5 mmol) and phenol (0.96 g, 0.8 mmol), K₂CO₃ (0.7 g, 5 mmol and KI (83 mg, 0.5 mmol) in acetone (30 mL) was refluxed overnight. After cooling to room temperature, the mixture was filtered and washed with DCM, and then the organic solvent was removed under vacuum. The residue was purified by silica gel with chromatography, and the product compound 5 was obtained as a dark green solid, 0.19 g (Yield: 40%).

A mixture of compound 5 (0.19 g, 0.2 mmol) and 3,5-dibromophenol (0.75 g, 3 mmol), K₂CO₃ (0.7 g, 5 mmol and KI (83 mg, 0.5 mmol) in acetone (30 mL) was refluxed overnight. After cooling to room temperature, the mixture was filtered and washed with DCM, and then the organic solvent was removed under vacuum. The residue was purified by silica gel with chromatography, and the monomer compound 6 was obtained as a dark green solid, 0.21 g (Yield: 95%).

Synthesis of Polymer P16

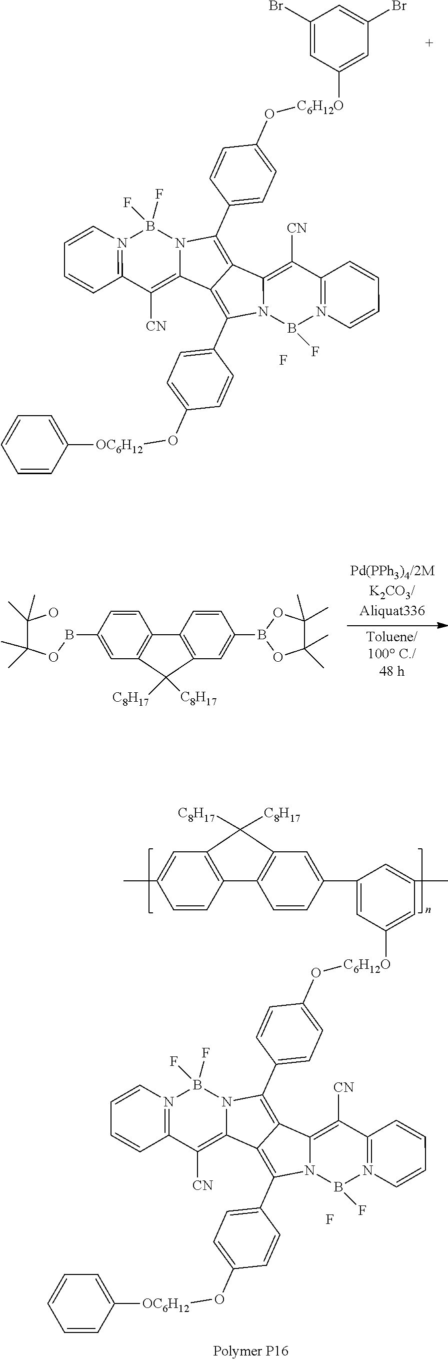

A mixture of diBODIPY-based monomer (0.1 mmol, 112.4 mg), (9,9-Dioctylfluorene-2,7-diboronic acid bis(1,3-propanediol) ester (0.1 mmol, 55.8 mg), Aliquat 336 (1 drop), Pd(PPh₃)₄ (5 mg, 0.005 mmol), 2 M aqueous K₂CO₃ (2 mL), and toluene (6 mL) was degassed 5 times under nitrogen gas. The resulting mixture was stirred at 100° C. for 48 h to afford polymer P16. The polymer was then end-capped via the addition of 0.1 M phenylboronic acid (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped polymer P16 with a yield of 75%.

### Example 21. Synthesis of Naphthalene Diimide Side Chain-Containing Monomer and Related Polymer

This example describes the synthesis of side chain naphthalene diimide-based monomer and narrow-band absorbing copolymer Polymer P17.

Synthesis of Naphthalene Diimide Based Monomer

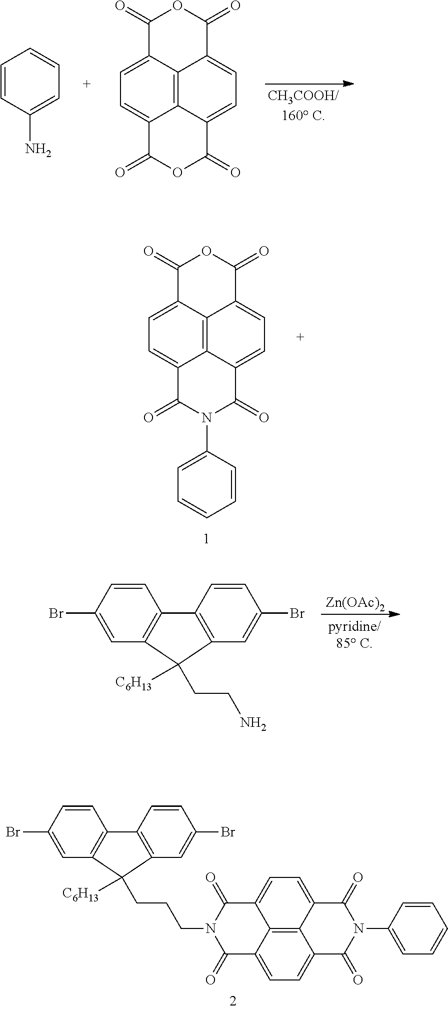

To a stirred suspension of naphthalene dianhydride (12.6 g, 30 mmol) in glacial acetic acid (150 mL) was slowly added aniline (0.47 g, 5 mmol) at room temperature. After being heated to reflux for 2 hours, the reaction mixture was filtered. After concentration under vacuum, the residue was purified by column chromatography and recrystallization with glacial acetic acid to get 0.51 g product (Yield, 30%).

Compound 1 (0.34 g, 1.0 mmol), 2,7-dibromo-9-hexyl-9-(3-aminopropyl)fluorene (0.9 g, 2 mmol) and zinc acetate dihydrate (200 mg, 0.5 mmol) were suspended in pyridine (100 mL), the mixture was stirred at 85° C. for 12 h. The solvent was evaporated, and the residue was purified by column chromatography to obtain 0.47 g solid compound 2 (Yield, 60%/).

Synthesis of Polymer P17

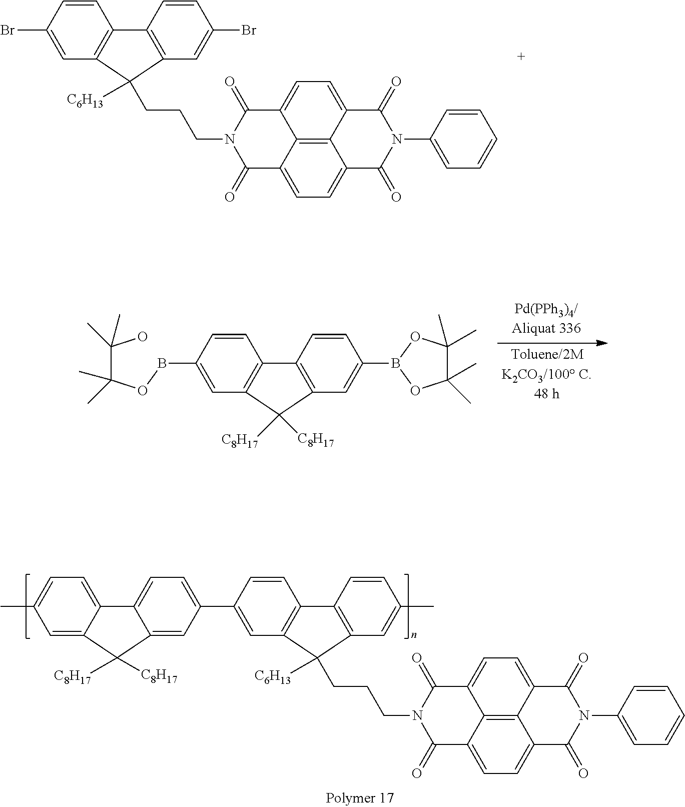

A mixture of naphthalene diimide-based monomer (0.1 mmol, 79 mg), (9,9-Dioctylfluorene-2,7-diboronic acid bis(1,3-propanediol) ester (0.1 mmol, 55.8 mg), Aliquat 336 (1 drop), Pd(PPh₃)₄ (5 mg, 0.005 mmol), 2 M aqueous K₂CO₃ (2 mL), and toluene (6 mL) was degassed 5 times under nitrogen gas. The resulting mixture was stirred at 100° C. for 48 h to afford polymer P17. The polymer was then end-capped via the addition of 0.1 M phenylboronic acid (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped polymer P17 with a yield of 78%.

### Example 22. Synthesis of Perylene Diimide Side Chain-Containing Monomer and Related Polymer

This example describes the synthesis of side chain perylene diimide-based monomer and narrow-band absorbing copolymer Polymer P17.

Synthesis of Perylene Diimide Based Monomer

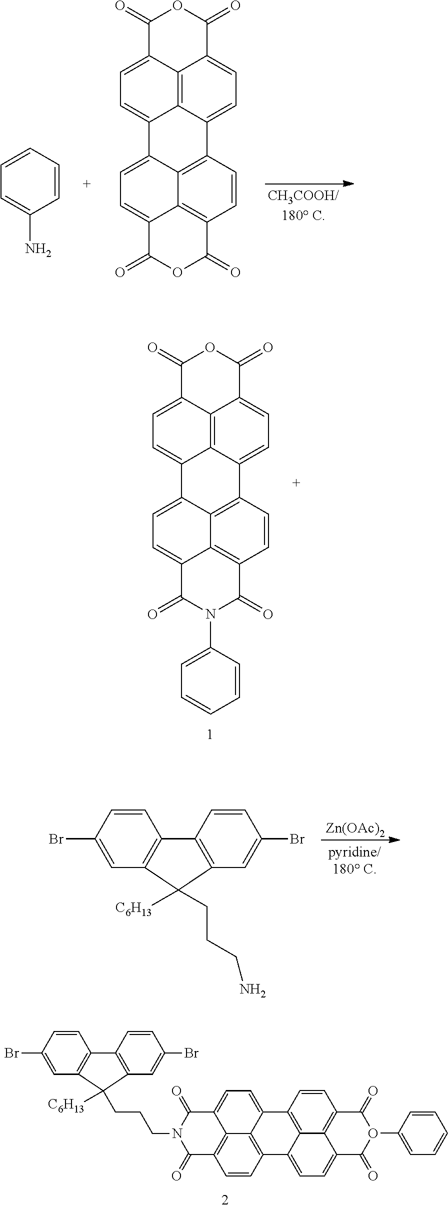

To a stirred suspension of perylene dianhydride (12.6 g, 30 mmol) in glacial acetic acid (200 mL) was slowly added aniline (0.93 g, 10 mmol) at room temperature. After being heated to reflux for 2 hours, the reaction mixture was filtered. After concentration under vacuum, the residue was purified by column chromatography and recrystallization with glacial acetic acid to get 1.1 g product (Yield, 25%).

Compound 1 (0.47 g, 1.0 mmol), 2,7-dibromo-9-hexyl-9-(3-aminopropyl)fluorene (0.9 g, 2 mmol) and zinc acetate dihydrate (200 mg, 0.5 mmol) were suspended in pyridine (100 mL), the mixture was stirred at 85° C. for 12 h. The solvent was evaporated, and the residue was purified by column chromatography to obtain 0.53 g solid compound 2 (Yield, 58%).

Synthesis of Polymer P18

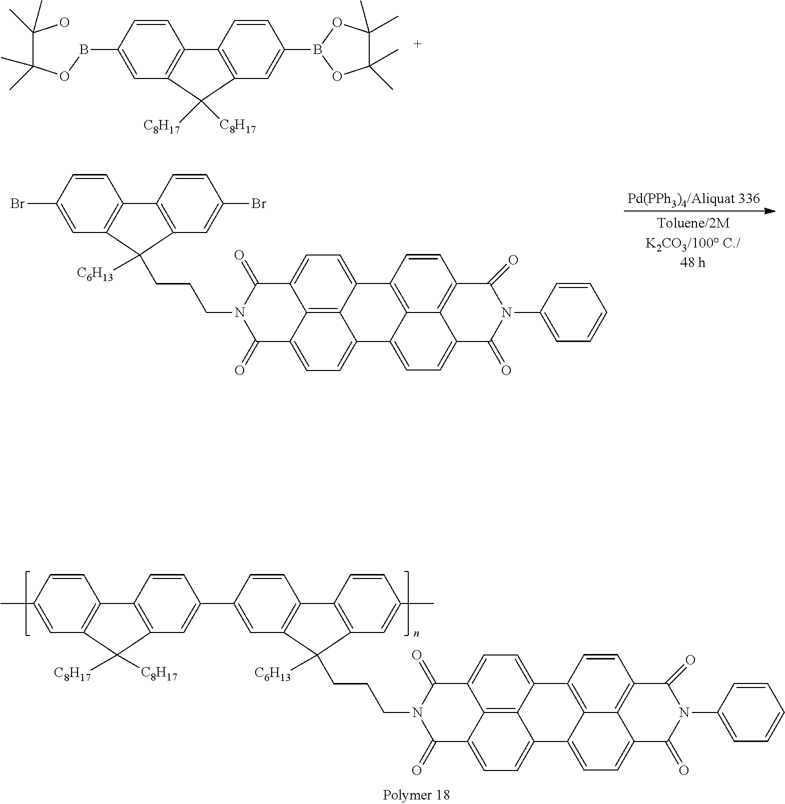

A mixture of perylene diimide-based monomer (0.1 mmol, 91.2 mg), (9,9-Dioctylfluorene-2,7-diboronic acid bis(1,3-propanediol) ester (0.1 mmol, 55.8 mg), Aliquat 336 (1 drop), Pd(PPh₃)₄ (5 mg, 0.005 mmol), 2 M aqueous K₂CO₃ (2 mL), and toluene (6 mL) was degassed 5 times under nitrogen gas. The resulting mixture was stirred at 100° C. for 48 h to afford polymer P18. The polymer was then end-capped via the addition of 0.1 M phenylboronic acid (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped polymer P18 with a yield of 74%.

### Example 23. Synthesis of Atto/Alexa/Rhodamine Dye Side Chain-Containing Monomer and Related Polymer

This example describes the synthesis of Atto/Alexa/rhodamine dye-based monomer and narrow-band absorbing copolymer Polymer P19.

Synthesis of Atto Dye Based Monomer

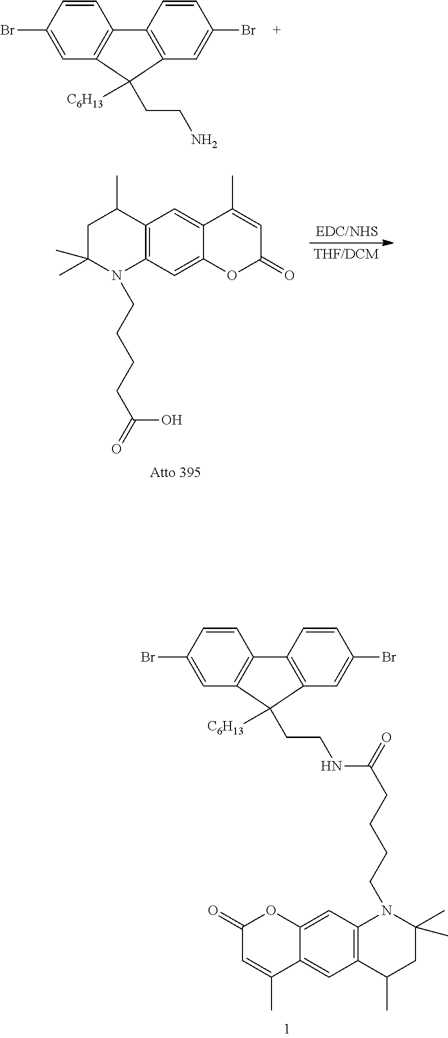

1-Ethyl-3-(3-dimethylaminopropyl)carbodiimide (EDC) hydrochloride (1.9 g, 10 mmol) soluble in anhydrous CH₂Cl₂ was added to anhydrous THF solution containing compound 3 (0.46 g, 1 mmol) and N-Hydroxysuccinimide (NHS, 0.23 g, 2 mmol) and then stirred at room temperature 2 hours. Then, 2,7-dibromo-9-hexyl-9-(3-aminopropyl)fluorene (0.9 g, 2 mmol) was added to the solution and reacted for additional 24 hours. After workup, 200 mL CH₂Cl₂ was added and washed with di-water for three times. After dried and evaporated the solvent, the crude product was purified by column chromatography to afford 0.55 g monomer solid (Yield, 69%)

Synthesis of Polymer P19

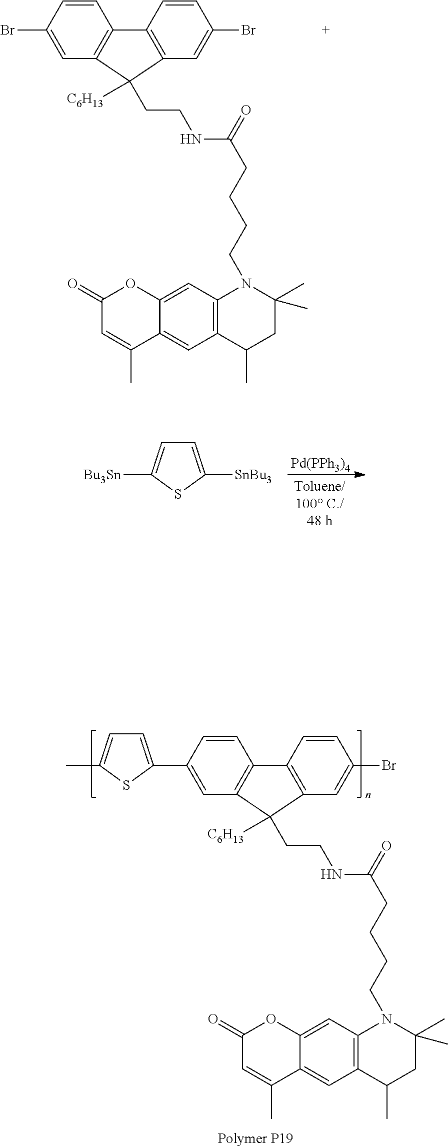

A mixture of Atto dye-based monomer (0.1 mmol, 79.0 mg), 2,5-bis(tributylstannyl)thiophene (0.1 mmol, 66.2 mg), Pd(PPh₃)₄ (5 mg, 0.005 mmol), and toluene (6 mL) was degassed 5 times under nitrogen gas. The resulting mixture was stirred at 100° C. for 48 h to afford polymer P19. The polymer was then end-capped via the addition of 0.1 M tributyl(thiophen-2-yl)stannane (1 mL) and bromobenzene (1 mL) to the solution. After cooling, the reaction mixture was poured into methanol and filtered. The precipitate was collected and dissolved in DCM, then the organic layer was washed with water and dried over anhydrous Na₂SO₄. The solution was concentrated, and after evaporating most of the solvent, the residue was precipitated in stirring methanol to afford a fiber-like solid, which was dried under vacuum to afford end-capped polymer P19 with a yield of 61%.

While illustrative embodiments have been illustrated and described, it will be appreciated that various changes can be made therein without departing from the spirit and scope of the disclosure.

1. A nanoparticle comprising a polymer, the polymer comprising:
an absorbing monomeric unit; and
an emitting monomeric unit;
wherein the nanoparticle has an absorbance width of less than 150 nm at 10% of the absorbance maximum, and
wherein the nanoparticle has a quantum yield of greater than 5%.

2. A nanoparticle comprising a polymer, the polymer comprising:
an absorbing monomeric unit comprising a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof, and
an emitting monomeric unit.

3. The nanoparticle of claim 1, wherein the polymer further comprises one or more monomeric units different from the absorbing monomeric unit and the emitting monomeric unit.

4. A nanoparticle comprising a polymer, the polymer comprising:
a first absorbing monomeric unit;
an emitting monomeric unit; and
one or more monomeric units different from the absorbing monomeric unit and the emitting monomeric unit;
wherein the nanoparticle has an absorbance width of less than 150 nm at 15% of the absorbance maximum.

5. The nanoparticle of claim 4, wherein the absorbing monomeric unit comprises a BODIPY, a BODIPY derivative, a diBODIPY, a diBODIPY derivative, an Atto dye, a rhodamine, a rhodamine derivative, a coumarin, a coumarin derivative, cyanine, a cyanine derivative, pyrene, a pyrene derivative, squaraine, a squaraine derivative, or any combination thereof.

6. (canceled)

7. The nanoparticle of claim 4, wherein the one or more monomeric units different from the absorbing monomeric unit and the emitting monomeric comprise a general monomeric unit, a functional monomeric unit, an energy transfer monomeric unit, a second absorbing monomeric unit, or any combination thereof.

8. The nanoparticle of claim 7, wherein the functional monomeric unit comprises a hydrophilic monomeric unit.

9. The nanoparticle of claim 4, wherein the polymer comprises a first absorbing monomeric unit, an emitting monomeric unit, an energy transfer unit, and an optional functional monomeric unit.

10-12. (canceled)

13. The nanoparticle of claim 1, wherein the nanoparticle comprises an absorption peak having a longer wavelength than 450 nm.

14. The nanoparticle of claim 1, wherein the nanoparticle has an absorption spectrum having a FWHM of 80 nm or less.

15-23. (canceled)

24. The nanoparticle of claim 1, further comprising a matrix polymer.

25-33. (canceled)

34. The nanoparticle of claim 1, wherein the nanoparticle has an absorbance width from 10 nm to 150 nm at 10% of the absorbance maximum.

35. The nanoparticle of claim 1, wherein the nanoparticle has a brightness of greater than 1.0×10⁻¹³ cm², calculated as the product of quantum yield and absorption cross-section.

36. The nanoparticle of claim 1, wherein the nanoparticle is bioconjugated to a biomolecule selected from a protein, a nucleic acid molecule, a lipid, a peptide, a carbohydrate, an aptamer, a drug, an antibody, an enzyme, a nucleic acid, or any combination thereof.

37-38. (canceled)

39. The nanoparticle of claim 1, wherein the nanoparticle does not comprise a β-phase structure or does not comprise a fluorene monomeric unit.

40. (canceled)

41. A method of making a nanoparticle of claim 1, the method comprising:
providing a solution comprising a polymer, the polymer comprising:
an absorbing monomeric unit; and
an emitting monomeric unit; and
collapsing the polymer to form the nanoparticles.

42-48. (canceled)

49. A method of analyzing a biomolecule, the method comprising optically detecting with a detector the presence or absence of the biomolecule, wherein the biomolecule is attached to the nanoparticle of claim 1.

50. (canceled)

51. The method of claim 49, wherein the detector is selected from a camera, an electron multiplier, a charge-coupled device (CCD) image sensor, a photomultiplier tube (PMT), an avalanche photodiode (APD), a single-photon avalanche diode (SPAD), and a complementary metal oxide semiconductor (CMOS) image sensor, a photo detector, electro detector, acoustical detector, magnetic detector, or the detector incorporates fluorescence microscopy imaging.

52. (canceled)

53. The method of claim 49, further comprises performing an assay selected from a digital assay, fluorescence activated sorting, and flow cytometry.

54-57. (canceled)

58. The method of claim 49, further comprising amplifying the biomolecule to produce an amplified product, the amplifying comprising performing polymerase chain reaction (PCR), isothermal nucleic acid amplification, rolling circle amplification (RCA), nucleic acid sequence based amplification (NASBA), loop-mediated amplification (LAMP), strand displacement amplification (SDA), or any combination thereof.

59. (canceled)
